SEQUENCE LISTING (342 sequences)

SEQ ID NO: 1 - protein, 6 aa, synthetic construct
VTNFRS

SEQ ID NO: 2 - protein, 7 aa, synthetic construct
LTSSGIE

SEQ ID NO: 3 - protein, 14 aa, synthetic construct
CAVEASSNTGKLIF

SEQ ID NO: 4 - protein, 112 aa, synthetic construct
EDKVVQSPLSLVVHEGDTVTLNCSYEVTNFRSLLWYKQEKKAPTFLFMLTSSGIEKKSGRLSSILDKKELFSILNITATQTGDSAVYLCAVEASSNTGKLIFGQGTTLQVKP

SEQ ID NO: 5 - protein, 274 aa, synthetic construct
MMKCPQALLAIFWLLLSWVSSEDKVVQSPLSLVVHEGDTVTLNCSYEVTNFRSLLWYKQEKKAPTFLFMLTSSGIEKKSGRLSSILDKKELFSILNITATQTGDSAVYLCAVEASSNTGKLIFGQGTTLQVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 6 - protein, 5 aa, synthetic construct
MNHNY

SEQ ID NO: 7 - protein, 6 aa, synthetic construct
SVGAGI

SEQ ID NO: 8 - protein, 13 aa, synthetic construct
CASSRTGGSEQYF

SEQ ID NO: 9 - protein, 112 aa, synthetic construct
NAGVTQTPKFRILKIGQSMTLQCAQDMNHNYMYWYRQDPGMGLKLIYYSVGAGITDKGEVPNGYNVSRSTTEDFPLRLELAAPSQTSVYFCASSRTGGSEQYFGPGTRLTVT

SEQ ID NO: 10 - protein, 310 aa, synthetic construct
MSISLLCCAAFPLLWAGPVNAGVTQTPKFRILKIGQSMTLQCAQDMNHNYMYWYRQDPGMGLKLIYYSVGAGITDKGEVPNGYNVSRSTTEDFPLRLELAAPSQTSVYFCASSRTGGSEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 11 - protein, 8 aa, synthetic construct
EVTNFRSL

SEQ ID NO: 12 - protein, 9 aa, synthetic construct
MLTSSGIEK

SEQ ID NO: 13 - protein, 14 aa, synthetic construct
CAVNSGAGSYQLTF

SEQ ID NO: 14 - protein, 112 aa, synthetic construct
EDKVVQSPLSLVVHEGDTVTLNCSYEVTNFRSLLWYKQEKKAPTFLFMLTSSGIEKKSGRLSSILDKKELFSILNITATQTGDSAVYLCAVNSGAGSYQLTFGKGTKLSVIP

SEQ ID NO: 15 - protein, 274 aa, synthetic construct
MMKCPQALLAIFWLLLSWVSSEDKVVQSPLSLVVHEGDTVTLNCSYEVTNFRSLLWYKQEKKAPTFLFMLTSSGIEKKSGRLSSILDKKELFSILNITATQTGDSAVYLCAVNSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 16 - protein, 7 aa, synthetic construct
TWNHNNM

SEQ ID NO: 17 - protein, 8 aa, synthetic construct
YSYGVQDT

SEQ ID NO: 18 - protein, 13 aa, synthetic construct
CASSSGGLYEQYF

SEQ ID NO: 19 - protein, 112 aa, synthetic construct
DAEITQSPRHKITETGRQVTLACHQTWNHNNMFWYRQDLGHGLRLIHYSYGVQDTNKGEVSDGYSVSRSNTEDLPLTLESAASSQTSVYFCASSSGGLYEQYFGPGTRLTVT

SEQ ID NO: 20 - protein, 310 aa, synthetic construct
MGTRLFFYVALCLLWAGHRDAEITQSPRHKITETGRQVTLACHQTWNHNNMFWYRQDLGHGLRLIHYSYGVQDTNKGEVSDGYSVSRSNTEDLPLTLESAASSQTSVYFCASSSGGLYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 21 - protein, 8 aa, synthetic construct
TVSPFSNL

SEQ ID NO: 22 - protein, 9 aa, synthetic construct
IMTFSENTK

SEQ ID NO: 23 - protein, 15 aa, synthetic construct
CVVGPSGGSNYKLTF

SEQ ID NO: 24 - protein, 114 aa, synthetic construct
KNQVEQSPQSLIILEGKNCTLQCNYTVSPFSNLRWYKQDTGRGPVSLTIMTFSENTKSNGRYTATLDADTKQSSLHITASQLSDSASYICVVGPSGGSNYKLTFGKGTLLTVNP

SEQ ID NO: 25 - protein, 276 aa, synthetic construct
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVGPSGGSNYKLTFGKGTLLTVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 26 - protein, 7 aa, synthetic construct
RSGDLSV

SEQ ID NO: 27 - protein, 8 aa, synthetic construct
QYYNGEER

SEQ ID NO: 28 - protein, 16 aa, synthetic construct
CASSGQGFLGNTEAFF

SEQ ID NO: 29 - protein, 115 aa, synthetic construct
DSGVTQTPKHLITATGQRVTLRCSPRSGDLSVYWYQQSLDQGLQFLIQYYNGEERAKGNILERFSAQQFPDLHSELNLSSLELGDSALYFCASSGQGFLGNTEAFFGQGTRLTVV

SEQ ID NO: 30 - protein, 311 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGQGFLGNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 31 - protein, 6 aa, synthetic construct
ATGYPS

SEQ ID NO: 32 - protein, 7 aa, synthetic construct
ATKADDK

SEQ ID NO: 33 - protein, 14 aa, synthetic construct
CALSEKTSYDKVIF

SEQ ID NO: 34 - protein, 113 aa, synthetic construct
GNSVTQMEGPVTLSEEAFLTINCTYTATGYPSLFWYVQYPGEGLQLLLKATKADDKGSNKGFEATYRKETTSFHLEKGSVQVSDSAVYFCALSEKTSYDKVIFGPGTSLSVIP

SEQ ID NO: 35 - protein, 273 aa, synthetic construct
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALSEKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 36 - protein, 5 aa, synthetic construct
MNHNY

SEQ ID NO: 37 - protein, 6 aa, synthetic construct
SVGAGI

SEQ ID NO: 38 - protein, 14 aa, synthetic construct
CASSYGPGGSEAFF

SEQ ID NO: 39 - protein, 113 aa, synthetic construct
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYGPGGSEAFFGQGTRLTVV

SEQ ID NO: 40 - protein, 309 aa, synthetic construct
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYGPGGSEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 41 - protein, 8 aa, synthetic construct
TATGYPSL

SEQ ID NO: 42 - protein, 9 aa, synthetic construct
KATKADDKG

SEQ ID NO: 43 - protein, 14 aa, synthetic construct
CALSVKTSYDKVIF

SEQ ID NO: 44 - protein, 113 aa, synthetic construct
GNSVTQMEGPVTLSEEAFLTINCTYTATGYPSLFWYVQYPGEGLQLLLKATKADDKGSNKGFEATYRKETTSFHLEKGSVQVSDSAVYFCALSVKTSYDKVIFGPGTSLSVIP

SEQ ID NO: 45 - protein, 273 aa, synthetic construct
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALSVKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 46 - protein, 7 aa, synthetic construct
DMNHNSM

SEQ ID NO: 47 - protein, 8 aa, synthetic construct
YSASEGTT

SEQ ID NO: 48 - protein, 14 aa, synthetic construct
CASSRDRGPETQYF

SEQ ID NO: 49 - protein, 113 aa, synthetic construct
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSRDRGPETQYFGPGTRLLVL

SEQ ID NO: 50 - protein, 311 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSRDRGPETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 51 - protein, 6 aa, synthetic construct
VSGLRG

SEQ ID NO: 52 - protein, 7 aa, synthetic construct
LYSAGEE

SEQ ID NO: 53 - protein, 12 aa, synthetic construct
CAVQATGYALNF

SEQ ID NO: 54 - protein, 109 aa, synthetic construct
EDQVTQSPEALRLQEGESSSLNCSYTVSGLRGLFWYRQDPGKGPEFLFTLYSAGEEKEKERLKATLTKKESFLHITAPKPEDSATYLCAVQATGYALNFGKGTSLLVTP

SEQ ID NO: 55 - protein, 271 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQATGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 56 - protein, 5 aa, synthetic construct
SGHTA

SEQ ID NO: 57 - protein, 6 aa, synthetic construct
FQGNSA

SEQ ID NO: 58 - protein, 14 aa, synthetic construct
CASNLGGSRQPQHF

SEQ ID NO: 59 - protein, 114 aa, synthetic construct
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASNLGGSRQPQHFGDGTRLSIL

SEQ ID NO: 60 - protein, 310 aa, synthetic construct
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASNLGGSRQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 61 - protein, 8 aa, synthetic construct
SDRGSQSF

SEQ ID NO: 62 - protein, 8 aa, synthetic construct
FIYSNGDK

SEQ ID NO: 63 - protein, 13 aa, synthetic construct
CAVIYSSASKIIF

SEQ ID NO: 64 - protein, 111 aa, synthetic construct
QKEVEQNSGPLSVPEGAIASLNCTYSDRGSQSFFWYRQYSGKSPELIMFIYSNGDKEDGRFTAQLNKASQYVSLLIRDSQPSDSATYLCAVIYSSASKIIFGSGTRLSIRP

SEQ ID NO: 65 - protein, 273 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVIYSSASKIIFGSGTRLSIRPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 66 - protein, 8 aa, synthetic construct
EGTSNPNL

SEQ ID NO: 67 - protein, 7 aa, synthetic construct
YSVGIGQ

SEQ ID NO: 68 - protein, 17 aa, synthetic construct
CAWSVQRLGRGGETQYF

SEQ ID NO: 69 - protein, 116 aa, synthetic construct
SQTIHQWPATLVQPVGSPLSLECTVEGTSNPNLYWYRQAAGRGLQLLFYSVGIGQISSEVPQNLSASRPQDRQFILSSKKLLLSDSGFYLCAWSVQRLGRGGETQYFGPGTRLLVL

SEQ ID NO: 70 - protein, 312 aa, synthetic construct
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSVQRLGRGGETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 71 - protein, 6 aa, synthetic construct
VSGLRG

SEQ ID NO: 72 - protein, 7 aa, synthetic construct
LYSAGEE

SEQ ID NO: 73 - protein, 14 aa, synthetic construct
CAVQLSDSNYKLTF

SEQ ID NO: 74 - protein, 111 aa, synthetic construct
EDQVTQSPEALRLQEGESSSLNCSYTVSGLRGLFWYRQDPGKGPEFLFTLYSAGEEKEKERLKATLTKKESFLHITAPKPEDSATYLCAVQLSDSNYKLTFGKGTLLTVNP

SEQ ID NO: 75 - protein, 273 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQLSDSNYKLTFGKGTLLTVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 76 - protein, 5 aa, synthetic construct
MNHNS

SEQ ID NO: 77 - protein, 6 aa, synthetic construct
SASEGT

SEQ ID NO: 78 - protein, 13 aa, synthetic construct
CASRGQVNEKLFF

SEQ ID NO: 79 - protein, 112 aa, synthetic construct
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASRGQVNEKLFFGSGTQLSVL

SEQ ID NO: 80 - protein, 308 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASRGQVNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 81 - protein, 9 aa, synthetic construct
STISGNEYV

SEQ ID NO: 82 - protein, 7 aa, synthetic construct
HGLKNNE

SEQ ID NO: 83 - protein, 15 aa, synthetic construct
CIVRFQSGGYQKVTF

SEQ ID NO: 84 - protein, 112 aa, synthetic construct
DAKTTQPTSMDCAEGRAANLPCNHSTISGNEYVYWYRQIHSQGPQYIIHGLKNNETNEMASLIITEDRKSSTLILPHATLRDTAVYYCIVRFQSGGYQKVTFGTGTKLQVIP

SEQ ID NO: 85 - protein, 270 aa, synthetic construct
MRLVARVTVFLTFGTIIDAKTTQPTSMDCAEGRAANLPCNHSTISGNEYVYWYRQIHSQGPQYIIHGLKNNETNEMASLIITEDRKSSTLILPHATLRDTAVYYCIVRFQSGGYQKVTFGTGTKLQVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 86 - protein, 7 aa, synthetic construct
ISGHNSL

SEQ ID NO: 87 - protein, 8 aa, synthetic construct
YFNNNVPI

SEQ ID NO: 88 - protein, 16 aa, synthetic construct
CASSIQAGGRYNEQFF

SEQ ID NO: 89 - protein, 116 aa, synthetic construct
DAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSIQAGGRYNEQFFGPGTRLTVL

SEQ ID NO: 90 - protein, 314 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSIQAGGRYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 91 - protein, 8 aa, synthetic construct
SNSAFQYF

SEQ ID NO: 92 - protein, 8 aa, synthetic construct
YTYSSGNK

SEQ ID NO: 93 - protein, 13 aa, synthetic construct
CAMRTDSWGKLQF

SEQ ID NO: 94 - protein, 111 aa, synthetic construct
QKEVEQDPGPLSVPEGAIVSLNCTYSNSAFQYFMWYRQYSRKGPELLMYTYSSGNKEDGRFTAQVDKSSKYISLFIRDSQPSDSATYLCAMRTDSWGKLQFGAGTQVVVTP

SEQ ID NO: 95 - protein, 274 aa, synthetic construct
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMRTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 96 - protein, 7 aa, synthetic construct
NLGHDTM

SEQ ID NO: 97 - protein, 8 aa, synthetic construct
SYNNKELI

SEQ ID NO: 98 - protein, 15 aa, synthetic construct
CASSQTGGAVDTQYF

SEQ ID NO: 99 - protein, 114 aa, synthetic construct
DTAVSQTPKYLVTQMGNDKSIKCEQNLGHDTMYWYKQDSKKFLKIMFSYNNKELIINETVPNRFSPKSPDKAHLNLHINSLELGDSAVYFCASSQTGGAVDTQYFGPGTRLTVL

SEQ ID NO: 100 - protein, 312 aa, synthetic construct
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQTGGAVDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 101 - protein, 8 aa, synthetic construct
  VARIANT 2: note = X can be any amino acid
  VARIANT 4: note = X can be any amino acid
DXEXNPGP

SEQ ID NO: 102 - protein, 18 aa, synthetic construct
EGRGSLLTCGDVEENPGP

SEQ ID NO: 103 - protein, 19 aa, synthetic construct
ATNFSLLKQAGDVEENPGP

SEQ ID NO: 104 - protein, 20 aa, synthetic construct
QCTNYALLKLAGDVESNPGP

SEQ ID NO: 105 - protein, 22 aa, synthetic construct
VKQTLNFDLLKLAGDVESNPGP

SEQ ID NO: 106 - protein, 141 aa, synthetic construct
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 107 - protein, 177 aa, synthetic construct
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 108 - protein, 179 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 109 - protein, 18 aa, synthetic construct
GAGSLQPLALEGSLQKRG

SEQ ID NO: 110 - protein, 8 aa, synthetic construct
TVSPFSNL

SEQ ID NO: 111 - protein, 9 aa, synthetic construct
IMTFSENTK

SEQ ID NO: 112 - protein, 14 aa, synthetic construct
CVVSASSNTGKLIF

SEQ ID NO: 113 - protein, 113 aa, synthetic construct
KNQVEQSPQSLIILEGKNCTLQCNYTVSPFSNLRWYKQDTGRGPVSLTIMTFSENTKSNGRYTATLDADTKQSSLHITASQLSDSASYICVVSASSNTGKLIFGQGTTLQVKP

SEQ ID NO: 114 - protein, 275 aa, synthetic construct
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSASSNTGKLIFGQGTTLQVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 115 - protein, 7 aa, synthetic construct
ISGHNSL

SEQ ID NO: 116 - protein, 8 aa, synthetic construct
YFNNNVPI

SEQ ID NO: 117 - protein, 15 aa, synthetic construct
CASSGQLVAYNEQFF

SEQ ID NO: 118 - protein, 115 aa, synthetic construct
DAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSGQLVAYNEQFFGPGTRLTVL

SEQ ID NO: 119 - protein, 313 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSGQLVAYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 120 - protein, 8 aa, synthetic construct
SNSASQSF

SEQ ID NO: 121 - protein, 7 aa, synthetic construct
SVYSSGN

SEQ ID NO: 122 - protein, 14 aa, synthetic construct
CVVLRNSGNTPLVF

SEQ ID NO: 123 - protein, 111 aa, synthetic construct
RKEVEQDPGPFNVPEGATVAFNCTYSNSASQSFFWYRQDCRKEPKLLMSVYSSGNEDGRFTAQLNRASQYISLLIRDSKLSDSATYLCVVLRNSGNTPLVFGKGTRLSVIA

SEQ ID NO: 124 - protein, 273 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVLRNSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 125 - protein, 7 aa, synthetic construct
TENHRYM

SEQ ID NO: 126 - protein, 8 aa, synthetic construct
YSYGVKDT

SEQ ID NO: 127 - protein, 15 aa, synthetic construct
CAIKPGQGNRGEQFF

SEQ ID NO: 128 - protein, 114 aa, synthetic construct
DAGITQSPRHKVTETGTPVTLRCHQTENHRYMYWYRQDPGHGLRLIHYSYGVKDTDKGEVSDGYSVSRSKTEDFLLTLESATSSQTSVYFCAIKPGQGNRGEQFFGPGTRLTVL

SEQ ID NO: 129 - protein, 312 aa, synthetic construct
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAIKPGQGNRGEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 130 - protein, 8 aa, synthetic construct
TNSMFDYF

SEQ ID NO: 131 - protein, 9 aa, synthetic construct
SISSIKDKN

SEQ ID NO: 132 - protein, 14 aa, synthetic construct
CAASAKTSGSRLTF

SEQ ID NO: 133 - protein, 113 aa, synthetic construct
DQQVKQNSPSLSVQEGRISILNCDYTNSMFDYFLWYKKYPAEGPTFLISISSIKDKNEDGRFTVFLNKSAKHLSLHIVPSQPGDSAVYFCAASAKTSGSRLTFGEGTQLTVNP

SEQ ID NO: 134 - protein, 280 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASAKTSGSRLTFGEGTQLTVNPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 135 - protein, 7 aa, synthetic construct
DMNHNYM

SEQ ID NO: 136 - protein, 8 aa, synthetic construct
YSVGAGIT

SEQ ID NO: 137 - protein, 13 aa, synthetic construct
CASRGGIQETQYF

SEQ ID NO: 138 - protein, 112 aa, synthetic construct
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASRGGIQETQYFGPGTRLLVL

SEQ ID NO: 139 - protein, 310 aa, synthetic construct
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASRGGIQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 140 - protein, 8 aa, synthetic construct
SNSASDYF

SEQ ID NO: 141 - protein, 9 aa, synthetic construct
DIRSNMDKR

SEQ ID NO: 142 - protein, 14 aa, synthetic construct
CAEISKTSYDKVIF

SEQ ID NO: 143 - protein, 113 aa, synthetic construct
GESVGLHLPTLSVQEGDNSIINCAYSNSASDYFIWYKQESGKGPQFIIDIRSNMDKRQGQRVTVLLNKTVKHLSLQIAATQPGDSAVYFCAEISKTSYDKVIFGPGTSLSVIP

SEQ ID NO: 144 - protein, 274 aa, synthetic construct
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAEISKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 145 - protein, 7 aa, synthetic construct
DMNHNYM

SEQ ID NO: 146 - protein, 8 aa, synthetic construct
YSVGAGIT

SEQ ID NO: 147 - protein, 15 aa, synthetic construct
CASSYGPGGRNTIYF

SEQ ID NO: 148 - protein, 114 aa, synthetic construct
NAGVTQTPKFRILKIGQSMTLQCAQDMNHNYMYWYRQDPGMGLKLIYYSVGAGITDKGEVPNGYNVSRSTTEDFPLRLELAAPSQTSVYFCASSYGPGGRNTIYFGEGSWLTVV

SEQ ID NO: 149 - protein, 310 aa, synthetic construct
MSISLLCCAAFPLLWAGPVNAGVTQTPKFRILKIGQSMTLQCAQDMNHNYMYWYRQDPGMGLKLIYYSVGAGITDKGEVPNGYNVSRSTTEDFPLRLELAAPSQTSVYFCASSYGPGGRNTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 150 - protein, 8 aa, synthetic construct
TDSAIYNL

SEQ ID NO: 151 - protein, 9 aa, synthetic construct
LIQSSQREQ

SEQ ID NO: 152 - protein, 14 aa, synthetic construct
CAVTGMDSSYKLIF

SEQ ID NO: 153 - protein, 113 aa, synthetic construct
KQEVTQIPAALSVPEGENLVLNCSFTDSAIYNLQWFRQDPGKGLTSLLLIQSSQREQTSGRLNASLDKSSGRSTLYIAASQPGDSATYLCAVTGMDSSYKLIFGSGTRLLVRP

SEQ ID NO: 154 - protein, 273 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVTGMDSSYKLIFGSGTRLLVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 155 - protein, 7 aa, synthetic construct
TWNHNNM

SEQ ID NO: 156 - protein, 8 aa, synthetic construct
YSYGVHDT

SEQ ID NO: 157 - protein, 13 aa, synthetic construct
CASSEGGSWGYTF

SEQ ID NO: 158 - protein, 112 aa, synthetic construct
DAEITQSPRHKITETGRQVTLACHQTWNHNNMFWYRQDLGHGLRLIHYSYGVHDTNKGEVSDGYSVSRSNTEDLPLTLESAASSQTSVYFCASSEGGSWGYTFGSGTRLTVV

SEQ ID NO: 159 - protein, 308 aa, synthetic construct
MGTRLFFYVALCLLWAGHRDAEITQSPRHKITETGRQVTLACHQTWNHNNMFWYRQDLGHGLRLIHYSYGVHDTNKGEVSDGYSVSRSNTEDLPLTLESAASSQTSVYFCASSEGGSWGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 160 - protein, 9 aa, synthetic construct
STISGNEYV

SEQ ID NO: 161 - protein, 7 aa, synthetic construct
HGLKNNE

SEQ ID NO: 162 - protein, 14 aa, synthetic construct
CIVRVLYNNNDMRF

SEQ ID NO: 163 - protein, 111 aa, synthetic construct
DAKTTQPPSMDCAEGRAANLPCNHSTISGNEYVYWYRQIHSQGPQYIIHGLKNNETNEMASLIITEDRKSSTLILPHATLRDTAVYYCIVRVLYNNNDMRFGAGTRLTVKP

SEQ ID NO: 164 - protein, 269 aa, synthetic construct
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRVLYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 165 - protein, 7 aa, synthetic construct
DMNHNYM

SEQ ID NO: 166 - protein, 8 aa, synthetic construct
YSVGAGIT

SEQ ID NO: 167 - protein, 15 aa, synthetic construct
CASSSTGPNTGELFF

SEQ ID NO: 168 - protein, 114 aa, synthetic construct
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASSSTGPNTGELFFGEGSRLTVL

SEQ ID NO: 169 - protein, 312 aa, synthetic construct
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSSTGPNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 170 - protein, 8 aa, synthetic construct
SVSNAYNF

SEQ ID NO: 171 - protein, 6 aa, synthetic construct
KGSKPS

SEQ ID NO: 172 - protein, 13 aa, synthetic construct
CAVGDTGGFKTIF

SEQ ID NO: 173 - protein, 106 aa, synthetic construct
KDQVFQPSTVASSEGAVVEIFCNHSVSNAYNFFWYLHFPGCAPRLLVKGSKPSQQGRYNMTYERFSSSLLILQVREADAAVYYCAVGDTGGFKTIFGAGTRLFVKA

SEQ ID NO: 174 - protein, 272 aa, synthetic construct
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVGDTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 175 - protein, 7 aa, synthetic construct
RSGDLSV

SEQ ID NO: 176 - protein, 8 aa, synthetic construct
HYYNGEER

SEQ ID NO: 177 - protein, 16 aa, synthetic construct
CASSEIAGFSTDTQYF

SEQ ID NO: 178 - protein, 115 aa, synthetic construct
DSGVTQTPKHLITATGQRVTLRCSPRSGDLSVYWYQQSLDQGLQFLIHYYNGEERAKGNILERFSAQQFPDLHSELNLSSLELGDSALYFCASSEIAGFSTDTQYFGPGTRLTVL

SEQ ID NO: 179 - protein, 313 aa, synthetic construct
MGFRLLCCVAFCLLGAGPVDSGVTQTPKHLITATGQRVTLRCSPRSGDLSVYWYQQSLDQGLQFLIHYYNGEERAKGNILERFSAQQFPDLHSELNLSSLELGDSALYFCASSEIAGFSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 180 - protein, 8 aa, synthetic construct
SNSASQSF

SEQ ID NO: 181 - protein, 7 aa, synthetic construct
SVYSSGN

SEQ ID NO: 182 - protein, 15 aa, synthetic construct
CVVTASNSGNTPLVF

SEQ ID NO: 183 - protein, 112 aa, synthetic construct
RKEVEQDPGPFNVPEGATVAFNCTYSNSASQSFFWYRQDCRKEPKLLMSVYSSGNEDGRFTAQLNRASQYISLLIRDSKLSDSATYLCVVTASNSGNTPLVFGKGTRLSVIA

SEQ ID NO: 184 - protein, 274 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVTASNSGNTPLVFGKGTRLSVIANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 185 - protein, 7 aa, synthetic construct
DSQVTMM

SEQ ID NO: 186 - protein, 9 aa, synthetic construct
TANQGSEAT

SEQ ID NO: 187 - protein, 11 aa, synthetic construct
CSVGGGDGYTF

SEQ ID NO: 188 - protein, 112 aa, synthetic construct
SAVISQKPSRDICQRGTSLTIQCQVDSQVTMMFWYRQQPGQSLTLIATANQGSEATYESGFVIDKFPISRPNLTFSTLTVSNMSPEDSSIYLCSVGGGDGYTFGSGTRLTVV

SEQ ID NO: 189 - protein, 304 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVGGGDGYTFGSGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 190 - protein, 8 aa, synthetic construct
SNSAFQYF

SEQ ID NO: 191 - protein, 8 aa, synthetic construct
YTYSSGNK

SEQ ID NO: 192 - protein, 13 aa, synthetic construct
CAMSEYSGYALNF

SEQ ID NO: 193 - protein, 111 aa, synthetic construct
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSEYSGYALNFGKGTSLLVTP

SEQ ID NO: 194 - protein, 274 aa, synthetic construct
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSEYSGYALNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 195 - protein, 7 aa, synthetic construct
NLNHDAM

SEQ ID NO: 196 - protein, 8 aa, synthetic construct
YSQIVNDF

SEQ ID NO: 197 - protein, 11 aa, synthetic construct
CASSNRDEQYF

SEQ ID NO: 198 - protein, 110 aa, synthetic construct
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSNRDEQYFGPGTRLTVT

SEQ ID NO: 199 - protein, 308 aa, synthetic construct
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSNRDEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 200 - protein, 8 aa, synthetic construct
TVSPFSNL

SEQ ID NO: 201 - protein, 9 aa, synthetic construct
IMTFSENTK

SEQ ID NO: 202 - protein, 14 aa, synthetic construct
CVVSAKTGANNLFF

SEQ ID NO: 203 - protein, 113 aa, synthetic construct
KNQVEQSPQSLIILEGKNCTLQCNYTVSPFSNLRWYKQDTGRGPVSLTIMTFSENTKSNGRYTATLDADTKQSSLHITASQLSDSASYICVVSAKTGANNLFFGTGTRLTVIP

SEQ ID NO: 204 - protein, 275 aa, synthetic construct
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSAKTGANNLFFGTGTRLTVIPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 205 - protein, 7 aa, synthetic construct
TWSHSYM

SEQ ID NO: 206 - protein, 8 aa, synthetic construct
YSAAADIT

SEQ ID NO: 207 - protein, 12 aa, synthetic construct
CASSETDYGYTF

SEQ ID NO: 208 - protein, 111 aa, synthetic construct
DAGITQSPRYKITETGRQVTLMCHQTWSHSYMFWYRQDLGHGLRLIYYSAAADITDKGEVPDGYVVSRSKTENFPLTLESATRSQTSVYFCASSETDYGYTFGSGTRLTVV

SEQ ID NO: 209 - protein, 307 aa, synthetic construct
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSETDYGYTFGSGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 210 - protein, 8 aa, synthetic construct
TVSGLRGL

SEQ ID NO: 211 - protein, 9 aa, synthetic construct
TLYSAGEEK

SEQ ID NO: 212 - protein, 14 aa, synthetic construct
CAVQVSRNTGKLIF

SEQ ID NO: 213 - protein, 111 aa, synthetic construct
EDQVTQSPEALRLQEGESSSLNCSYTVSGLRGLFWYRQDPGKGPEFLFTLYSAGEEKEKERLKATLTKKESFLHITAPKPEDSATYLCAVQVSRNTGKLIFGQGTTLQVKP

SEQ ID NO: 214 - protein, 273 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQVSRNTGKLIFGQGTTLQVKPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 215 - protein, 7 aa, synthetic construct
DMNHNSM

SEQ ID NO: 216 - protein, 8 aa, synthetic construct
YSASEGTT

SEQ ID NO: 217 - protein, 15 aa, synthetic construct
CASSELGQPIYGYTF

SEQ ID NO: 218 - protein, 114 aa, synthetic construct
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSELGQPIYGYTFGSGTRLTVV

SEQ ID NO: 219 - protein, 310 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSELGQPIYGYTFGSGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 220 - protein, 8 aa, synthetic construct
TATGYPSL

SEQ ID NO: 221 - protein, 9 aa, synthetic construct
KATKADDKG

SEQ ID NO: 222 - protein, 14 aa, synthetic construct
CALSSKTSYDKVIF

SEQ ID NO: 223 - protein, 113 aa, synthetic construct
GDSVTQMEGPVTLSEEAFLTINCTYTATGYPSLFWYVQYPGEGLQLLLKATKADDKGSNKGFEATYRKETTSFHLEKGSVQVSDSAVYFCALSSKTSYDKVIFGPGTSLSVIP

SEQ ID NO: 224 - protein, 273 aa, synthetic construct
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALSSKTSYDKVIFGPGTSLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 225 - protein, 7 aa, synthetic construct
DMNHNSM

SEQ ID NO: 226 - protein, 8 aa, synthetic construct
YSASEGTT

SEQ ID NO: 227 - protein, 14 aa, synthetic construct
CASRDRNSGNTIYF

SEQ ID NO: 228 - protein, 113 aa, synthetic construct
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASRDRNSGNTIYFGEGSWLTVV

SEQ ID NO: 229 - protein, 309 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASRDRNSGNTIYFGEGSWLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 230 - protein, 8 aa, synthetic construct
SNSASDYF

SEQ ID NO: 231 - protein, 9 aa, synthetic construct
DIRSNMDKR

SEQ ID NO: 232 - protein, 14 aa, synthetic construct
CAESSAGGYNKLIF

SEQ ID NO: 233 - protein, 113 aa, synthetic construct
GESVGLHLPTLSVQEGDNSIINCAYSNSASDYFIWYKQESGKGPQFIIDIRSNMDKRQGQRVTVLLNKTVKHLSLQIAATQPGDSAVYFCAESSAGGYNKLIFGAGTRLAVHP

SEQ ID NO: 234 - protein, 274 aa, synthetic construct
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAESSAGGYNKLIFGAGTRLAVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 235 - protein, 7 aa, synthetic construct
ISGHNSL

SEQ ID NO: 236 - protein, 8 aa, synthetic construct
YFNNNVPI

SEQ ID NO: 237 - protein, 14 aa, synthetic construct
CASRGQVGGYGYTF

SEQ ID NO: 238 - protein, 114 aa, synthetic construct
DAGVIQSPRHEVTEMGQEVTLRCKPISGHNSLFWYRQTMMRGLELLIYFNNNVPIDDSGMPEDRFSAKMPNASFSTLKIQPSEPRDSAVYFCASRGQVGGYGYTFGSGTRLTVV

SEQ ID NO: 239 - protein, 310 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASRGQVGGYGYTFGSGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 240 - protein, 8 aa, synthetic construct
ENTAFDYF

SEQ ID NO: 241 - protein, 9 aa, synthetic construct
AIRPDVSEK

SEQ ID NO: 242 - protein, 12 aa, synthetic construct
CAASSGTYKYIF

SEQ ID NO: 243 - protein, 111 aa, synthetic construct
QQQVKQSPQSLIVQKGGISIINCAYENTAFDYFPWYQQFPGKGPALLIAIRPDVSEKKEGRFTISFNKSAKQFSLHIMDSQPGDSATYFCAASSGTYKYIFGTGTRLKVLA

SEQ ID NO: 244 - protein, 280 aa, synthetic construct
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAASSGTYKYIFGTGTRLKVLANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 245 - protein, 7 aa, synthetic construct
DMNHEYM

SEQ ID NO: 246 - protein, 8 aa, synthetic construct
YSVGEGTT

SEQ ID NO: 247 - protein, 14 aa, synthetic construct
CASSFAGANYGYTF

SEQ ID NO: 248 - protein, 113 aa, synthetic construct
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSFAGANYGYTFGSGTRLTVV

SEQ ID NO: 249 - protein, 309 aa, synthetic construct
MSLGLLCCAAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSFAGANYGYTFGSGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 250 - protein, 8 aa, synthetic construct
SNSASQSF

SEQ ID NO: 251 - protein, 7 aa, synthetic construct
SVYSSGN

SEQ ID NO: 252 - protein, 15 aa, synthetic construct
CVVSGSNSGNTPLVF

SEQ ID NO: 253 - protein, 112 aa, synthetic construct
RKEVEQDPGPFNVPEGATVAFNCTYSNSASQSFFWYRQDCRKEPKLLMSVYSSGNEDGRFTAQLNRASQYISLLIRDSKLSDSATYLCVVSGSNSGNTPLVFGKGTRLSVIA

SEQ ID NO: 254 - protein, 274 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVSGSNSGNTPLVFGKGTRLSVIANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 255 - protein, 7 aa, synthetic construct
DSQVTMM

SEQ ID NO: 256 - protein, 9 aa, synthetic construct
TANQGSEAT

SEQ ID NO: 257 - protein, 13 aa, synthetic construct
CSVGPTSGNTIYF

SEQ ID NO: 258 - protein, 114 aa, synthetic construct
SAVISQKPSRDICQRGTSLTIQCQVDSQVTMMFWYRQQPGQSLTLIATANQGSEATYESGFVIDKFPISRPNLTFSTLTVSNMSPEDSSIYLCSVGPTSGNTIYFGEGSWLTVV

SEQ ID NO: 259 - protein, 306 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVGPTSGNTIYFGEGSWLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 260 - protein, 274 aa, synthetic construct
MMKCPQALLAIFWLLLSWVSSEDKVVQSPLSLVVHEGDTVTLNCSYEVTNFRSLLWYKQEKKAPTFLFMLTSSGIEKKSGRLSSILDKKELFSILNITATQTGDSAVYLCAVEASSNTGKLIFGQGTTLQVKPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 261 - protein, 310 aa, synthetic construct
MSISLLCCAAFPLLWAGPVNAGVTQTPKFRILKIGQSMTLQCAQDMNHNYMYWYRQDPGMGLKLIYYSVGAGITDKGEVPNGYNVSRSTTEDFPLRLELAAPSQTSVYFCASSRTGGSEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 262 - protein, 274 aa, synthetic construct
MMKCPQALLAIFWLLLSWVSSEDKVVQSPLSLVVHEGDTVTLNCSYEVTNFRSLLWYKQEKKAPTFLFMLTSSGIEKKSGRLSSILDKKELFSILNITATQTGDSAVYLCAVNSGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 263 - protein, 310 aa, synthetic construct
MGTRLFFYVALCLLWAGHRDAEITQSPRHKITETGRQVTLACHQTWNHNNMFWYRQDLGHGLRLIHYSYGVQDTNKGEVSDGYSVSRSNTEDLPLTLESAASSQTSVYFCASSSGGLYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 264 - protein, 276 aa, synthetic construct
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVGPSGGSNYKLTFGKGTLLTVNPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 265 - protein, 311 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGQGFLGNTEAFFGQGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 266 - protein, 273 aa, synthetic construct
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALSEKTSYDKVIFGPGTSLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 267 - protein, 309 aa, synthetic construct
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYGPGGSEAFFGQGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 268 - protein, 273 aa, synthetic construct
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALSVKTSYDKVIFGPGTSLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 269 - protein, 311 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSRDRGPETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 270 - protein, 271 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQATGYALNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 271 - protein, 310 aa, synthetic construct
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASNLGGSRQPQHFGDGTRLSILEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 272 - protein, 273 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVIYSSASKIIFGSGTRLSIRPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 273 - protein, 312 aa, synthetic construct
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSVQRLGRGGETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 274 - protein, 273 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQLSDSNYKLTFGKGTLLTVNPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 275 - protein, 308 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASRGQVNEKLFFGSGTQLSVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 276 - protein, 270 aa, synthetic construct
MRLVARVTVFLTFGTIIDAKTTQPTSMDCAEGRAANLPCNHSTISGNEYVYWYRQIHSQGPQYIIHGLKNNETNEMASLIITEDRKSSTLILPHATLRDTAVYYCIVRFQSGGYQKVTFGTGTKLQVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 277 - protein, 314 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSIQAGGRYNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 278 - protein, 274 aa, synthetic construct
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMRTDSWGKLQFGAGTQVVVTPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 279 - protein, 312 aa, synthetic construct
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQTGGAVDTQYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 280 - protein, 275 aa, synthetic construct
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSASSNTGKLIFGQGTTLQVKPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 281 - protein, 313 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSGQLVAYNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 282 - protein, 273 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVLRNSGNTPLVFGKGTRLSVIANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 283 - protein, 312 aa, synthetic construct
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAIKPGQGNRGEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 284 - protein, 280 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASAKTSGSRLTFGEGTQLTVNPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 285 - protein, 310 aa, synthetic construct
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASRGGIQETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 286 - protein, 274 aa, synthetic construct
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAEISKTSYDKVIFGPGTSLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 287 - protein, 310 aa, synthetic construct
MSISLLCCAAFPLLWAGPVNAGVTQTPKFRILKIGQSMTLQCAQDMNHNYMYWYRQDPGMGLKLIYYSVGAGITDKGEVPNGYNVSRSTTEDFPLRLELAAPSQTSVYFCASSYGPGGRNTIYFGEGSWLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 288 - protein, 273 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVTGMDSSYKLIFGSGTRLLVRPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 289 - protein, 308 aa, synthetic construct
MGTRLFFYVALCLLWAGHRDAEITQSPRHKITETGRQVTLACHQTWNHNNMFWYRQDLGHGLRLIHYSYGVHDTNKGEVSDGYSVSRSNTEDLPLTLESAASSQTSVYFCASSEGGSWGYTFGSGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 290 - protein, 269 aa, synthetic construct
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRVLYNNNDMRFGAGTRLTVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 291 - protein, 312 aa, synthetic construct
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSSTGPNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 292 - protein, 272 aa, synthetic construct
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVGDTGGFKTIFGAGTRLFVKANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 293 - protein, 313 aa, synthetic construct
MGFRLLCCVAFCLLGAGPVDSGVTQTPKHLITATGQRVTLRCSPRSGDLSVYWYQQSLDQGLQFLIHYYNGEERAKGNILERFSAQQFPDLHSELNLSSLELGDSALYFCASSEIAGFSTDTQYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 294 - protein, 274 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVTASNSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 295 - protein, 304 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVGGGDGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 296 - protein, 274 aa, synthetic construct
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSEYSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 297 - protein, 308 aa, synthetic construct
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSNRDEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 298 - protein, 275 aa, synthetic construct
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSAKTGANNLFFGTGTRLTVIPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 299 - protein, 307 aa, synthetic construct
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSETDYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 300 - protein, 273 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQVSRNTGKLIFGQGTTLQVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 301 - protein, 310 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSELGQPIYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 302 - protein, 273 aa, synthetic construct
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALSSKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 303 - protein, 309 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASRDRNSGNTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 304 - protein, 274 aa, synthetic construct
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAESSAGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 305 - protein, 310 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASRGQVGGYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 306 - protein, 280 aa, synthetic construct
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAASSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 307 - protein, 309 aa, synthetic construct
MSLGLLCCAAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSFAGANYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 308 - protein, 274 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVSGSNSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 309 - protein, 306 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVGPTSGNTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 310 - protein, 606 aa, synthetic construct
MSISLLCCAAFPLLWAGPVNAGVTQTPKFRILKIGQSMTLQCAQDMNHNYMYWYRQDPGMGLKLIYYSVGAGITDKGEVPNGYNVSRSTTEDFPLRLELAAPSQTSVYFCASSRTGGSEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMMKCPQALLAIFWLLLSWVSSEDKVVQSPLSLVVHEGDTVTLNCSYEVTNFRSLLWYKQEKKAPTFLFMLTSSGIEKKSGRLSSILDKKELFSILNITATQTGDSAVYLCAVEASSNTGKLIFGQGTTLQVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 311 - protein, 606 aa, synthetic construct
MSISLLCCAAFPLLWAGPVNAGVTQTPKFRILKIGQSMTLQCAQDMNHNYMYWYRQDPGMGLKLIYYSVGAGITDKGEVPNGYNVSRSTTEDFPLRLELAAPSQTSVYFCASSRTGGSEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMMKCPQALLAIFWLLLSWVSSEDKVVQSPLSLVVHEGDTVTLNCSYEVTNFRSLLWYKQEKKAPTFLFMLTSSGIEKKSGRLSSILDKKELFSILNITATQTGDSAVYLCAVEASSNTGKLIFGQGTTLQVKPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 312 - protein, 604 aa, synthetic construct
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYGPGGSEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALSEKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 313 - protein, 604 aa, synthetic construct
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYGPGGSEAFFGQGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALSEKTSYDKVIFGPGTSLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 314 - protein, 603 aa, synthetic construct
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASNLGGSRQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQATGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 315 - protein, 603 aa, synthetic construct
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASNLGGSRQPQHFGDGTRLSILEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQATGYALNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 316 - protein, 603 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASRGQVNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQLSDSNYKLTFGKGTLLTVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 317 - protein, 603 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASRGQVNEKLFFGSGTQLSVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQLSDSNYKLTFGKGTLLTVNPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 318 - protein, 253 aa, synthetic construct
EDKVVQSPLSLVVHEGDTVTLNCSYEVTNFRSLLWYKQEKKAPTFLFMLTSSGIEKKSGRLSSILDKKELFSILNITATQTGDSAVYLCAVEASSNTGKLIFGQGTTLQVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 319 - protein, 253 aa, synthetic construct
EDKVVQSPLSLVVHEGDTVTLNCSYEVTNFRSLLWYKQEKKAPTFLFMLTSSGIEKKSGRLSSILDKKELFSILNITATQTGDSAVYLCAVEASSNTGKLIFGQGTTLQVKPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 320 - protein, 331 aa, synthetic construct
MSISLLCCAAFPLLWAGPVNAGVTQTPKFRILKIGQSMTLQCAQDMNHNYMYWYRQDPGMGLKLIYYSVGAGITDKGEVPNGYNVSRSTTEDFPLRLELAAPSQTSVYFCASSRTGGSEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPG

SEQ ID NO: 321 - protein, 291 aa, synthetic construct
NAGVTQTPKFRILKIGQSMTLQCAQDMNHNYMYWYRQDPGMGLKLIYYSVGAGITDKGEVPNGYNVSRSTTEDFPLRLELAAPSQTSVYFCASSRTGGSEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 322 - protein, 312 aa, synthetic construct
NAGVTQTPKFRILKIGQSMTLQCAQDMNHNYMYWYRQDPGMGLKLIYYSVGAGITDKGEVPNGYNVSRSTTEDFPLRLELAAPSQTSVYFCASSRTGGSEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPG

SEQ ID NO: 323 - protein, 331 aa, synthetic construct
MSISLLCCAAFPLLWAGPVNAGVTQTPKFRILKIGQSMTLQCAQDMNHNYMYWYRQDPGMGLKLIYYSVGAGITDKGEVPNGYNVSRSTTEDFPLRLELAAPSQTSVYFCASSRTGGSEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPG

SEQ ID NO: 324 - protein, 291 aa, synthetic construct
NAGVTQTPKFRILKIGQSMTLQCAQDMNHNYMYWYRQDPGMGLKLIYYSVGAGITDKGEVPNGYNVSRSTTEDFPLRLELAAPSQTSVYFCASSRTGGSEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 325 - protein, 312 aa, synthetic construct
NAGVTQTPKFRILKIGQSMTLQCAQDMNHNYMYWYRQDPGMGLKLIYYSVGAGITDKGEVPNGYNVSRSTTEDFPLRLELAAPSQTSVYFCASSRTGGSEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPG

SEQ ID NO: 326 - DNA, 1821 bp, synthetic construct
  source 1..1821: mol_type = other DNA
atgagcatcagcctcctgtgctgtgcagcctttcctctcctgtgggcaggtccagtgaatgctggtgtcactcagaccccaaaattccgcatcctgaagataggacagagcatgacactgcagtgtgcccaggatatgaaccataactacatgtactggtatcgacaagacccaggcatggggctgaagctgatttattattcagttggtgctggtatcactgacaaaggagaagtcccgaatggctacaacgtctccagatcaaccacagaggatttcccgctcaggctggagttggctgctccctcccagacatctgtgtacttctgtgccagcagccggacagggggaagcgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcggcagcggcgccaccaacttctccctgctgaagcaggccggcgacgtggaagaaaaccctggccccatgatgaagtgtccacaggctttactagctatcttttggcttctactgagctgggtgagcagtgaagacaaggtggtacaaagccctctatctctggttgtccacgagggagacactgtaactctcaattgcagttatgaagtgactaactttcgaagcctactatggtacaagcaggaaaagaaagctcccacatttctatttatgctaacttcaagtggaattgaaaagaagtcaggaagactaagtagcatattagataagaaagaacttttcagcatcctgaacatcacagccacccagaccggagactcggccgtctacctctgtgctgtggaggcctctagcaacacaggcaaactaatctttgggcaagggacaactttacaagtaaaaccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 327 - protein, 8 aa, synthetic construct
EVTNFRSL

SEQ ID NO: 328 - protein, 9 aa, synthetic construct
MLTSSGIEK

SEQ ID NO: 329 - protein, 7 aa, synthetic construct
DMNHNYM

SEQ ID NO: 330 - protein, 8 aa, synthetic construct
YSVGAGIT

SEQ ID NO: 331 - protein, 8 aa, synthetic construct
TATGYPSL

SEQ ID NO: 332 - protein, 9 aa, synthetic construct
KATKADDKG

SEQ ID NO: 333 - protein, 7 aa, synthetic construct
DMNHNYM

SEQ ID NO: 334 - protein, 8 aa, synthetic construct
YSVGAGIT

SEQ ID NO: 335 - protein, 8 aa, synthetic construct
TVSGLRGL

SEQ ID NO: 336 - protein, 9 aa, synthetic construct
TLYSAGEEK

SEQ ID NO: 337 - protein, 7 aa, synthetic construct
ISGHTAL

SEQ ID NO: 338 - protein, 8 aa, synthetic construct
YFQGNSAP

SEQ ID NO: 339 - protein, 8 aa, synthetic construct
TVSGLRGL

SEQ ID NO: 340 - protein, 9 aa, synthetic construct
TLYSAGEEK

SEQ ID NO: 341 - protein, 7 aa, synthetic construct
DMNHNSM

SEQ ID NO: 342 - protein, 8 aa, synthetic construct
YSASEGTT

(19) United States
(12) Patent Application Publication
(10) Pub. No.: US 2025/0295772 A1
(43) Pub. Date: Sep. 25, 2025

(54) CELL THERAPIES FOR TYPE 1 DIABETES

(71) Applicant: Abata Therapeutics, Inc., Watertown, MA (US)

(72) Inventors: Geetha MYLVAGANAM, Watertown, MA (US); Devapregasan MOODLEY, Watertown, MA (US); Jeremy BURNS, Watertown, MA (US); Yanbo ZHANG, Watertown, MA (US); Timothy NELSON, Watertown, MA (US)

(21) Appl. No.: 19/229,739

(22) Filed: Jun. 5, 2025

Related U.S. Application Data
(63) Continuation of application No. PCT/US2023/084859, filed on Dec. 19, 2023.
(60) Provisional application No. 63/434,062, filed on Dec. 20, 2022.
(60) Provisional application No. 63/459,843, filed on Apr. 17, 2023.
(60) Provisional application No. 63/578,226, filed on Aug. 23, 2023.

(51) Int. Cl. A61K 40/11; A61K 35/17; A61K 40/32; A61K 40/42; C07K 14/725; C12N 5/0783

(52) U.S. Cl. CPC A61K 40/11; A61K 35/17; A61K 40/32; A61K 40/4237; C07K 14/7051; C12N 5/0636; A61K 2239/15; C07K 2319/30; C12N 2510/00

(57) ABSTRACT

The present disclosure is directed to T cell receptors targeting proinsulin and isolated cell populations comprising regulatory T cells exogenously expressing the T cell receptors used to treat Type 1 Diabetes.

### CROSS-REFERENCE TO RELATED APPLICATIONS

This application is a continuation of International Application No. PCT/US2023/084859, filed Dec. 19, 2023, which claims the benefit under 35 U.S.C. § 119(e) of U.S. provisional application No. 63/434,062, filed on Dec. 20, 2022, U.S. provisional application No. 63/459,843, filed on Apr. 17, 2023, and U.S. provisional application No. 63/578,226, filed on Aug. 23, 2023, the content of each of which is incorporated by reference herein in its entirety.

### REFERENCE TO THE ELECTRONIC SEQUENCE LISTING

The Sequence Listing XML associated with this application is provided in XML file format and is hereby incorporated by reference into the specification. The name of the XML file containing the Sequence Listing XML is ABTH_006_03US_SeqList_ST26.xml. The XML file is 341,609 bytes, and created on Jun. 5, 2025, and is being submitted electronically via USPTO Patent Center.

### BACKGROUND

Type 1 Diabetes is a T cell-mediated autoimmune disease resulting in islet β cell destruction, hypoinsulinemia, and altered glucose homeostasis. The activity of effector T cells and regulatory T cells, which jointly function in immune homeostasis, are dysregulated in patients having Type 1 Diabetes. It has been shown in the clinic that modulating T cells affects disease progression in these patients. There is evidence that therapeutic intervention in Type 1 Diabetes patients with the genetic human leukocyte antigen (HLA) haplotype DR3-DQ2 (over half of all patients), who are early in autoimmune pathogenesis, can prevent the onset of symptomatic disease and insulin dependence.

Approximately 64,000 patients are newly diagnosed with Type 1 Diabetes each year, and currently, curative treatments for Type 1 Diabetes do not exist. Available therapies rely on the treatment of symptoms often involving immunosuppressive reagents that can have severe side effects. There are no approved therapies that reset immune tolerance and halt the loss of beta cell function in those patients. Regulatory T cells have potential for the treatment of this disease and other autoimmune diseases because they can selectively target diseased cell types and tissues, generating a local immune response via an antigen-specific mechanism.

### SUMMARY

Provided herein, in some aspects, is a T cell therapy (e.g., Treg cell therapy) that uses a TCR to target the pancreas and draining lymph nodes in patients with Type 1 Diabetes (TID), which limits ongoing B-cell destruction and preserve residual B-cell mass. This therapy has the added potential to promote repair of damaged tissue and establish long lived tissue residency. Suppressing T cell function has been shown to delay TID onset, however, no therapeutic exists to halt the autoimmunity that drive TID and durably recalibrate the immune response. The targeted approach to suppressing pancreatic islet-associated inflammation, as described herein in some embodiments, offers a significant and long-lasting clinical benefit to patients. This pioneering approach to patients with TID, in some embodiments, leverages the natural role of T cells in the immune system to treat TID.

The present disclosure is based, at least in part, on the development of T cell receptors (TCRs) specific to proinsulin (e.g., complexed with a major histocompatibility complex (MHC) molecule) that can be transduced into T cells (e.g., regulatory T cells) and expressed at the surface of those T cells. T cells expressing the TCRs of the disclosure effectively activate cells (e.g., conventional T cells) in the presence of proinsulin. Accordingly, these TCRs provide a mechanism for directing T cells (e.g., regulatory T cells) and the immune system to cells and tissues expressing proinsulin (e.g., for treatment of Type 1 Diabetes).

Some aspects relate to an engineered T cell receptor that specifically binds a preproinsulin 73-90 peptide complexed with an MHC molecule.

In some embodiments, an engineered T cell receptor comprises an alpha chain and a beta chain, wherein the alpha chain comprises a CDR3-alpha sequence comprising the amino acid sequence of any one of SEQ ID NOs: 3, 33, 53, and 73 and/or the beta chain comprises a CDR3-beta sequence comprising the amino acid sequence of any one of SEQ ID NOs: 8, 38, 58, and 78.

In some embodiments, an alpha chain comprises a CDR3-alpha sequence comprising the amino acid sequence of SEQ ID NO: 3 and a beta chain comprises a CDR3-beta sequence comprising the amino acid sequence of SEQ ID NO: 8.

In some embodiments, an alpha chain comprises a CDR1-alpha sequence comprising the amino acid sequence of SEQ ID NO: 1 and a CDR2-alpha sequence comprising the amino acid sequence of SEQ ID NO: 2, and a beta chain comprises a CDR1-beta sequence comprising the amino acid sequence of SEQ ID NO: 6 and a CDR2-beta sequence comprising the amino acid sequence of SEQ ID NO: 7.

In some embodiments, an alpha chain comprises an alpha variable domain comprising an amino acid sequence having at least 90% identity to the amino acid sequence of SEQ ID NO: 4, and a beta chain comprises a beta variable domain comprising an amino acid sequence having at least 90% identity to the amino acid sequence of SEQ ID NO: 9. In some embodiments, an alpha chain comprises an alpha variable domain comprising an amino acid sequence having at least 95% identity to the amino acid sequence of SEQ ID NO: 4, and a beta chain comprises a beta variable domain comprising an amino acid sequence having at least 95% identity to the amino acid sequence of SEQ ID NO: 9. In some embodiments, an alpha chain comprises an alpha variable domain comprising an amino acid sequence having 100% identity to the amino acid sequence of SEQ ID NO: 4, and a beta chain comprises a beta variable domain comprising an amino acid sequence having 100% identity to the amino acid sequence of SEQ ID NO: 9.

In some embodiments, an alpha chain comprises an amino acid sequence having at least 90% identity to the amino acid sequence of SEQ ID NO: 5, and a beta chain comprises an amino acid sequence having at least 90% identity to the amino acid sequence of SEQ ID NO: 10. In some embodiments, an alpha chain comprises an amino acid sequence having at least 95% identity to the amino acid sequence of SEQ ID NO: 5, and a beta chain comprises an amino acid sequence having at least 95% identity to the amino acid sequence of SEQ ID NO: 10. In some embodiments, an alpha chain comprises an amino acid sequence having 100% identity to the amino acid sequence of SEQ ID NO: 5, and a beta chain comprises an amino acid sequence having 100% identity to the amino acid sequence of SEQ ID NO: 10.

In some embodiments, an alpha chain comprises an amino acid sequence having at least 90% identity to the amino acid sequence of SEQ ID NO: 260, and a beta chain comprises an amino acid sequence having at least 90% identity to the amino acid sequence of SEQ ID NO: 261. In some embodiments, an alpha chain comprises an amino acid sequence having at least 95% identity to the amino acid sequence of SEQ ID NO: 260, and a beta chain comprises an amino acid sequence having at least 95% identity to the amino acid sequence of SEQ ID NO: 261. In some embodiments, an alpha chain comprises an amino acid sequence having 100% identity to the amino acid sequence of SEQ ID NO: 260, and a beta chain comprises an amino acid sequence having 100% identity to the amino acid sequence of SEQ ID NO: 261.

In some embodiments, an engineered T cell receptor comprises the sequence of SEQ ID NO: 310. In some embodiments, an engineered T cell receptor comprises the sequence of SEQ ID NO: 311.

In some embodiments, an alpha chain comprises a CDR3-alpha sequence comprising the amino acid sequence of SEQ ID NO: 33 and a beta chain comprises a CDR3-beta sequence comprising the amino acid sequence of SEQ ID NO: 38.

In some embodiments, an alpha chain comprises a CDR1-alpha sequence comprising the amino acid sequence of SEQ ID NO: 31 and a CDR2-alpha sequence comprising the amino acid sequence of SEQ ID NO: 32, and a beta chain comprises a CDR1-beta sequence comprising the amino acid sequence of SEQ ID NO: 36 and a CDR2-beta sequence comprising the amino acid sequence of SEQ ID NO: 37.

In some embodiments, an alpha chain comprises an alpha variable domain comprising an amino acid sequence having at least 90% identity to the amino acid sequence of SEQ ID NO: 34, and a beta chain comprises a beta variable domain comprising an amino acid sequence having at least 90% identity to the amino acid sequence of SEQ ID NO: 39. In some embodiments, an alpha chain comprises an alpha variable domain comprising an amino acid sequence having at least 95% identity to the amino acid sequence of SEQ ID NO: 34, and a beta chain comprises a beta variable domain comprising an amino acid sequence having at least 95% identity to the amino acid sequence of SEQ ID NO: 39. In some embodiments, an alpha chain comprises an alpha variable domain comprising an amino acid sequence having 100% identity to the amino acid sequence of SEQ ID NO: 34, and a beta chain comprises a beta variable domain comprising an amino acid sequence having 100% identity to the amino acid sequence of SEQ ID NO: 39.

In some embodiments, an alpha chain comprises an amino acid sequence having at least 90% identity to the amino acid sequence of SEQ ID NO: 35, and a beta chain comprises an amino acid sequence having at least 90% identity to the amino acid sequence of SEQ ID NO: 40. In some embodiments, an alpha chain comprises an amino acid sequence having at least 95% identity to the amino acid sequence of SEQ ID NO: 35, and a beta chain comprises an amino acid sequence having at least 95% identity to the amino acid sequence of SEQ ID NO: 40. In some embodiments, an alpha chain comprises an amino acid sequence having 100% identity to the amino acid sequence of SEQ ID NO: 35, and a beta chain comprises an amino acid sequence having 100% identity to the amino acid sequence of SEQ ID NO: 40.

In some embodiments, an alpha chain comprises an amino acid sequence having at least 90% identity to the amino acid sequence of SEQ ID NO: 266, and a beta chain comprises an amino acid sequence having at least 90% identity to the amino acid sequence of SEQ ID NO: 267. In some embodiments, an alpha chain comprises an amino acid sequence having at least 95% identity to the amino acid sequence of SEQ ID NO: 266, and a beta chain comprises an amino acid sequence having at least 95% identity to the amino acid sequence of SEQ ID NO: 267. In some embodiments, an alpha chain comprises an amino acid sequence having 100% identity to the amino acid sequence of SEQ ID NO: 266, and a beta chain comprises an amino acid sequence having 100% identity to the amino acid sequence of SEQ ID NO: 267.

In some embodiments, an engineered T cell receptor comprises the sequence of SEQ ID NO: 312 or 313.

In some embodiments, an alpha chain comprises a CDR3-alpha sequence comprising the amino acid sequence of SEQ ID NO: 53 and a beta chain comprises a CDR3-beta sequence comprising the amino acid sequence of SEQ ID NO: 58.

In some embodiments, an alpha chain comprises a CDR1-alpha sequence comprising the amino acid sequence of SEQ ID NO: 51 and a CDR2-alpha sequence comprising the amino acid sequence of SEQ ID NO: 52, and a beta chain comprises a CDR1-beta sequence comprising the amino acid sequence of SEQ ID NO: 56 and a CDR2-beta sequence comprising the amino acid sequence of SEQ ID NO: 57.

In some embodiments, an alpha chain comprises an alpha variable domain comprising an amino acid sequence having at least 90% identity to the amino acid sequence of SEQ ID NO: 54, and a beta chain comprises a beta variable domain comprising an amino acid sequence having at least 90% identity to the amino acid sequence of SEQ ID NO: 59. In some embodiments, an alpha chain comprises an alpha variable domain comprising an amino acid sequence having at least 95% identity to the amino acid sequence of SEQ ID NO: 54, and a beta chain comprises a beta variable domain comprising an amino acid sequence having at least 95% identity to the amino acid sequence of SEQ ID NO: 59. In some embodiments, an alpha chain comprises an alpha variable domain comprising an amino acid sequence having 100% identity to the amino acid sequence of SEQ ID NO: 54, and a beta chain comprises a beta variable domain comprising an amino acid sequence having 100% identity to the amino acid sequence of SEQ ID NO: 59.

In some embodiments, an alpha chain comprises an amino acid sequence having at least 90% identity to the amino acid sequence of SEQ ID NO: 55, and a beta chain comprises an amino acid sequence having at least 90% identity to the amino acid sequence of SEQ ID NO: 60. In some embodiments, an alpha chain comprises an amino acid sequence having at least 95% identity to the amino acid sequence of SEQ ID NO: 55, and a beta chain comprises an amino acid sequence having at least 95% identity to the amino acid sequence of SEQ ID NO: 60. In some embodiments, an alpha chain comprises an amino acid sequence having 100% identity to the amino acid sequence of SEQ ID NO: 55, and a beta chain comprises an amino acid sequence having 100% identity to the amino acid sequence of SEQ ID NO: 60.

In some embodiments, an alpha chain comprises an amino acid sequence having at least 90% identity to the amino acid sequence of SEQ ID NO: 270, and a beta chain comprises an amino acid sequence having at least 90% identity to the amino acid sequence of SEQ ID NO: 271. In some embodiments, an alpha chain comprises an amino acid sequence having at least 95% identity to the amino acid sequence of SEQ ID NO: 270, and a beta chain comprises an amino acid sequence having at least 95% identity to the amino acid sequence of SEQ ID NO: 271. In some embodiments, an alpha chain comprises an amino acid sequence having 100% identity to the amino acid sequence of SEQ ID NO: 270, and a beta chain comprises an amino acid sequence having 100% identity to the amino acid sequence of SEQ ID NO: 271.

In some embodiments, an engineered T cell receptor comprises the sequence of SEQ ID NO: 314 or 315.

In some embodiments, an alpha chain comprises a CDR3-alpha sequence comprising the amino acid sequence of SEQ ID NO: 73 and a beta chain comprises a CDR3-beta sequence comprising the amino acid sequence of SEQ ID NO: 78.

In some embodiments, an alpha chain comprises a CDR1-alpha sequence comprising the amino acid sequence of SEQ ID NO: 71 and a CDR2-alpha sequence comprising the amino acid sequence of SEQ ID NO: 72, and a beta chain comprises a CDR1-beta sequence comprising the amino acid sequence of SEQ ID NO: 76 and a CDR2-beta sequence comprising the amino acid sequence of SEQ ID NO: 77.

In some embodiments, an alpha chain comprises an alpha variable domain comprising an amino acid sequence having at least 90% identity to the amino acid sequence of SEQ ID NO: 74, and a beta chain comprises a beta variable domain comprising an amino acid sequence having at least 90% identity to the amino acid sequence of SEQ ID NO: 79. In some embodiments, an alpha chain comprises an alpha variable domain comprising an amino acid sequence having at least 95% identity to the amino acid sequence of SEQ ID NO: 74, and a beta chain comprises a beta variable domain comprising an amino acid sequence having at least 95% identity to the amino acid sequence of SEQ ID NO: 79. In some embodiments, an alpha chain comprises an alpha variable domain comprising an amino acid sequence having 100% identity to the amino acid sequence of SEQ ID NO: 74, and a beta chain comprises a beta variable domain comprising an amino acid sequence having 100% identity to the amino acid sequence of SEQ ID NO: 79.

In some embodiments, an alpha chain comprises an amino acid sequence having at least 90% identity to the amino acid sequence of SEQ ID NO: 75, and a beta chain comprises an amino acid sequence having at least 90% identity to the amino acid sequence of SEQ ID NO: 80. In some embodiments, an alpha chain comprises an amino acid sequence having at least 95% identity to the amino acid sequence of SEQ ID NO: 75, and a beta chain comprises an amino acid sequence having at least 95% identity to the amino acid sequence of SEQ ID NO: 80. In some embodiments, an alpha chain comprises an amino acid sequence having 100% identity to the amino acid sequence of SEQ ID NO: 75, and a beta chain comprises an amino acid sequence having 100% identity to the amino acid sequence of SEQ ID NO: 80.

In some embodiments, an alpha chain comprises an amino acid sequence having at least 90% identity to the amino acid sequence of SEQ ID NO: 274, and a beta chain comprises an amino acid sequence having at least 90% identity to the amino acid sequence of SEQ ID NO: 275. In some embodiments, an alpha chain comprises an amino acid sequence having at least 95% identity to the amino acid sequence of SEQ ID NO: 274, and a beta chain comprises an amino acid sequence having at least 95% identity to the amino acid sequence of SEQ ID NO: 275. In some embodiments, an alpha chain comprises an amino acid sequence having 100% identity to the amino acid sequence of SEQ ID NO: 274, and a beta chain comprises an amino acid sequence having 100% identity to the amino acid sequence of SEQ ID NO: 275.

In some embodiments, an engineered T cell receptor comprises the sequence of SEQ ID NO: 316 or 317.

In some embodiments, an engineered T cell receptor is encoded as a single polypeptide.

In some embodiments, an engineered T cell receptor comprises a self-cleaving peptide sequence positioned between the alpha chain and the beta chain.

In some embodiments, a self-cleaving peptide sequence is a 2A peptide sequence, for example, a P2A, E2A, F2A, or T2A peptide sequence.

In some embodiments, a preproinsulin 73-90 peptide comprises the amino acid sequence of GAGSLQPLALEGSLQKRG (SEQ ID NO: 109).

In some embodiments, an MHC molecule comprises a HLA-DRBI*04:01 molecule.

Some aspects relate to an engineered nucleic acid encoding an engineered T cell receptor of any one of the preceding paragraphs.

In some embodiments, an engineered nucleic acid is a viral vector, optionally a lentiviral vector.

In some embodiments, an engineered nucleic acid comprises an EF-1 alpha promoter or an MND promoter operably linked to a sequence encoding the engineered T cell receptor.

In some embodiments, an engineered nucleic acid further comprises an enhancer element, optionally an optimized post-transcriptional regulatory element (oPRE) or a woodchuck hepatitis virus post-transcriptional regulatory element (WPRE), further optionally WPRE-mut6.

Other aspects relate to a regulatory T cell comprising an engineered T cell receptor that specifically binds a preproinsulin 73-90 peptide complexed with the MHC molecule.

In some embodiments, an engineered T cell receptor is the engineered T cell receptor of any one of the preceding paragraphs.

In some embodiments, an engineered T cell receptor expresses the engineered nucleic acid of any one of the preceding paragraphs.

In some embodiments, a regulatory T cell is phenotypically stable.

In some embodiments, a regulatory T cell has not been gene edited at an endogenous FOXP3 locus.

Yet other aspects relate to a method of treating Type 1 Diabetes in a subject in need thereof, the method comprising administering to the subject a therapeutically effective amount of composition comprising the regulatory T cell of any one of the preceding paragraphs.

In some embodiments, a regulatory T cell is autologous to the subject.

In some embodiments, a subject has Type 1 Diabetes, for example, Stage 3 Type 1 Diabetes.

In some embodiments, a subject is an HLA-DRB1*04:01-positive subject.

Some aspects relate to a method of treating Type 1 Diabetes in a subject in need thereof, the method comprising: administering to a subject diagnosed with Type 1 Diabetes a therapeutically effective amount of a population of cells comprising regulatory T cells, wherein (i) regulatory T cells of the population express an engineered T cell receptor that specifically binds preproinsulin 73-90 (also referred to interchangeably as pre-proinsulin 73-90 or PPI₇₃₋₉₀) complexed with an MHC molecule in the pancreas and/or pancreatic draining lymph node(s) of the subject, and (ii) fewer than 10% of the regulatory T cells of the population have been gene edited at the endogenous FOXP3 locus.

Other aspects relate to a method of treating Type 1 Diabetes in a subject in need thereof, the method comprising: administering to a subject diagnosed with Type 1 Diabetes a therapeutically effective amount of population of cells comprising regulatory T cells, wherein regulatory T cells of the population express the engineered T cell receptor of any one of Table 1, wherein the regulatory T cells specifically bind preproinsulin 73-90 complexed with an MHC molecule in the pancreas and/or pancreatic draining lymph node(s) of the subject.

In some embodiments, the population of cells is autologous to the subject.

In some embodiments, fewer than 1% of the regulatory T cells of a population have been gene edited at the endogenous FOXP3 locus. For example, fewer than 0.5% or fewer than 0.1% of the regulatory T cells of a population have been gene edited at the endogenous FOXP3 locus

In some embodiments, a subject is an HLA-DRB1*04:01-positive subject.

In some embodiments, a subject is diagnosed with Stage 3 Type 1 Diabetes.

In some embodiments, a method further comprises selecting a subject who is HLA-DRB1*04:01 positive and has been diagnosed with Type 1 Diabetes, for example, Stage 3 Type 1 Diabetes; and administering to the subject a population of cells comprising regulatory T cells described herein.

In some embodiments, regulatory T cells of the population are phenotypically stable regulatory T cells. In some embodiments, at least 80% of the cells of a population are phenotypically stable regulatory T cells. For example, at least 85%, at least 90%, at least 95%, at least 96%, at least 97%, or at least 98% of the cells of a population may be phenotypically stable regulatory T cells.

In some embodiments, no more than 20% of the cells of a population are conventional CD4⁺ T cells. For example, no more than 15%, no more than 10%, or no more than 5% of the cells of a population may be conventional CD4⁺ T cells.

In some embodiments, no more than 5% of the cells of a population are CD8⁺ T cells. For example, no more than 1% of the cells of a population may be CD8⁺ T cells.

In some embodiments, no more than 10% of the cells of a population are non-regulatory T cells that express an engineered T cell receptor. For example, no more than 5% or no more than 1% of the cells of a population may be non-regulatory T cells that express the engineered T cell receptor,

In some embodiments, at least 40% of the cells of a population are CD4⁺CD25^{hi/+}CD127^{-/low} regulatory T cells that express an engineered T cell receptor. For example, at least 50%, at least 60%, at least 70%, at least 80%, or at least 90% of the cells of a population may be CD4⁺CD25^{hi/+}CD127^{-/low} regulatory T cells that express an engineered T cell receptor.

In some embodiments, at least 70% of the cells of the population are viable. For example, at least 75%, at least 80%, at least 85%, at least 90%, or at least 95% of the cells of a population may be viable.

In some embodiments, a method further comprises, prior to administering cells, isolating cells from a subject, sorting the cells to obtain phenotypically stable regulatory T cells, and expanding the phenotypically stable regulatory T cells, thereby producing a phenotypically stable regulatory T cells of the population of cells.

Some aspects relate to a method of preventing β cell destruction and/or preserving β cell mass in a subject in need thereof, comprising administering to the subject a composition comprising a cell population expressing a TCR provided herein, wherein the TCR specifically binds preproinsulin 73-90 complexed with an MHC molecule (e.g., an MHC class II antigen) in the pancreas and/or draining lymph node(s) of the subject, thereby preventing β cell destruction and/or preserving β cell mass in the subject.

Other aspects relate to a method of repairing pancreatic tissue damaged tissue in a subject in need thereof, comprising administering to the subject a composition comprising the cell population expressing a TCR provided herein, wherein the TCR specifically binds preproinsulin 73-90 complexed with an MHC molecule in the pancreas and/or draining lymph node(s) of the subject, thereby repairing pancreatic tissue damaged tissue in the subject.

Yet other aspects relate to a method of preventing autoimmunity associated with Type 1 Diabetes in a subject in need thereof, comprising administering to the subject a composition comprising the cell population expressing a TCR provided herein, wherein the TCR specifically binds preproinsulin 73-90 complexed with an MHC molecule in the pancreas and/or draining lymph node(s) of the subject, thereby preventing autoimmunity associated with Type 1 Diabetes in the subject.

Some aspects relate to a method of suppressing pancreatic islet-associated inflammation in a subject in need thereof, comprising administering to the subject a composition comprising the cell population of claim 247, wherein the TCR specifically binds preproinsulin 73-90 complexed with an MHC molecule in the pancreas and/or draining lymph node(s) of the subject, thereby suppressing pancreatic islet-associated inflammation in the subject.

In some embodiments, a subject has Type 1 Diabetes. In some embodiments, a subject has Stage 3 Type 1 Diabetes (e.g., new onset Stage 3 Type 1 Diabetes).

In some embodiments, a composition further comprises a pharmaceutically acceptable excipient or a cryopreservative.

In some embodiments, administering comprises intravenous administration. In some embodiments, administering comprises one or more infusions (e.g., of an isolated cell population or pharmaceutical composition). In some embodiments, administering comprises a single infusion (e.g., of an isolated cell population or pharmaceutical composition). In some embodiments, administering comprises more than one infusion of an isolated cell population or pharmaceutical composition.

In some embodiments, a subject has an HLA-DRBI genetic haplotype (expresses major histocompatibility complex, class II, DR beta 1 (HLA-DRBI). In some embodiments, a subject has an HLA-DRB1*04:01 genetic haplotype.

In some embodiments, a subject has new-onset/adult-onset Type 1 Diabetes. In some embodiments, a subject has new onset/adult-onset Stage 3 Type 1 Diabetes.

In some embodiments, a subject has residual beta cell function.

Some aspects of the disclosure provide one or more amino acid sequences encoding a TCR that binds (e.g., specifically binds) to a proinsulin peptide (e.g., preproinsulin 73-90) complexed with an MHC molecule. In some embodiments, the MHC molecule is an MHC class II antigen.

Some aspects of the disclosure provide a TCR that (a) comprises one or more amino acid sequences set forth in Table I (e.g., a CDR sequence belonging to any one of TCR-A, TCR-B, TCR-C, TCR-D, TCR-E, TCR-F, TCR-G, TCR-H, TCR-I, TCR-J, TCR-K, TCR-L, TCR-M, TCR-N, TCR-O, TCR-P, TCR-Q, TCR-R, TCR-S, TCR-T, TCR-U, TCR-V, TCR-X, TCR-Y and TCR-Z), and (b) binds (e.g., specifically binds) to a proinsulin peptide (e.g., preproinsulin 73-90) complexed with an MHC molecule. In some embodiments, a proinsulin peptide is preproinsulin 73-90.

In some embodiments, a preproinsulin 73-90) peptide comprises the amino acid sequence of GAGSLQPLALEGSLQKRG (SEQ ID NO: 109). In some embodiments, an MHC molecule comprises an HLA-DRB1*04:01 molecule. In some embodiments, an MHC molecule comprises an HLA-DRA*0101 molecule.

In some embodiments, a TCR is encoded as a single polypeptide. A polypeptide encoding a TCR may comprise, for example, an N-terminal beta domain and a C-terminal alpha domain. In some embodiments, a polypeptide comprises a self-cleaving peptide sequence positioned between a TCR alpha chain and a TCR beta chain. In some embodiments, a self-cleaving peptide sequence is a 2A peptide sequence. A 2A peptide sequence may be, for example, a P2A, E2A, F2A, or T2A peptide sequence. In some embodiments, a 2A peptide sequence is a P2A peptide sequence.

In some embodiments, a TCR alpha chain comprises a murine constant region. In some embodiments, a TCR beta chain comprises a murine constant region.

In some embodiments, a TCR comprises one or more amino acid substitutions to cysteine residues in the alpha chain constant region and the beta chain constant region, and wherein the cysteine residues form or are capable of forming one or more disulfide bridges. In some embodiments, a TCR alpha chain constant region comprises a T48C amino acid substitution relative to an alpha chain constant region comprising the amino acid sequence of SEQ ID NO: 106, and wherein the beta chain constant region comprises a S57C amino acid substitution relative to a beta chain constant region comprising the amino acid sequence of SEQ ID NO: 108.

Some aspects of disclosure provide a nucleic acid encoding any one of the TCRs described herein. In some embodiments, a nucleic acid is a vector (e.g., a viral vector or
a lentiviral vector).

In some embodiments, a nucleic acid comprises a promoter operably linked to a coding sequence encoding a TCR. A promoter may be, for example, an EF-1 alpha promoter or an MND promoter. In some embodiments, a nucleic acid further comprises an enhancer element. An enhancer element may be for example, an optimized post-transcriptional regulatory element (oPRE) or a woodchuck hepatitis virus post-transcriptional regulatory element (WPRE). In some embodiments, an enhancer element comprises WPRE-mut6. In some embodiments, a coding sequence is codon-optimized.

Some aspects of the disclosure provide an engineered cell comprising a TCR (e.g., engineered TCR) that binds (e.g., specifically binds) to a proinsulin peptide (e.g., preproinsulin 73-90) complexed with an MHC molecule. In some embodiments, a proinsulin peptide is preproinsulin 73-90. In some embodiments, a TCR is any one of the TCRs described herein. In some embodiments, an engineered cell is a T cell, for example, a regulatory T cell. In some embodiments, an engineered cell is from a subject (e.g., isolated from), for example, a subject having (e.g., diagnosed with) or suspected of having Type 1 Diabetes.

Some aspects of the disclosure provide a cell population comprising a plurality of engineered cells (e.g., regulatory T cells) comprising any one of the TCRs described herein.

Some aspects of the disclosure provide a pharmaceutical composition comprising a cell population described herein and a pharmaceutically acceptable excipient. Other aspects of the disclosure provide a composition comprising a cell population described herein and a cryopreservative.

Some aspects of the disclosure provide a method comprising administering to a subject a population of cells, a pharmaceutical composition, or a composition described herein, wherein the subject has Type 1 Diabetes. In some embodiments, a population of cells, a pharmaceutical composition, or a composition is administered in an effective amount to alleviate one or more symptom of Type 1 Diabetes. In some embodiments, administering comprises intravenous administration. In some embodiments, administering comprises one or more infusion (e.g., of a population of cells, pharmaceutical composition, or a composition described herein). In some embodiments, administering comprises a single infusion. In some embodiments, the administering comprises a single infusion of a population of cells (e.g., an isolated cell population). In some embodiments, administering comprises a single infusion of a pharmaceutical composition.

In some embodiments, a TCR specifically binds preproinsulin 73-90 complexed with an MHC molecule (e.g., MHC class II antigen) in the pancreas and/or draining lymph node(s) of a subject.

In some embodiments, a cell population, a pharmaceutical composition, or a composition is administered in an amount effective to prevent β cell destruction and/or preserve β cell mass in the pancreas of a subject.

In some embodiments, a cell population, a pharmaceutical composition, or a composition is administered in an amount effective to repair pancreatic tissue damage in a subject.

In some embodiments, a cell population, a pharmaceutical composition, or a composition is administered in an amount effective to preventing autoimmunity associated with Type 1 Diabetes in a subject.

In some embodiments, a cell population, the pharmaceutical composition, or a composition is administered in an amount effective to suppress pancreatic islet-associated inflammation in a subject.

### BRIEF DESCRIPTION OF THE DRAWINGS

FIGS. 1A-1B provide graphs showing the ability of TCRs of the disclosure to activate TCR-null Jurkat cells in the presence of preproinsulin peptide as illustrated by CD69 expression levels.

FIG. 2 provides graphs showing activation of TCR-null Jurkat cells by TCRs of the disclosure as determined by CD69 levels at the concentrations of preproinsulin peptide shown.

FIG. 3 provides graphs showing activation of conventional CD4⁺ T cells by TCRs of the disclosure as determined by CD69 levels at the concentrations of preproinsulin peptide shown.

FIGS. 4A-4B provide graphs showing activation of conventional CD4⁺ T cells by TCRs of the disclosure as determined by the proportion of TCR+ cells expressing CD69 at the concentrations of preproinsulin peptide shown.

FIGS. 5A-5C provide graphs showing activation of conventional CD4⁺ T cells by TCRs of the disclosure as determined by the proportion of TCR+ cells expressing CD69 at the concentrations of preproinsulin peptide shown, where the TCRs either have unmodified or disulfide-modified constant domain sequences.

FIG. 6 provides graphs showing activation of regulatory T cells by TCRs of the disclosure as determined by the proportion of TCR+ cells expressing CD69 at the concentrations of preproinsulin peptide shown.

FIGS. 7A-7E provides graphs showing suppressive activity of regulatory T cells by TCRs of the disclosure as determined by IL-10 production and CD69 upregulation at varying concentrations of preproinsulin peptide.

FIGS. 8A-8C provide graphs showing suppressive activity of regulatory T cells expressing TCRs of the disclosure towards conventional T cells as determined by capacity to suppress proliferation of conventional T cells.

FIG. 9 provides graphs showing suppressive activity of regulatory T cells expressing TCRs of the disclosure towards CD8⁺ T cells and dendritic cells.

FIG. 10 provides graphs showing the results of X scan analysis with TCRs of the disclosure demonstrating on target specificity.

FIG. 11 provides graphs showing suppressive activity of regulatory T cells expressing TCRs of the disclosure towards conventional T cells.

FIG. 12 provides a graph showing that Jurkat cells transduced a TCR of the disclosure only showed upregulation of CD69 when co-cultured with cells that express HLA-DRB*04:01 (e.g., no upregulation when co-cultured with cells that do not express HLA-DRB*04:01).

FIG. 13 shows the percent TSDR hypomethylation of 6 different runs produced by the methods described in Example 6.

### DETAILED DESCRIPTION

Provided herein, in some aspects, is an autologous T cell (e.g., regulatory T cell) therapy for the treatment of Type 1 Diabetes (TID), for example, in patients having an HLA-DRB1*04:01 genetic haplotype. The therapy, in some embodiments, is a targeted therapy for TID patients who have remaining/residual beta cell function in the pancreas (e.g., still produce insulin). In some embodiments, a patient's own regulatory T cells are minimally engineered to express a TCR that specifically recognizes, in the pancreas and draining lymph nodes, immunogenic protein fragments of an autoantigen that drives the development of effector T cells, which lead to beta cell destruction. The engineering of these regulatory T cells has been shown to enhance the therapeutic potential of the cells, as evidenced by the increased retention, activation, proliferation, and differentiation of the regulatory T cells when they encounter cognate antigen-major histocompatibility complex (MHC) (e.g., MHC class II antigen) in the pancreas, for example, in inflamed islets of Langerhans and pancreatic draining lymph nodes. The therapy described herein, in some embodiments, offers a strong safety profile and a highly localized anti-inflammatory effect at the site of disease. In some aspects, engineered regulatory T cells provided herein (expressing a TCR of the disclosure) exhibit antigen-specificity, dose-dependent regulatory T cell functionality, anti-inflammatory cytokine production, and suppression of the production of inflammatory factors (e.g., T cell-derived cytokines). In some embodiments, engineered regulatory T cells also retain their regulatory T cell phenotype under inflammatory conditions (e.g., in the presence of one or more inflammatory cytokines).

The present disclosure provides compositions of T cell receptors (TCRs), compositions of engineered regulatory T cells comprising said TCRs, and methods of using the engineered regulatory T cells for treatment of Type 1 Diabetes (e.g., Stage 3 Type 1 Diabetes). Although 40% of Caucasians in the US have an HLA-DR3 or -DR4 allele, at least one of these alleles is present in 95% of patients with Type 1 Diabetes. Risk of diabetes is also influenced by DRB1*04 variants with DRB1*04:01 having an odds ratio of 8.4. TCRs of the disclosure are capable of specifically binding to proinsulin peptides presented by an MHC molecule, for example, MHC class II antigen, such as HLA-DRBI, e.g., HLA-DRB1*04:01, with nanomolar to low micromolar binding affinities. Engineering of regulatory T cells to express the TCRs of the disclosure enables the T cells to specifically target discrete cell types and tissues associated with Type 1 Diabetes to prevent immune-mediated destruction, restore homeostasis, and promote repair in affected tissues.

While many of the aspects and embodiments described herein are directed to regulatory T cells, the TCRs of the disclosure may be expressed in other T cell types, depending on the intended use of the T cell expressing TCR (e.g., for targeting the T cells to a preproinsulin peptide, such as preproinsulin peptide 73-90).

### T Cell Receptors

In some embodiments, the disclosure provides a T cell receptor (TCR) that enables antigen-specific targeting (e.g., by engineered regulatory T cells). A TCR is (includes) a transmembrane heterodimer that includes an alpha chain and beta chain linked by a disulfide bond. Within these chains are complementary determining regions (CDRs) that determine the target peptide to which the TCR will bind. TCRs activate T cells in which they reside leading to a plethora of immune responses. Antigen presenting cells digest certain proteins (antigens) and display their fragments (peptides) on major histocompatibility complexes (MHC). This peptide-MHC (pMHC) complex binds to the TCR while other co-stimulatory molecules are activated, leading to T cell activation, proliferation, differentiation, apoptosis, and/or cytokine release.

A TCR binds specifically to a target peptide (e.g., a proinsulin peptide) complexed with an MHC molecule. A TCR is considered to bind “specifically” to a target peptide complexed with an MHC molecule if the TCR has a higher binding affinity for the target peptide complexed with an MHC molecule relative to a non-target peptide complexed with an MHC molecule. A TCR may bind to a target peptide complexed with an MHC molecule with a binding affinity of at least 10⁻⁴ M, 10⁻⁵ M, 10⁻⁶ M, 10⁻⁷ M, 10⁻⁸ M, 10⁻⁹ M, or 10-10 M (e.g., 10⁻⁴ M to 10⁻¹⁰ M). In some embodiments, a TCR is considered to bind “specifically” to a target peptide complexed with an MHC molecule if a T cell expressing the TCR becomes activated (e.g., as assessed by increased CD69 expression) when contacted with the target peptide complexed with an MHC molecule or becomes more highly activated relative to a non-target peptide complexed with an MHC molecule. In some embodiments, a peptide is presented by a cell expressing the MHC molecule. In some embodiments, a TCR binds specifically to a proinsulin peptide (e.g., a preproinsulin peptide). In some embodiments, a TCR binds specifically to a preproinsulin 73-90 peptide (e.g., comprising the amino acid sequence of SEQ ID NO: 109). Preproinsulin is an islet autoantigen found in subjects with Type 1 Diabetes and it is abundantly and selectively expressed by pancreatic beta cells.

In some embodiments, a TCR specifically binds preproinsulin 73-90 complexed with MHC molecule in the pancreas of a subject. In some embodiments, a TCR specifically binds preproinsulin 73-90 complexed with MHC molecule in the draining lymph node(s) of a subject. Lymph node draining is a process in which the lymphatic system moves lymph to the lymph nodes throughout the body.

In some embodiments, a TCR comprises one or more amino acid sequences as described in Table 1 (e.g., one or more amino acid sequences belonging to any one of TCR-A, TCR-B, TCR-C, TCR-D, TCR-E, TCR-F, TCR-G, TCR-H, TCR-I, TCR-J, TCR-K, TCR-L, TCR-M, TCR-N, TCR-O, TCR-P, TCR-Q, TCR-R, TCR-S, TCR-T, TCR-U, TCR-V, TCR-X, TCR-Y and TCR-Z).

A TCR may comprise the amino acid sequence of any alpha chain CDR1, CDR2, or CDR3 as provided in Table 1. In some embodiments, an alpha CDR1 of the TCR is any one of SEQ ID NOs: 1, 11, 21, 31, 41, 51, 61, 71, 81, 91, 110, 120, 130, 140, 150, 160, 170, 180, 190, 200, 210, 220, 230, 240, or 250. In some embodiments, an alpha CDR2 of the TCR is any one of SEQ ID NOs: 2, 12, 22, 32, 42, 52, 62, 72, 82, 92, 111, 121, 131, 141, 151, 161, 171, 181, 191, 201, 211, 221, 231, 241, or 251. In some embodiments, an alpha CDR3 of the TCR is any one of SEQ ID NOs: 3, 13, 23, 33, 43, 53, 63, 73, 83, 93, 112, 122, 132, 142, 152, 162, 172, 182, 192, 202, 212, 222, 232, 242, or 252. A TCR may comprise the amino acid sequence of any beta chain CDR1, CDR2, or CDR3 as provided in Table 1. In some embodiments, a beta CDR1 of the TCR is any one of SEQ ID NOs: 6, 16, 26, 36, 46, 56, 66, 76, 86, 96, 115, 125, 135, 145, 155, 165, 175, 185, 195, 205, 215, 225, 235, 245, or 255. In some embodiments, a beta CDR2 of the TCR is any one of SEQ ID NOs: 7, 17, 27, 37, 47, 57, 67, 77, 87, 97, 116, 126, 136, 146, 156, 166, 176, 186, 196, 206, 216, 226, 236, 246, or 256. In some embodiments, a beta CDR3 of the TCR is any one of SEQ ID NOs: 8, 18, 28, 38, 48, 58, 68, 78, 88, 98, 117. 127, 137, 147, 157, 167, 177, 187, 197, 207, 217, 227, 237, 247, or 257.

In some embodiments, the alpha chain variable region of a TCR comprises an amino acid sequence having at least 90%, 95%, or 100% identity to the amino acid sequence of SEQ ID NO: 4, 14, 24, 34, 44, 54, 64, 74, 84, 94, 113, 123, 133, 143, 153, 163, 173, 183, 193, 203, 213, 223, 233, 243, or 253. In some embodiments, the beta chain variable region of a TCR comprises an amino acid sequence having at least 90%, 95%, or 100% identity to the amino acid sequence of SEQ ID NO: 9, 19, 29, 39, 49, 59, 69, 79, 89, 99, 118, 128, 138, 148, 158, 168, 178, 188, 198, 208, 218, 228, 238, 248, or 258. In some embodiments, the alpha chain of a TCR comprises an amino acid sequence having at least 90%, 95%, or 100% identity to the amino acid sequence of SEQ ID NO: 5, 15, 25, 35, 45, 55, 65, 75, 85, 95, 114, 124, 134, 144, 154, 164, 174, 184, 194, 204, 214, 224, 234, 244, 254, 260, 262, 264, 266, 268, 270, 272, 274, 276, 278, 280, 282, 284, 286, 288, 290, 292, 294, 296, 298, 300, 302, 304, 306, or 308. In some embodiments, the beta chain of a TCR comprises an amino acid sequence having at least 90%, 95%, or 100% identity to the amino acid sequence of SEQ ID NO: 10, 20, 30, 40, 50, 60, 70, 80, 90, 100, 119, 129, 139, 149, 159, 169, 179, 189, 199, 209, 219, 229, 239, 249, 259, 261, 263, 265, 267, 269, 271, 273, 275, 277, 279, 281, 283, 285, 287, 289, 291, 293, 295, 297, 299, 301, 303, 305, 307, or 309. In some embodiments, a TCR alpha or beta variable region comprises a V-region and a J-region. In some embodiments, a TCR beta variable region comprises a V-region, a D-region, and a J-region.

In some embodiments, the alpha chain variable region of a TCR comprises an amino acid sequence having at least 90%, 95%, or 100% identity to the amino acid sequence of SEQ ID NO: 4; and the beta chain variable region of a TCR comprises an amino acid sequence having at least 90%, 95%, or 100% identity to the amino acid sequence of SEQ ID NO: 9. In some embodiments, the alpha chain of a TCR comprises an amino acid sequence having at least 90%, 95%, or 100% identity to the amino acid sequence of SEQ ID NO: 5; and the beta chain of a TCR comprises an amino acid sequence having at least 90%, 95%, or 100% identity to the amino acid sequence of SEQ ID NO: 10. In some embodiments, the alpha chain of a TCR comprises an amino acid sequence having at least 90%, 95%, or 100% identity to the amino acid sequence of SEQ ID NO: 260; and the beta chain of a TCR comprises an amino acid sequence having at least 90%, 95%, or 100% identity to the amino acid sequence of SEQ ID NO: 261.

In some embodiments, the alpha chain variable region of a TCR comprises an amino acid sequence having at least 90%, 95%, or 100% identity to the amino acid sequence of SEQ ID NO: 14; and the beta chain variable region of a TCR comprises an amino acid sequence having at least 90%, 95%, or 100% identity to the amino acid sequence of SEQ ID NO: 19. In some embodiments, the alpha chain of a TCR comprises an amino acid sequence having at least 90%, 95%, or 100% identity to the amino acid sequence of SEQ ID NO: 15; and the beta chain of a TCR comprises an amino acid sequence having at least 90%, 95%, or 100% identity to the amino acid sequence of SEQ ID NO: 20. In some embodiments, the alpha chain of a TCR comprises an amino acid sequence having at least 90%, 95%, or 100% identity to the amino acid sequence of SEQ ID NO: 262; and the beta chain of a TCR comprises an amino acid sequence having at least 90%, 95%, or 100% identity to the amino acid sequence of SEQ ID NO: 263.

In some embodiments, the alpha chain variable region of a TCR comprises an amino acid sequence having at least 90%, 95%, or 100% identity to the amino acid sequence of SEQ ID NO: 24; and the beta chain variable region of a TCR comprises an amino acid sequence having at least 90%, 95%, or 100% identity to the amino acid sequence of SEQ ID NO: 29. In some embodiments, the alpha chain of a TCR comprises an amino acid sequence having at least 90%, 95%, or 100% identity to the amino acid sequence of SEQ ID NO: 25; and the beta chain of a TCR comprises an amino acid sequence having at least 90%, 95%, or 100% identity to the amino acid sequence of SEQ ID NO: 30. In some embodiments, the alpha chain of a TCR comprises an amino acid sequence having at least 90%, 95%, or 100% identity to the amino acid sequence of SEQ ID NO: 264; and the beta chain of a TCR comprises an amino acid sequence having at least 90%, 95%, or 100% identity to the amino acid sequence of SEQ ID NO: 265.

In some embodiments, the alpha chain variable region of a TCR comprises an amino acid sequence having at least 90%, 95%, or 100% identity to the amino acid sequence of SEQ ID NO: 34; and the beta chain variable region of a TCR comprises an amino acid sequence having at least 90%, 95%, or 100% identity to the amino acid sequence of SEQ ID NO: 39. In some embodiments, the alpha chain of a TCR comprises an amino acid sequence having at least 90%, 95%, or 100% identity to the amino acid sequence of SEQ ID NO: 35; and the beta chain of a TCR comprises an amino acid sequence having at least 90%, 95%, or 100% identity to the amino acid sequence of SEQ ID NO: 40. In some embodiments, the alpha chain of a TCR comprises an amino acid sequence having at least 90%, 95%, or 100% identity to the amino acid sequence of SEQ ID NO: 266; and the beta chain of a TCR comprises an amino acid sequence having at least 90%, 95%, or 100% identity to the amino acid sequence of SEQ ID NO: 267.

In some embodiments, the alpha chain variable region of a TCR comprises an amino acid sequence having at least 90%, 95%, or 100% identity to the amino acid sequence of SEQ ID NO: 44; and the beta chain variable region of a TCR comprises an amino acid sequence having at least 90%, 95%, or 100% identity to the amino acid sequence of SEQ ID NO: 49. In some embodiments, the alpha chain of a TCR comprises an amino acid sequence having at least 90%, 95%, or 100% identity to the amino acid sequence of SEQ ID NO: 45; and the beta chain of a TCR comprises an amino acid sequence having at least 90%, 95%, or 100% identity to the amino acid sequence of SEQ ID NO: 50. In some embodiments, the alpha chain of a TCR comprises an amino acid sequence having at least 90%, 95%, or 100% identity to the amino acid sequence of SEQ ID NO: 268; and the beta chain of a TCR comprises an amino acid sequence having at least 90%, 95%, or 100% identity to the amino acid sequence of SEQ ID NO: 269.

In some embodiments, the alpha chain variable region of a TCR comprises an amino acid sequence having at least 90%, 95%, or 100% identity to the amino acid sequence of SEQ ID NO: 54; and the beta chain variable region of a TCR comprises an amino acid sequence having at least 90%, 95%, or 100% identity to the amino acid sequence of SEQ ID NO: 59. In some embodiments, the alpha chain of a TCR comprises an amino acid sequence having at least 90%, 95%, or 100% identity to the amino acid sequence of SEQ ID NO: 55; and the beta chain of a TCR comprises an amino acid sequence having at least 90%, 95%, or 100% identity to the amino acid sequence of SEQ ID NO: 60. In some embodiments, the alpha chain of a TCR comprises an amino acid sequence having at least 90%, 95%, or 100% identity to the amino acid sequence of SEQ ID NO: 270; and the beta chain of a TCR comprises an amino acid sequence having at least 90%, 95%, or 100% identity to the amino acid sequence of SEQ ID NO: 271.

In some embodiments, the alpha chain variable region of a TCR comprises an amino acid sequence having at least 90%, 95%, or 100% identity to the amino acid sequence of SEQ ID NO: 64; and the beta chain variable region of a TCR comprises an amino acid sequence having at least 90%, 95%, or 100% identity to the amino acid sequence of SEQ ID NO: 69. In some embodiments, the alpha chain of a TCR comprises an amino acid sequence having at least 90%, 95%, or 100% identity to the amino acid sequence of SEQ ID NO: 65; and the beta chain of a TCR comprises an amino acid sequence having at least 90%, 95%, or 100% identity to the amino acid sequence of SEQ ID NO: 70. In some embodiments, the alpha chain of a TCR comprises an amino acid sequence having at least 90%, 95%, or 100% identity to the amino acid sequence of SEQ ID NO: 272; and the beta chain of a TCR comprises an amino acid sequence having at least 90%, 95%, or 100% identity to the amino acid sequence of SEQ ID NO: 273.

In some embodiments, the alpha chain variable region of a TCR comprises an amino acid sequence having at least 90%, 95%, or 100% identity to the amino acid sequence of SEQ ID NO: 74; and the beta chain variable region of a TCR comprises an amino acid sequence having at least 90%, 95%, or 100% identity to the amino acid sequence of SEQ ID NO: 79. In some embodiments, the alpha chain of a TCR comprises an amino acid sequence having at least 90%, 95%, or 100% identity to the amino acid sequence of SEQ ID NO: 75; and the beta chain of a TCR comprises an amino acid sequence having at least 90%, 95%, or 100% identity to the amino acid sequence of SEQ ID NO: 80. In some embodiments, the alpha chain of a TCR comprises an amino acid sequence having at least 90%, 95%, or 100% identity to the amino acid sequence of SEQ ID NO: 274; and the beta chain of a TCR comprises an amino acid sequence having at least 90%, 95%, or 100% identity to the amino acid sequence of SEQ ID NO: 275.

In some embodiments, the alpha chain variable region of a TCR comprises an amino acid sequence having at least 90%, 95%, or 100% identity to the amino acid sequence of SEQ ID NO: 84; and the beta chain variable region of a TCR comprises an amino acid sequence having at least 90%, 95%, or 100% identity to the amino acid sequence of SEQ ID NO: 89. In some embodiments, the alpha chain of a TCR comprises an amino acid sequence having at least 90%, 95%, or 100% identity to the amino acid sequence of SEQ ID NO: 85; and the beta chain of a TCR comprises an amino acid sequence having at least 90%, 95%, or 100% identity to the amino acid sequence of SEQ ID NO: 90. In some embodiments, the alpha chain of a TCR comprises an amino acid sequence having at least 90%, 95%, or 100% identity to the amino acid sequence of SEQ ID NO: 276; and the beta chain of a TCR comprises an amino acid sequence having at least 90%, 95%, or 100% identity to the amino acid sequence of SEQ ID NO: 277.

In some embodiments, the alpha chain variable region of a TCR comprises an amino acid sequence having at least 90%, 95%, or 100% identity to the amino acid sequence of SEQ ID NO: 94; and the beta chain variable region of a TCR comprises an amino acid sequence having at least 90%, 95%, or 100% identity to the amino acid sequence of SEQ ID NO: 99. In some embodiments, the alpha chain of a TCR comprises an amino acid sequence having at least 90%, 95%, or 100% identity to the amino acid sequence of SEQ ID NO: 95; and the beta chain of a TCR comprises an amino acid sequence having at least 90%, 95%, or 100% identity to the amino acid sequence of SEQ ID NO: 100. In some embodiments, the alpha chain of a TCR comprises an amino acid sequence having at least 90%, 95%, or 100% identity to the amino acid sequence of SEQ ID NO: 278; and the beta chain of a TCR comprises an amino acid sequence having at least 90%, 95%, or 100% identity to the amino acid sequence of SEQ ID NO: 279.

In some embodiments, the alpha chain variable region of a TCR comprises an amino acid sequence having at least 90%, 95%, or 100% identity to the amino acid sequence of SEQ ID NO: 113; and the beta chain variable region of a TCR comprises an amino acid sequence having at least 90%, 95%, or 100% identity to the amino acid sequence of SEQ ID NO: 118. In some embodiments, the alpha chain of a TCR comprises an amino acid sequence having at least 90%, 95%, or 100% identity to the amino acid sequence of SEQ ID NO: 114; and the beta chain of a TCR comprises an amino acid sequence having at least 90%, 95%, or 100% identity to the amino acid sequence of SEQ ID NO: 119. In some embodiments, the alpha chain of a TCR comprises an amino acid sequence having at least 90%, 95%, or 100% identity to the amino acid sequence of SEQ ID NO: 280; and the beta chain of a TCR comprises an amino acid sequence having at least 90%, 95%, or 100% identity to the amino acid sequence of SEQ ID NO: 281.

In some embodiments, the alpha chain variable region of a TCR comprises an amino acid sequence having at least 90%, 95%, or 100% identity to the amino acid sequence of SEQ ID NO: 123; and the beta chain variable region of a TCR comprises an amino acid sequence having at least 90%, 95%, or 100% identity to the amino acid sequence of SEQ ID NO: 128. In some embodiments, the alpha chain of a TCR comprises an amino acid sequence having at least 90%, 95%, or 100% identity to the amino acid sequence of SEQ ID NO: 124; and the beta chain of a TCR comprises an amino acid sequence having at least 90%, 95%, or 100% identity to the amino acid sequence of SEQ ID NO: 129. In some embodiments, the alpha chain of a TCR comprises an amino acid sequence having at least 90%, 95%, or 100% identity to the amino acid sequence of SEQ ID NO: 282; and the beta chain of a TCR comprises an amino acid sequence having at least 90%, 95%, or 100% identity to the amino acid sequence of SEQ ID NO: 283.

In some embodiments, the alpha chain variable region of a TCR comprises an amino acid sequence having at least 90%, 95%, or 100% identity to the amino acid sequence of SEQ ID NO: 133; and the beta chain variable region of a TCR comprises an amino acid sequence having at least 90%, 95%, or 100% identity to the amino acid sequence of SEQ ID NO: 138. In some embodiments, the alpha chain of a TCR comprises an amino acid sequence having at least 90%, 95%, or 100% identity to the amino acid sequence of SEQ ID NO: 134; and the beta chain of a TCR comprises an amino acid sequence having at least 90%, 95%, or 100% identity to the amino acid sequence of SEQ ID NO: 139. In some embodiments, the alpha chain of a TCR comprises an amino acid sequence having at least 90%, 95%, or 100% identity to the amino acid sequence of SEQ ID NO: 284; and the beta chain of a TCR comprises an amino acid sequence having at least 90%, 95%, or 100% identity to the amino acid sequence of SEQ ID NO: 285.

In some embodiments, the alpha chain variable region of a TCR comprises an amino acid sequence having at least 90%, 95%, or 100% identity to the amino acid sequence of SEQ ID NO: 143; and the beta chain variable region of a TCR comprises an amino acid sequence having at least 90%, 95%, or 100% identity to the amino acid sequence of SEQ ID NO: 148. In some embodiments, the alpha chain of a TCR comprises an amino acid sequence having at least 90%, 95%, or 100% identity to the amino acid sequence of SEQ ID NO: 144; and the beta chain of a TCR comprises an amino acid sequence having at least 90%, 95%, or 100% identity to the amino acid sequence of SEQ ID NO: 149. In some embodiments, the alpha chain of a TCR comprises an amino acid sequence having at least 90%, 95%, or 100% identity to the amino acid sequence of SEQ ID NO: 286; and the beta chain of a TCR comprises an amino acid sequence having at least 90%, 95%, or 100% identity to the amino acid sequence of SEQ ID NO: 287.

In some embodiments, the alpha chain variable region of a TCR comprises an amino acid sequence having at least 90%, 95%, or 100% identity to the amino acid sequence of SEQ ID NO: 153; and the beta chain variable region of a TCR comprises an amino acid sequence having at least 90%, 95%, or 100% identity to the amino acid sequence of SEQ ID NO: 158. In some embodiments, the alpha chain of a TCR comprises an amino acid sequence having at least 90%, 95%, or 100% identity to the amino acid sequence of SEQ ID NO: 154; and the beta chain of a TCR comprises an amino acid sequence having at least 90%, 95%, or 100% identity to the amino acid sequence of SEQ ID NO: 159. In some embodiments, the alpha chain of a TCR comprises an amino acid sequence having at least 90%, 95%, or 100% identity to the amino acid sequence of SEQ ID NO: 288; and the beta chain of a TCR comprises an amino acid sequence having at least 90%, 95%, or 100% identity to the amino acid sequence of SEQ ID NO: 289.

In some embodiments, the alpha chain variable region of a TCR comprises an amino acid sequence having at least 90%, 95%, or 100% identity to the amino acid sequence of SEQ ID NO: 163; and the beta chain variable region of a TCR comprises an amino acid sequence having at least 90%, 95%, or 100% identity to the amino acid sequence of SEQ ID NO: 168. In some embodiments, the alpha chain of a TCR comprises an amino acid sequence having at least 90%, 95%, or 100% identity to the amino acid sequence of SEQ ID NO: 164; and the beta chain of a TCR comprises an amino acid sequence having at least 90%, 95%, or 100% identity to the amino acid sequence of SEQ ID NO: 169. In some embodiments, the alpha chain of a TCR comprises an amino acid sequence having at least 90%, 95%, or 100% identity to the amino acid sequence of SEQ ID NO: 290; and the beta chain of a TCR comprises an amino acid sequence having at least 90%, 95%, or 100% identity to the amino acid sequence of SEQ ID NO: 291.

In some embodiments, the alpha chain variable region of a TCR comprises an amino acid sequence having at least 90%, 95%, or 100% identity to the amino acid sequence of SEQ ID NO: 173; and the beta chain variable region of a TCR comprises an amino acid sequence having at least 90%, 95%, or 100% identity to the amino acid sequence of SEQ ID NO: 178. In some embodiments, the alpha chain of a TCR comprises an amino acid sequence having at least 90%, 95%, or 100% identity to the amino acid sequence of SEQ ID NO: 174; and the beta chain of a TCR comprises an amino acid sequence having at least 90%, 95%, or 100% identity to the amino acid sequence of SEQ ID NO: 179. In some embodiments, the alpha chain of a TCR comprises an amino acid sequence having at least 90%, 95%, or 100% identity to the amino acid sequence of SEQ ID NO: 292; and the beta chain of a TCR comprises an amino acid sequence having at least 90%, 95%, or 100% identity to the amino acid sequence of SEQ ID NO: 293.

In some embodiments, the alpha chain variable region of a TCR comprises an amino acid sequence having at least 90%, 95%, or 100% identity to the amino acid sequence of SEQ ID NO: 183; and the beta chain variable region of a TCR comprises an amino acid sequence having at least 90%, 95%, or 100% identity to the amino acid sequence of SEQ ID NO: 188. In some embodiments, the alpha chain of a TCR comprises an amino acid sequence having at least 90%, 95%, or 100% identity to the amino acid sequence of SEQ ID NO: 184; and the beta chain of a TCR comprises an amino acid sequence having at least 90%, 95%, or 100% identity to the amino acid sequence of SEQ ID NO: 189. In some embodiments, the alpha chain of a TCR comprises an amino acid sequence having at least 90%, 95%, or 100% identity to the amino acid sequence of SEQ ID NO: 294; and the beta chain of a TCR comprises an amino acid sequence having at least 90%, 95%, or 100% identity to the amino acid sequence of SEQ ID NO: 295.

In some embodiments, the alpha chain variable region of a TCR comprises an amino acid sequence having at least 90%, 95%, or 100% identity to the amino acid sequence of SEQ ID NO: 193; and the beta chain variable region of a TCR comprises an amino acid sequence having at least 90%, 95%, or 100% identity to the amino acid sequence of SEQ ID NO: 198. In some embodiments, the alpha chain of a TCR comprises an amino acid sequence having at least 90%, 95%, or 100% identity to the amino acid sequence of SEQ ID NO: 194; and the beta chain of a TCR comprises an amino acid sequence having at least 90%, 95%, or 100% identity to the amino acid sequence of SEQ ID NO: 19. In some embodiments, the alpha chain of a TCR comprises an amino acid sequence having at least 90%, 95%, or 100% identity to the amino acid sequence of SEQ ID NO: 296; and the beta chain of a TCR comprises an amino acid sequence having at least 90%, 95%, or 100% identity to the amino acid sequence of SEQ ID NO: 297.

In some embodiments, the alpha chain variable region of a TCR comprises an amino acid sequence having at least 90%, 95%, or 100% identity to the amino acid sequence of SEQ ID NO: 203; and the beta chain variable region of a TCR comprises an amino acid sequence having at least 90%, 95%, or 100% identity to the amino acid sequence of SEQ ID NO: 208. In some embodiments, the alpha chain of a TCR comprises an amino acid sequence having at least 90%, 95%, or 100% identity to the amino acid sequence of SEQ ID NO: 204; and the beta chain of a TCR comprises an amino acid sequence having at least 90%, 95%, or 100% identity to the amino acid sequence of SEQ ID NO: 209. In some embodiments, the alpha chain of a TCR comprises an amino acid sequence having at least 90%, 95%, or 100% identity to the amino acid sequence of SEQ ID NO: 298; and the beta chain of a TCR comprises an amino acid sequence having at least 90%, 95%, or 100% identity to the amino acid sequence of SEQ ID NO: 299.

In some embodiments, the alpha chain variable region of a TCR comprises an amino acid sequence having at least 90%, 95%, or 100% identity to the amino acid sequence of SEQ ID NO: 213; and the beta chain variable region of a TCR comprises an amino acid sequence having at least 90%, 95%, or 100% identity to the amino acid sequence of SEQ ID NO: 218. In some embodiments, the alpha chain of a TCR comprises an amino acid sequence having at least 90%, 95%, or 100% identity to the amino acid sequence of SEQ ID NO: 214; and the beta chain of a TCR comprises an amino acid sequence having at least 90%, 95%, or 100% identity to the amino acid sequence of SEQ ID NO: 219. In some embodiments, the alpha chain of a TCR comprises an amino acid sequence having at least 90%, 95%, or 100% identity to the amino acid sequence of SEQ ID NO: 300; and the beta chain of a TCR comprises an amino acid sequence having at least 90%, 95%, or 100% identity to the amino acid sequence of SEQ ID NO: 301.

In some embodiments, the alpha chain variable region of a TCR comprises an amino acid sequence having at least 90%, 95%, or 100% identity to the amino acid sequence of SEQ ID NO: 223; and the beta chain variable region of a TCR comprises an amino acid sequence having at least 90%, 95%, or 100% identity to the amino acid sequence of SEQ ID NO: 228. In some embodiments, the alpha chain of a TCR comprises an amino acid sequence having at least 90%, 95%, or 100% identity to the amino acid sequence of SEQ ID NO: 224; and the beta chain of a TCR comprises an amino acid sequence having at least 90%, 95%, or 100% identity to the amino acid sequence of SEQ ID NO: 229. In some embodiments, the alpha chain of a TCR comprises an amino acid sequence having at least 90%, 95%, or 100% identity to the amino acid sequence of SEQ ID NO: 302; and the beta chain of a TCR comprises an amino acid sequence having at least 90%, 95%, or 100% identity to the amino acid sequence of SEQ ID NO: 303.

In some embodiments, the alpha chain variable region of a TCR comprises an amino acid sequence having at least 90%, 95%, or 100% identity to the amino acid sequence of SEQ ID NO: 233; and the beta chain variable region of a TCR comprises an amino acid sequence having at least 90%, 95%, or 100% identity to the amino acid sequence of SEQ ID NO: 238. In some embodiments, the alpha chain of a TCR comprises an amino acid sequence having at least 90%, 95%, or 100% identity to the amino acid sequence of SEQ ID NO: 234; and the beta chain of a TCR comprises an amino acid sequence having at least 90%, 95%, or 100% identity to the amino acid sequence of SEQ ID NO: 239. In some embodiments, the alpha chain of a TCR comprises an amino acid sequence having at least 90%, 95%, or 100% identity to the amino acid sequence of SEQ ID NO: 304; and the beta chain of a TCR comprises an amino acid sequence having at least 90%, 95%, or 100% identity to the amino acid sequence of SEQ ID NO: 305.

In some embodiments, the alpha chain variable region of a TCR comprises an amino acid sequence having at least 90%, 95%, or 100% identity to the amino acid sequence of SEQ ID NO: 243; and the beta chain variable region of a TCR comprises an amino acid sequence having at least 90%, 95%, or 100% identity to the amino acid sequence of SEQ ID NO: 248. In some embodiments, the alpha chain of a TCR comprises an amino acid sequence having at least 90%, 95%, or 100% identity to the amino acid sequence of SEQ ID NO: 244; and the beta chain of a TCR comprises an amino acid sequence having at least 90%, 95%, or 100% identity to the amino acid sequence of SEQ ID NO: 249. In some embodiments, the alpha chain of a TCR comprises an amino acid sequence having at least 90%, 95%, or 100% identity to the amino acid sequence of SEQ ID NO: 306; and the beta chain of a TCR comprises an amino acid sequence having at least 90%, 95%, or 100% identity to the amino acid sequence of SEQ ID NO: 307.

In some embodiments, the alpha chain variable region of a TCR comprises an amino acid sequence having at least 90%, 95%, or 100% identity to the amino acid sequence of SEQ ID NO: 253; and the beta chain variable region of a TCR comprises an amino acid sequence having at least 90%, 95%, or 100% identity to the amino acid sequence of SEQ ID NO: 258. In some embodiments, the alpha chain of a TCR comprises an amino acid sequence having at least 90%, 95%, or 100% identity to the amino acid sequence of SEQ ID NO: 254; and the beta chain of a TCR comprises an amino acid sequence having at least 90%, 95%, or 100% identity to the amino acid sequence of SEQ ID NO: 259. In some embodiments, the alpha chain of a TCR comprises an amino acid sequence having at least 90%, 95%, or 100% identity to the amino acid sequence of SEQ ID NO: 308; and the beta chain of a TCR comprises an amino acid sequence having at least 90%, 95%, or 100% identity to the amino acid sequence of SEQ ID NO: 309.

In some embodiments, a TCR comprises the amino acid sequences of SEQ ID NOs: 1-3 (e.g., TCRa CDRs 1-3), an amino acid sequence having at least 90% identity to the amino acid sequence of SEQ ID NO: 4 (e.g., TCRa variable domain), the amino acid sequences of SEQ ID NOS: 6-8 (e.g., TCRb CDRs 1-3), and an amino acid sequence having at least 90% identity to the amino acid sequence of SEQ ID NO: 9 (e.g., TCRb variable domain). In some embodiments, a TCR comprises the amino acid sequences of SEQ ID NOs: 1-3 (e.g., TCRa CDRs 1-3), an amino acid sequence having at least 90% identity to the amino acid sequence of SEQ ID NO: 5 (e.g., TCRa full length protein), the amino acid sequences of SEQ ID NOs: 6-8 (e.g., TCRb CDRs 1-3), and an amino acid sequence having at least 90% identity to the amino acid sequence of SEQ ID NO: 10 (e.g., TCRb full length protein). In some embodiments, a TCR comprises the amino acid sequences of SEQ ID NOs: 1-3 (e.g., TCRa CDRs 1-3), an amino acid sequence having at least 90% identity to the amino acid sequence of SEQ ID NO: 260 (e.g., TCRa full length protein), the amino acid sequences of SEQ ID NOs: 6-8 (e.g., TCRb CDRs 1-3), and an amino acid sequence having at least 90% identity to the amino acid sequence of SEQ ID NO: 261 (e.g., TCRb full length protein).

In some embodiments, a TCR comprises the amino acid sequences of SEQ ID NOs: 1-3 (e.g., TCRa CDRs 1-3) and an amino acid sequence having at least 90%, at least 95%, or at least 98% identity to the amino acid sequence of SEQ ID NO: 318 or 319 (e.g., TCRa full length). In some embodiments, a TCR comprises the amino acid sequences of SEQ ID NOs: 6-8 (e.g., TCRb CDRs 1-3) and an amino acid sequence having at least 90%, at least 95%, or at least 98% identity to the amino acid sequence of SEQ ID NO: 320, 321, 322, 323, 324, or 325 (e.g., TCRb full length). In some embodiments, a TCR comprises the amino acid sequences of SEQ ID NOs: 1-3 (e.g., TCRa CDRs 1-3), an amino acid sequence having at least 90%, at least 95%, or at least 98% identity to the amino acid sequence of SEQ ID NO: 318 (e.g., TCRa full length), the amino acid sequences of SEQ ID NOs: 6-8 (e.g., TCRb CDRs 1-3) and an amino acid sequence having at least 90%, at least 95%, or at least 98% identity to the amino acid sequence of SEQ ID NO: 321 or 322 (e.g., TCRb full length). In some embodiments, a TCR comprises the amino acid sequences of SEQ ID NOs: 1-3 (e.g., TCRa CDRs 1-3), an amino acid sequence having at least 90%, at least 95%, or at least 98% identity to the amino acid sequence of SEQ ID NO: 5 (e.g., TCRa full length), the amino acid sequences of SEQ ID NOs: 6-8 (e.g., TCRb CDRs 1-3) and an amino acid sequence having at least 90%, at least 95%, or at least 98% identity to the amino acid sequence of SEQ ID NO: 320 (e.g., TCRb full length). In some embodiments, a TCR comprises the amino acid sequences of SEQ ID NOs: 1-3 (e.g., TCRa CDRs 1-3), an amino acid sequence having at least 90%, at least 95%, or at least 98% identity to the amino acid sequence of SEQ ID NO: 319 (e.g., TCRa full length), the amino acid sequences of SEQ ID NOs: 6-8 (e.g., TCRb CDRs 1-3) and an amino acid sequence having at least 90%, at least 95%, or at least 98% identity to the amino acid sequence of SEQ ID NO: 324 or 325 (e.g., TCRb full length). In some embodiments, a TCR comprises the amino acid sequences of SEQ ID NOs: 1-3 (e.g., TCRa CDRs 1-3), an amino acid sequence having at least 90%, at least 95%, or at least 98% identity to the amino acid sequence of SEQ ID NO: 260 (e.g., TCRa full length protein), the amino acid sequences of SEQ ID NOs: 6-8 (e.g., TCRb CDRs 1-3) and an amino acid sequence having at least 90%, at least 95%, or at least 98% identity to the amino acid sequence of SEQ ID NO: 323 (e.g., TCRb full length protein).

In some embodiments, a TCR comprises the amino acid sequences of SEQ ID NOs: 1-3 (e.g., TCRa CDRs 1-3), an amino acid sequence having at least 95% identity to the amino acid sequence of SEQ ID NO: 4 (e.g., TCRa variable domain), the amino acid sequences of SEQ ID NOs: 6-8 (e.g., TCRb CDRs 1-3), and an amino acid sequence having at least 95% identity to the amino acid sequence of SEQ ID NO: 9 (e.g., TCRb variable domain). In some embodiments, a TCR comprises the amino acid sequences of SEQ ID NOs: 1-3 (e.g., TCRa CDRs 1-3), an amino acid sequence having at least 95% identity to the amino acid sequence of SEQ ID NO: 5 (e.g., TCRa full length protein), the amino acid sequences of SEQ ID NOs: 6-8 (e.g., TCRb CDRs 1-3), and an amino acid sequence having at least 95% identity to the amino acid sequence of SEQ ID NO: 10 (e.g., TCRb full length protein). In some embodiments, a TCR comprises the amino acid sequences of SEQ ID NOs: 1-3 (e.g., TCRa CDRs 1-3), an amino acid sequence having at least 95% identity to the amino acid sequence of SEQ ID NO: 260 (e.g., TCRa full length protein), the amino acid sequences of SEQ ID NOs: 6-8 (e.g., TCRb CDRs 1-3), and an amino acid sequence having at least 95% identity to the amino acid sequence of SEQ ID NO: 261 (e.g., TCRb full length protein).

In some embodiments, a TCR comprises the amino acid sequences of SEQ ID NOs: 1-3 (e.g., TCRa CDRs 1-3), an amino acid sequence having at least 98% identity to the amino acid sequence of SEQ ID NO: 4 (e.g., TCRa variable domain), the amino acid sequences of SEQ ID NOs: 6-8 (e.g., TCRb CDRs 1-3), and an amino acid sequence having at least 98% identity to the amino acid sequence of SEQ ID NO: 9 (e.g., TCRb variable domain). In some embodiments, a TCR comprises the amino acid sequences of SEQ ID NOs: 1-3 (e.g., TCRa CDRs 1-3), an amino acid sequence having at least 98% identity to the amino acid sequence of SEQ ID NO: 5 (e.g., TCRa full length protein), the amino acid sequences of SEQ ID NOs: 6-8 (e.g., TCRb CDRs 1-3), and an amino acid sequence having at least 98% identity to the amino acid sequence of SEQ ID NO: 10 (e.g., TCRb full length protein). In some embodiments, a TCR comprises the amino acid sequences of SEQ ID NOs: 1-3 (e.g., TCRa CDRs 1-3), an amino acid sequence having at least 98% identity to the amino acid sequence of SEQ ID NO: 260 (e.g., TCRa full length protein), the amino acid sequences of SEQ ID NOs: 6-8 (e.g., TCRb CDRs 1-3), and an amino acid sequence having at least 98% identity to the amino acid sequence of SEQ ID NO: 261 (e.g., TCRb full length protein).

In some embodiments, a TCR comprises the amino acid sequences of SEQ ID NOS: 11-13 (e.g., TCRa CDRs 1-3), an amino acid sequence having at least 90% identity to the amino acid sequence of SEQ ID NO: 14 (e.g., TCRa variable domain), the amino acid sequences of SEQ ID NOs: 16-18 (e.g., TCRb CDRs 1-3), and an amino acid sequence having at least 90% identity to the amino acid sequence of SEQ ID NO: 19 (e.g., TCRb variable domain). In some embodiments, a TCR comprises the amino acid sequences of SEQ ID NOs: 11-13 (e.g., TCRa CDRs 1-3), an amino acid sequence having at least 90% identity to the amino acid sequence of SEQ ID NO: 15 (e.g., TCRa full length protein), the amino acid sequences of SEQ ID NOS: 16-18 (e.g., TCRb CDRs 1-3), and an amino acid sequence having at least 90% identity to the amino acid sequence of SEQ ID NO: 20 (e.g., TCRb full length protein). In some embodiments, a TCR comprises the amino acid sequences of SEQ ID NOs: 11-13 (e.g., TCRa CDRs 1-3), an amino acid sequence having at least 90% identity to the amino acid sequence of SEQ ID NO: 262 (e.g., TCRa full length protein), the amino acid sequences of SEQ ID NOs: 16-18 (e.g., TCRb CDRs 1-3), and an amino acid sequence having at least 90% identity to the amino acid sequence of SEQ ID NO: 263 (e.g., TCRb full length protein).

In some embodiments, a TCR comprises the amino acid sequences of SEQ ID NOs: 11-13 (e.g., TCRa CDRs 1-3), an amino acid sequence having at least 95% identity to the amino acid sequence of SEQ ID NO: 14 (e.g., TCRa variable domain), the amino acid sequences of SEQ ID NOs: 16-18 (e.g., TCRb CDRs 1-3), and an amino acid sequence having at least 95% identity to the amino acid sequence of SEQ ID NO: 19 (e.g., TCRb variable domain). In some embodiments, a TCR comprises the amino acid sequences of SEQ ID NOs: 11-13 (e.g., TCRa CDRs 1-3), an amino acid sequence having at least 95% identity to the amino acid sequence of SEQ ID NO: 15 (e.g., TCRa full length protein), the amino acid sequences of SEQ ID NOS: 16-18 (e.g., TCRb CDRs 1-3), and an amino acid sequence having at least 95% identity to the amino acid sequence of SEQ ID NO: 20 (e.g., TCRb full length protein). In some embodiments, a TCR comprises the amino acid sequences of SEQ ID NOs: 11-13 (e.g., TCRa CDRs 1-3), an amino acid sequence having at least 95% identity to the amino acid sequence of SEQ ID NO: 262 (e.g., TCRa full length protein), the amino acid sequences of SEQ ID NOs: 16-18 (e.g., TCRb CDRs 1-3), and an amino acid sequence having at least 95% identity to the amino acid sequence of SEQ ID NO: 263 (e.g., TCRb full length protein).

In some embodiments, a TCR comprises the amino acid sequences of SEQ ID NOS: 11-13 (e.g., TCRa CDRs 1-3), an amino acid sequence having at least 98% identity to the amino acid sequence of SEQ ID NO: 14 (e.g., TCRa variable domain), the amino acid sequences of SEQ ID NOs: 16-18 (e.g., TCRb CDRs 1-3), and an amino acid sequence having at least 98% identity to the amino acid sequence of SEQ ID NO: 19 (e.g., TCRb variable domain). In some embodiments, a TCR comprises the amino acid sequences of SEQ ID NOs: 11-13 (e.g., TCRa CDRs 1-3), an amino acid sequence having at least 98% identity to the amino acid sequence of SEQ ID NO: 15 (e.g., TCRa full length protein), the amino acid sequences of SEQ ID NOs: 16-18 (e.g., TCRb CDRs 1-3), and an amino acid sequence having at least 98% identity to the amino acid sequence of SEQ ID NO: 20 (e.g., TCRb full length protein). In some embodiments, a TCR comprises the amino acid sequences of SEQ ID NOs: 11-13 (e.g., TCRa CDRs 1-3), an amino acid sequence having at least 98% identity to the amino acid sequence of SEQ ID NO: 262 (e.g., TCRa full length protein), the amino acid sequences of SEQ ID NOs: 16-18 (e.g., TCRb CDRs 1-3), and an amino acid sequence having at least 98% identity to the amino acid sequence of SEQ ID NO: 263 (e.g., TCRb full length protein).

In some embodiments, a TCR comprises the amino acid sequences of SEQ ID NOS: 21-23 (e.g., TCRa CDRs 1-3), an amino acid sequence having at least 90% identity to the amino acid sequence of SEQ ID NO: 24 (e.g., TCRa variable domain), the amino acid sequences of SEQ ID NOs: 26-28 (e.g., TCRb CDRs 1-3), and an amino acid sequence having at least 90% identity to the amino acid sequence of SEQ ID NO: 29 (e.g., TCRb variable domain). In some embodiments, a TCR comprises the amino acid sequences of SEQ ID NOs: 21-23 (e.g., TCRa CDRs 1-3), an amino acid sequence having at least 90% identity to the amino acid sequence of SEQ ID NO: 25 (e.g., TCRa full length protein), the amino acid sequences of SEQ ID NOs: 26-28 (e.g., TCRb CDRs 1-3), and an amino acid sequence having at least 90% identity to the amino acid sequence of SEQ ID NO: 30 (e.g., TCRb full length protein). In some embodiments, a TCR comprises the amino acid sequences of SEQ ID NOs: 21-23 (e.g., TCRa CDRs 1-3), an amino acid sequence having at least 90% identity to the amino acid sequence of SEQ ID NO: 264 (e.g., TCRa full length protein), the amino acid sequences of SEQ ID NOs: 26-28 (e.g., TCRb CDRs 1-3), and an amino acid sequence having at least 90% identity to the amino acid sequence of SEQ ID NO: 265 (e.g., TCRb full length protein).

In some embodiments, a TCR comprises the amino acid sequences of SEQ ID NOS: 21-23 (e.g., TCRa CDRs 1-3), an amino acid sequence having at least 95% identity to the amino acid sequence of SEQ ID NO: 24 (e.g., TCRa variable domain), the amino acid sequences of SEQ ID NOs: 26-28 (e.g., TCRb CDRs 1-3), and an amino acid sequence having at least 95% identity to the amino acid sequence of SEQ ID NO: 29 (e.g., TCRb variable domain). In some embodiments, a TCR comprises the amino acid sequences of SEQ ID NOs: 21-23 (e.g., TCRa CDRs 1-3), an amino acid sequence having at least 95% identity to the amino acid sequence of SEQ ID NO: 25 (e.g., TCRa full length protein), the amino acid sequences of SEQ ID NOs: 26-28 (e.g., TCRb CDRs 1-3), and an amino acid sequence having at least 95% identity to the amino acid sequence of SEQ ID NO: 30 (e.g., TCRb full length protein). In some embodiments, a TCR comprises the amino acid sequences of SEQ ID NOs: 21-23 (e.g., TCRa CDRs 1-3), an amino acid sequence having at least 95% identity to the amino acid sequence of SEQ ID NO: 264 (e.g., TCRa full length protein), the amino acid sequences of SEQ ID NOs: 26-28 (e.g., TCRb CDRs 1-3), and an amino acid sequence having at least 95% identity to the amino acid sequence of SEQ ID NO: 265 (e.g., TCRb full length protein).

In some embodiments, a TCR comprises the amino acid sequences of SEQ ID NOs: 21-23 (e.g., TCRa CDRs 1-3), an amino acid sequence having at least 98% identity to the amino acid sequence of SEQ ID NO: 24 (e.g., TCRa variable domain), the amino acid sequences of SEQ ID NOs: 26-28 (e.g., TCRb CDRs 1-3), and an amino acid sequence having at least 98% identity to the amino acid sequence of SEQ ID NO: 29 (e.g., TCRb variable domain). In some embodiments, a TCR comprises the amino acid sequences of SEQ ID NOs: 21-23 (e.g., TCRa CDRs 1-3), an amino acid sequence having at least 98% identity to the amino acid sequence of SEQ ID NO: 25 (e.g., TCRa full length protein), the amino acid sequences of SEQ ID NOs: 26-28 (e.g., TCRb CDRs 1-3), and an amino acid sequence having at least 98% identity to the amino acid sequence of SEQ ID NO: 30 (e.g., TCRb full length protein). In some embodiments, a TCR comprises the amino acid sequences of SEQ ID NOs: 21-23 (e.g., TCRa CDRs 1-3), an amino acid sequence having at least 98% identity to the amino acid sequence of SEQ ID NO: 264 (e.g., TCRa full length protein), the amino acid sequences of SEQ ID NOs: 26-28 (e.g., TCRb CDRs 1-3), and an amino acid sequence having at least 98% identity to the amino acid sequence of SEQ ID NO: 265 (e.g., TCRb full length protein).

In some embodiments, a TCR comprises the amino acid sequences of SEQ ID NOS: 31-33 (e.g., TCRa CDRs 1-3), an amino acid sequence having at least 90% identity to the amino acid sequence of SEQ ID NO: 34 (e.g., TCRa variable domain), the amino acid sequences of SEQ ID NOs: 36-38 (e.g., TCRb CDRs 1-3), and an amino acid sequence having at least 90% identity to the amino acid sequence of SEQ ID NO: 39 (e.g., TCRb variable domain). In some embodiments, a TCR comprises the amino acid sequences of SEQ ID NOs: 31-33 (e.g., TCRa CDRs 1-3), an amino acid sequence having at least 90% identity to the amino acid sequence of SEQ ID NO: 35 (e.g., TCRa full length protein), the amino acid sequences of SEQ ID NOs: 36-38 (e.g., TCRb CDRs 1-3), and an amino acid sequence having at least 90% identity to the amino acid sequence of SEQ ID NO: 40 (e.g., TCRb full length protein). In some embodiments, a TCR comprises the amino acid sequences of SEQ ID NOs: 31-33 (e.g., TCRa CDRs 1-3), an amino acid sequence having at least 90% identity to the amino acid sequence of SEQ ID NO: 266 (e.g., TCRa full length protein), the amino acid sequences of SEQ ID NOs: 36-38 (e.g., TCRb CDRs 1-3), and an amino acid sequence having at least 90% identity to the amino acid sequence of SEQ ID NO: 267 (e.g., TCRb full length protein).

In some embodiments, a TCR comprises the amino acid sequences of SEQ ID NOs: 31-33 (e.g., TCRa CDRs 1-3), an amino acid sequence having at least 95% identity to the amino acid sequence of SEQ ID NO: 34 (e.g., TCRa variable domain), the amino acid sequences of SEQ ID NOs: 36-38 (e.g., TCRb CDRs 1-3), and an amino acid sequence having at least 95% identity to the amino acid sequence of SEQ ID NO: 39 (e.g., TCRb variable domain). In some embodiments, a TCR comprises the amino acid sequences of SEQ ID NOs: 31-33 (e.g., TCRa CDRs 1-3), an amino acid sequence having at least 95% identity to the amino acid sequence of SEQ ID NO: 35 (e.g., TCRa full length protein), the amino acid sequences of SEQ ID NOs: 36-38 (e.g., TCRb CDRs 1-3), and an amino acid sequence having at least 95% identity to the amino acid sequence of SEQ ID NO: 40 (e.g., TCRb full length protein). In some embodiments, a TCR comprises the amino acid sequences of SEQ ID NOs: 31-33 (e.g., TCRa CDRs 1-3), an amino acid sequence having at least 95% identity to the amino acid sequence of SEQ ID NO: 266 (e.g., TCRa full length protein), the amino acid sequences of SEQ ID NOs: 36-38 (e.g., TCRb CDRs 1-3), and an amino acid sequence having at least 95% identity to the amino acid sequence of SEQ ID NO: 267 (e.g., TCRb full length protein).

In some embodiments, a TCR comprises the amino acid sequences of SEQ ID NOS: 31-33 (e.g., TCRa CDRs 1-3), an amino acid sequence having at least 98% identity to the amino acid sequence of SEQ ID NO: 34 (e.g., TCRa variable domain), the amino acid sequences of SEQ ID NOs: 36-38 (e.g., TCRb CDRs 1-3), and an amino acid sequence having at least 98% identity to the amino acid sequence of SEQ ID NO: 39 (e.g., TCRb variable domain). In some embodiments, a TCR comprises the amino acid sequences of SEQ ID NOs: 31-33 (e.g., TCRa CDRs 1-3), an amino acid sequence having at least 98% identity to the amino acid sequence of SEQ ID NO: 35 (e.g., TCRa full length protein), the amino acid sequences of SEQ ID NOs: 36-38 (e.g., TCRb CDRs 1-3), and an amino acid sequence having at least 98% identity to the amino acid sequence of SEQ ID NO: 40 (e.g., TCRb full length protein). In some embodiments, a TCR comprises the amino acid sequences of SEQ ID NOs: 31-33 (e.g., TCRa CDRs 1-3), an amino acid sequence having at least 98% identity to the amino acid sequence of SEQ ID NO: 266 (e.g., TCRa full length protein), the amino acid sequences of SEQ ID NOs: 36-38 (e.g., TCRb CDRs 1-3), and an amino acid sequence having at least 98% identity to the amino acid sequence of SEQ ID NO: 267 (e.g., TCRb full length protein).

In some embodiments, a TCR comprises the amino acid sequences of SEQ ID NOS: 41-43 (e.g., TCRa CDRs 1-3), an amino acid sequence having at least 90% identity to the amino acid sequence of SEQ ID NO: 44 (e.g., TCRa variable domain), the amino acid sequences of SEQ ID NOs: 46-48 (e.g., TCRb CDRs 1-3), and an amino acid sequence having at least 90% identity to the amino acid sequence of SEQ ID NO: 49 (e.g., TCRb variable domain). In some embodiments, a TCR comprises the amino acid sequences of SEQ ID NOs: 41-43 (e.g., TCRa CDRs 1-3), an amino acid sequence having at least 90% identity to the amino acid sequence of SEQ ID NO: 45 (e.g., TCRa full length protein), the amino acid sequences of SEQ ID NOs: 41-43 (e.g., TCRb CDRs 1-3), and an amino acid sequence having at least 90% identity to the amino acid sequence of SEQ ID NO: 50 (e.g., TCRb full length protein). In some embodiments, a TCR comprises the amino acid sequences of SEQ ID NOs: 41-43 (e.g., TCRa CDRs 1-3), an amino acid sequence having at least 90% identity to the amino acid sequence of SEQ ID NO: 268 (e.g., TCRa full length protein), the amino acid sequences of SEQ ID NOs: 41-43 (e.g., TCRb CDRs 1-3), and an amino acid sequence having at least 90% identity to the amino acid sequence of SEQ ID NO: 269 (e.g., TCRb full length protein).

In some embodiments, a TCR comprises the amino acid sequences of SEQ ID NOs: 41-43 (e.g., TCRa CDRs 1-3), an amino acid sequence having at least 95% identity to the amino acid sequence of SEQ ID NO: 44 (e.g., TCRa variable domain), the amino acid sequences of SEQ ID NOs: 46-48 (e.g., TCRb CDRs 1-3), and an amino acid sequence having at least 95% identity to the amino acid sequence of SEQ ID NO: 49 (e.g., TCRb variable domain). In some embodiments, a TCR comprises the amino acid sequences of SEQ ID NOs: 41-43 (e.g., TCRa CDRs 1-3), an amino acid sequence having at least 95% identity to the amino acid sequence of SEQ ID NO: 45 (e.g., TCRa full length protein), the amino acid sequences of SEQ ID NOs: 41-43 (e.g., TCRb CDRs 1-3), and an amino acid sequence having at least 95% identity to the amino acid sequence of SEQ ID NO: 50 (e.g., TCRb full length protein). In some embodiments, a TCR comprises the amino acid sequences of SEQ ID NOs: 41-43 (e.g., TCRa CDRs 1-3), an amino acid sequence having at least 95% identity to the amino acid sequence of SEQ ID NO: 268 (e.g., TCRa full length protein), the amino acid sequences of SEQ ID NOs: 41-43 (e.g., TCRb CDRs 1-3), and an amino acid sequence having at least 95% identity to the amino acid sequence of SEQ ID NO: 269 (e.g., TCRb full length protein).

In some embodiments, a TCR comprises the amino acid sequences of SEQ ID NOS: 41-43 (e.g., TCRa CDRs 1-3), an amino acid sequence having at least 98% identity to the amino acid sequence of SEQ ID NO: 44 (e.g., TCRa variable domain), the amino acid sequences of SEQ ID NOs: 46-48 (e.g., TCRb CDRs 1-3), and an amino acid sequence having at least 98% identity to the amino acid sequence of SEQ ID NO: 49 (e.g., TCRb variable domain). In some embodiments, a TCR comprises the amino acid sequences of SEQ ID NOs: 41-43 (e.g., TCRa CDRs 1-3), an amino acid sequence having at least 98% identity to the amino acid sequence of SEQ ID NO: 45 (e.g., TCRa full length protein), the amino acid sequences of SEQ ID NOs: 41-43 (e.g., TCRb CDRs 1-3), and an amino acid sequence having at least 98% identity to the amino acid sequence of SEQ ID NO: 50 (e.g., TCRb full length protein). In some embodiments, a TCR comprises the amino acid sequences of SEQ ID NOs: 41-43 (e.g., TCRa CDRs 1-3), an amino acid sequence having at least 98% identity to the amino acid sequence of SEQ ID NO: 268 (e.g., TCRa full length protein), the amino acid sequences of SEQ ID NOs: 41-43 (e.g., TCRb CDRs 1-3), and an amino acid sequence having at least 98% identity to the amino acid sequence of SEQ ID NO: 269 (e.g., TCRb full length protein).

In some embodiments, a TCR comprises the amino acid sequences of SEQ ID NOS: 51-53 (e.g., TCRa CDRs 1-3), an amino acid sequence having at least 90% identity to the amino acid sequence of SEQ ID NO: 54 (e.g., TCRa variable domain), the amino acid sequences of SEQ ID NOs: 51-53 (e.g., TCRb CDRs 1-3), and an amino acid sequence having at least 90% identity to the amino acid sequence of SEQ ID NO: 55 (e.g., TCRb variable domain). In some embodiments, a TCR comprises the amino acid sequences of SEQ ID NOs: 51-53 (e.g., TCRa CDRs 1-3), an amino acid sequence having at least 90% identity to the amino acid sequence of SEQ ID NO: 59 (e.g., TCRa full length protein), the amino acid sequences of SEQ ID NOS: 51-53 (e.g., TCRb CDRs 1-3), and an amino acid sequence having at least 90% identity to the amino acid sequence of SEQ ID NO: 60 (e.g., TCRb full length protein). In some embodiments, a TCR comprises the amino acid sequences of SEQ ID NOs: 51-53 (e.g., TCRa CDRs 1-3), an amino acid sequence having at least 90% identity to the amino acid sequence of SEQ ID NO: 270) (e.g., TCRa full length protein), the amino acid sequences of SEQ ID NOs: 51-53 (e.g., TCRb CDRs 1-3), and an amino acid sequence having at least 90% identity to the amino acid sequence of SEQ ID NO: 271 (e.g., TCRb full length protein).

In some embodiments, a TCR comprises the amino acid sequences of SEQ ID NOS: 51-53 (e.g., TCRa CDRs 1-3), an amino acid sequence having at least 95% identity to the amino acid sequence of SEQ ID NO: 54 (e.g., TCRa variable domain), the amino acid sequences of SEQ ID NOs: 51-53 (e.g., TCRb CDRs 1-3), and an amino acid sequence having at least 95% identity to the amino acid sequence of SEQ ID NO: 55 (e.g., TCRb variable domain). In some embodiments, a TCR comprises the amino acid sequences of SEQ ID NOs: 51-53 (e.g., TCRa CDRs 1-3), an amino acid sequence having at least 95% identity to the amino acid sequence of SEQ ID NO: 59 (e.g., TCRa full length protein), the amino acid sequences of SEQ ID NOs: 51-53 (e.g., TCRb CDRs 1-3), and an amino acid sequence having at least 95% identity to the amino acid sequence of SEQ ID NO: 60 (e.g., TCRb full length protein). In some embodiments, a TCR comprises the amino acid sequences of SEQ ID NOs: 51-53 (e.g., TCRa CDRs 1-3), an amino acid sequence having at least 95% identity to the amino acid sequence of SEQ ID NO: 270) (e.g., TCRa full length protein), the amino acid sequences of SEQ ID NOs: 51-53 (e.g., TCRb CDRs 1-3), and an amino acid sequence having at least 95% identity to the amino acid sequence of SEQ ID NO: 271 (e.g., TCRb full length protein).

In some embodiments, a TCR comprises the amino acid sequences of SEQ ID NOs: 51-53 (e.g., TCRa CDRs 1-3), an amino acid sequence having at least 98% identity to the amino acid sequence of SEQ ID NO: 54 (e.g., TCRa variable domain), the amino acid sequences of SEQ ID NOs: 51-53 (e.g., TCRb CDRs 1-3), and an amino acid sequence having at least 98% identity to the amino acid sequence of SEQ ID NO: 55 (e.g., TCRb variable domain). In some embodiments, a TCR comprises the amino acid sequences of SEQ ID NOs: 51-53 (e.g., TCRa CDRs 1-3), an amino acid sequence having at least 98% identity to the amino acid sequence of SEQ ID NO: 59 (e.g., TCRa full length protein), the amino acid sequences of SEQ ID NOs: 51-53 (e.g., TCRb CDRs 1-3), and an amino acid sequence having at least 98% identity to the amino acid sequence of SEQ ID NO: 60 (e.g., TCRb full length protein). In some embodiments, a TCR comprises the amino acid sequences of SEQ ID NOs: 51-53 (e.g., TCRa CDRs 1-3), an amino acid sequence having at least 98% identity to the amino acid sequence of SEQ ID NO: 270) (e.g., TCRa full length protein), the amino acid sequences of SEQ ID NOs: 51-53 (e.g., TCRb CDRs 1-3), and an amino acid sequence having at least 98% identity to the amino acid sequence of SEQ ID NO: 271 (e.g., TCRb full length protein).

In some embodiments, a TCR comprises the amino acid sequences of SEQ ID NOS: 61-63 (e.g., TCRa CDRs 1-3), an amino acid sequence having at least 90% identity to the amino acid sequence of SEQ ID NO: 64 (e.g., TCRa variable domain), the amino acid sequences of SEQ ID NOs: 66-68 (e.g., TCRb CDRs 1-3), and an amino acid sequence having at least 90% identity to the amino acid sequence of SEQ ID NO: 69 (e.g., TCRb variable domain). In some embodiments, a TCR comprises the amino acid sequences of SEQ ID NOs: 61-63 (e.g., TCRa CDRs 1-3), an amino acid sequence having at least 90% identity to the amino acid sequence of SEQ ID NO: 65 (e.g., TCRa full length protein), the amino acid sequences of SEQ ID NOs: 61-63 (e.g., TCRb CDRs 1-3), and an amino acid sequence having at least 90% identity to the amino acid sequence of SEQ ID NO: 70 (e.g., TCRb full length protein). In some embodiments, a TCR comprises the amino acid sequences of SEQ ID NOs: 61-63 (e.g., TCRa CDRs 1-3), an amino acid sequence having at least 90% identity to the amino acid sequence of SEQ ID NO: 272 (e.g., TCRa full length protein), the amino acid sequences of SEQ ID NOs: 61-63 (e.g., TCRb CDRs 1-3), and an amino acid sequence having at least 90% identity to the amino acid sequence of SEQ ID NO: 273 (e.g., TCRb full length protein).

In some embodiments, a TCR comprises the amino acid sequences of SEQ ID NOs: 61-63 (e.g., TCRa CDRs 1-3), an amino acid sequence having at least 95% identity to the amino acid sequence of SEQ ID NO: 64 (e.g., TCRa variable domain), the amino acid sequences of SEQ ID NOs: 66-68 (e.g., TCRb CDRs 1-3), and an amino acid sequence having at least 95% identity to the amino acid sequence of SEQ ID NO: 69 (e.g., TCRb variable domain). In some embodiments, a TCR comprises the amino acid sequences of SEQ ID NOs: 61-63 (e.g., TCRa CDRs 1-3), an amino acid sequence having at least 95% identity to the amino acid sequence of SEQ ID NO: 65 (e.g., TCRa full length protein), the amino acid sequences of SEQ ID NOs: 61-63 (e.g., TCRb CDRs 1-3), and an amino acid sequence having at least 95% identity to the amino acid sequence of SEQ ID NO: 70 (e.g., TCRb full length protein). In some embodiments, a TCR comprises the amino acid sequences of SEQ ID NOs: 61-63 (e.g., TCRa CDRs 1-3), an amino acid sequence having at least 95% identity to the amino acid sequence of SEQ ID NO: 272 (e.g., TCRa full length protein), the amino acid sequences of SEQ ID NOs: 61-63 (e.g., TCRb CDRs 1-3), and an amino acid sequence having at least 95% identity to the amino acid sequence of SEQ ID NO: 273 (e.g., TCRb full length protein).

In some embodiments, a TCR comprises the amino acid sequences of SEQ ID NOS: 61-63 (e.g., TCRa CDRs 1-3), an amino acid sequence having at least 98% identity to the amino acid sequence of SEQ ID NO: 64 (e.g., TCRa variable domain), the amino acid sequences of SEQ ID NOs: 66-68 (e.g., TCRb CDRs 1-3), and an amino acid sequence having at least 98% identity to the amino acid sequence of SEQ ID NO: 69 (e.g., TCRb variable domain). In some embodiments, a TCR comprises the amino acid sequences of SEQ ID NOs: 61-63 (e.g., TCRa CDRs 1-3), an amino acid sequence having at least 98% identity to the amino acid sequence of SEQ ID NO: 65 (e.g., TCRa full length protein), the amino acid sequences of SEQ ID NOs: 61-63 (e.g., TCRb CDRs 1-3), and an amino acid sequence having at least 98% identity to the amino acid sequence of SEQ ID NO: 70 (e.g., TCRb full length protein). In some embodiments, a TCR comprises the amino acid sequences of SEQ ID NOs: 61-63 (e.g., TCRa CDRs 1-3), an amino acid sequence having at least 98% identity to the amino acid sequence of SEQ ID NO: 272 (e.g., TCRa full length protein), the amino acid sequences of SEQ ID NOs: 61-63 (e.g., TCRb CDRs 1-3), and an amino acid sequence having at least 98% identity to the amino acid sequence of SEQ ID NO: 273 (e.g., TCRb full length protein).

In some embodiments, a TCR comprises the amino acid sequences of SEQ ID NOS: 71-73 (e.g., TCRa CDRs 1-3), an amino acid sequence having at least 90% identity to the amino acid sequence of SEQ ID NO: 74 (e.g., TCRa variable domain), the amino acid sequences of SEQ ID NOs: 76-78 (e.g., TCRb CDRs 1-3), and an amino acid sequence having at least 90% identity to the amino acid sequence of SEQ ID NO: 79 (e.g., TCRb variable domain). In some embodiments, a TCR comprises the amino acid sequences of SEQ ID NOs: 71-73 (e.g., TCRa CDRs 1-3), an amino acid sequence having at least 90% identity to the amino acid sequence of SEQ ID NO: 75 (e.g., TCRa full length protein), the amino acid sequences of SEQ ID NOS: 76-78 (e.g., TCRb CDRs 1-3), and an amino acid sequence having at least 90% identity to the amino acid sequence of SEQ ID NO: 80 (e.g., TCRb full length protein). In some embodiments, a TCR comprises the amino acid sequences of SEQ ID NOs: 71-73 (e.g., TCRa CDRs 1-3), an amino acid sequence having at least 90% identity to the amino acid sequence of SEQ ID NO: 274 (e.g., TCRa full length protein), the amino acid sequences of SEQ ID NOs: 76-78 (e.g., TCRb CDRs 1-3), and an amino acid sequence having at least 90% identity to the amino acid sequence of SEQ ID NO: 275 (e.g., TCRb full length protein).

In some embodiments, a TCR comprises the amino acid sequences of SEQ ID NOS: 71-73 (e.g., TCRa CDRs 1-3), an amino acid sequence having at least 95% identity to the amino acid sequence of SEQ ID NO: 74 (e.g., TCRa variable domain), the amino acid sequences of SEQ ID NOs: 76-78 (e.g., TCRb CDRs 1-3), and an amino acid sequence having at least 95% identity to the amino acid sequence of SEQ ID NO: 79 (e.g., TCRb variable domain). In some embodiments, a TCR comprises the amino acid sequences of SEQ ID NOs: 71-73 (e.g., TCRa CDRs 1-3), an amino acid sequence having at least 95% identity to the amino acid sequence of SEQ ID NO: 75 (e.g., TCRa full length protein), the amino acid sequences of SEQ ID NOs: 76-78 (e.g., TCRb CDRs 1-3), and an amino acid sequence having at least 95% identity to the amino acid sequence of SEQ ID NO: 80 (e.g., TCRb full length protein). In some embodiments, a TCR comprises the amino acid sequences of SEQ ID NOs: 71-73 (e.g., TCRa CDRs 1-3), an amino acid sequence having at least 95% identity to the amino acid sequence of SEQ ID NO: 274 (e.g., TCRa full length protein), the amino acid sequences of SEQ ID NOs: 76-78 (e.g., TCRb CDRs 1-3), and an amino acid sequence having at least 95% identity to the amino acid sequence of SEQ ID NO: 275 (e.g., TCRb full length protein).

In some embodiments, a TCR comprises the amino acid sequences of SEQ ID NOS: 71-73 (e.g., TCRa CDRs 1-3), an amino acid sequence having at least 98% identity to the amino acid sequence of SEQ ID NO: 74 (e.g., TCRa variable domain), the amino acid sequences of SEQ ID NOs: 76-78 (e.g., TCRb CDRs 1-3), and an amino acid sequence having at least 98% identity to the amino acid sequence of SEQ ID NO: 79 (e.g., TCRb variable domain). In some embodiments, a TCR comprises the amino acid sequences of SEQ ID NOs: 71-73 (e.g., TCRa CDRs 1-3), an amino acid sequence having at least 98% identity to the amino acid sequence of SEQ ID NO: 75 (e.g., TCRa full length protein), the amino acid sequences of SEQ ID NOS: 76-78 (e.g., TCRb CDRs 1-3), and an amino acid sequence having at least 98% identity to the amino acid sequence of SEQ ID NO: 80 (e.g., TCRb full length protein). In some embodiments, a TCR comprises the amino acid sequences of SEQ ID NOs: 71-73 (e.g., TCRa CDRs 1-3), an amino acid sequence having at least 98% identity to the amino acid sequence of SEQ ID NO: 274 (e.g., TCRa full length protein), the amino acid sequences of SEQ ID NOs: 76-78 (e.g., TCRb CDRs 1-3), and an amino acid sequence having at least 98% identity to the amino acid sequence of SEQ ID NO: 275 (e.g., TCRb full length protein).

In some embodiments, a TCR comprises the amino acid sequences of SEQ ID NOS: 81-83 (e.g., TCRa CDRs 1-3), an amino acid sequence having at least 90% identity to the amino acid sequence of SEQ ID NO: 84 (e.g., TCRa variable domain), the amino acid sequences of SEQ ID NOs: 86-88 (e.g., TCRb CDRs 1-3), and an amino acid sequence having at least 90% identity to the amino acid sequence of SEQ ID NO: 89 (e.g., TCRb variable domain). In some embodiments, a TCR comprises the amino acid sequences of SEQ ID NOs: 81-83 (e.g., TCRa CDRs 1-3), an amino acid sequence having at least 90% identity to the amino acid sequence of SEQ ID NO: 85 (e.g., TCRa full length protein), the amino acid sequences of SEQ ID NOS: 86-88 (e.g., TCRb CDRs 1-3), and an amino acid sequence having at least 90% identity to the amino acid sequence of SEQ ID NO: 90 (e.g., TCRb full length protein). In some embodiments, a TCR comprises the amino acid sequences of SEQ ID NOs: 81-83 (e.g., TCRa CDRs 1-3), an amino acid sequence having at least 90% identity to the amino acid sequence of SEQ ID NO: 276 (e.g., TCRa full length protein), the amino acid sequences of SEQ ID NOs: 86-88 (e.g., TCRb CDRs 1-3), and an amino acid sequence having at least 90% identity to the amino acid sequence of SEQ ID NO: 277 (e.g., TCRb full length protein).

In some embodiments, a TCR comprises the amino acid sequences of SEQ ID NOS: 81-83 (e.g., TCRa CDRs 1-3), an amino acid sequence having at least 95% identity to the amino acid sequence of SEQ ID NO: 84 (e.g., TCRa variable domain), the amino acid sequences of SEQ ID NOs: 86-88 (e.g., TCRb CDRs 1-3), and an amino acid sequence having at least 95% identity to the amino acid sequence of SEQ ID NO: 89 (e.g., TCRb variable domain). In some embodiments, a TCR comprises the amino acid sequences of SEQ ID NOs: 81-83 (e.g., TCRa CDRs 1-3), an amino acid sequence having at least 95% identity to the amino acid sequence of SEQ ID NO: 85 (e.g., TCRa full length protein), the amino acid sequences of SEQ ID NOS: 86-88 (e.g., TCRb CDRs 1-3), and an amino acid sequence having at least 95% identity to the amino acid sequence of SEQ ID NO: 90 (e.g., TCRb full length protein). In some embodiments, a TCR comprises the amino acid sequences of SEQ ID NOs: 81-83 (e.g., TCRa CDRs 1-3), an amino acid sequence having at least 95% identity to the amino acid sequence of SEQ ID NO: 276 (e.g., TCRa full length protein), the amino acid sequences of SEQ ID NOs: 86-88 (e.g., TCRb CDRs 1-3), and an amino acid sequence having at least 95% identity to the amino acid sequence of SEQ ID NO: 277 (e.g., TCRb full length protein).

In some embodiments, a TCR comprises the amino acid sequences of SEQ ID NOS: 81-83 (e.g., TCRa CDRs 1-3), an amino acid sequence having at least 98% identity to the amino acid sequence of SEQ ID NO: 84 (e.g., TCRa variable domain), the amino acid sequences of SEQ ID NOs: 86-88 (e.g., TCRb CDRs 1-3), and an amino acid sequence having at least 98% identity to the amino acid sequence of SEQ ID NO: 89 (e.g., TCRb variable domain). In some embodiments, a TCR comprises the amino acid sequences of SEQ ID NOs: 81-83 (e.g., TCRa CDRs 1-3), an amino acid sequence having at least 98% identity to the amino acid sequence of SEQ ID NO: 85 (e.g., TCRa full length protein), the amino acid sequences of SEQ ID NOs: 86-88 (e.g., TCRb CDRs 1-3), and an amino acid sequence having at least 98% identity to the amino acid sequence of SEQ ID NO: 90 (e.g., TCRb full length protein). In some embodiments, a TCR comprises the amino acid sequences of SEQ ID NOs: 81-83 (e.g., TCRa CDRs 1-3), an amino acid sequence having at least 98% identity to the amino acid sequence of SEQ ID NO: 276 (e.g., TCRa full length protein), the amino acid sequences of SEQ ID NOs: 86-88 (e.g., TCRb CDRs 1-3), and an amino acid sequence having at least 98% identity to the amino acid sequence of SEQ ID NO: 277 (e.g., TCRb full length protein).

In some embodiments, a TCR comprises the amino acid sequences of SEQ ID NOS: 91-93 (e.g., TCRa CDRs 1-3), an amino acid sequence having at least 90% identity to the amino acid sequence of SEQ ID NO: 94 (e.g., TCRa variable domain), the amino acid sequences of SEQ ID NOs: 96-98 (e.g., TCRb CDRs 1-3), and an amino acid sequence having at least 90% identity to the amino acid sequence of SEQ ID NO: 99 (e.g., TCRb variable domain). In some embodiments, a TCR comprises the amino acid sequences of SEQ ID NOs: 91-93 (e.g., TCRa CDRs 1-3), an amino acid sequence having at least 90% identity to the amino acid sequence of SEQ ID NO: 95 (e.g., TCRa full length protein), the amino acid sequences of SEQ ID NOS: 96-98 (e.g., TCRb CDRs 1-3), and an amino acid sequence having at least 90% identity to the amino acid sequence of SEQ ID NO: 100 (e.g., TCRb full length protein). In some embodiments, a TCR comprises the amino acid sequences of SEQ ID NOs: 91-93 (e.g., TCRa CDRs 1-3), an amino acid sequence having at least 90% identity to the amino acid sequence of SEQ ID NO: 278 (e.g., TCRa full length protein), the amino acid sequences of SEQ ID NOs: 96-98 (e.g., TCRb CDRs 1-3), and an amino acid sequence having at least 90% identity to the amino acid sequence of SEQ ID NO: 279 (e.g., TCRb full length protein).

In some embodiments, a TCR comprises the amino acid sequences of SEQ ID NOS: 91-93 (e.g., TCRa CDRs 1-3), an amino acid sequence having at least 95% identity to the amino acid sequence of SEQ ID NO: 94 (e.g., TCRa variable domain), the amino acid sequences of SEQ ID NOs: 96-98 (e.g., TCRb CDRs 1-3), and an amino acid sequence having at least 95% identity to the amino acid sequence of SEQ ID NO: 99 (e.g., TCRb variable domain). In some embodiments, a TCR comprises the amino acid sequences of SEQ ID NOs: 91-93 (e.g., TCRa CDRs 1-3), an amino acid sequence having at least 95% identity to the amino acid sequence of SEQ ID NO: 95 (e.g., TCRa full length protein), the amino acid sequences of SEQ ID NOs: 96-98 (e.g., TCRb CDRs 1-3), and an amino acid sequence having at least 95% identity to the amino acid sequence of SEQ ID NO: 100 (e.g., TCRb full length protein). In some embodiments, a TCR comprises the amino acid sequences of SEQ ID NOs: 91-93 (e.g., TCRa CDRs 1-3), an amino acid sequence having at least 95% identity to the amino acid sequence of SEQ ID NO: 278 (e.g., TCRa full length protein), the amino acid sequences of SEQ ID NOs: 96-98 (e.g., TCRb CDRs 1-3), and an amino acid sequence having at least 95% identity to the amino acid sequence of SEQ ID NO: 279 (e.g., TCRb full length protein).

In some embodiments, a TCR comprises the amino acid sequences of SEQ ID NOS: 91-93 (e.g., TCRa CDRs 1-3), an amino acid sequence having at least 98% identity to the amino acid sequence of SEQ ID NO: 94 (e.g., TCRa variable domain), the amino acid sequences of SEQ ID NOs: 96-98 (e.g., TCRb CDRs 1-3), and an amino acid sequence having at least 98% identity to the amino acid sequence of SEQ ID NO: 99 (e.g., TCRb variable domain). In some embodiments, a TCR comprises the amino acid sequences of SEQ ID NOs: 91-93 (e.g., TCRa CDRs 1-3), an amino acid sequence having at least 98% identity to the amino acid sequence of SEQ ID NO: 95 (e.g., TCRa full length protein), the amino acid sequences of SEQ ID NOs: 96-98 (e.g., TCRb CDRs 1-3), and an amino acid sequence having at least 98% identity to the amino acid sequence of SEQ ID NO: 100 (e.g., TCRb full length protein). In some embodiments, a TCR comprises the amino acid sequences of SEQ ID NOs: 91-93 (e.g., TCRa CDRs 1-3), an amino acid sequence having at least 98% identity to the amino acid sequence of SEQ ID NO: 278 (e.g., TCRa full length protein), the amino acid sequences of SEQ ID NOs: 96-98 (e.g., TCRb CDRs 1-3), and an amino acid sequence having at least 98% identity to the amino acid sequence of SEQ ID NO: 279 (e.g., TCRb full length protein).

In some embodiments, a TCR comprises the amino acid sequences of SEQ ID NOS: 110-112 (e.g., TCRa CDRs 1-3), an amino acid sequence having at least 90% identity to the amino acid sequence of SEQ ID NO: 113 (e.g., TCRa variable domain), the amino acid sequences of SEQ ID NOs: 115-117 (e.g., TCRb CDRs 1-3), and an amino acid sequence having at least 90% identity to the amino acid sequence of SEQ ID NO: 118 (e.g., TCRb variable domain). In some embodiments, a TCR comprises the amino acid sequences of SEQ ID NOs: 110-112 (e.g., TCRa CDRs 1-3), an amino acid sequence having at least 90% identity to the amino acid sequence of SEQ ID NO: 114 (e.g., TCRa full length protein), the amino acid sequences of SEQ ID NOs: 115-117 (e.g., TCRb CDRs 1-3), and an amino acid sequence having at least 90% identity to the amino acid sequence of SEQ ID NO: 119 (e.g., TCRb full length protein). In some embodiments, a TCR comprises the amino acid sequences of SEQ ID NOs: 110-112 (e.g., TCRa CDRs 1-3), an amino acid sequence having at least 90% identity to the amino acid sequence of SEQ ID NO: 280 (e.g., TCRa full length protein), the amino acid sequences of SEQ ID NOs: 115-117 (e.g., TCRb CDRs 1-3), and an amino acid sequence having at least 90% identity to the amino acid sequence of SEQ ID NO: 281 (e.g., TCRb full length protein).

In some embodiments, a TCR comprises the amino acid sequences of SEQ ID NOs: 110-112 (e.g., TCRa CDRs 1-3), an amino acid sequence having at least 95% identity to the amino acid sequence of SEQ ID NO: 113 (e.g., TCRa variable domain), the amino acid sequences of SEQ ID NOs: 115-117 (e.g., TCRb CDRs 1-3), and an amino acid sequence having at least 95% identity to the amino acid sequence of SEQ ID NO: 118 (e.g., TCRb variable domain). In some embodiments, a TCR comprises the amino acid sequences of SEQ ID NOs: 110-112 (e.g., TCRa CDRs 1-3), an amino acid sequence having at least 95% identity to the amino acid sequence of SEQ ID NO: 114 (e.g., TCRa full length protein), the amino acid sequences of SEQ ID NOs: 115-117 (e.g., TCRb CDRs 1-3), and an amino acid sequence having at least 95% identity to the amino acid sequence of SEQ ID NO: 119 (e.g., TCRb full length protein). In some embodiments, a TCR comprises the amino acid sequences of SEQ ID NOs: 110-112 (e.g., TCRa CDRs 1-3), an amino acid sequence having at least 95% identity to the amino acid sequence of SEQ ID NO: 280 (e.g., TCRa full length protein), the amino acid sequences of SEQ ID NOs: 115-117 (e.g., TCRb CDRs 1-3), and an amino acid sequence having at least 95% identity to the amino acid sequence of SEQ ID NO: 281 (e.g., TCRb full length protein).

In some embodiments, a TCR comprises the amino acid sequences of SEQ ID NOS: 110-112 (e.g., TCRa CDRs 1-3), an amino acid sequence having at least 98% identity to the amino acid sequence of SEQ ID NO: 113 (e.g., TCRa variable domain), the amino acid sequences of SEQ ID NOs: 115-117 (e.g., TCRb CDRs 1-3), and an amino acid sequence having at least 98% identity to the amino acid sequence of SEQ ID NO: 118 (e.g., TCRb variable domain). In some embodiments, a TCR comprises the amino acid sequences of SEQ ID NOs: 110-112 (e.g., TCRa CDRs 1-3), an amino acid sequence having at least 98% identity to the amino acid sequence of SEQ ID NO: 114 (e.g., TCRa full length protein), the amino acid sequences of SEQ ID NOs: 115-117 (e.g., TCRb CDRs 1-3), and an amino acid sequence having at least 98% identity to the amino acid sequence of SEQ ID NO: 119 (e.g., TCRb full length protein). In some embodiments, a TCR comprises the amino acid sequences of SEQ ID NOs: 110-112 (e.g., TCRa CDRs 1-3), an amino acid sequence having at least 98% identity to the amino acid sequence of SEQ ID NO: 280 (e.g., TCRa full length protein), the amino acid sequences of SEQ ID NOs: 115-117 (e.g., TCRb CDRs 1-3), and an amino acid sequence having at least 98% identity to the amino acid sequence of SEQ ID NO: 281 (e.g., TCRb full length protein).

In some embodiments, a TCR comprises the amino acid sequences of SEQ ID NOs: 120-122 (e.g., TCRa CDRs 1-3), an amino acid sequence having at least 90% identity to the amino acid sequence of SEQ ID NO: 123 (e.g., TCRa variable domain), the amino acid sequences of SEQ ID NOs: 125-127 (e.g., TCRb CDRs 1-3), and an amino acid sequence having at least 90% identity to the amino acid sequence of SEQ ID NO: 128 (e.g., TCRb variable domain). In some embodiments, a TCR comprises the amino acid sequences of SEQ ID NOs: 120-122 (e.g., TCRa CDRs 1-3), an amino acid sequence having at least 90% identity to the amino acid sequence of SEQ ID NO: 124 (e.g., TCRa full length protein), the amino acid sequences of SEQ ID NOs: 125-127 (e.g., TCRb CDRs 1-3), and an amino acid sequence having at least 90% identity to the amino acid sequence of SEQ ID NO: 129 (e.g., TCRb full length protein). In some embodiments, a TCR comprises the amino acid sequences of SEQ ID NOs: 120-122 (e.g., TCRa CDRs 1-3), an amino acid sequence having at least 90% identity to the amino acid sequence of SEQ ID NO: 282 (e.g., TCRa full length protein), the amino acid sequences of SEQ ID NOs: 125-127 (e.g., TCRb CDRs 1-3), and an amino acid sequence having at least 90% identity to the amino acid sequence of SEQ ID NO: 283 (e.g., TCRb full length protein).

In some embodiments, a TCR comprises the amino acid sequences of SEQ ID NOS: 120-122 (e.g., TCRa CDRs 1-3), an amino acid sequence having at least 95% identity to the amino acid sequence of SEQ ID NO: 123 (e.g., TCRa variable domain), the amino acid sequences of SEQ ID NOs: 125-127 (e.g., TCRb CDRs 1-3), and an amino acid sequence having at least 95% identity to the amino acid sequence of SEQ ID NO: 128 (e.g., TCRb variable domain). In some embodiments, a TCR comprises the amino acid sequences of SEQ ID NOs: 120-122 (e.g., TCRa CDRs 1-3), an amino acid sequence having at least 95% identity to the amino acid sequence of SEQ ID NO: 124 (e.g., TCRa full length protein), the amino acid sequences of SEQ ID NOs: 125-127 (e.g., TCRb CDRs 1-3), and an amino acid sequence having at least 95% identity to the amino acid sequence of SEQ ID NO: 129 (e.g., TCRb full length protein). In some embodiments, a TCR comprises the amino acid sequences of SEQ ID NOs: 120-122 (e.g., TCRa CDRs 1-3), an amino acid sequence having at least 95% identity to the amino acid sequence of SEQ ID NO: 282 (e.g., TCRa full length protein), the amino acid sequences of SEQ ID NOs: 125-127 (e.g., TCRb CDRs 1-3), and an amino acid sequence having at least 95% identity to the amino acid sequence of SEQ ID NO: 283 (e.g., TCRb full length protein).

In some embodiments, a TCR comprises the amino acid sequences of SEQ ID NOS: 120-122 (e.g., TCRa CDRs 1-3), an amino acid sequence having at least 98% identity to the amino acid sequence of SEQ ID NO: 123 (e.g., TCRa variable domain), the amino acid sequences of SEQ ID NOs: 125-127 (e.g., TCRb CDRs 1-3), and an amino acid sequence having at least 98% identity to the amino acid sequence of SEQ ID NO: 128 (e.g., TCRb variable domain). In some embodiments, a TCR comprises the amino acid sequences of SEQ ID NOs: 120-122 (e.g., TCRa CDRs 1-3), an amino acid sequence having at least 98% identity to the amino acid sequence of SEQ ID NO: 124 (e.g., TCRa full length protein), the amino acid sequences of SEQ ID NOs: 125-127 (e.g., TCRb CDRs 1-3), and an amino acid sequence having at least 98% identity to the amino acid sequence of SEQ ID NO: 129 (e.g., TCRb full length protein). In some embodiments, a TCR comprises the amino acid sequences of SEQ ID NOs: 120-122 (e.g., TCRa CDRs 1-3), an amino acid sequence having at least 98% identity to the amino acid sequence of SEQ ID NO: 282 (e.g., TCRa full length protein), the amino acid sequences of SEQ ID NOs: 125-127 (e.g., TCRb CDRs 1-3), and an amino acid sequence having at least 98% identity to the amino acid sequence of SEQ ID NO: 283 (e.g., TCRb full length protein).

In some embodiments, a TCR comprises the amino acid sequences of SEQ ID NOs: 130-132 (e.g., TCRa CDRs 1-3), an amino acid sequence having at least 90% identity to the amino acid sequence of SEQ ID NO: 133 (e.g., TCRa variable domain), the amino acid sequences of SEQ ID NOs: 135-137 (e.g., TCRb CDRs 1-3), and an amino acid sequence having at least 90% identity to the amino acid sequence of SEQ ID NO: 138 (e.g., TCRb variable domain). In some embodiments, a TCR comprises the amino acid sequences of SEQ

ID NOs: 130-132 (e.g., TCRa CDRs 1-3), an amino acid sequence having at least 90% identity to the amino acid sequence of SEQ ID NO: 134 (e.g., TCRa full length protein), the amino acid sequences of SEQ ID NOs: 135-137 (e.g., TCRb CDRs 1-3), and an amino acid sequence having at least 90% identity to the amino acid sequence of SEQ ID NO: 139 (e.g., TCRb full length protein). In some embodiments, a TCR comprises the amino acid sequences of SEQ ID NOs: 130-132 (e.g., TCRa CDRs 1-3), an amino acid sequence having at least 90% identity to the amino acid sequence of SEQ ID NO: 284 (e.g., TCRa full length protein), the amino acid sequences of SEQ ID NOs: 135-137 (e.g., TCRb CDRs 1-3), and an amino acid sequence having at least 90% identity to the amino acid sequence of SEQ ID NO: 285 (e.g., TCRb full length protein).

In some embodiments, a TCR comprises the amino acid sequences of SEQ ID NOS: 130-132 (e.g., TCRa CDRs 1-3), an amino acid sequence having at least 95% identity to the amino acid sequence of SEQ ID NO: 133 (e.g., TCRa variable domain), the amino acid sequences of SEQ ID NOs: 135-137 (e.g., TCRb CDRs 1-3), and an amino acid sequence having at least 95% identity to the amino acid sequence of SEQ ID NO: 138 (e.g., TCRb variable domain). In some embodiments, a TCR comprises the amino acid sequences of SEQ ID NOs: 130-132 (e.g., TCRa CDRs 1-3), an amino acid sequence having at least 95% identity to the amino acid sequence of SEQ ID NO: 134 (e.g., TCRa full length protein), the amino acid sequences of SEQ ID NOs: 135-137 (e.g., TCRb CDRs 1-3), and an amino acid sequence having at least 95% identity to the amino acid sequence of SEQ ID NO: 139 (e.g., TCRb full length protein). In some embodiments, a TCR comprises the amino acid sequences of SEQ ID NOs: 130-132 (e.g., TCRa CDRs 1-3), an amino acid sequence having at least 95% identity to the amino acid sequence of SEQ ID NO: 284 (e.g., TCRa full length protein), the amino acid sequences of SEQ ID NOs: 135-137 (e.g., TCRb CDRs 1-3), and an amino acid sequence having at least 95% identity to the amino acid sequence of SEQ ID NO: 285 (e.g., TCRb full length protein).

In some embodiments, a TCR comprises the amino acid sequences of SEQ ID NOS: 130-132 (e.g., TCRa CDRs 1-3), an amino acid sequence having at least 98% identity to the amino acid sequence of SEQ ID NO: 133 (e.g., TCRa variable domain), the amino acid sequences of SEQ ID NOs: 135-137 (e.g., TCRb CDRs 1-3), and an amino acid sequence having at least 98% identity to the amino acid sequence of SEQ ID NO: 138 (e.g., TCRb variable domain). In some embodiments, a TCR comprises the amino acid sequences of SEQ ID NOs: 130-132 (e.g., TCRa CDRs 1-3), an amino acid sequence having at least 98% identity to the amino acid sequence of SEQ ID NO: 134 (e.g., TCRa full length protein), the amino acid sequences of SEQ ID NOs: 135-137 (e.g., TCRb CDRs 1-3), and an amino acid sequence having at least 98% identity to the amino acid sequence of SEQ ID NO: 139 (e.g., TCRb full length protein). In some embodiments, a TCR comprises the amino acid sequences of SEQ ID NOs: 130-132 (e.g., TCRa CDRs 1-3), an amino acid sequence having at least 98% identity to the amino acid sequence of SEQ ID NO: 284 (e.g., TCRa full length protein), the amino acid sequences of SEQ ID NOs: 135-137 (e.g., TCRb CDRs 1-3), and an amino acid sequence having at least 98% identity to the amino acid sequence of SEQ ID NO: 285 (e.g., TCRb full length protein).

In some embodiments, a TCR comprises the amino acid sequences of SEQ ID NOS: 140-142 (e.g., TCRa CDRs 1-3), an amino acid sequence having at least 90% identity to the amino acid sequence of SEQ ID NO: 143 (e.g., TCRa variable domain), the amino acid sequences of SEQ ID NOs: 145-147 (e.g., TCRb CDRs 1-3), and an amino acid sequence having at least 90% identity to the amino acid sequence of SEQ ID NO: 148 (e.g., TCRb variable domain). In some embodiments, a TCR comprises the amino acid sequences of SEQ ID NOs: 140-142 (e.g., TCRa CDRs 1-3), an amino acid sequence having at least 90% identity to the amino acid sequence of SEQ ID NO: 144 (e.g., TCRa full length protein), the amino acid sequences of SEQ ID NOs: 145-147 (e.g., TCRb CDRs 1-3), and an amino acid sequence having at least 90% identity to the amino acid sequence of SEQ ID NO: 149 (e.g., TCRb full length protein). In some embodiments, a TCR comprises the amino acid sequences of SEQ ID NOs: 140-142 (e.g., TCRa CDRs 1-3), an amino acid sequence having at least 90% identity to the amino acid sequence of SEQ ID NO: 286 (e.g., TCRa full length protein), the amino acid sequences of SEQ ID NOs: 145-147 (e.g., TCRb CDRs 1-3), and an amino acid sequence having at least 90% identity to the amino acid sequence of SEQ ID NO: 287 (e.g., TCRb full length protein).

In some embodiments, a TCR comprises the amino acid sequences of SEQ ID NOS: 140-142 (e.g., TCRa CDRs 1-3), an amino acid sequence having at least 95% identity to the amino acid sequence of SEQ ID NO: 143 (e.g., TCRa variable domain), the amino acid sequences of SEQ ID NOs: 145-147 (e.g., TCRb CDRs 1-3), and an amino acid sequence having at least 95% identity to the amino acid sequence of SEQ ID NO: 148 (e.g., TCRb variable domain). In some embodiments, a TCR comprises the amino acid sequences of SEQ ID NOs: 140-142 (e.g., TCRa CDRs 1-3), an amino acid sequence having at least 95% identity to the amino acid sequence of SEQ ID NO: 144 (e.g., TCRa full length protein), the amino acid sequences of SEQ ID NOs: 145-147 (e.g., TCRb CDRs 1-3), and an amino acid sequence having at least 95% identity to the amino acid sequence of SEQ ID NO: 149 (e.g., TCRb full length protein). In some embodiments, a TCR comprises the amino acid sequences of SEQ ID NOs: 140-142 (e.g., TCRa CDRs 1-3), an amino acid sequence having at least 95% identity to the amino acid sequence of SEQ ID NO: 286 (e.g., TCRa full length protein), the amino acid sequences of SEQ ID NOs: 145-147 (e.g., TCRb CDRs 1-3), and an amino acid sequence having at least 95% identity to the amino acid sequence of SEQ ID NO: 287 (e.g., TCRb full length protein).

In some embodiments, a TCR comprises the amino acid sequences of SEQ ID NOS: 140-142 (e.g., TCRa CDRs 1-3), an amino acid sequence having at least 98% identity to the amino acid sequence of SEQ ID NO: 143 (e.g., TCRa variable domain), the amino acid sequences of SEQ ID NOs: 145-147 (e.g., TCRb CDRs 1-3), and an amino acid sequence having at least 98% identity to the amino acid sequence of SEQ ID NO: 148 (e.g., TCRb variable domain). In some embodiments, a TCR comprises the amino acid sequences of SEQ ID NOs: 140-142 (e.g., TCRa CDRs 1-3), an amino acid sequence having at least 98% identity to the amino acid sequence of SEQ ID NO: 144 (e.g., TCRa full length protein), the amino acid sequences of SEQ ID NOs: 145-147 (e.g., TCRb CDRs 1-3), and an amino acid sequence having at least 98% identity to the amino acid sequence of SEQ ID NO: 149 (e.g., TCRb full length protein). In some embodiments, a TCR comprises the amino acid sequences of SEQ ID NOs: 140-142 (e.g., TCRa CDRs 1-3), an amino acid sequence having at least 98% identity to the amino acid sequence of SEQ ID NO: 286 (e.g., TCRa full length protein), the amino acid sequences of SEQ ID NOs: 145-147 (e.g., TCRb CDRs 1-3), and an amino acid sequence having at least 98% identity to the amino acid sequence of SEQ ID NO: 287 (e.g., TCRb full length protein).

In some embodiments, a TCR comprises the amino acid sequences of SEQ ID NOs: 150-152 (e.g., TCRa CDRs 1-3), an amino acid sequence having at least 90% identity to the amino acid sequence of SEQ ID NO: 153 (e.g., TCRa variable domain), the amino acid sequences of SEQ ID NOs: 155-157 (e.g., TCRb CDRs 1-3), and an amino acid sequence having at least 90% identity to the amino acid sequence of SEQ ID NO: 158 (e.g., TCRb variable domain). In some embodiments, a TCR comprises the amino acid sequences of SEQ ID NOs: 150-152 (e.g., TCRa CDRs 1-3), an amino acid sequence having at least 90% identity to the amino acid sequence of SEQ ID NO: 154 (e.g., TCRa full length protein), the amino acid sequences of SEQ ID NOs: 155-157 (e.g., TCRb CDRs 1-3), and an amino acid sequence having at least 90% identity to the amino acid sequence of SEQ ID NO: 159 (e.g., TCRb full length protein). In some embodiments, a TCR comprises the amino acid sequences of SEQ ID NOs: 150-152 (e.g., TCRa CDRs 1-3), an amino acid sequence having at least 90% identity to the amino acid sequence of SEQ ID NO: 288 (e.g., TCRa full length protein), the amino acid sequences of SEQ ID NOs: 155-157 (e.g., TCRb CDRs 1-3), and an amino acid sequence having at least 90% identity to the amino acid sequence of SEQ ID NO: 289 (e.g., TCRb full length protein).

In some embodiments, a TCR comprises the amino acid sequences of SEQ ID NOs: 150-152 (e.g., TCRa CDRs 1-3), an amino acid sequence having at least 95% identity to the amino acid sequence of SEQ ID NO: 153 (e.g., TCRa variable domain), the amino acid sequences of SEQ ID NOs: 155-157 (e.g., TCRb CDRs 1-3), and an amino acid sequence having at least 95% identity to the amino acid sequence of SEQ ID NO: 158 (e.g., TCRb variable domain). In some embodiments, a TCR comprises the amino acid sequences of SEQ ID NOs: 150-152 (e.g., TCRa CDRs 1-3), an amino acid sequence having at least 95% identity to the amino acid sequence of SEQ ID NO: 154 (e.g., TCRa full length protein), the amino acid sequences of SEQ ID NOs: 155-157 (e.g., TCRb CDRs 1-3), and an amino acid sequence having at least 95% identity to the amino acid sequence of SEQ ID NO: 159 (e.g., TCRb full length protein). In some embodiments, a TCR comprises the amino acid sequences of SEQ ID NOs: 150-152 (e.g., TCRa CDRs 1-3), an amino acid sequence having at least 95% identity to the amino acid sequence of SEQ ID NO: 288 (e.g., TCRa full length protein), the amino acid sequences of SEQ ID NOs: 155-157 (e.g., TCRb CDRs 1-3), and an amino acid sequence having at least 95% identity to the amino acid sequence of SEQ ID NO: 289 (e.g., TCRb full length protein).

In some embodiments, a TCR comprises the amino acid sequences of SEQ ID NOS: 150-152 (e.g., TCRa CDRs 1-3), an amino acid sequence having at least 98% identity to the amino acid sequence of SEQ ID NO: 153 (e.g., TCRa variable domain), the amino acid sequences of SEQ ID NOs: 155-157 (e.g., TCRb CDRs 1-3), and an amino acid sequence having at least 98% identity to the amino acid sequence of SEQ ID NO: 158 (e.g., TCRb variable domain). In some embodiments, a TCR comprises the amino acid sequences of SEQ ID NOs: 150-152 (e.g., TCRa CDRs 1-3), an amino acid sequence having at least 98% identity to the amino acid sequence of SEQ ID NO: 154 (e.g., TCRa full length protein), the amino acid sequences of SEQ ID NOs: 155-157 (e.g., TCRb CDRs 1-3), and an amino acid sequence having at least 98% identity to the amino acid sequence of SEQ ID NO: 159 (e.g., TCRb full length protein). In some embodiments, a TCR comprises the amino acid sequences of SEQ ID NOs: 150-152 (e.g., TCRa CDRs 1-3), an amino acid sequence having at least 98% identity to the amino acid sequence of SEQ ID NO: 290 (e.g., TCRa full length protein), the amino acid sequences of SEQ ID NOs: 155-157 (e.g., TCRb CDRs 1-3), and an amino acid sequence having at least 98% identity to the amino acid sequence of SEQ ID NO: 291 (e.g., TCRb full length protein).

In some embodiments, a TCR comprises the amino acid sequences of SEQ ID NOs: 160-162 (e.g., TCRa CDRs 1-3), an amino acid sequence having at least 90% identity to the amino acid sequence of SEQ ID NO: 163 (e.g., TCRa variable domain), the amino acid sequences of SEQ ID NOs: 165-167 (e.g., TCRb CDRs 1-3), and an amino acid sequence having at least 90% identity to the amino acid sequence of SEQ ID NO: 168 (e.g., TCRb variable domain). In some embodiments, a TCR comprises the amino acid sequences of SEQ ID NOs: 160-162 (e.g., TCRa CDRs 1-3), an amino acid sequence having at least 90% identity to the amino acid sequence of SEQ ID NO: 164 (e.g., TCRa full length protein), the amino acid sequences of SEQ ID NOs: 165-167 (e.g., TCRb CDRs 1-3), and an amino acid sequence having at least 90% identity to the amino acid sequence of SEQ ID NO: 169 (e.g., TCRb full length protein). In some embodiments, a TCR comprises the amino acid sequences of SEQ ID NOs: 160-162 (e.g., TCRa CDRs 1-3), an amino acid sequence having at least 90% identity to the amino acid sequence of SEQ ID NO: 290 (e.g., TCRa full length protein), the amino acid sequences of SEQ ID NOs: 165-167 (e.g., TCRb CDRs 1-3), and an amino acid sequence having at least 90% identity to the amino acid sequence of SEQ ID NO: 291 (e.g., TCRb full length protein).

In some embodiments, a TCR comprises the amino acid sequences of SEQ ID NOs: 160-162 (e.g., TCRa CDRs 1-3), an amino acid sequence having at least 95% identity to the amino acid sequence of SEQ ID NO: 163 (e.g., TCRa variable domain), the amino acid sequences of SEQ ID NOs: 165-167 (e.g., TCRb CDRs 1-3), and an amino acid sequence having at least 95% identity to the amino acid sequence of SEQ ID NO: 168 (e.g., TCRb variable domain). In some embodiments, a TCR comprises the amino acid sequences of SEQ ID NOs: 160-162 (e.g., TCRa CDRs 1-3), an amino acid sequence having at least 95% identity to the amino acid sequence of SEQ ID NO: 164 (e.g., TCRa full length protein), the amino acid sequences of SEQ ID NOs: 165-167 (e.g., TCRb CDRs 1-3), and an amino acid sequence having at least 95% identity to the amino acid sequence of SEQ ID NO: 169 (e.g., TCRb full length protein). In some embodiments, a TCR comprises the amino acid sequences of SEQ ID NOs: 160-162 (e.g., TCRa CDRs 1-3), an amino acid sequence having at least 95% identity to the amino acid sequence of SEQ ID NO: 290 (e.g., TCRa full length protein), the amino acid sequences of SEQ ID NOs: 165-167 (e.g., TCRb CDRs 1-3), and an amino acid sequence having at least 95% identity to the amino acid sequence of SEQ ID NO: 291 (e.g., TCRb full length protein).

In some embodiments, a TCR comprises the amino acid sequences of SEQ ID NOS: 160-162 (e.g., TCRa CDRs 1-3), an amino acid sequence having at least 98% identity to the amino acid sequence of SEQ ID NO: 163 (e.g., TCRa variable domain), the amino acid sequences of SEQ ID NOs: 165-167 (e.g., TCRb CDRs 1-3), and an amino acid sequence having at least 98% identity to the amino acid sequence of SEQ ID NO: 168 (e.g., TCRb variable domain). In some embodiments, a TCR comprises the amino acid sequences of SEQ ID NOs: 160-162 (e.g., TCRa CDRs 1-3), an amino acid sequence having at least 98% identity to the amino acid sequence of SEQ ID NO: 164 (e.g., TCRa full length protein), the amino acid sequences of SEQ ID NOs: 165-167 (e.g., TCRb CDRs 1-3), and an amino acid sequence having at least 98% identity to the amino acid sequence of SEQ ID NO: 169 (e.g., TCRb full length protein). In some embodiments, a TCR comprises the amino acid sequences of SEQ ID NOs: 160-162 (e.g., TCRa CDRs 1-3), an amino acid sequence having at least 98% identity to the amino acid sequence of SEQ ID NO: 290 (e.g., TCRa full length protein), the amino acid sequences of SEQ ID NOs: 165-167 (e.g., TCRb CDRs 1-3), and an amino acid sequence having at least 98% identity to the amino acid sequence of SEQ ID NO: 291 (e.g., TCRb full length protein).

In some embodiments, a TCR comprises the amino acid sequences of SEQ ID NOs: 170-172 (e.g., TCRa CDRs 1-3), an amino acid sequence having at least 90% identity to the amino acid sequence of SEQ ID NO: 173 (e.g., TCRa variable domain), the amino acid sequences of SEQ ID NOs: 175-177 (e.g., TCRb CDRs 1-3), and an amino acid sequence having at least 90% identity to the amino acid sequence of SEQ ID NO: 178 (e.g., TCRb variable domain). In some embodiments, a TCR comprises the amino acid sequences of SEQ ID NOs: 170-172 (e.g., TCRa CDRs 1-3), an amino acid sequence having at least 90% identity to the amino acid sequence of SEQ ID NO: 174 (e.g., TCRa full length protein), the amino acid sequences of SEQ ID NOs: 175-177 (e.g., TCRb CDRs 1-3), and an amino acid sequence having at least 90% identity to the amino acid sequence of SEQ ID NO: 179 (e.g., TCRb full length protein). In some embodiments, a TCR comprises the amino acid sequences of SEQ ID NOs: 170-172 (e.g., TCRa CDRs 1-3), an amino acid sequence having at least 90% identity to the amino acid sequence of SEQ ID NO: 292 (e.g., TCRa full length protein), the amino acid sequences of SEQ ID NOs: 175-177 (e.g., TCRb CDRs 1-3), and an amino acid sequence having at least 90% identity to the amino acid sequence of SEQ ID NO: 293 (e.g., TCRb full length protein).

In some embodiments, a TCR comprises the amino acid sequences of SEQ ID NOs: 170-172 (e.g., TCRa CDRs 1-3), an amino acid sequence having at least 95% identity to the amino acid sequence of SEQ ID NO: 173 (e.g., TCRa variable domain), the amino acid sequences of SEQ ID NOs: 175-177 (e.g., TCRb CDRs 1-3), and an amino acid sequence having at least 95% identity to the amino acid sequence of SEQ ID NO: 178 (e.g., TCRb variable domain). In some embodiments, a TCR comprises the amino acid sequences of SEQ ID NOs: 170-172 (e.g., TCRa CDRs 1-3), an amino acid sequence having at least 95% identity to the amino acid sequence of SEQ ID NO: 174 (e.g., TCRa full length protein), the amino acid sequences of SEQ ID NOs: 175-177 (e.g., TCRb CDRs 1-3), and an amino acid sequence having at least 95% identity to the amino acid sequence of SEQ ID NO: 179 (e.g., TCRb full length protein). In some embodiments, a TCR comprises the amino acid sequences of SEQ ID NOs: 170-172 (e.g., TCRa CDRs 1-3), an amino acid sequence having at least 95% identity to the amino acid sequence of SEQ ID NO: 292 (e.g., TCRa full length protein), the amino acid sequences of SEQ ID NOs: 175-177 (e.g., TCRb CDRs 1-3), and an amino acid sequence having at least 95% identity to the amino acid sequence of SEQ ID NO: 293 (e.g., TCRb full length protein).

In some embodiments, a TCR comprises the amino acid sequences of SEQ ID NOs: 170-172 (e.g., TCRa CDRs 1-3), an amino acid sequence having at least 98% identity to the amino acid sequence of SEQ ID NO: 173 (e.g., TCRa variable domain), the amino acid sequences of SEQ ID NOs: 175-177 (e.g., TCRb CDRs 1-3), and an amino acid sequence having at least 98% identity to the amino acid sequence of SEQ ID NO: 178 (e.g., TCRb variable domain). In some embodiments, a TCR comprises the amino acid sequences of SEQ ID NOs: 170-172 (e.g., TCRa CDRs 1-3), an amino acid sequence having at least 98% identity to the amino acid sequence of SEQ ID NO: 174 (e.g., TCRa full length protein), the amino acid sequences of SEQ ID NOs: 175-177 (e.g., TCRb CDRs 1-3), and an amino acid sequence having at least 98% identity to the amino acid sequence of SEQ ID NO: 179 (e.g., TCRb full length protein). In some embodiments, a TCR comprises the amino acid sequences of SEQ ID NOs: 170-172 (e.g., TCRa CDRs 1-3), an amino acid sequence having at least 98% identity to the amino acid sequence of SEQ ID NO: 292 (e.g., TCRa full length protein), the amino acid sequences of SEQ ID NOs: 175-177 (e.g., TCRb CDRs 1-3), and an amino acid sequence having at least 98% identity to the amino acid sequence of SEQ ID NO: 293 (e.g., TCRb full length protein).

In some embodiments, a TCR comprises the amino acid sequences of SEQ ID NOS: 180-182 (e.g., TCRa CDRs 1-3), an amino acid sequence having at least 90% identity to the amino acid sequence of SEQ ID NO: 183 (e.g., TCRa variable domain), the amino acid sequences of SEQ ID NOs: 185-187 (e.g., TCRb CDRs 1-3), and an amino acid sequence having at least 90% identity to the amino acid sequence of SEQ ID NO: 188 (e.g., TCRb variable domain). In some embodiments, a TCR comprises the amino acid sequences of SEQ ID NOs: 180-182 (e.g., TCRa CDRs 1-3), an amino acid sequence having at least 90% identity to the amino acid sequence of SEQ ID NO: 294 (e.g., TCRa full length protein), the amino acid sequences of SEQ ID NOs: 185-187 (e.g., TCRb CDRs 1-3), and an amino acid sequence having at least 90% identity to the amino acid sequence of SEQ ID NO: 295 (e.g., TCRb full length protein). In some embodiments, a TCR comprises the amino acid sequences of SEQ ID NOs: 180-182 (e.g., TCRa CDRs 1-3), an amino acid sequence having at least 90% identity to the amino acid sequence of SEQ ID NO: 184 (e.g., TCRa full length protein), the amino acid sequences of SEQ ID NOs: 185-187 (e.g., TCRb CDRs 1-3), and an amino acid sequence having at least 90% identity to the amino acid sequence of SEQ ID NO: 189 (e.g., TCRb full length protein).

In some embodiments, a TCR comprises the amino acid sequences of SEQ ID NOS: 180-182 (e.g., TCRa CDRs 1-3), an amino acid sequence having at least 95% identity to the amino acid sequence of SEQ ID NO: 183 (e.g., TCRa variable domain), the amino acid sequences of SEQ ID NOs: 185-187 (e.g., TCRb CDRs 1-3), and an amino acid sequence having at least 95% identity to the amino acid sequence of SEQ ID NO: 188 (e.g., TCRb variable domain). In some embodiments, a TCR comprises the amino acid sequences of SEQ ID NOs: 180-182 (e.g., TCRa CDRs 1-3), an amino acid sequence having at least 95% identity to the amino acid sequence of SEQ ID NO: 294 (e.g., TCRa full length protein), the amino acid sequences of SEQ ID NOs: 185-187 (e.g., TCRb CDRs 1-3), and an amino acid sequence having at least 95% identity to the amino acid sequence of SEQ ID NO: 295 (e.g., TCRb full length protein). In some embodiments, a TCR comprises the amino acid sequences of SEQ ID NOs: 180-182 (e.g., TCRa CDRs 1-3), an amino acid sequence having at least 95% identity to the amino acid sequence of SEQ ID NO: 184 (e.g., TCRa full length protein), the amino acid sequences of SEQ ID NOs: 185-187 (e.g., TCRb CDRs 1-3), and an amino acid sequence having at least 95% identity to the amino acid sequence of SEQ ID NO: 189 (e.g., TCRb full length protein).

In some embodiments, a TCR comprises the amino acid sequences of SEQ ID NOs: 180-182 (e.g., TCRa CDRs 1-3), an amino acid sequence having at least 98% identity to the amino acid sequence of SEQ ID NO: 183 (e.g., TCRa variable domain), the amino acid sequences of SEQ ID NOs: 185-187 (e.g., TCRb CDRs 1-3), and an amino acid sequence having at least 98% identity to the amino acid sequence of SEQ ID NO: 188 (e.g., TCRb variable domain). In some embodiments, a TCR comprises the amino acid sequences of SEQ ID NOs: 180-182 (e.g., TCRa CDRs 1-3), an amino acid sequence having at least 98% identity to the amino acid sequence of SEQ ID NO: 294 (e.g., TCRa full length protein), the amino acid sequences of SEQ ID NOs: 185-187 (e.g., TCRb CDRs 1-3), and an amino acid sequence having at least 98% identity to the amino acid sequence of SEQ ID NO: 295 (e.g., TCRb full length protein). In some embodiments, a TCR comprises the amino acid sequences of SEQ ID NOs: 180-182 (e.g., TCRa CDRs 1-3), an amino acid sequence having at least 98% identity to the amino acid sequence of SEQ ID NO: 184 (e.g., TCRa full length protein), the amino acid sequences of SEQ ID NOs: 185-187 (e.g., TCRb CDRs 1-3), and an amino acid sequence having at least 98% identity to the amino acid sequence of SEQ ID NO: 189 (e.g., TCRb full length protein).

In some embodiments, a TCR comprises the amino acid sequences of SEQ ID NOS: 190-192 (e.g., TCRa CDRs 1-3), an amino acid sequence having at least 90% identity to the amino acid sequence of SEQ ID NO: 193 (e.g., TCRa variable domain), the amino acid sequences of SEQ ID NOs: 195-197 (e.g., TCRb CDRs 1-3), and an amino acid sequence having at least 90% identity to the amino acid sequence of SEQ ID NO: 198 (e.g., TCRb variable domain). In some embodiments, a TCR comprises the amino acid sequences of SEQ ID NOs: 190-192 (e.g., TCRa CDRs 1-3), an amino acid sequence having at least 90% identity to the amino acid sequence of SEQ ID NO: 296 (e.g., TCRa full length protein), the amino acid sequences of SEQ ID NOs: 195-197 (e.g., TCRb CDRs 1-3), and an amino acid sequence having at least 90% identity to the amino acid sequence of SEQ ID NO: 297 (e.g., TCRb full length protein). In some embodiments, a TCR comprises the amino acid sequences of SEQ ID NOs: 190-192 (e.g., TCRa CDRs 1-3), an amino acid sequence having at least 90% identity to the amino acid sequence of SEQ ID NO: 194 (e.g., TCRa full length protein), the amino acid sequences of SEQ ID NOs: 195-197 (e.g., TCRb CDRs 1-3), and an amino acid sequence having at least 90% identity to the amino acid sequence of SEQ ID NO: 199 (e.g., TCRb full length protein).

In some embodiments, a TCR comprises the amino acid sequences of SEQ ID NOS: 190-192 (e.g., TCRa CDRs 1-3), an amino acid sequence having at least 95% identity to the amino acid sequence of SEQ ID NO: 193 (e.g., TCRa variable domain), the amino acid sequences of SEQ ID NOs: 195-197 (e.g., TCRb CDRs 1-3), and an amino acid sequence having at least 95% identity to the amino acid sequence of SEQ ID NO: 198 (e.g., TCRb variable domain). In some embodiments, a TCR comprises the amino acid sequences of SEQ ID NOs: 190-192 (e.g., TCRa CDRs 1-3), an amino acid sequence having at least 95% identity to the amino acid sequence of SEQ ID NO: 296 (e.g., TCRa full length protein), the amino acid sequences of SEQ ID NOs: 195-197 (e.g., TCRb CDRs 1-3), and an amino acid sequence having at least 95% identity to the amino acid sequence of SEQ ID NO: 297 (e.g., TCRb full length protein). In some embodiments, a TCR comprises the amino acid sequences of SEQ ID NOs: 190-192 (e.g., TCRa CDRs 1-3), an amino acid sequence having at least 95% identity to the amino acid sequence of SEQ ID NO: 194 (e.g., TCRa full length protein), the amino acid sequences of SEQ ID NOs: 195-197 (e.g., TCRb CDRs 1-3), and an amino acid sequence having at least 95% identity to the amino acid sequence of SEQ ID NO: 199 (e.g., TCRb full length protein).

In some embodiments, a TCR comprises the amino acid sequences of SEQ ID NOS: 190-192 (e.g., TCRa CDRs 1-3), an amino acid sequence having at least 98% identity to the amino acid sequence of SEQ ID NO: 193 (e.g., TCRa variable domain), the amino acid sequences of SEQ ID NOs: 195-197 (e.g., TCRb CDRs 1-3), and an amino acid sequence having at least 98% identity to the amino acid sequence of SEQ ID NO: 198 (e.g., TCRb variable domain). In some embodiments, a TCR comprises the amino acid sequences of SEQ ID NOs: 190-192 (e.g., TCRa CDRs 1-3), an amino acid sequence having at least 98% identity to the amino acid sequence of SEQ ID NO: 296 (e.g., TCRa full length protein), the amino acid sequences of SEQ ID NOs: 195-197 (e.g., TCRb CDRs 1-3), and an amino acid sequence having at least 98% identity to the amino acid sequence of SEQ ID NO: 297 (e.g., TCRb full length protein). In some embodiments, a TCR comprises the amino acid sequences of SEQ ID NOs: 190-192 (e.g., TCRa CDRs 1-3), an amino acid sequence having at least 98% identity to the amino acid sequence of SEQ ID NO: 194 (e.g., TCRa full length protein), the amino acid sequences of SEQ ID NOs: 195-197 (e.g., TCRb CDRs 1-3), and an amino acid sequence having at least 98% identity to the amino acid sequence of SEQ ID NO: 199 (e.g., TCRb full length protein).

In some embodiments, a TCR comprises the amino acid sequences of SEQ ID NOS: 200-202 (e.g., TCRa CDRs 1-3), an amino acid sequence having at least 90% identity to the amino acid sequence of SEQ ID NO: 203 (e.g., TCRa variable domain), the amino acid sequences of SEQ ID NOs: 205-207 (e.g., TCRb CDRs 1-3), and an amino acid sequence having at least 90% identity to the amino acid sequence of SEQ ID NO: 208 (e.g., TCRb variable domain). In some embodiments, a TCR comprises the amino acid sequences of SEQ ID NOs: 200-202 (e.g., TCRa CDRs 1-3), an amino acid sequence having at least 90% identity to the amino acid sequence of SEQ ID NO: 298 (e.g., TCRa full length protein), the amino acid sequences of SEQ ID NOs: 205-207 (e.g., TCRb CDRs 1-3), and an amino acid sequence having at least 90% identity to the amino acid sequence of SEQ ID NO: 299 (e.g., TCRb full length protein). In some embodiments, a TCR comprises the amino acid sequences of SEQ ID NOs: 200-202 (e.g., TCRa CDRs 1-3), an amino acid sequence having at least 90% identity to the amino acid sequence of SEQ ID NO: 204 (e.g., TCRa full length protein), the amino acid sequences of SEQ ID NOs: 205-207 (e.g., TCRb CDRs 1-3), and an amino acid sequence having at least 90% identity to the amino acid sequence of SEQ ID NO: 209 (e.g., TCRb full length protein).

In some embodiments, a TCR comprises the amino acid sequences of SEQ ID NOS: 200-202 (e.g., TCRa CDRs 1-3), an amino acid sequence having at least 95% identity to the amino acid sequence of SEQ ID NO: 203 (e.g., TCRa variable domain), the amino acid sequences of SEQ ID NOs: 205-207 (e.g., TCRb CDRs 1-3), and an amino acid sequence having at least 95% identity to the amino acid sequence of SEQ ID NO: 208 (e.g., TCRb variable domain). In some embodiments, a TCR comprises the amino acid sequences of SEQ ID NOs: 200-202 (e.g., TCRa CDRs 1-3), an amino acid sequence having at least 95% identity to the amino acid sequence of SEQ ID NO: 298 (e.g., TCRa full length protein), the amino acid sequences of SEQ ID NOs: 205-207 (e.g., TCRb CDRs 1-3), and an amino acid sequence having at least 95% identity to the amino acid sequence of SEQ ID NO: 299 (e.g., TCRb full length protein). In some embodiments, a TCR comprises the amino acid sequences of SEQ ID NOs: 200-202 (e.g., TCRa CDRs 1-3), an amino acid sequence having at least 95% identity to the amino acid sequence of SEQ ID NO: 204 (e.g., TCRa full length protein), the amino acid sequences of SEQ ID NOs: 205-207 (e.g., TCRb CDRs 1-3), and an amino acid sequence having at least 95% identity to the amino acid sequence of SEQ ID NO: 209 (e.g., TCRb full length protein).

In some embodiments, a TCR comprises the amino acid sequences of SEQ ID NOS: 200-202 (e.g., TCRa CDRs 1-3), an amino acid sequence having at least 98% identity to the amino acid sequence of SEQ ID NO: 203 (e.g., TCRa variable domain), the amino acid sequences of SEQ ID NOs: 205-207 (e.g., TCRb CDRs 1-3), and an amino acid sequence having at least 98% identity to the amino acid sequence of SEQ ID NO: 208 (e.g., TCRb variable domain). In some embodiments, a TCR comprises the amino acid sequences of SEQ ID NOs: 200-202 (e.g., TCRa CDRs 1-3), an amino acid sequence having at least 98% identity to the amino acid sequence of SEQ ID NO: 298 (e.g., TCRa full length protein), the amino acid sequences of SEQ ID NOs: 205-207 (e.g., TCRb CDRs 1-3), and an amino acid sequence having at least 98% identity to the amino acid sequence of SEQ ID NO: 299 (e.g., TCRb full length protein). In some embodiments, a TCR comprises the amino acid sequences of SEQ ID NOs: 200-202 (e.g., TCRa CDRs 1-3), an amino acid sequence having at least 98% identity to the amino acid sequence of SEQ ID NO: 204 (e.g., TCRa full length protein), the amino acid sequences of SEQ ID NOs: 205-207 (e.g., TCRb CDRs 1-3), and an amino acid sequence having at least 98% identity to the amino acid sequence of SEQ ID NO: 209 (e.g., TCRb full length protein).

In some embodiments, a TCR comprises the amino acid sequences of SEQ ID NOS: 210-212 (e.g., TCRa CDRs 1-3), an amino acid sequence having at least 90% identity to the amino acid sequence of SEQ ID NO: 213 (e.g., TCRa variable domain), the amino acid sequences of SEQ ID NOs: 215-217 (e.g., TCRb CDRs 1-3), and an amino acid sequence having at least 90% identity to the amino acid sequence of SEQ ID NO: 218 (e.g., TCRb variable domain). In some embodiments, a TCR comprises the amino acid sequences of SEQ ID NOs: 210-212 (e.g., TCRa CDRs 1-3), an amino acid sequence having at least 90% identity to the amino acid sequence of SEQ ID NO: 300 (e.g., TCRa full length protein), the amino acid sequences of SEQ ID NOs: 215-217 (e.g., TCRb CDRs 1-3), and an amino acid sequence having at least 90% identity to the amino acid sequence of SEQ ID NO: 301 (e.g., TCRb full length protein). In some embodiments, a TCR comprises the amino acid sequences of SEQ ID NOs: 210-212 (e.g., TCRa CDRs 1-3), an amino acid sequence having at least 90% identity to the amino acid sequence of SEQ ID NO: 214 (e.g., TCRa full length protein), the amino acid sequences of SEQ ID NOs: 215-217 (e.g., TCRb CDRs 1-3), and an amino acid sequence having at least 90% identity to the amino acid sequence of SEQ ID NO: 219 (e.g., TCRb full length protein).

In some embodiments, a TCR comprises the amino acid sequences of SEQ ID NOS: 210-212 (e.g., TCRa CDRs 1-3), an amino acid sequence having at least 95% identity to the amino acid sequence of SEQ ID NO: 213 (e.g., TCRa variable domain), the amino acid sequences of SEQ ID NOs: 215-217 (e.g., TCRb CDRs 1-3), and an amino acid sequence having at least 95% identity to the amino acid sequence of SEQ ID NO: 218 (e.g., TCRb variable domain). In some embodiments, a TCR comprises the amino acid sequences of SEQ ID NOs: 210-212 (e.g., TCRa CDRs 1-3), an amino acid sequence having at least 95% identity to the amino acid sequence of SEQ ID NO: 300 (e.g., TCRa full length protein), the amino acid sequences of SEQ ID NOs: 215-217 (e.g., TCRb CDRs 1-3), and an amino acid sequence having at least 95% identity to the amino acid sequence of SEQ ID NO: 301 (e.g., TCRb full length protein). In some embodiments, a TCR comprises the amino acid sequences of SEQ ID NOs: 210-212 (e.g., TCRa CDRs 1-3), an amino acid sequence having at least 95% identity to the amino acid sequence of SEQ ID NO: 214 (e.g., TCRa full length protein), the amino acid sequences of SEQ ID NOs: 215-217 (e.g., TCRb CDRs 1-3), and an amino acid sequence having at least 95% identity to the amino acid sequence of SEQ ID NO: 219 (e.g., TCRb full length protein).

In some embodiments, a TCR comprises the amino acid sequences of SEQ ID NOS: 210-212 (e.g., TCRa CDRs 1-3), an amino acid sequence having at least 98% identity to the amino acid sequence of SEQ ID NO: 213 (e.g., TCRa variable domain), the amino acid sequences of SEQ ID NOs: 215-217 (e.g., TCRb CDRs 1-3), and an amino acid sequence having at least 98% identity to the amino acid sequence of SEQ ID NO: 218 (e.g., TCRb variable domain). In some embodiments, a TCR comprises the amino acid sequences of SEQ ID NOs: 210-212 (e.g., TCRa CDRs 1-3), an amino acid sequence having at least 98% identity to the amino acid sequence of SEQ ID NO: 300 (e.g., TCRa full length protein), the amino acid sequences of SEQ ID NOs: 215-217 (e.g., TCRb CDRs 1-3), and an amino acid sequence having at least 98% identity to the amino acid sequence of SEQ ID NO: 301 (e.g., TCRb full length protein). In some embodiments, a TCR comprises the amino acid sequences of SEQ ID NOs: 210-212 (e.g., TCRa CDRs 1-3), an amino acid sequence having at least 98% identity to the amino acid sequence of SEQ ID NO: 214 (e.g., TCRa full length protein), the amino acid sequences of SEQ ID NOs: 215-217 (e.g., TCRb CDRs 1-3), and an amino acid sequence having at least 98% identity to the amino acid sequence of SEQ ID NO: 219 (e.g., TCRb full length protein).

In some embodiments, a TCR comprises the amino acid sequences of SEQ ID NOS: 220-222 (e.g., TCRa CDRs 1-3), an amino acid sequence having at least 90% identity to the amino acid sequence of SEQ ID NO: 223 (e.g., TCRa variable domain), the amino acid sequences of SEQ ID NOs: 225-227 (e.g., TCRb CDRs 1-3), and an amino acid sequence having at least 90% identity to the amino acid sequence of SEQ ID NO: 228 (e.g., TCRb variable domain). In some embodiments, a TCR comprises the amino acid sequences of SEQ ID NOs: 220-222 (e.g., TCRa CDRs 1-3), an amino acid sequence having at least 90% identity to the amino acid sequence of SEQ ID NO: 302 (e.g., TCRa full length protein), the amino acid sequences of SEQ ID NOs: 225-227 (e.g., TCRb CDRs 1-3), and an amino acid sequence having at least 90% identity to the amino acid sequence of SEQ ID NO: 303 (e.g., TCRb full length protein). In some embodiments, a TCR comprises the amino acid sequences of SEQ ID NOs: 220-222 (e.g., TCRa CDRs 1-3), an amino acid sequence having at least 90% identity to the amino acid sequence of SEQ ID NO: 224 (e.g., TCRa full length protein), the amino acid sequences of SEQ ID NOs: 225-227 (e.g., TCRb CDRs 1-3), and an amino acid sequence having at least 90% identity to the amino acid sequence of SEQ ID NO: 229 (e.g., TCRb full length protein).

In some embodiments, a TCR comprises the amino acid sequences of SEQ ID NOS: 220-222 (e.g., TCRa CDRs 1-3), an amino acid sequence having at least 95% identity to the amino acid sequence of SEQ ID NO: 223 (e.g., TCRa variable domain), the amino acid sequences of SEQ ID NOs: 225-227 (e.g., TCRb CDRs 1-3), and an amino acid sequence having at least 95% identity to the amino acid sequence of SEQ ID NO: 228 (e.g., TCRb variable domain). In some embodiments, a TCR comprises the amino acid sequences of SEQ ID NOs: 220-222 (e.g., TCRa CDRs 1-3), an amino acid sequence having at least 95% identity to the amino acid sequence of SEQ ID NO: 302 (e.g., TCRa full length protein), the amino acid sequences of SEQ ID NOs: 225-227 (e.g., TCRb CDRs 1-3), and an amino acid sequence having at least 95% identity to the amino acid sequence of SEQ ID NO: 303 (e.g., TCRb full length protein). In some embodiments, a TCR comprises the amino acid sequences of SEQ ID NOs: 220-222 (e.g., TCRa CDRs 1-3), an amino acid sequence having at least 95% identity to the amino acid sequence of SEQ ID NO: 224 (e.g., TCRa full length protein), the amino acid sequences of SEQ ID NOs: 225-227 (e.g., TCRb CDRs 1-3), and an amino acid sequence having at least 95% identity to the amino acid sequence of SEQ ID NO: 229 (e.g., TCRb full length protein).

In some embodiments, a TCR comprises the amino acid sequences of SEQ ID NOs: 220-222 (e.g., TCRa CDRs 1-3), an amino acid sequence having at least 98% identity to the amino acid sequence of SEQ ID NO: 223 (e.g., TCRa variable domain), the amino acid sequences of SEQ ID NOs: 225-227 (e.g., TCRb CDRs 1-3), and an amino acid sequence having at least 98% identity to the amino acid sequence of SEQ ID NO: 228 (e.g., TCRb variable domain). In some embodiments, a TCR comprises the amino acid sequences of SEQ ID NOs: 220-222 (e.g., TCRa CDRs 1-3), an amino acid sequence having at least 98% identity to the amino acid sequence of SEQ ID NO: 302 (e.g., TCRa full length protein), the amino acid sequences of SEQ ID NOs: 225-227 (e.g., TCRb CDRs 1-3), and an amino acid sequence having at least 98% identity to the amino acid sequence of SEQ ID NO: 303 (e.g., TCRb full length protein). In some embodiments, a TCR comprises the amino acid sequences of SEQ ID NOs: 220-222 (e.g., TCRa CDRs 1-3), an amino acid sequence having at least 98% identity to the amino acid sequence of SEQ ID NO: 224 (e.g., TCRa full length protein), the amino acid sequences of SEQ ID NOs: 225-227 (e.g., TCRb CDRs 1-3), and an amino acid sequence having at least 98% identity to the amino acid sequence of SEQ ID NO: 229 (e.g., TCRb full length protein).

In some embodiments, a TCR comprises the amino acid sequences of SEQ ID NOs: 230-232 (e.g., TCRa CDRs 1-3), an amino acid sequence having at least 90% identity to the amino acid sequence of SEQ ID NO: 233 (e.g., TCRa variable domain), the amino acid sequences of SEQ ID NOs: 235-237 (e.g., TCRb CDRs 1-3), and an amino acid sequence having at least 90% identity to the amino acid sequence of SEQ ID NO: 238 (e.g., TCRb variable domain). In some embodiments, a TCR comprises the amino acid sequences of SEQ ID NOs: 230-232 (e.g., TCRa CDRs 1-3), an amino acid sequence having at least 90% identity to the amino acid sequence of SEQ ID NO: 304 (e.g., TCRa full length protein), the amino acid sequences of SEQ ID NOs: 235-237 (e.g., TCRb CDRs 1-3), and an amino acid sequence having at least 90% identity to the amino acid sequence of SEQ ID NO: 305 (e.g., TCRb full length protein). In some embodiments, a TCR comprises the amino acid sequences of SEQ ID NOs: 230-232 (e.g., TCRa CDRs 1-3), an amino acid sequence having at least 90% identity to the amino acid sequence of SEQ ID NO: 234 (e.g., TCRa full length protein), the amino acid sequences of SEQ ID NOs: 235-237 (e.g., TCRb CDRs 1-3), and an amino acid sequence having at least 90% identity to the amino acid sequence of SEQ ID NO: 239 (e.g., TCRb full length protein).

In some embodiments, a TCR comprises the amino acid sequences of SEQ ID NOs: 230-232 (e.g., TCRa CDRs 1-3), an amino acid sequence having at least 95% identity to the amino acid sequence of SEQ ID NO: 233 (e.g., TCRa variable domain), the amino acid sequences of SEQ ID NOs: 235-237 (e.g., TCRb CDRs 1-3), and an amino acid sequence having at least 95% identity to the amino acid sequence of SEQ ID NO: 238 (e.g., TCRb variable domain). In some embodiments, a TCR comprises the amino acid sequences of SEQ ID NOs: 230-232 (e.g., TCRa CDRs 1-3), an amino acid sequence having at least 95% identity to the amino acid sequence of SEQ ID NO: 304 (e.g., TCRa full length protein), the amino acid sequences of SEQ ID NOs: 235-237 (e.g., TCRb CDRs 1-3), and an amino acid sequence having at least 95% identity to the amino acid sequence of SEQ ID NO: 305 (e.g., TCRb full length protein). In some embodiments, a TCR comprises the amino acid sequences of SEQ ID NOs: 230-232 (e.g., TCRa CDRs 1-3), an amino acid sequence having at least 95% identity to the amino acid sequence of SEQ ID NO: 234 (e.g., TCRa full length protein), the amino acid sequences of SEQ ID NOs: 235-237 (e.g., TCRb CDRs 1-3), and an amino acid sequence having at least 95% identity to the amino acid sequence of SEQ ID NO: 239 (e.g., TCRb full length protein).

In some embodiments, a TCR comprises the amino acid sequences of SEQ ID NOS: 230-232 (e.g., TCRa CDRs 1-3), an amino acid sequence having at least 98% identity to the amino acid sequence of SEQ ID NO: 233 (e.g., TCRa variable domain), the amino acid sequences of SEQ ID NOs: 235-237 (e.g., TCRb CDRs 1-3), and an amino acid sequence having at least 98% identity to the amino acid sequence of SEQ ID NO: 238 (e.g., TCRb variable domain). In some embodiments, a TCR comprises the amino acid sequences of SEQ ID NOs: 230-232 (e.g., TCRa CDRs 1-3), an amino acid sequence having at least 98% identity to the amino acid sequence of SEQ ID NO: 304 (e.g., TCRa full length protein), the amino acid sequences of SEQ ID NOs: 235-237 (e.g., TCRb CDRs 1-3), and an amino acid sequence having at least 98% identity to the amino acid sequence of SEQ ID NO: 305 (e.g., TCRb full length protein). In some embodiments, a TCR comprises the amino acid sequences of SEQ ID NOs: 230-232 (e.g., TCRa CDRs 1-3), an amino acid sequence having at least 98% identity to the amino acid sequence of SEQ ID NO: 234 (e.g., TCRa full length protein), the amino acid sequences of SEQ ID NOs: 235-237 (e.g., TCRb CDRs 1-3), and an amino acid sequence having at least 98% identity to the amino acid sequence of SEQ ID NO: 239 (e.g., TCRb full length protein).

In some embodiments, a TCR comprises the amino acid sequences of SEQ ID NOS: 240-242 (e.g., TCRa CDRs 1-3), an amino acid sequence having at least 90% identity to the amino acid sequence of SEQ ID NO: 243 (e.g., TCRa variable domain), the amino acid sequences of SEQ ID NOs: 245-247 (e.g., TCRb CDRs 1-3), and an amino acid sequence having at least 90% identity to the amino acid sequence of SEQ ID NO: 248 (e.g., TCRb variable domain). In some embodiments, a TCR comprises the amino acid sequences of SEQ ID NOs: 240-242 (e.g., TCRa CDRs 1-3), an amino acid sequence having at least 90% identity to the amino acid sequence of SEQ ID NO: 306 (e.g., TCRa full length protein), the amino acid sequences of SEQ ID NOs: 245-247 (e.g., TCRb CDRs 1-3), and an amino acid sequence having at least 90% identity to the amino acid sequence of SEQ ID NO: 307 (e.g., TCRb full length protein). In some embodiments, a TCR comprises the amino acid sequences of SEQ ID NOs: 240-242 (e.g., TCRa CDRs 1-3), an amino acid sequence having at least 90% identity to the amino acid sequence of SEQ ID NO: 244 (e.g., TCRa full length protein), the amino acid sequences of SEQ ID NOs: 245-247 (e.g., TCRb CDRs 1-3), and an amino acid sequence having at least 90% identity to the amino acid sequence of SEQ ID NO: 249 (e.g., TCRb full length protein).

In some embodiments, a TCR comprises the amino acid sequences of SEQ ID NOs: 240-242 (e.g., TCRa CDRs 1-3), an amino acid sequence having at least 95% identity to the amino acid sequence of SEQ ID NO: 243 (e.g., TCRa variable domain), the amino acid sequences of SEQ ID NOs: 245-247 (e.g., TCRb CDRs 1-3), and an amino acid sequence having at least 95% identity to the amino acid sequence of SEQ ID NO: 248 (e.g., TCRb variable domain). In some embodiments, a TCR comprises the amino acid sequences of SEQ ID NOs: 240-242 (e.g., TCRa CDRs 1-3), an amino acid sequence having at least 95% identity to the amino acid sequence of SEQ ID NO: 306 (e.g., TCRa full length protein), the amino acid sequences of SEQ ID NOs: 245-247 (e.g., TCRb CDRs 1-3), and an amino acid sequence having at least 95% identity to the amino acid sequence of SEQ ID NO: 307 (e.g., TCRb full length protein). In some embodiments, a TCR comprises the amino acid sequences of SEQ ID NOs: 240-242 (e.g., TCRa CDRs 1-3), an amino acid sequence having at least 95% identity to the amino acid sequence of SEQ ID NO: 244 (e.g., TCRa full length protein), the amino acid sequences of SEQ ID NOs: 245-247 (e.g., TCRb CDRs 1-3), and an amino acid sequence having at least 95% identity to the amino acid sequence of SEQ ID NO: 249 (e.g., TCRb full length protein).

In some embodiments, a TCR comprises the amino acid sequences of SEQ ID NOS: 240-242 (e.g., TCRa CDRs 1-3), an amino acid sequence having at least 98% identity to the amino acid sequence of SEQ ID NO: 243 (e.g., TCRa variable domain), the amino acid sequences of SEQ ID NOs: 245-247 (e.g., TCRb CDRs 1-3), and an amino acid sequence having at least 98% identity to the amino acid sequence of SEQ ID NO: 248 (e.g., TCRb variable domain). In some embodiments, a TCR comprises the amino acid sequences of SEQ ID NOs: 240-242 (e.g., TCRa CDRs 1-3), an amino acid sequence having at least 98% identity to the amino acid sequence of SEQ ID NO: 306 (e.g., TCRa full length protein), the amino acid sequences of SEQ ID NOs: 245-247 (e.g., TCRb CDRs 1-3), and an amino acid sequence having at least 98% identity to the amino acid sequence of SEQ ID NO: 307 (e.g., TCRb full length protein). In some embodiments, a TCR comprises the amino acid sequences of SEQ ID NOs: 240-242 (e.g., TCRa CDRs 1-3), an amino acid sequence having at least 98% identity to the amino acid sequence of SEQ ID NO: 244 (e.g., TCRa full length protein), the amino acid sequences of SEQ ID NOs: 245-247 (e.g., TCRb CDRs 1-3), and an amino acid sequence having at least 98% identity to the amino acid sequence of SEQ ID NO: 249 (e.g., TCRb full length protein).

In some embodiments, a TCR comprises the amino acid sequences of SEQ ID NOS: 250-252 (e.g., TCRa CDRs 1-3), an amino acid sequence having at least 90% identity to the amino acid sequence of SEQ ID NO: 253 (e.g., TCRa variable domain), the amino acid sequences of SEQ ID NOs: 255-257 (e.g., TCRb CDRs 1-3), and an amino acid sequence having at least 90% identity to the amino acid sequence of SEQ ID NO: 258 (e.g., TCRb variable domain). In some embodiments, a TCR comprises the amino acid sequences of SEQ ID NOs: 250-252 (e.g., TCRa CDRs 1-3), an amino acid sequence having at least 90% identity to the amino acid sequence of SEQ ID NO: 308 (e.g., TCRa full length protein), the amino acid sequences of SEQ ID NOs: 255-257 (e.g., TCRb CDRs 1-3), and an amino acid sequence having at least 90% identity to the amino acid sequence of SEQ ID NO: 309 (e.g., TCRb full length protein). In some embodiments, a TCR comprises the amino acid sequences of SEQ ID NOs: 250-252 (e.g., TCRa CDRs 1-3), an amino acid sequence having at least 90% identity to the amino acid sequence of SEQ ID NO: 254 (e.g., TCRa full length protein), the amino acid sequences of SEQ ID NOs: 255-257 (e.g., TCRb CDRs 1-3), and an amino acid sequence having at least 90% identity to the amino acid sequence of SEQ ID NO: 259 (e.g., TCRb full length protein).

In some embodiments, a TCR comprises the amino acid sequences of SEQ ID NOS: 250-252 (e.g., TCRa CDRs 1-3), an amino acid sequence having at least 95% identity to the amino acid sequence of SEQ ID NO: 253 (e.g., TCRa variable domain), the amino acid sequences of SEQ ID NOs: 255-257 (e.g., TCRb CDRs 1-3), and an amino acid sequence having at least 95% identity to the amino acid sequence of SEQ ID NO: 258 (e.g., TCRb variable domain). In some embodiments, a TCR comprises the amino acid sequences of SEQ ID NOs: 250-252 (e.g., TCRa CDRs 1-3), an amino acid sequence having at least 95% identity to the amino acid sequence of SEQ ID NO: 308 (e.g., TCRa full length protein), the amino acid sequences of SEQ ID NOs: 255-257 (e.g., TCRb CDRs 1-3), and an amino acid sequence having at least 95% identity to the amino acid sequence of SEQ ID NO: 309 (e.g., TCRb full length protein). In some embodiments, a TCR comprises the amino acid sequences of SEQ ID NOs: 250-252 (e.g., TCRa CDRs 1-3), an amino acid sequence having at least 95% identity to the amino acid sequence of SEQ ID NO: 254 (e.g., TCRa full length protein), the amino acid sequences of SEQ ID NOs: 255-257 (e.g., TCRb CDRs 1-3), and an amino acid sequence having at least 95% identity to the amino acid sequence of SEQ ID NO: 259 (e.g., TCRb full length protein).

In some embodiments, a TCR comprises the amino acid sequences of SEQ ID NOS: 250-252 (e.g., TCRa CDRs 1-3), an amino acid sequence having at least 98% identity to the amino acid sequence of SEQ ID NO: 253 (e.g., TCRa variable domain), the amino acid sequences of SEQ ID NOs: 255-257 (e.g., TCRb CDRs 1-3), and an amino acid sequence having at least 98% identity to the amino acid sequence of SEQ ID NO: 258 (e.g., TCRb variable domain). In some embodiments, a TCR comprises the amino acid sequences of SEQ ID NOs: 250-252 (e.g., TCRa CDRs 1-3), an amino acid sequence having at least 98% identity to the amino acid sequence of SEQ ID NO: 308 (e.g., TCRa full length protein), the amino acid sequences of SEQ ID NOs: 255-257 (e.g., TCRb CDRs 1-3), and an amino acid sequence having at least 98% identity to the amino acid sequence of SEQ ID NO: 309 (e.g., TCRb full length protein). In some embodiments, a TCR comprises the amino acid sequences of SEQ ID NOs: 250-252 (e.g., TCRa CDRs 1-3), an amino acid sequence having at least 98% identity to the amino acid sequence of SEQ ID NO: 254 (e.g., TCRa full length protein), the amino acid sequences of SEQ ID NOs: 255-257 (e.g., TCRb CDRs 1-3), and an amino acid sequence having at least 98% identity to the amino acid sequence of SEQ ID NO: 259 (e.g., TCRb full length protein).

A regulatory T cell (or other T cell) may comprise any one or more of the TCRs or TCR components (e.g., TCRa CDR1, TCRa CDR2, TCRa CDR3, TCRa variable domain, TCRa full, TCRa full (DS), TCRb CDR1, TCRb CDR2, TCRb CDR3, TCRb variable domain, TCRb full, TCRb full (DS) and/or Full ORF) provided in Table 1. In some embodiments, a TCR further comprises a ribosome skip P2A element (e.g., SEQ ID NO: 103). In some embodiments, the orientation of a TCR is beta chain-P2A-alpha chain. In some embodiments, the orientation of a TCR is alpha chain-P2A-beta chain.

TABLE 1

| | | | SEQ |
|---|---|---|---|
| | | | ID |
| | Name | Amino Acid Sequence | NO: |
| TCR-A | TCRa | VTNFRS | 1 |
| | CDR1 | | |
| | TCRa | EVTNFRSL | 327 |
| | CDR1 | | |
| | (alternate) | | |
| | TCRa | LTSSGIE | 2 |
| | CDR2 | | |
| | TCRa | MLTSSGIEK | 328 |
| | CDR2 | | |
| | (alternate) | | |
| | TCRa | CAVEASSNTGKLIF | 3 |
| | CDR3 | | |
| | TCRa | EDKVVQSPLSLVVHEGDTVTLNCSYEVTNFRSLLWYKQEKKAPTFLFMLTSS | 4 |
| | variable | GIEKKSGRLSSILDKKELFSILNITATQTGDSAVYLCAVEASSNTGKLIFGQ | |
| | domain | GTTLQVKP | |
| | TCRa full | MMKCPQALLAIFWLLLSWVSSEDKVVQSPLSLVVHEGDTVTLNCSYEVTNFR | 5 |
| | | SLLWYKQEKKAPTFLFMLTSSGIEKKSGRLSSILDKKELFSILNITATQTGD | |
| | | SAVYLCAVEASSNTGKLIFGQGTTLQVKPDIQNPDPAVYQLRDSKSSDKSVC | |
| | | LFTDFDSQTNVSQSKDSDVYITDKTVLDMRSMDFKSNSAVAWSNKSDFACAN | |
| | | AFNNSIIPEDTFFPSPESSCDVKLVEKSFETDTNLNFQNLSVIGFRILLLKV | |
| | | AGFNLLMTLRLWSS | |
| | TCRa full | EDKVVQSPLSLVVHEGDTVTLNCSYEVTNFRSLLWYKQEKKAPTFLFMLTSS | 318 |
| | (without | GIEKKSGRLSSILDKKELFSILNITATQTGDSAVYLCAVEASSNTGKLIFGQ | |
| | leader) | GTTLQVKPDIQNPDPAVYQLRDSKSSDKSVCLFTDFDSQTNVSQSKDSDVYI | |
| | | TDKTVLDMRSMDFKSNSAVAWSNKSDFACANAFNNSIIPEDTFFPSPESSCD | |
| | | VKLVEKSFETDTNLNFQNLSVIGFRILLLKVAGFNLLMTLRLWSS | |
| | TCRa full | MMKCPQALLAIFWLLLSWVSSEDKVVQSPLSLVVHEGDTVTLNCSYEVTNFR | 260 |
| | (DS) | SLLWYKQEKKAPTFLFMLTSSGIEKKSGRLSSILDKKELFSILNITATQTGD | |
| | | SAVYLCAVEASSNTGKLIFGQGTTLQVKPDIQNPDPAVYQLRDSKSSDKSVC | |
| | | LFTDFDSQTNVSQSKDSDVYITDKCVLDMRSMDFKSNSAVAWSNKSDFACAN | |
| | | AFNNSIIPEDTFFPSPESSCDVKLVEKSFETDTNLNFQNLSVIGFRILLLKV | |
| | | AGFNLLMTLRLWSS | |
| | TCRa full | EDKVVQSPLSLVVHEGDTVTLNCSYEVTNFRSLLWYKQEKKAPTFLFMLTSS | 319 |
| | (DS) | GIEKKSGRLSSILDKKELFSILNITATQTGDSAVYLCAVEASSNTGKLIFGQ | |
| | without | GTTLQVKPDIQNPDPAVYQLRDSKSSDKSVCLFTDFDSQTNVSQSKDSDVYI | |
| | leader | TDKCVLDMRSMDFKSNSAVAWSNKSDFACANAFNNSIIPEDTFFPSPESSCD | |
| | | VKLVEKSFETDTNLNFQNLSVIGFRILLLKVAGFNLLMTLRLWSS | |
| | TCRb | MNHNY | 6 |
| | CDR1 | | |
| | TCRb | DMNHNYM | 329 |
| | CDR1 | | |
| | (alternate) | | |
| | TCRb | SVGAGI | 7 |
| | CDR2 | | |
| | TCRb | YSVGAGIT | 330 |
| | CDR2 | | |
| | (alternate) | | |
| | TCRb | CASSRTGGSEQYF | 8 |
| | CDR3 | | |
| | TCRb | NAGVTQTPKFRILKIGQSMTLQCAQDMNHNYMYWYRQDPGMGLKLIYYSVGA | 9 |
| | variable | GITDKGEVPNGYNVSRSTTEDFPLRLELAAPSQTSVYFCASSRTGGSEQYFG | |
| | domain | PGTRLTVT | |
| | TCRb full | MSISLLCCAAFPLLWAGPVNAGVTQTPKFRILKIGQSMTLQCAQDMNHNYMY | 10 |
| | | WYRQDPGMGLKLIYYSVGAGITDKGEVPNGYNVSRSTTEDFPLRLELAAPSQ | |
| | | TSVYFCASSRTGGSEQYFGPGTRLTVTEDLKNVFPPEVAVFEPSEAEISHTQ | |
| | | KATLVCLATGFYPDHVELSWWVNGKEVHSGVSTDPQPLKEQPALNDSRYCLS | |
| | | SRLRVSATFWQNPRNHFRCQVQFYGLSENDEWTQDRAKPVTQIVSAEAWGRA | |
| | | DCGFTSESYQQGVLSATILYEILLGKATLYAVLVSALVLMAMVKRKDSRG | |
| | TCRb full | MSISLLCCAAFPLLWAGPVNAGVTQTPKFRILKIGQSMTLQCAQDMNHNYMY | 320 |
| | with P2A | WYRQDPGMGLKLIYYSVGAGITDKGEVPNGYNVSRSTTEDFPLRLELAAPSQ | |
| | overhang | TSVYFCASSRTGGSEQYFGPGTRLTVTEDLKNVFPPEVAVFEPSEAEISHTQ | |
| | | KATLVCLATGFYPDHVELSWWVNGKEVHSGVSTDPQPLKEQPALNDSRYCLS | |
| | | SRLRVSATFWQNPRNHFRCQVQFYGLSENDEWTQDRAKPVTQIVSAEAWGRA | |
| | | DCGFTSESYQQGVLSATILYEILLGKATLYAVLVSALVLMAMVKRKDSRG | |
| | | GSGATNFSLLKQAGDVEENPG | |
| | TCRb full | NAGVTQTPKFRILKIGQSMTLQCAQDMNHNYMYWYRQDPGMGLKLIYYSVGA | 321 |
| | without | GITDKGEVPNGYNVSRSTTEDFPLRLELAAPSQTSVYFCASSRTGGSEQYFG | |
| | leader | PGTRLTVTEDLKNVFPPEVAVFEPSEAEISHTQKATLVCLATGFYPDHVELS | |
| | | WWVNGKEVHSGVSTDPQPLKEQPALNDSRYCLSSRLRVSATFWQNPRNHFRC | |
| | | QVQFYGLSENDEWTQDRAKPVTQIVSAEAWGRADCGFTSESYQQGVLSATIL | |
| | | YEILLGKATLYAVLVSALVLMAMVKRKDSRG | |
| | TCRb full | NAGVTQTPKFRILKIGQSMTLQCAQDMNHNYMYWYRQDPGMGLKLIYYSVGA | 322 |
| | without | GITDKGEVPNGYNVSRSTTEDFPLRLELAAPSQTSVYFCASSRTGGSEQYFG | |
| | leader and | PGTRLTVTEDLKNVFPPEVAVFEPSEAEISHTQKATLVCLATGFYPDHVELS | |
| | with P2A | WWVNGKEVHSGVSTDPQPLKEQPALNDSRYCLSSRLRVSATFWQNPRNHFRC | |
| | overhang | QVQFYGLSENDEWTQDRAKPVTQIVSAEAWGRADCGFTSESYQQGVLSATIL | |
| | | YEILLGKATLYAVLVSALVLMAMVKRKDSRG | |
| | | GSGATNFSLLKQAGDVEENPG | |
| | TCRb full | MSISLLCCAAFPLLWAGPVNAGVTQTPKFRILKIGQSMTLQCAQDMNHNYMY | 261 |
| | (DS) | WYRQDPGMGLKLIYYSVGAGITDKGEVPNGYNVSRSTTEDFPLRLELAAPSQ | |
| | | TSVYFCASSRTGGSEQYFGPGTRLTVTEDLKNVFPPEVAVFEPSEAEISHTQ | |
| | | KATLVCLATGFYPDHVELSWWVNGKEVHSGVCTDPQPLKEQPALNDSRYCLS | |
| | | SRLRVSATFWQNPRNHFRCQVQFYGLSENDEWTQDRAKPVTQIVSAEAWGRA | |
| | | DCGFTSESYQQGVLSATILYEILLGKATLYAVLVSALVLMAMVKRKDSRG | |
| | TCRb full | MSISLLCCAAFPLLWAGPVNAGVTQTPKFRILKIGQSMTLQCAQDMNHNYMY | 323 |
| | (DS) with | WYRQDPGMGLKLIYYSVGAGITDKGEVPNGYNVSRSTTEDFPLRLELAAPSQ | |
| | | TSVYFCASSRTGGSEQYFGPGTRLTVTEDLKNVFPPEVAVFEPSEAEISHTQ | |
| | P2A | KATLVCLATGFYPDHVELSWWVNGKEVHSGVCTDPQPLKEQPALNDSRYCLS | |
| | overhang | SRLRVSATFWQNPRNHFRCQVQFYGLSENDEWTQDRAKPVTQIVSAEAWGRA | |
| | | DCGFTSESYQQGVLSATILYEILLGKATLYAVLVSALVLMAMVKRKDSRG | |
| | | GSGATNFSLLKQAGDVEENPG | |
| | TCRb full | NAGVTQTPKFRILKIGQSMTLQCAQDMNHNYMYWYRQDPGMGLKLIYYSVGA | 324 |
| | (DS) | GITDKGEVPNGYNVSRSTTEDFPLRLELAAPSQTSVYFCASSRTGGSEQYFG | |
| | without | PGTRLTVTEDLKNVFPPEVAVFEPSEAEISHTQKATLVCLATGFYPDHVELS | |
| | leader | WWVNGKEVHSGVCTDPQPLKEQPALNDSRYCLSSRLRVSATFWQNPRNHFRC | |
| | | QVQFYGLSENDEWTQDRAKPVTQIVSAEAWGRADCGFTSESYQQGVLSATIL | |
| | | YEILLGKATLYAVLVSALVLMAMVKRKDSRG | |
| | TCRb full | NAGVTQTPKFRILKIGQSMTLQCAQDMNHNYMYWYRQDPGMGLKLIYYSVGA | 325 |
| | (DS) | GITDKGEVPNGYNVSRSTTEDFPLRLELAAPSQTSVYFCASSRTGGSEQYFG | |
| | without | PGTRLTVTEDLKNVFPPEVAVFEPSEAEISHTQKATLVCLATGFYPDHVELS | |
| | leader and | WWVNGKEVHSGVCTDPQPLKEQPALNDSRYCLSSRLRVSATFWQNPRNHFRC | |
| | with P2A | QVQFYGLSENDEWTQDRAKPVTQIVSAEAWGRADCGFTSESYQQGVLSATIL | |
| | overhang | YEILLGKATLYAVLVSALVLMAMVKRKDSRG | |
| | | GSGATNFSLLKQAGDVEENPG | |
| | Full length | MSISLLCCAAFPLLWAGPVNAGVTQTPKFRILKIGQSMTLQCAQDMNHNYMY | 310 |
| | TCR | WYRQDPGMGLKLIYYSVGAGITDKGEVPNGYNVSRSTTEDFPLRLELAAPSQ | |
| | | TSVYFCASSRTGGSEQYFGPGTRLTVTEDLKNVFPPEVAVFEPSEAEISHTQ | |
| | | KATLVCLATGFYPDHVELSWWVNGKEVHSGVSTDPQPLKEQPALNDSRYCLS | |
| | | SRLRVSATFWQNPRNHFRCQVQFYGLSENDEWTQDRAKPVTQIVSAEAWGRA | |
| | | DCGFTSESYQQGVLSATILYEILLGKATLYAVLVSALVLMAMVKRKDSRGGS | |
| | | GATNFSLLKQAGDVEENPGPMMKCPQALLAIFWLLLSWVSSEDKVVQSPLSL | |
| | | VVHEGDTVTLNCSYEVTNFRSLLWYKQEKKAPTFLFMLTSSGIEKKSGRLSS | |
| | | ILDKKELFSILNITATQTGDSAVYLCAVEASSNTGKLIFGQGTTLQVKPDIQ | |
| | | NPDPAVYQLRDSKSSDKSVCLFTDFDSQTNVSQSKDSDVYITDKTVLDMRSM | |
| | | DFKSNSAVAWSNKSDFACANAFNNSIIPEDTFFPSPESSCDVKLVEKSFETD | |
| | | TNLNFQNLSVIGFRILLLKVAGFNLLMTLRLWSS | |
| | Full length | MSISLLCCAAFPLLWAGPVNAGVTQTPKFRILKIGQSMTLQCAQDMNHNYMY | 311 |
| | TCR (DS) | WYRQDPGMGLKLIYYSVGAGITDKGEVPNGYNVSRSTTEDFPLRLELAAPSQ | |
| | | TSVYFCASSRTGGSEQYFGPGTRLTVTEDLKNVFPPEVAVFEPSEAEISHTQ | |
| | | KATLVCLATGFYPDHVELSWWVNGKEVHSGVCTDPQPLKEQPALNDSRYCLS | |
| | | SRLRVSATFWQNPRNHFRCQVQFYGLSENDEWTQDRAKPVTQIVSAEAWGRA | |
| | | DCGFTSESYQQGVLSATILYEILLGKATLYAVLVSALVLMAMVKRKDSRGGS | |
| | | GATNFSLLKQAGDVEENPGPMMKCPQALLAIFWLLLSWVSSEDKVVQSPLSL | |
| | | VVHEGDTVTLNCSYEVTNFRSLLWYKQEKKAPTFLFMLTSSGIEKKSGRLSS | |
| | | ILDKKELFSILNITATQTGDSAVYLCAVEASSNTGKLIFGQGTTLQVKPDIQ | |
| | | NPDPAVYQLRDSKSSDKSVCLFTDFDSQTNVSQSKDSDVYITDKCVLDMRSM | |
| | | DFKSNSAVAWSNKSDFACANAFNNSIIPEDTFFPSPESSCDVKLVEKSFETD | |
| | | TNLNFQNLSVIGFRILLLKVAGFNLLMTLRLWSS | |
| | Full Length | ATGAGCATCA GCCTCCTGTG CTGTGCAGCC TTTCCTCTCC | 326 |
| | TCR (DS) | TGTGGGCAGG TCCAGTGAAT GCTGGTGTCA CTCAGACCCC | |
| | nucleotide | AAAATTCCGC ATCCTGAAGA TAGGACAGAG CATGACACTG | |
| | sequence | CAGTGTGCCC AGGATATGAA CCATAACTAC ATGTACTGGT | |
| | | ATCGACAAGA CCCAGGCATG GGGCTGAAGC TGATTTATTA | |
| | | TTCAGTTGGT GCTGGTATCA CTGACAAAGG AGAAGTCCCG | |
| | | AATGGCTACA ACGTCTCCAG ATCAACCACA GAGGATTTCC | |
| | | CGCTCAGGCT GGAGTTGGCT GCTCCCTCCC AGACATCTGT | |
| | | GTACTTCTGT GCCAGCAGCC GGACAGGGGG AAGCGAGCAG | |
| | | TACTTCGGGC CGGGCACCAG GCTCACGGTC ACAGAGGACC | |
| | | TGAAAAACGT GTTCCCACCC GAGGTCGCTG TGTTTGAGCC | |
| | | ATCAGAAGCA GAGATCTCCC ACACCCAAAA GGCCACACTG | |
| | | GTGTGCCTGG CCACAGGCTT CTACCCCGAC CACGTGGAGC | |
| | | TGAGCTGGTG GGTGAATGGG AAGGAGGTGC ACAGTGGGGT | |
| | | CTGCACAGAC CCGCAGCCCC TCAAGGAGCA GCCCGCCCTC | |
| | | AATGACTCCA GATACTGCCT GAGCAGCCGC CTGAGGGTCT | |
| | | CGGCCACCTT CTGGCAGAAC CCCCGCAACC ACTTCCGCTG | |
| | | TCAAGTCCAG TTCTACGGGC TCTCGGAGAA TGACGAGTGG | |
| | | ACCCAGGATA GGGCCAAACC TGTCACCCAG ATCGTCAGCG | |
| | | CCGAGGCCTG GGGTAGAGCA GACTGTGGCT TCACCTCCGA | |
| | | GTCTTACCAG CAAGGGGTCC TGTCTGCCAC CATCCTCTAT | |
| | | GAGATCTTGC TAGGGAAGGC CACCTTGTAT GCCGTGCTGG | |
| | | TCAGTGCCCT CGTGCTGATG GCCATGGTCA AGAGAAAGGA | |
| | | TTCCAGAGGC GGCAGCGGCG CCACCAACTT CTCCCTGCTG | |
| | | AAGCAGGCCG GCGACGTGGA AGAAAACCCT GGCCCCATGA | |
| | | TGAAGTGTCC ACAGGCTTTA CTAGCTATCT TTTGGCTTCT | |
| | | ACTGAGCTGG GTGAGCAGTG AAGACAAGGT GGTACAAAGC | |
| | | CCTCTATCTC TGGTTGTCCA CGAGGGAGAC ACTGTAACTC | |
| | | TCAATTGCAG TTATGAAGTG ACTAACTTTC GAAGCCTACT | |
| | | ATGGTACAAG CAGGAAAAGA AAGCTCCCAC ATTTCTATTT | |
| | | ATGCTAACTT CAAGTGGAAT TGAAAAGAAG TCAGGAAGAC | |
| | | TAAGTAGCAT ATTAGATAAG AAAGAACTTT TCAGCATCCT | |
| | | GAACATCACA GCCACCCAGA CCGGAGACTC GGCCGTCTAC | |
| | | CTCTGTGCTG TGGAGGCCTC TAGCAACACA GGCAAACTAA | |
| | | TCTTTGGGCA AGGGACAACT TTACAAGTAA AACCAGATAT | |
| | | CCAGAACCCT GACCCTGCCG TGTACCAGCT GAGAGACTCT | |
| | | AAATCCAGTG ACAAGTCTGT CTGCCTATTC ACCGATTTTG | |
| | | ATTCTCAAAC AAATGTGTCA CAAAGTAAGG ATTCTGATGT | |
| | | GTATATCACA GACAAATGTG TGCTAGACAT GAGGTCTATG | |
| | | GACTTCAAGA GCAACAGTGC TGTGGCCTGG AGCAACAAAT | |
| | | CTGACTTTGC ATGTGCAAAC GCCTTCAACA ACAGCATTAT | |
| | | TCCAGAAGAC ACCTTCTTCC CCAGCCCAGA AAGTTCCTGT | |
| | | GATGTCAAGC TGGTCGAGAA AAGCTTTGAA ACAGATACGA | |
| | | ACCTAAACTT TCAAAACCTG TCAGTGATTG GGTTCCGAAT | |
| | | CCTCCTCCTG AAAGTGGCCG GGTTTAATCT GCTCATGACG | |
| | | CTGCGGCTGT GGTCCAGCTG A | |
| TCR-B | TCRa | EVTNFRSL | 11 |
| | CDR1 | | |
| | TCRa | MLTSSGIEK | 12 |
| | CDR2 | | |
| | TCRa | CAVNSGAGSYQLTF | 13 |
| | CDR3 | | |
| | TCRa | EDKVVQSPLSLVVHEGDTVTLNCSYEVTNFRSLLWYKQEKKAPTFLFMLTSS | 14 |
| | variable | GIEKKSGRLSSILDKKELFSILNITATQTGDSAVYLCAVNSGAGSYQLTFGK | |
| | domain | GTKLSVIP | |
| | TCRa full | MMKCPQALLAIFWLLLSWVSSEDKVVQSPLSLVVHEGDTVTLNCSYEVTNFR | 15 |
| | | SLLWYKQEKKAPTFLFMLTSSGIEKKSGRLSSILDKKELFSILNITATQTGD | |
| | | SAVYLCAVNSGAGSYQLTFGKGTKLSVIPNIQNPDPAVYQLRDSKSSDKSVC | |
| | | LFTDFDSQTNVSQSKDSDVYITDKTVLDMRSMDFKSNSAVAWSNKSDFACAN | |
| | | AFNNSIIPEDTFFPSPESSCDVKLVEKSFETDTNLNFQNLSVIGFRILLLKV | |
| | | AGFNLLMTLRLWSS | |
| | TCRa full | MMKCPQALLAIFWLLLSWVSSEDKVVQSPLSLVVHEGDTVTLNCSYEVTNFR | 262 |
| | (DS) | SLLWYKQEKKAPTFLFMLTSSGIEKKSGRLSSILDKKELFSILNITATQTGD | |
| | | SAVYLCAVNSGAGSYQLTFGKGTKLSVIPNIQNPDPAVYQLRDSKSSDKSVC | |
| | | LFTDFDSQTNVSQSKDSDVYITDKCVLDMRSMDFKSNSAVAWSNKSDFACAN | |
| | | AFNNSIIPEDTFFPSPESSCDVKLVEKSFETDTNLNFQNLSVIGFRILLLKV | |
| | | AGFNLLMTLRLWSS | |
| | TCRb | TWNHNNM | 16 |
| | CDR1 | | |
| | TCRb | YSYGVQDT | 17 |
| | CDR2 | | |
| | TCRb | CASSSGGLYEQYF | 18 |
| | CDR3 | | |
| | TCRb | DAEITQSPRHKITETGRQVTLACHQTWNHNNMFWYRQDLGHGLRLIHYSYGV | 19 |
| | variable | QDTNKGEVSDGYSVSRSNTEDLPLTLESAASSQTSVYFCASSSGGLYEQYFG | |
| | domain | PGTRLTVT | |
| | TCRb full | MGTRLFFYVALCLLWAGHRDAEITQSPRHKITETGRQVTLACHQTWNHNNMF | 20 |
| | | WYRQDLGHGLRLIHYSYGVQDTNKGEVSDGYSVSRSNTEDLPLTLESAASSQ | |
| | | TSVYFCASSSGGLYEQYFGPGTRLTVTEDLKNVFPPEVAVFEPSEAEISHTQ | |
| | | KATLVCLATGFYPDHVELSWWVNGKEVHSGVSTDPQPLKEQPALNDSRYCLS | |
| | | SRLRVSATFWQNPRNHFRCQVQFYGLSENDEWTQDRAKPVTQIVSAEAWGRA | |
| | | DCGFTSESYQQGVLSATILYEILLGKATLYAVLVSALVLMAMVKRKDSRG | |
| | TCRb full | MGTRLFFYVALCLLWAGHRDAEITQSPRHKITETGRQVTLACHQTWNHNNMF | 263 |
| | (DS) | WYRQDLGHGLRLIHYSYGVQDTNKGEVSDGYSVSRSNTEDLPLTLESAASSQ | |
| | | TSVYFCASSSGGLYEQYFGPGTRLTVTEDLKNVFPPEVAVFEPSEAEISHTQ | |
| | | KATLVCLATGFYPDHVELSWWVNGKEVHSGVCTDPQPLKEQPALNDSRYCLS | |
| | | SRLRVSATFWQNPRNHFRCQVQFYGLSENDEWTQDRAKPVTQIVSAEAWGRA | |
| | | DCGFTSESYQQGVLSATILYEILLGKATLYAVLVSALVLMAMVKRKDSRG | |
| TCR-C | TCRa | TVSPFSNL | 21 |
| | CDR1 | | |
| | TCRa | IMTFSENTK | 22 |
| | CDR2 | | |
| | TCRa | CVVGPSGGSNYKLTF | 23 |
| | CDR3 | | |
| | TCRa | KNQVEQSPQSLIILEGKNCTLQCNYTVSPFSNLRWYKQDTGRGPVSLTIMTF | 24 |
| | variable | SENTKSNGRYTATLDADTKQSSLHITASQLSDSASYICVVGPSGGSNYKLTF | |
| | domain | GKGTLLTVNP | |
| | TCRa full | MKKHLTTFLVILWLYFYRGNGKNQVEQSPQSLIILEGKNCTLQCNYTVSPFS | 25 |
| | | NLRWYKQDTGRGPVSLTIMTFSENTKSNGRYTATLDADTKQSSLHITASQLS | |
| | | DSASYICVVGPSGGSNYKLTFGKGTLLTVNPNIQNPDPAVYQLRDSKSSDKS | |
| | | VCLFTDFDSQTNVSQSKDSDVYITDKTVLDMRSMDFKSNSAVAWSNKSDFAC | |
| | | ANAFNNSIIPEDTFFPSPESSCDVKLVEKSFETDTNLNFQNLSVIGFRILLL | |
| | | KVAGFNLLMTLRLWSS | |
| | TCRa full | MKKHLTTFLVILWLYFYRGNGKNQVEQSPQSLIILEGKNCTLQCNYTVSPFS | 264 |
| | (DS) | NLRWYKQDTGRGPVSLTIMTFSENTKSNGRYTATLDADTKQSSLHITASQLS | |
| | | DSASYICVVGPSGGSNYKLTFGKGTLLTVNPNIQNPDPAVYQLRDSKSSDKS | |
| | | VCLFTDFDSQTNVSQSKDSDVYITDKCVLDMRSMDFKSNSAVAWSNKSDFAC | |
| | | ANAFNNSIIPEDTFFPSPESSCDVKLVEKSFETDTNLNFQNLSVIGFRILLL | |
| | | KVAGFNLLMTLRLWSS | |
| | TCRb | RSGDLSV | 26 |
| | CDR1 | | |
| | TCRb | QYYNGEER | 27 |
| | CDR2 | | |
| | TCRb | CASSGQGFLGNTEAFF | 28 |
| | CDR3 | | |
| | TCRb | DSGVTQTPKHLITATGQRVTLRCSPRSGDLSVYWYQQSLDQGLQFLIQYYNG | 29 |
| | variable | EERAKGNILERFSAQQFPDLHSELNLSSLELGDSALYFCASSGQGFLGNTEA | |
| | domain | FFGQGTRLTVV | |
| | TCRb full | MGFRLLCCVAFCLLGAGPVDSGVTQTPKHLITATGQRVTLRCSPRSGDLSVY | 30 |
| | | WYQQSLDQGLQFLIQYYNGEERAKGNILERFSAQQFPDLHSELNLSSLELGD | |
| | | SALYFCASSGQGFLGNTEAFFGQGTRLTVVEDLNKVFPPEVAVFEPSEAEIS | |
| | | HTQKATLVCLATGFFPDHVELSWWVNGKEVHSGVSTDPQPLKEQPALNDSRY | |
| | | CLSSRLRVSATFWQNPRNHFRCQVQFYGLSENDEWTQDRAKPVTQIVSAEAW | |
| | | GRADCGFTSVSYQQGVLSATILYEILLGKATLYAVLVSALVLMAMVKRKDF | |
| | TCRb full | MGFRLLCCVAFCLLGAGPVDSGVTQTPKHLITATGQRVTLRCSPRSGDLSVY | 265 |
| | (DS) | WYQQSLDQGLQFLIQYYNGEERAKGNILERFSAQQFPDLHSELNLSSLELGD | |
| | | SALYFCASSGQGFLGNTEAFFGQGTRLTVVEDLNKVFPPEVAVFEPSEAEIS | |
| | | HTQKATLVCLATGFFPDHVELSWWVNGKEVHSGVCTDPQPLKEQPALNDSRY | |
| | | CLSSRLRVSATFWQNPRNHFRCQVQFYGLSENDEWTQDRAKPVTQIVSAEAW | |
| | | GRADCGFTSVSYQQGVLSATILYEILLGKATLYAVLVSALVLMAMVKRKDF | |
| TCR-D | TCRa | ATGYPS | 31 |
| | CDR1 | | |
| | TCRa | TATGYPSL | 331 |
| | CDR1 | | |
| | (alternate) | | |
| | TCRa | ATKADDK | 32 |
| | CDR2 | | |
| | TCRa | KATKADDKG | 332 |
| | CDR2 | | |
| | (alternate) | | |
| | TCRa | CALSEKTSYDKVIF | 33 |
| | CDR3 | | |
| | TCRa | GNSVTQMEGPVTLSEEAFLTINCTYTATGYPSLFWYVQYPGEGLQLLLKATK | 34 |
| | variable | ADDKGSNKGFEATYRKETTSFHLEKGSVQVSDSAVYFCALSEKTSYDKVIFG | |
| | domain | PGTSLSVIP | |
| | TCRa full | MNYSPGLVSLILLLLGRTRGNSVTQMEGPVTLSEEAFLTINCTYTATGYPSL | 35 |
| | | FWYVQYPGEGLQLLLKATKADDKGSNKGFEATYRKETTSFHLEKGSVQVSDS | |
| | | AVYFCALSEKTSYDKVIFGPGTSLSVIPNIQNPDPAVYQLRDSKSSDKSVCL | |
| | | FTDFDSQTNVSQSKDSDVYITDKTVLDMRSMDFKSNSAVAWSNKSDFACANA | |
| | | FNNSIIPEDTFFPSPESSCDVKLVEKSFETDTNLNFQNLSVIGFRILLLKVA | |
| | | GFNLLMTLRLWSS | |
| | TCRa full | MNYSPGLVSLILLLLGRTRGNSVTQMEGPVTLSEEAFLTINCTYTATGYPSL | 266 |
| | (DS) | FWYVQYPGEGLQLLLKATKADDKGSNKGFEATYRKETTSFHLEKGSVQVSDS | |
| | | AVYFCALSEKTSYDKVIFGPGTSLSVIPNIQNPDPAVYQLRDSKSSDKSVCL | |
| | | FTDFDSQTNVSQSKDSDVYITDKCVLDMRSMDFKSNSAVAWSNKSDFACANA | |
| | | FNNSIIPEDTFFPSPESSCDVKLVEKSFETDTNLNFQNLSVIGFRILLLKVA | |
| | | GFNLLMTLRLWSS | |
| | TCRb | MNHNY | 36 |
| | CDR1 | | |
| | TCRb | DMNHNYM | 333 |
| | CDR1 | | |
| | (alternate) | | |
| | TCRb | SVGAGI | 37 |
| | CDR2 | | |
| | TCRb | YSVGAGIT | 334 |
| | CDR2 | | |
| | (alternate) | | |
| | TCRb | CASSYGPGGSEAFF | 38 |
| | CDR3 | | |
| | TCRb | NAGVTQTPKFRILKIGQSMTLQCTQDMNHNYMYWYRQDPGMGLKLIYYSVGA | 39 |
| | variable | GITDKGEVPNGYNVSRSTTEDFPLRLELAAPSQTSVYFCASSYGPGGSEAFF | |
| | domain | GQGTRLTVV | |
| | TCRb full | MSISLLCCAAFPLLWAGPVNAGVTQTPKFRILKIGQSMTLQCTQDMNHNYMY | 40 |
| | | WYRQDPGMGLKLIYYSVGAGITDKGEVPNGYNVSRSTTEDFPLRLELAAPSQ | |
| | | TSVYFCASSYGPGGSEAFFGQGTRLTVVEDLNKVFPPEVAVFEPSEAEISHT | |
| | | QKATLVCLATGFFPDHVELSWWVNGKEVHSGVSTDPQPLKEQPALNDSRYCL | |
| | | SSRLRVSATFWQNPRNHFRCQVQFYGLSENDEWTQDRAKPVTQIVSAEAWGR | |
| | | ADCGFTSVSYQQGVLSATILYEILLGKATLYAVLVSALVLMAMVKRKDF | |
| | TCRb full | MSISLLCCAAFPLLWAGPVNAGVTQTPKFRILKIGQSMTLQCTQDMNHNYMY | 267 |
| | (DS) | WYRQDPGMGLKLIYYSVGAGITDKGEVPNGYNVSRSTTEDFPLRLELAAPSQ | |
| | | TSVYFCASSYGPGGSEAFFGQGTRLTVVEDLNKVFPPEVAVFEPSEAEISHT | |
| | | QKATLVCLATGFFPDHVELSWWVNGKEVHSGVCTDPQPLKEQPALNDSRYCL | |
| | | SSRLRVSATFWQNPRNHFRCQVQFYGLSENDEWTQDRAKPVTQIVSAEAWGR | |
| | | ADCGFTSVSYQQGVLSATILYEILLGKATLYAVLVSALVLMAMVKRKDF | |
| | Full length | MSISLLCCAAFPLLWAGPVNAGVTQTPKFRILKIGQSMTLQCTQDMNHNYMY | 312 |
| | TCR | WYRQDPGMGLKLIYYSVGAGITDKGEVPNGYNVSRSTTEDFPLRLELAAPSQ | |
| | | TSVYFCASSYGPGGSEAFFGQGTRLTVVEDLNKVFPPEVAVFEPSEAEISHT | |
| | | QKATLVCLATGFFPDHVELSWWVNGKEVHSGVSTDPQPLKEQPALNDSRYCL | |
| | | SSRLRVSATFWQNPRNHFRCQVQFYGLSENDEWTQDRAKPVTQIVSAEAWGR | |
| | | ADCGFTSVSYQQGVLSATILYEILLGKATLYAVLVSALVLMAMVKRKDFGSG | |
| | | ATNFSLLKQAGDVEENPGPMNYSPGLVSLILLLLGRTRGNSVTQMEGPVTLS | |
| | | EEAFLTINCTYTATGYPSLFWYVQYPGEGLQLLLKATKADDKGSNKGFEATY | |
| | | RKETTSFHLEKGSVQVSDSAVYFCALSEKTSYDKVIFGPGTSLSVIPNIQNP | |
| | | DPAVYQLRDSKSSDKSVCLFTDFDSQTNVSQSKDSDVYITDKTVLDMRSMDF | |
| | | KSNSAVAWSNKSDFACANAFNNSIIPEDTFFPSPESSCDVKLVEKSFETDTN | |
| | | LNFQNLSVIGFRILLLKVAGFNLLMTLRLWSS | |
| | Full length | MSISLLCCAAFPLLWAGPVNAGVTQTPKFRILKIGQSMTLQCTQDMNHNYMY | 313 |
| | TCR | WYRQDPGMGLKLIYYSVGAGITDKGEVPNGYNVSRSTTEDFPLRLELAAPSQ | |
| | (DS) | TSVYFCASSYGPGGSEAFFGQGTRLTVVEDLNKVFPPEVAVFEPSEAEISHT | |
| | | QKATLVCLATGFFPDHVELSWWVNGKEVHSGVCTDPQPLKEQPALNDSRYCL | |
| | | SSRLRVSATFWQNPRNHFRCQVQFYGLSENDEWTQDRAKPVTQIVSAEAWGR | |
| | | ADCGFTSVSYQQGVLSATILYEILLGKATLYAVLVSALVLMAMVKRKDF | |
| | | GSGATNFSLLKQAGDVEENPGPMNYSPGLVSLILLLLGRTRGNSVTQMEGPV | |
| | | TLSEEAFLTINCTYTATGYPSLFWYVQYPGEGLQLLLKATKADDKGSNKGFE | |
| | | ATYRKETTSFHLEKGSVQVSDSAVYFCALSEKTSYDKVIFGPGTSLSVIPNI | |
| | | QNPDPAVYQLRDSKSSDKSVCLFTDFDSQTNVSQSKDSDVYITDKCVLDMRS | |
| | | MDFKSNSAVAWSNKSDFACANAFNNSIIPEDTFFPSPESSCDVKLVEKSFET | |
| | | DTNLNFQNLSVIGFRILLLKVAGFNLLMTLRLWSS | |
| TCR-E | TCRa | TATGYPSL | 41 |
| | CDR1 | | |
| | TCRa | KATKADDKG | 42 |
| | CDR2 | | |
| | TCRa | CALSVKTSYDKVIF | 43 |
| | CDR3 | | |
| | TCRa | GNSVTQMEGPVTLSEEAFLTINCTYTATGYPSLFWYVQYPGEGLQLLLKATK | 44 |
| | variable | ADDKGSNKGFEATYRKETTSFHLEKGSVQVSDSAVYFCALSVKTSYDKVIFG | |
| | domain | PGTSLSVIP | |
| | TCRa full | MNYSPGLVSLILLLLGRTRGNSVTQMEGPVTLSEEAFLTINCTYTATGYPSL | 45 |
| | | FWYVQYPGEGLQLLLKATKADDKGSNKGFEATYRKETTSFHLEKGSVQVSDS | |
| | | AVYFCALSVKTSYDKVIFGPGTSLSVIPNIQNPDPAVYQLRDSKSSDKSVCL | |
| | | FTDFDSQTNVSQSKDSDVYITDKTVLDMRSMDFKSNSAVAWSNKSDFACANA | |
| | | FNNSIIPEDTFFPSPESSCDVKLVEKSFETDTNLNFQNLSVIGFRILLLKVA | |
| | | GFNLLMTLRLWSS | |
| | TCRa full | MNYSPGLVSLILLLLGRTRGNSVTQMEGPVTLSEEAFLTINCTYTATGYPSL | 268 |
| | (DS) | FWYVQYPGEGLQLLLKATKADDKGSNKGFEATYRKETTSFHLEKGSVQVSDS | |
| | | AVYFCALSVKTSYDKVIFGPGTSLSVIPNIQNPDPAVYQLRDSKSSDKSVCL | |
| | | FTDFDSQTNVSQSKDSDVYITDKCVLDMRSMDFKSNSAVAWSNKSDFACANA | |
| | | FNNSIIPEDTFFPSPESSCDVKLVEKSFETDTNLNFQNLSVIGFRILLLKVA | |
| | | GFNLLMTLRLWSS | |
| | TCRb | DMNHNSM | 46 |
| | CDR1 | | |
| | TCRb | YSASEGTT | 47 |
| | CDR2 | | |
| | TCRb | CASSRDRGPETQYF | 48 |
| | CDR3 | | |
| | TCRb | NAGVTQTPKFQVLKTGQSMTLQCAQDMNHNSMYWYRQDPGMGLRLIYYSASE | 49 |
| | variable | GTTDKGEVPNGYNVSRLNKREFSLRLESAAPSQTSVYFCASSRDRGPETQYF | |
| | domain | GPGTRLLVL | |
| | TCRb full | MSIGLLCCVAFSLLWASPVNAGVTQTPKFQVLKTGQSMTLQCAQDMNHNSMY | 50 |
| | | WYRQDPGMGLRLIYYSASEGTTDKGEVPNGYNVSRLNKREFSLRLESAAPSQ | |
| | | TSVYFCASSRDRGPETQYFGPGTRLLVLEDLKNVFPPEVAVFEPSEAEISHT | |
| | | QKATLVCLATGFYPDHVELSWWVNGKEVHSGVSTDPQPLKEQPALNDSRYCL | |
| | | SSRLRVSATFWQNPRNHFRCQVQFYGLSENDEWTQDRAKPVTQIVSAEAWGR | |
| | | ADCGFTSESYQQGVLSATILYEILLGKATLYAVLVSALVLMAMVKRKDSRG | |
| | TCRb full | MSIGLLCCVAFSLLWASPVNAGVTQTPKFQVLKTGQSMTLQCAQDMNHNSMY | 269 |
| | (DS) | WYRQDPGMGLRLIYYSASEGTTDKGEVPNGYNVSRLNKREFSLRLESAAPSQ | |
| | | TSVYFCASSRDRGPETQYFGPGTRLLVLEDLKNVFPPEVAVFEPSEAEISHT | |
| | | QKATLVCLATGFYPDHVELSWWVNGKEVHSGVCTDPQPLKEQPALNDSRYCL | |
| | | SSRLRVSATFWQNPRNHFRCQVQFYGLSENDEWTQDRAKPVTQIVSAEAWGR | |
| | | ADCGFTSESYQQGVLSATILYEILLGKATLYAVLVSALVLMAMVKRKDSRG | |
| TCR-F | TCRa | VSGLRG | 51 |
| | CDR1 | | |
| | TCRa | TVSGLRGL | 335 |
| | CDR1 | | |
| | (alternate) | | |
| | TCRa | LYSAGEE | 52 |
| | CDR2 | | |
| | TCRa | TLYSAGEEK | 336 |
| | CDR2 | | |
| | (alternate) | | |
| | TCRa | CAVQATGYALNF | 53 |
| | CDR3 | | |
| | TCRa | EDQVTQSPEALRLQEGESSSLNCSYTVSGLRGLFWYRQDPGKGPEFLFTLYS | 54 |
| | variable | AGEEKEKERLKATLTKKESFLHITAPKPEDSATYLCAVQATGYALNFGKGTS | |
| | domain | LLVTP | |
| | TCRa full | MEKMLECAFIVLWLQLGWLSGEDQVTQSPEALRLQEGESSSLNCSYTVSGLR | 55 |
| | | GLFWYRQDPGKGPEFLFTLYSAGEEKEKERLKATLTKKESFLHITAPKPEDS | |
| | | ATYLCAVQATGYALNFGKGTSLLVTPHIQNPDPAVYQLRDSKSSDKSVCLFT | |
| | | DFDSQTNVSQSKDSDVYITDKTVLDMRSMDFKSNSAVAWSNKSDFACANAFN | |
| | | NSIIPEDTFFPSPESSCDVKLVEKSFETDTNLNFQNLSVIGFRILLLKVAGF | |
| | | NLLMTLRLWSS | |
| | TCRa full | MEKMLECAFIVLWLQLGWLSGEDQVTQSPEALRLQEGESSSLNCSYTVSGLR | 270 |
| | (DS) | GLFWYRQDPGKGPEFLFTLYSAGEEKEKERLKATLTKKESFLHITAPKPEDS | |
| | | ATYLCAVQATGYALNFGKGTSLLVTPHIQNPDPAVYQLRDSKSSDKSVCLFT | |
| | | DFDSQTNVSQSKDSDVYITDKCVLDMRSMDFKSNSAVAWSNKSDFACANAFN | |
| | | NSIIPEDTFFPSPESSCDVKLVEKSFETDTNLNFQNLSVIGFRILLLKVAGF | |
| | | NLLMTLRLWSS | |
| | TCRb | SGHTA | 56 |
| | CDR1 | | |
| | TCRb | ISGHTAL | 337 |
| | CDR1 | | |
| | (alternate) | | |
| | TCRb | FQGNSA | 57 |
| | CDR2 | | |
| | TCRb | YFQGNSAP | 338 |
| | CDR2 | | |
| | (alternate) | | |
| | TCRb | CASNLGGSRQPQHF | 58 |
| | CDR3 | | |
| | TCRb | GAGVSQSPSNKVTEKGKDVELRCDPISGHTALYWYRQSLGQGLEFLIYFQGN | 59 |
| | variable | SAPDKSGLPSDRFSAERTGGSVSTLTIQRTQQEDSAVYLCASNLGGSRQPQH | |
| | domain | FGDGTRLSIL | |
| | TCRb full | MGTRLLFWVAFCLLGADHTGAGVSQSPSNKVTEKGKDVELRCDPISGHTALY | 60 |
| | | WYRQSLGQGLEFLIYFQGNSAPDKSGLPSDRFSAERTGGSVSTLTIQRTQQE | |
| | | DSAVYLCASNLGGSRQPQHFGDGTRLSILEDLNKVFPPEVAVFEPSEAEISH | |
| | | TQKATLVCLATGFFPDHVELSWWVNGKEVHSGVSTDPQPLKEQPALNDSRYC | |
| | | LSSRLRVSATFWQNPRNHFRCQVQFYGLSENDEWTQDRAKPVTQIVSAEAWG | |
| | | RADCGFTSVSYQQGVLSATILYEILLGKATLYAVLVSALVLMAMVKRKDF | |
| | TCRb full | MGTRLLFWVAFCLLGADHTGAGVSQSPSNKVTEKGKDVELRCDPISGHTALY | 271 |
| | (DS) | WYRQSLGQGLEFLIYFQGNSAPDKSGLPSDRFSAERTGGSVSTLTIQRTQQE | |
| | | DSAVYLCASNLGGSRQPQHFGDGTRLSILEDLNKVFPPEVAVFEPSEAEISH | |
| | | TQKATLVCLATGFFPDHVELSWWVNGKEVHSGVCTDPQPLKEQPALNDSRYC | |
| | | LSSRLRVSATFWQNPRNHFRCQVQFYGLSENDEWTQDRAKPVTQIVSAEAWG | |
| | | RADCGFTSVSYQQGVLSATILYEILLGKATLYAVLVSALVLMAMVKRKDF | |
| | Full length | MGTRLLFWVAFCLLGADHTGAGVSQSPSNKVTEKGKDVELRCDPISGHTALY | 314 |
| | TCR | WYRQSLGQGLEFLIYFQGNSAPDKSGLPSDRFSAERTGGSVSTLTIQRTQQE | |
| | | DSAVYLCASNLGGSRQPQHFGDGTRLSILEDLNKVFPPEVAVFEPSEAEISH | |
| | | TQKATLVCLATGFFPDHVELSWWVNGKEVHSGVSTDPQPLKEQPALNDSRYC | |
| | | LSSRLRVSATFWQNPRNHFRCQVQFYGLSENDEWTQDRAKPVTQIVSAEAWG | |
| | | RADCGFTSVSYQQGVLSATILYEILLGKATLYAVLVSALVLMAMVKRKDFGS | |
| | | GATNFSLLKQAGDVEENPGPMEKMLECAFIVLWLQLGWLSGEDQVTQSPEAL | |
| | | RLQEGESSSLNCSYTVSGLRGLFWYRQDPGKGPEFLFTLYSAGEEKEKERLK | |
| | | ATLTKKESFLHITAPKPEDSATYLCAVQATGYALNFGKGTSLLVTPHIQNPD | |
| | | PAVYQLRDSKSSDKSVCLFTDFDSQTNVSQSKDSDVYITDKTVLDMRSMDFK | |
| | | SNSAVAWSNKSDFACANAFNNSIIPEDTFFPSPESSCDVKLVEKSFETDTNL | |
| | | NFQNLSVIGFRILLLKVAGFNLLMTLRLWSS | |
| | Full length | MGTRLLFWVAFCLLGADHTGAGVSQSPSNKVTEKGKDVELRCDPISGHTALY | 315 |
| | TCR | WYRQSLGQGLEFLIYFQGNSAPDKSGLPSDRFSAERTGGSVSTLTIQRTQQE | |
| | (DS) | DSAVYLCASNLGGSRQPQHFGDGTRLSILEDLNKVFPPEVAVFEPSEAEISH | |
| | | TQKATLVCLATGFFPDHVELSWWVNGKEVHSGVCTDPQPLKEQPALNDSRYC | |
| | | LSSRLRVSATFWQNPRNHFRCQVQFYGLSENDEWTQDRAKPVTQIVSAEAWG | |
| | | RADCGFTSVSYQQGVLSATILYEILLGKATLYAVLVSALVLMAMVKRKDF | |
| | | GSGATNFSLLKQAGDVEENPGPMEKMLECAFIVLWLQLGWLSGEDQVTQSPE | |
| | | ALRLQEGESSSLNCSYTVSGLRGLFWYRQDPGKGPEFLFTLYSAGEEKEKER | |
| | | LKATLTKKESFLHITAPKPEDSATYLCAVQATGYALNFGKGTSLLVTPHIQN | |
| | | PDPAVYQLRDSKSSDKSVCLFTDFDSQTNVSQSKDSDVYITDKCVLDMRSMD | |
| | | FKSNSAVAWSNKSDFACANAFNNSIIPEDTFFPSPESSCDVKLVEKSFETDT | |
| | | NLNFQNLSVIGFRILLLKVAGFNLLMTLRLWSS | |
| TCR-G | TCRa | SDRGSQSF | 61 |
| | CDR1 | | |
| | TCRa | FIYSNGDK | 62 |
| | CDR2 | | |
| | TCRa | CAVIYSSASKIIF | 63 |
| | CDR3 | | |
| | TCRa | QKEVEQNSGPLSVPEGAIASLNCTYSDRGSQSFFWYRQYSGKSPELIMFIYS | 64 |
| | variable | NGDKEDGRFTAQLNKASQYVSLLIRDSQPSDSATYLCAVIYSSASKIIFGSG | |
| | domain | TRLSIRP | |
| | TCRa full | MKSLRVLLVILWLQLSWVWSQQKEVEQNSGPLSVPEGAIASLNCTYSDRGSQ | 65 |
| | | SFFWYRQYSGKSPELIMFIYSNGDKEDGRFTAQLNKASQYVSLLIRDSQPSD | |
| | | SATYLCAVIYSSASKIIFGSGTRLSIRPNIQNPDPAVYQLRDSKSSDKSVCL | |
| | | FTDFDSQTNVSQSKDSDVYITDKTVLDMRSMDFKSNSAVAWSNKSDFACANA | |
| | | FNNSIIPEDTFFPSPESSCDVKLVEKSFETDTNLNFQNLSVIGFRILLLKVA | |
| | | GFNLLMTLRLWSS | |
| | TCRa full | MKSLRVLLVILWLQLSWVWSQQKEVEQNSGPLSVPEGAIASLNCTYSDRGSQ | 272 |
| | (DS) | SFFWYRQYSGKSPELIMFIYSNGDKEDGRFTAQLNKASQYVSLLIRDSQPSD | |
| | | SATYLCAVIYSSASKIIFGSGTRLSIRPNIQNPDPAVYQLRDSKSSDKSVCL | |
| | | FTDFDSQTNVSQSKDSDVYITDKCVLDMRSMDFKSNSAVAWSNKSDFACANA | |
| | | FNNSIIPEDTFFPSPESSCDVKLVEKSFETDTNLNFQNLSVIGFRILLLKVA | |
| | | GFNLLMTLRLWSS | |
| | TCRb | EGTSNPNL | 66 |
| | CDR1 | | |
| | TCRb | YSVGIGQ | 67 |
| | CDR2 | | |
| | TCRb | CAWSVQRLGRGGETQYF | 68 |
| | CDR3 | | |
| | TCRb | SQTIHQWPATLVQPVGSPLSLECTVEGTSNPNLYWYRQAAGRGLQLLFYSVG | 69 |
| | variable | IGQISSEVPQNLSASRPQDRQFILSSKKLLLSDSGFYLCAWSVQRLGRGGET | |
| | domain | QYFGPGTRLLVL | |
| | TCRb full | MLCSLLALLLGTFFGVRSQTIHQWPATLVQPVGSPLSLECTVEGTSNPNLYW | 70 |
| | | YRQAAGRGLQLLFYSVGIGQISSEVPQNLSASRPQDRQFILSSKKLLLSDSG | |
| | | FYLCAWSVQRLGRGGETQYFGPGTRLLVLEDLKNVFPPEVAVFEPSEAEISH | |
| | | TQKATLVCLATGFYPDHVELSWWVNGKEVHSGVSTDPQPLKEQPALNDSRYC | |
| | | LSSRLRVSATFWQNPRNHFRCQVQFYGLSENDEWTQDRAKPVTQIVSAEAWG | |
| | | RADCGFTSESYQQGVLSATILYEILLGKATLYAVLVSALVLMAMVKRKDSRG | |
| | TCRb full | MLCSLLALLLGTFFGVRSQTIHQWPATLVQPVGSPLSLECTVEGTSNPNLYW | 273 |
| | (DS) | YRQAAGRGLQLLFYSVGIGQISSEVPQNLSASRPQDRQFILSSKKLLLSDSG | |
| | | FYLCAWSVQRLGRGGETQYFGPGTRLLVLEDLKNVFPPEVAVFEPSEAEISH | |
| | | TQKATLVCLATGFYPDHVELSWWVNGKEVHSGVCTDPQPLKEQPALNDSRYC | |
| | | LSSRLRVSATFWQNPRNHFRCQVQFYGLSENDEWTQDRAKPVTQIVSAEAWG | |
| | | RADCGFTSESYQQGVLSATILYEILLGKATLYAVLVSALVLMAMVKRKDSRG | |
| TCR-H | TCRa | VSGLRG | 71 |
| | CDR1 | | |
| | TCRa | TVSGLRGL | 339 |
| | CDR1 | | |
| | (alternate) | | |
| | TCRa | LYSAGEE | 72 |
| | CDR2 | | |
| | TCRa | TLYSAGEEK | 340 |
| | CDR2 | | |
| | (alternate) | | |
| | TCRa | CAVQLSDSNYKLTF | 73 |
| | CDR3 | | |
| | TCRa | EDQVTQSPEALRLQEGESSSLNCSYTVSGLRGLFWYRQDPGKGPEFLFTLYS | 74 |
| | variable | AGEEKEKERLKATLTKKESFLHITAPKPEDSATYLCAVQLSDSNYKLTFGKG | |
| | domain | TLLTVNP | |
| | TCRa full | MEKMLECAFIVLWLQLGWLSGEDQVTQSPEALRLQEGESSSLNCSYTVSGLR | 75 |
| | | GLFWYRQDPGKGPEFLFTLYSAGEEKEKERLKATLTKKESFLHITAPKPEDS | |
| | | ATYLCAVQLSDSNYKLTFGKGTLLTVNPNIQNPDPAVYQLRDSKSSDKSVCL | |
| | | FTDFDSQTNVSQSKDSDVYITDKTVLDMRSMDFKSNSAVAWSNKSDFACANA | |
| | | FNNSIIPEDTFFPSPESSCDVKLVEKSFETDTNLNFQNLSVIGFRILLLKVA | |
| | | GFNLLMTLRLWSS | |
| | TCRa full | MEKMLECAFIVLWLQLGWLSGEDQVTQSPEALRLQEGESSSLNCSYTVSGLR | 274 |
| | (DS) | GLFWYRQDPGKGPEFLFTLYSAGEEKEKERLKATLTKKESFLHITAPKPEDS | |
| | | ATYLCAVQLSDSNYKLTFGKGTLLTVNPNIQNPDPAVYQLRDSKSSDKSVCL | |
| | | FTDFDSQTNVSQSKDSDVYITDKCVLDMRSMDFKSNSAVAWSNKSDFACANA | |
| | | FNNSIIPEDTFFPSPESSCDVKLVEKSFETDTNLNFQNLSVIGFRILLLKVA | |
| | | GFNLLMTLRLWSS | |
| | TCRb | MNHNS | 76 |
| | CDR1 | | |
| | TCRb | DMNHNSM | 341 |
| | CDR1 | | |
| | (alternate) | | |
| | TCRb | SASEGT | 77 |
| | CDR2 | | |
| | TCRb | YSASEGTT | 342 |
| | CDR2 | | |
| | (alternate) | | |
| | TCRb | CASRGQVNEKLFF | 78 |
| | CDR3 | | |
| | TCRb | NAGVTQTPKFQVLKTGQSMTLQCAQDMNHNSMYWYRQDPGMGLRLIYYSASE | 79 |
| | variable | GTTDKGEVPNGYNVSRLNKREFSLRLESAAPSQTSVYFCASRGQVNEKLFFG | |
| | domain | SGTQLSVL | |
| | TCRb full | MSIGLLCCVAFSLLWASPVNAGVTQTPKFQVLKTGQSMTLQCAQDMNHNSMY | 80 |
| | | WYRQDPGMGLRLIYYSASEGTTDKGEVPNGYNVSRLNKREFSLRLESAAPSQ | |
| | | TSVYFCASRGQVNEKLFFGSGTQLSVLEDLNKVFPPEVAVFEPSEAEISHTQ | |
| | | KATLVCLATGFFPDHVELSWWVNGKEVHSGVSTDPQPLKEQPALNDSRYCLS | |
| | | SRLRVSATFWQNPRNHFRCQVQFYGLSENDEWTQDRAKPVTQIVSAEAWGRA | |
| | | DCGFTSVSYQQGVLSATILYEILLGKATLYAVLVSALVLMAMVKRKDF | |
| | TCRb full | MSIGLLCCVAFSLLWASPVNAGVTQTPKFQVLKTGQSMTLQCAQDMNHNSMY | 275 |
| | (DS) | WYRQDPGMGLRLIYYSASEGTTDKGEVPNGYNVSRLNKREFSLRLESAAPSQ | |
| | | TSVYFCASRGQVNEKLFFGSGTQLSVLEDLNKVFPPEVAVFEPSEAEISHTQ | |
| | | KATLVCLATGFFPDHVELSWWVNGKEVHSGVCTDPQPLKEQPALNDSRYCLS | |
| | | SRLRVSATFWQNPRNHFRCQVQFYGLSENDEWTQDRAKPVTQIVSAEAWGRA | |
| | | DCGFTSVSYQQGVLSATILYEILLGKATLYAVLVSALVLMAMVKRKDF | |
| | Full length | MSIGLLCCVAFSLLWASPVNAGVTQTPKFQVLKTGQSMTLQCAQDMNHNSMY | 316 |
| | TCR | WYRQDPGMGLRLIYYSASEGTTDKGEVPNGYNVSRLNKREFSLRLESAAPSQ | |
| | | TSVYFCASRGQVNEKLFFGSGTQLSVLEDLNKVFPPEVAVFEPSEAEISHTQ | |
| | | KATLVCLATGFFPDHVELSWWVNGKEVHSGVSTDPQPLKEQPALNDSRYCLS | |
| | | SRLRVSATFWQNPRNHFRCQVQFYGLSENDEWTQDRAKPVTQIVSAEAWGRA | |
| | | DCGFTSVSYQQGVLSATILYEILLGKATLYAVLVSALVLMAMVKRKDFGSGA | |
| | | TNFSLLKQAGDVEENPGPMEKMLECAFIVLWLQLGWLSGEDQVTQSPEALRL | |
| | | QEGESSSLNCSYTVSGLRGLFWYRQDPGKGPEFLFTLYSAGEEKEKERLKAT | |
| | | LTKKESFLHITAPKPEDSATYLCAVQLSDSNYKLTFGKGTLLTVNPNIQNPD | |
| | | PAVYQLRDSKSSDKSVCLFTDFDSQTNVSQSKDSDVYITDKTVLDMRSMDFK | |
| | | SNSAVAWSNKSDFACANAFNNSIIPEDTFFPSPESSCDVKLVEKSFETDTNL | |
| | | NFQNLSVIGFRILLLKVAGFNLLMTLRLWSS | |
| | Full length | MSIGLLCCVAFSLLWASPVNAGVTQTPKFQVLKTGQSMTLQCAQDMNHNSMY | 317 |
| | TCR | WYRQDPGMGLRLIYYSASEGTTDKGEVPNGYNVSRLNKREFSLRLESAAPSQ | |
| | (DS) | TSVYFCASRGQVNEKLFFGSGTQLSVLEDLNKVFPPEVAVFEPSEAEISHTQ | |
| | | KATLVCLATGFFPDHVELSWWVNGKEVHSGVCTDPQPLKEQPALNDSRYCLS | |
| | | SRLRVSATFWQNPRNHFRCQVQFYGLSENDEWTQDRAKPVTQIVSAEAWGRA | |
| | | DCGFTSVSYQQGVLSATILYEILLGKATLYAVLVSALVLMAMVKRKDF | |
| | | GSGATNFSLLKQAGDVEENPGPMEKMLECAFIVLWLQLGWLSGEDQVTQSPE | |
| | | ALRLQEGESSSLNCSYTVSGLRGLFWYRQDPGKGPEFLFTLYSAGEEKEKER | |
| | | LKATLTKKESFLHITAPKPEDSATYLCAVQLSDSNYKLTFGKGTLLTVNPNI | |
| | | QNPDPAVYQLRDSKSSDKSVCLFTDFDSQTNVSQSKDSDVYITDKCVLDMRS | |
| | | MDFKSNSAVAWSNKSDFACANAFNNSIIPEDTFFPSPESSCDVKLVEKSFET | |
| | | DTNLNFQNLSVIGFRILLLKVAGFNLLMTLRLWSS | |
| TCR-I | TCRa | STISGNEYV | 81 |
| | CDR1 | | |
| | TCRa | HGLKNNE | 82 |
| | CDR2 | | |
| | TCRa | CIVRFQSGGYQKVTF | 83 |
| | CDR3 | | |
| | TCRa | DAKTTQPTSMDCAEGRAANLPCNHSTISGNEYVYWYRQIHSQGPQYIIHGLK | 84 |
| | variable | NNETNEMASLIITEDRKSSTLILPHATLRDTAVYYCIVRFQSGGYQKVTFGT | |
| | domain | GTKLQVIP | |
| | TCRa full | MRLVARVTVFLTFGTIIDAKTTQPTSMDCAEGRAANLPCNHSTISGNEYVYW | 85 |
| | | YRQIHSQGPQYIIHGLKNNETNEMASLIITEDRKSSTLILPHATLRDTAVYY | |
| | | CIVRFQSGGYQKVTFGTGTKLQVIPNIQNPDPAVYQLRDSKSSDKSVCLFTD | |
| | | FDSQTNVSQSKDSDVYITDKTVLDMRSMDFKSNSAVAWSNKSDFACANAFNN | |
| | | SIIPEDTFFPSPESSCDVKLVEKSFETDTNLNFQNLSVIGFRILLLKVAGEN | |
| | | LLMTLRLWSS | |
| | TCRa full | MRLVARVTVFLTFGTIIDAKTTQPTSMDCAEGRAANLPCNHSTISGNEYVYW | 276 |
| | (DS) | YRQIHSQGPQYIIHGLKNNETNEMASLIITEDRKSSTLILPHATLRDTAVYY | |
| | | CIVRFQSGGYQKVTFGTGTKLQVIPNIQNPDPAVYQLRDSKSSDKSVCLFTD | |
| | | FDSQTNVSQSKDSDVYITDKCVLDMRSMDFKSNSAVAWSNKSDFACANAFNN | |
| | | SIIPEDTFFPSPESSCDVKLVEKSFETDTNLNFQNLSVIGFRILLLKVAGFN | |
| | | LLMTLRLWSS | |
| | TCRb | ISGHNSL | 86 |
| | CDR1 | | |
| | TCRb | YFNNNVPI | 87 |
| | CDR2 | | |
| | TCRb | CASSIQAGGRYNEQFF | 88 |
| | CDR3 | | |
| | TCRb | DAGVIQSPRHEVTEMGQEVTLRCKPISGHNSLFWYRQTMMRGLELLIYFNNN | 89 |
| | variable | VPIDDSGMPEDRFSAKMPNASFSTLKIQPSEPRDSAVYFCASSIQAGGRYNE | |
| | domain | QFFGPGTRLTVL | |
| | TCRb full | MDSWTFCCVSLCILVAKHTDAGVIQSPRHEVTEMGQEVTLRCKPISGHNSLF | 90 |
| | | WYRQTMMRGLELLIYFNNNVPIDDSGMPEDRFSAKMPNASFSTLKIQPSEPR | |
| | | DSAVYFCASSIQAGGRYNEQFFGPGTRLTVLEDLKNVFPPEVAVFEPSEAEI | |
| | | SHTQKATLVCLATGFYPDHVELSWWVNGKEVHSGVSTDPQPLKEQPALNDSR | |
| | | YCLSSRLRVSATFWQNPRNHFRCQVQFYGLSENDEWTQDRAKPVTQIVSAEA | |
| | | WGRADCGFTSESYQQGVLSATILYEILLGKATLYAVLVSALVLMAMVKRKDS | |
| | | RG | |
| | TCRb full | MDSWTFCCVSLCILVAKHTDAGVIQSPRHEVTEMGQEVTLRCKPISGHNSLF | 277 |
| | (DS) | WYRQTMMRGLELLIYFNNNVPIDDSGMPEDRFSAKMPNASFSTLKIQPSEPR | |
| | | DSAVYFCASSIQAGGRYNEQFFGPGTRLTVLEDLKNVFPPEVAVFEPSEAEI | |
| | | SHTQKATLVCLATGFYPDHVELSWWVNGKEVHSGVCTDPQPLKEQPALNDSR | |
| | | YCLSSRLRVSATFWQNPRNHFRCQVQFYGLSENDEWTQDRAKPVTQIVSAEA | |
| | | WGRADCGFTSESYQQGVLSATILYEILLGKATLYAVLVSALVLMAMVKRKDS | |
| | | RG | |
| TCR-J | TCRa | SNSAFQYF | 91 |
| | CDR1 | | |
| | TCRa | YTYSSGNK | 92 |
| | CDR2 | | |
| | TCRa | CAMRTDSWGKLQF | 93 |
| | CDR3 | | |
| | TCRa | QKEVEQDPGPLSVPEGAIVSLNCTYSNSAFQYFMWYRQYSRKGPELLMYTYS | 94 |
| | variable | SGNKEDGRFTAQVDKSSKYISLFIRDSQPSDSATYLCAMRTDSWGKLQFGAG | |
| | domain | TQVVVTP | |
| | TCRa full | MMKSLRVLLVILWLQLSWVWSQQKEVEQDPGPLSVPEGAIVSLNCTYSNSAF | 95 |
| | | QYFMWYRQYSRKGPELLMYTYSSGNKEDGRFTAQVDKSSKYISLFIRDSQPS | |
| | | DSATYLCAMRTDSWGKLQFGAGTQVVVTPDIQNPDPAVYQLRDSKSSDKSVC | |
| | | LFTDFDSQTNVSQSKDSDVYITDKTVLDMRSMDFKSNSAVAWSNKSDFACAN | |
| | | AFNNSIIPEDTFFPSPESSCDVKLVEKSFETDTNLNFQNLSVIGFRILLLKV | |
| | | AGFNLLMTLRLWSS | |
| | TCRa full | MMKSLRVLLVILWLQLSWVWSQQKEVEQDPGPLSVPEGAIVSLNCTYSNSAF | 278 |
| | (DS) | QYFMWYRQYSRKGPELLMYTYSSGNKEDGRFTAQVDKSSKYISLFIRDSQPS | |
| | | DSATYLCAMRTDSWGKLQFGAGTQVVVTPDIQNPDPAVYQLRDSKSSDKSVC | |
| | | LFTDFDSQTNVSQSKDSDVYITDKCVLDMRSMDFKSNSAVAWSNKSDFACAN | |
| | | AFNNSIIPEDTFFPSPESSCDVKLVEKSFETDTNLNFQNLSVIGFRILLLKV | |
| | | AGFNLLMTLRLWSS | |
| | TCRb | NLGHDTM | 96 |
| | CDR1 | | |
| | TCRb | SYNNKELI | 97 |
| | CDR2 | | |
| | TCRb | CASSQTGGAVDTQYF | 98 |
| | CDR3 | | |
| | TCRb | DTAVSQTPKYLVTQMGNDKSIKCEQNLGHDTMYWYKQDSKKFLKIMFSYNNK | 99 |
| | variable | ELIINETVPNRFSPKSPDKAHLNLHINSLELGDSAVYFCASSQTGGAVDTQY | |
| | domain | FGPGTRLTVL | |
| | TCRb full | MGCRLLCCVVFCLLQAGPLDTAVSQTPKYLVTQMGNDKSIKCEQNLGHDTMY | 100 |
| | | WYKQDSKKFLKIMFSYNNKELIINETVPNRFSPKSPDKAHLNLHINSLELGD | |
| | | SAVYFCASSQTGGAVDTQYFGPGTRLTVLEDLKNVFPPEVAVFEPSEAEISH | |
| | | TQKATLVCLATGFYPDHVELSWWVNGKEVHSGVSTDPQPLKEQPALNDSRYC | |
| | | LSSRLRVSATFWQNPRNHFRCQVQFYGLSENDEWTQDRAKPVTQIVSAEAWG | |
| | | RADCGFTSESYQQGVLSATILYEILLGKATLYAVLVSALVLMAMVKRKDSRG | |
| | TCRb full | MGCRLLCCVVFCLLQAGPLDTAVSQTPKYLVTQMGNDKSIKCEQNLGHDTMY | 279 |
| | (DS) | WYKQDSKKFLKIMFSYNNKELIINETVPNRFSPKSPDKAHLNLHINSLELGD | |
| | | SAVYFCASSQTGGAVDTQYFGPGTRLTVLEDLKNVFPPEVAVFEPSEAEISH | |
| | | TQKATLVCLATGFYPDHVELSWWVNGKEVHSGVCTDPQPLKEQPALNDSRYC | |
| | | LSSRLRVSATFWQNPRNHFRCQVQFYGLSENDEWTQDRAKPVTQIVSAEAWG | |
| | | RADCGFTSESYQQGVLSATILYEILLGKATLYAVLVSALVLMAMVKRKDSRG | |
| TCR-K | TCRa | TVSPFSNL | 110 |
| | CDR1 | | |
| | TCRa | IMTFSENTK | 111 |
| | CDR2 | | |
| | TCRa | CVVSASSNTGKLIF | 112 |
| | CDR3 | | |
| | TCRa | KNQVEQSPQSLIILEGKNCTLQCNYTVSPFSNLRWYKQDTGRGPVSLTIMTF | 113 |
| | variable | SENTKSNGRYTATLDADTKQSSLHITASQLSDSASYICVVSASSNTGKLIFG | |
| | domain | QGTTLQVKP | |
| | TCRa full | MKKHLTTFLVILWLYFYRGNGKNQVEQSPQSLIILEGKNCTLQCNYTVSPFS | 114 |
| | | NLRWYKQDTGRGPVSLTIMTFSENTKSNGRYTATLDADTKQSSLHITASQLS | |
| | | DSASYICVVSASSNTGKLIFGQGTTLQVKPDIQNPDPAVYQLRDSKSSDKSV | |
| | | CLFTDFDSQTNVSQSKDSDVYITDKTVLDMRSMDFKSNSAVAWSNKSDFACA | |
| | | NAFNNSIIPEDTFFPSPESSCDVKLVEKSFETDTNLNFQNLSVIGFRILLLK | |
| | | VAGFNLLMTLRLWSS | |
| | TCRa full | MKKHLTTFLVILWLYFYRGNGKNQVEQSPQSLIILEGKNCTLQCNYTVSPFS | 280 |
| | (DS) | NLRWYKQDTGRGPVSLTIMTFSENTKSNGRYTATLDADTKQSSLHITASQLS | |
| | | DSASYICVVSASSNTGKLIFGQGTTLQVKPDIQNPDPAVYQLRDSKSSDKSV | |
| | | CLFTDFDSQTNVSQSKDSDVYITDKCVLDMRSMDFKSNSAVAWSNKSDFACA | |
| | | NAFNNSIIPEDTFFPSPESSCDVKLVEKSFETDTNLNFQNLSVIGFRILLLK | |
| | | VAGFNLLMTLRLWSS | |
| | TCRb | ISGHNSL | 115 |
| | CDR1 | | |
| | TCRb | YFNNNVPI | 116 |
| | CDR2 | | |
| | TCRb | CASSGQLVAYNEQFF | 117 |
| | CDR3 | | |
| | TCRb | DAGVIQSPRHEVTEMGQEVTLRCKPISGHNSLFWYRQTMMRGLELLIYFNNN | 118 |
| | variable | VPIDDSGMPEDRFSAKMPNASFSTLKIQPSEPRDSAVYFCASSGQLVAYNEQ | |
| | domain | FFGPGTRLTVL | |
| | TCRb full | MDSWTFCCVSLCILVAKHTDAGVIQSPRHEVTEMGQEVTLRCKPISGHNSLF | 119 |
| | | WYRQTMMRGLELLIYFNNNVPIDDSGMPEDRFSAKMPNASFSTLKIQPSEPR | |
| | | DSAVYFCASSGQLVAYNEQFFGPGTRLTVLEDLKNVFPPEVAVFEPSEAEIS | |
| | | HTQKATLVCLATGFYPDHVELSWWVNGKEVHSGVSTDPQPLKEQPALNDSRY | |
| | | CLSSRLRVSATFWQNPRNHFRCQVQFYGLSENDEWTQDRAKPVTQIVSAEAW | |
| | | GRADCGFTSESYQQGVLSATILYEILLGKATLYAVLVSALVLMAMVKRKDSR | |
| | | G | |
| | TCRb full | MDSWTFCCVSLCILVAKHTDAGVIQSPRHEVTEMGQEVTLRCKPISGHNSLF | 281 |
| | (DS) | WYRQTMMRGLELLIYFNNNVPIDDSGMPEDRFSAKMPNASFSTLKIQPSEPR | |
| | | DSAVYFCASSGQLVAYNEQFFGPGTRLTVLEDLKNVFPPEVAVFEPSEAEIS | |
| | | HTQKATLVCLATGFYPDHVELSWWVNGKEVHSGVCTDPQPLKEQPALNDSRY | |
| | | CLSSRLRVSATFWQNPRNHFRCQVQFYGLSENDEWTQDRAKPVTQIVSAEAW | |
| | | GRADCGFTSESYQQGVLSATILYEILLGKATLYAVLVSALVLMAMVKRKDSR | |
| | | G | |
| TCR-L | TCRa | SNSASQSF | 120 |
| | CDR1 | | |
| | TCRa | SVYSSGN | 121 |
| | CDR2 | | |
| | TCRa | CVVLRNSGNTPLVF | 122 |
| | CDR3 | | |
| | TCRa | RKEVEQDPGPFNVPEGATVAFNCTYSNSASQSFFWYRQDCRKEPKLLMSVYS | 123 |
| | variable | SGNEDGRFTAQLNRASQYISLLIRDSKLSDSATYLCVVLRNSGNTPLVFGKG | |
| | domain | TRLSVIA | |
| | TCRa full | MISLRVLLVILWLQLSWVWSQRKEVEQDPGPFNVPEGATVAFNCTYSNSASQ | 124 |
| | | SFFWYRQDCRKEPKLLMSVYSSGNEDGRFTAQLNRASQYISLLIRDSKLSDS | |
| | | ATYLCVVLRNSGNTPLVFGKGTRLSVIANIQNPDPAVYQLRDSKSSDKSVCL | |
| | | FTDFDSQTNVSQSKDSDVYITDKTVLDMRSMDFKSNSAVAWSNKSDFACANA | |
| | | FNNSIIPEDTFFPSPESSCDVKLVEKSFETDTNLNFQNLSVIGFRILLLKVA | |
| | | GFNLLMTLRLWSS | |
| | TCRa full | MISLRVLLVILWLQLSWVWSQRKEVEQDPGPFNVPEGATVAFNCTYSNSASQ | 282 |
| | (DS) | SFFWYRQDCRKEPKLLMSVYSSGNEDGRFTAQLNRASQYISLLIRDSKLSDS | |
| | | ATYLCVVLRNSGNTPLVFGKGTRLSVIANIQNPDPAVYQLRDSKSSDKSVCL | |
| | | FTDFDSQTNVSQSKDSDVYITDKCVLDMRSMDFKSNSAVAWSNKSDFACANA | |
| | | FNNSIIPEDTFFPSPESSCDVKLVEKSFETDTNLNFQNLSVIGFRILLLKVA | |
| | | GFNLLMTLRLWSS | |
| | TCRb | TENHRYM | 125 |
| | CDR1 | | |
| | TCRb | YSYGVKDT | 126 |
| | CDR2 | | |
| | TCRb | CAIKPGQGNRGEQFF | 127 |
| | CDR3 | | |
| | TCRb | DAGITQSPRHKVTETGTPVTLRCHQTENHRYMYWYRQDPGHGLRLIHYSYGV | 128 |
| | variable | KDTDKGEVSDGYSVSRSKTEDFLLTLESATSSQTSVYFCAIKPGQGNRGEQF | |
| | domain | FGPGTRLTVL | |
| | TCRb full | MGTRLFFYVALCLLWTGHMDAGITQSPRHKVTETGTPVTLRCHQTENHRYMY | 129 |
| | | WYRQDPGHGLRLIHYSYGVKDTDKGEVSDGYSVSRSKTEDFLLTLESATSSQ | |
| | | TSVYFCAIKPGQGNRGEQFFGPGTRLTVLEDLKNVFPPEVAVFEPSEAEISH | |
| | | TQKATLVCLATGFYPDHVELSWWVNGKEVHSGVSTDPQPLKEQPALNDSRYC | |
| | | LSSRLRVSATFWQNPRNHFRCQVQFYGLSENDEWTQDRAKPVTQIVSAEAWG | |
| | | RADCGFTSESYQQGVLSATILYEILLGKATLYAVLVSALVLMAMVKRKDSRG | |
| | TCRb full | MGTRLFFYVALCLLWTGHMDAGITQSPRHKVTETGTPVTLRCHQTENHRYMY | 283 |
| | (DS) | WYRQDPGHGLRLIHYSYGVKDTDKGEVSDGYSVSRSKTEDFLLTLESATSSQ | |
| | | TSVYFCAIKPGQGNRGEQFFGPGTRLTVLEDLKNVFPPEVAVFEPSEAEISH | |
| | | TQKATLVCLATGFYPDHVELSWWVNGKEVHSGVCTDPQPLKEQPALNDSRYC | |
| | | LSSRLRVSATFWQNPRNHFRCQVQFYGLSENDEWTQDRAKPVTQIVSAEAWG | |
| | | RADCGFTSESYQQGVLSATILYEILLGKATLYAVLVSALVLMAMVKRKDSRG | |
| TCR-M | TCRa | TNSMFDYF | 130 |
| | CDR1 | | |
| | TCRa | SISSIKDKN | 131 |
| | CDR2 | | |
| | TCRa | CAASAKTSGSRLTF | 132 |
| | CDR3 | | |
| | TCRa | DQQVKQNSPSLSVQEGRISILNCDYTNSMFDYFLWYKKYPAEGPTFLISISS | 133 |
| | variable | IKDKNEDGRFTVFLNKSAKHLSLHIVPSQPGDSAVYFCAASAKTSGSRLTFG | |
| | domain | EGTQLTVNP | |
| | TCRa full | MAMLLGASVLILWLQPDWVNSQQKNDDQQVKQNSPSLSVQEGRISILNCDYT | 134 |
| | | NSMFDYFLWYKKYPAEGPTFLISISSIKDKNEDGRFTVFLNKSAKHLSLHIV | |
| | | PSQPGDSAVYFCAASAKTSGSRLTFGEGTQLTVNPDIQNPDPAVYQLRDSKS | |
| | | SDKSVCLFTDFDSQTNVSQSKDSDVYITDKTVLDMRSMDFKSNSAVAWSNKS | |
| | | DFACANAFNNSIIPEDTFFPSPESSCDVKLVEKSFETDTNLNFQNLSVIGFR | |
| | | ILLLKVAGFNLLMTLRLWSS | |
| | TCRa full | MAMLLGASVLILWLQPDWVNSQQKNDDQQVKQNSPSLSVQEGRISILNCDYT | 284 |
| | (DS) | NSMFDYFLWYKKYPAEGPTFLISISSIKDKNEDGRFTVFLNKSAKHLSLHIV | |
| | | PSQPGDSAVYFCAASAKTSGSRLTFGEGTQLTVNPDIQNPDPAVYQLRDSKS | |
| | | SDKSVCLFTDFDSQTNVSQSKDSDVYITDKCVLDMRSMDFKSNSAVAWSNKS | |
| | | DFACANAFNNSIIPEDTFFPSPESSCDVKLVEKSFETDTNLNFQNLSVIGER | |
| | | ILLLKVAGFNLLMTLRLWSS | |
| | TCRb | DMNHNYM | 135 |
| | CDR1 | | |
| | TCRb | YSVGAGIT | 136 |
| | CDR2 | | |
| | TCRb | CASRGGIQETQYF | 137 |
| | CDR3 | | |
| | TCRb | NAGVTQTPKFRILKIGQSMTLQCTQDMNHNYMYWYRQDPGMGLKLIYYSVGA | 138 |
| | variable | GITDKGEVPNGYNVSRSTTEDFPLRLELAAPSQTSVYFCASRGGIQETQYFG | |
| | domain | PGTRLLVL | |
| | TCRb full | MSISLLCCAAFPLLWAGPVNAGVTQTPKFRILKIGQSMTLQCTQDMNHNYMY | 139 |
| | | WYRQDPGMGLKLIYYSVGAGITDKGEVPNGYNVSRSTTEDFPLRLELAAPSQ | |
| | | TSVYFCASRGGIQETQYFGPGTRLLVLEDLKNVFPPEVAVFEPSEAEISHTQ | |
| | | KATLVCLATGFYPDHVELSWWVNGKEVHSGVSTDPQPLKEQPALNDSRYCLS | |
| | | SRLRVSATFWQNPRNHFRCQVQFYGLSENDEWTQDRAKPVTQIVSAEAWGRA | |
| | | DCGFTSESYQQGVLSATILYEILLGKATLYAVLVSALVLMAMVKRKDSRG | |
| | TCRb full | MSISLLCCAAFPLLWAGPVNAGVTQTPKFRILKIGQSMTLQCTQDMNHNYMY | 285 |
| | (DS) | WYRQDPGMGLKLIYYSVGAGITDKGEVPNGYNVSRSTTEDFPLRLELAAPSQ | |
| | | TSVYFCASRGGIQETQYFGPGTRLLVLEDLKNVFPPEVAVFEPSEAEISHTQ | |
| | | KATLVCLATGFYPDHVELSWWVNGKEVHSGVCTDPQPLKEQPALNDSRYCLS | |
| | | SRLRVSATFWQNPRNHFRCQVQFYGLSENDEWTQDRAKPVTQIVSAEAWGRA | |
| | | DCGFTSESYQQGVLSATILYEILLGKATLYAVLVSALVLMAMVKRKDSRG | |
| TCR-N | TCRa | SNSASDYF | 140 |
| | CDR1 | | |
| | TCRa | DIRSNMDKR | 141 |
| | CDR2 | | |
| | TCRa | CAEISKTSYDKVIF | 142 |
| | CDR3 | | |
| | TCRa | GESVGLHLPTLSVQEGDNSIINCAYSNSASDYFIWYKQESGKGPQFIIDIRS | 143 |
| | variable | NMDKRQGQRVTVLLNKTVKHLSLQIAATQPGDSAVYFCAEISKTSYDKVIFG | |
| | domain | PGTSLSVIP | |
| | TCRa full | MAGIRALFMYLWLQLDWVSRGESVGLHLPTLSVQEGDNSIINCAYSNSASDY | 144 |
| | | FIWYKQESGKGPQFIIDIRSNMDKRQGQRVTVLLNKTVKHLSLQIAATQPGD | |
| | | SAVYFCAEISKTSYDKVIFGPGTSLSVIPNIQNPDPAVYQLRDSKSSDKSVC | |
| | | LFTDFDSQTNVSQSKDSDVYITDKTVLDMRSMDFKSNSAVAWSNKSDFACAN | |
| | | AFNNSIIPEDTFFPSPESSCDVKLVEKSFETDTNLNFQNLSVIGFRILLLKV | |
| | | AGFNLLMTLRLWSS | |
| | TCRa full | MAGIRALFMYLWLQLDWVSRGESVGLHLPTLSVQEGDNSIINCAYSNSASDY | 286 |
| | (DS) | FIWYKQESGKGPQFIIDIRSNMDKRQGQRVTVLLNKTVKHLSLQIAATQPGD | |
| | | SAVYFCAEISKTSYDKVIFGPGTSLSVIPNIQNPDPAVYQLRDSKSSDKSVC | |
| | | LFTDFDSQTNVSQSKDSDVYITDKCVLDMRSMDFKSNSAVAWSNKSDFACAN | |
| | | AFNNSIIPEDTFFPSPESSCDVKLVEKSFETDTNLNFQNLSVIGFRILLLKV | |
| | | AGFNLLMTLRLWSS | |
| | TCRb | DMNHNYM | 145 |
| | CDR1 | | |
| | TCRb | YSVGAGIT | 146 |
| | CDR2 | | |
| | TCRb | CASSYGPGGRNTIYF | 147 |
| | CDR3 | | |
| | TCRb | NAGVTQTPKFRILKIGQSMTLQCAQDMNHNYMYWYRQDPGMGLKLIYYSVGA | 148 |
| | variable | GITDKGEVPNGYNVSRSTTEDFPLRLELAAPSQTSVYFCASSYGPGGRNTIY | |
| | domain | FGEGSWLTVV | |
| | TCRb full | MSISLLCCAAFPLLWAGPVNAGVTQTPKFRILKIGQSMTLQCAQDMNHNYMY | 149 |
| | | WYRQDPGMGLKLIYYSVGAGITDKGEVPNGYNVSRSTTEDFPLRLELAAPSQ | |
| | | TSVYFCASSYGPGGRNTIYFGEGSWLTVVEDLNKVFPPEVAVFEPSEAEISH | |
| | | TQKATLVCLATGFFPDHVELSWWVNGKEVHSGVSTDPQPLKEQPALNDSRYC | |
| | | LSSRLRVSATFWQNPRNHFRCQVQFYGLSENDEWTQDRAKPVTQIVSAEAWG | |
| | | RADCGFTSVSYQQGVLSATILYEILLGKATLYAVLVSALVLMAMVKRKDF | |
| | TCRb full | MSISLLCCAAFPLLWAGPVNAGVTQTPKFRILKIGQSMTLQCAQDMNHNYMY | 287 |
| | (DS) | WYRQDPGMGLKLIYYSVGAGITDKGEVPNGYNVSRSTTEDFPLRLELAAPSQ | |
| | | TSVYFCASSYGPGGRNTIYFGEGSWLTVVEDLNKVFPPEVAVFEPSEAEISH | |
| | | TQKATLVCLATGFFPDHVELSWWVNGKEVHSGVCTDPQPLKEQPALNDSRYC | |
| | | LSSRLRVSATFWQNPRNHFRCQVQFYGLSENDEWTQDRAKPVTQIVSAEAWG | |
| | | RADCGFTSVSYQQGVLSATILYEILLGKATLYAVLVSALVLMAMVKRKDF | |
| TCR-O | TCRa | TDSAIYNL | 150 |
| | CDR1 | | |
| | TCRa | LIQSSQREQ | 151 |
| | CDR2 | | |
| | TCRa | CAVTGMDSSYKLIF | 152 |
| | CDR3 | | |
| | TCRa | KQEVTQIPAALSVPEGENLVLNCSFTDSAIYNLQWFRQDPGKGLTSLLLIQS | 153 |
| | variable | SQREQTSGRLNASLDKSSGRSTLYIAASQPGDSATYLCAVTGMDSSYKLIFG | |
| | domain | SGTRLLVRP | |
| | TCRa full | METLLGLLILWLQLQWVSSKQEVTQIPAALSVPEGENLVLNCSFTDSAIYNL | 154 |
| | | QWFRQDPGKGLTSLLLIQSSQREQTSGRLNASLDKSSGRSTLYIAASQPGDS | |
| | | ATYLCAVTGMDSSYKLIFGSGTRLLVRPDIQNPDPAVYQLRDSKSSDKSVCL | |
| | | FTDFDSQTNVSQSKDSDVYITDKTVLDMRSMDFKSNSAVAWSNKSDFACANA | |
| | | FNNSIIPEDTFFPSPESSCDVKLVEKSFETDTNLNFQNLSVIGFRILLLKVA | |
| | | GFNLLMTLRLWSS | |
| | TCRa full | METLLGLLILWLQLQWVSSKQEVTQIPAALSVPEGENLVLNCSFTDSAIYNL | 288 |
| | (DS) | QWFRQDPGKGLTSLLLIQSSQREQTSGRLNASLDKSSGRSTLYIAASQPGDS | |
| | | ATYLCAVTGMDSSYKLIFGSGTRLLVRPDIQNPDPAVYQLRDSKSSDKSVCL | |
| | | FTDFDSQTNVSQSKDSDVYITDKCVLDMRSMDFKSNSAVAWSNKSDFACANA | |
| | | FNNSIIPEDTFFPSPESSCDVKLVEKSFETDTNLNFQNLSVIGFRILLLKVA | |
| | | GFNLLMTLRLWSS | |
| | TCRb | TWNHNNM | 155 |
| | CDR1 | | |
| | TCRb | YSYGVHDT | 156 |
| | CDR2 | | |
| | TCRb | CASSEGGSWGYTF | 157 |
| | CDR3 | | |
| | TCRb | DAEITQSPRHKITETGRQVTLACHQTWNHNNMFWYRQDLGHGLRLIHYSYGV | 158 |
| | variable | HDTNKGEVSDGYSVSRSNTEDLPLTLESAASSQTSVYFCASSEGGSWGYTFG | |
| | domain | SGTRLTVV | |
| | TCRb full | MGTRLFFYVALCLLWAGHRDAEITQSPRHKITETGRQVTLACHQTWNHNNMF | 159 |
| | | WYRQDLGHGLRLIHYSYGVHDTNKGEVSDGYSVSRSNTEDLPLTLESAASSQ | |
| | | TSVYFCASSEGGSWGYTFGSGTRLTVVEDLNKVFPPEVAVFEPSEAEISHTQ | |
| | | KATLVCLATGFFPDHVELSWWVNGKEVHSGVSTDPQPLKEQPALNDSRYCLS | |
| | | SRLRVSATFWQNPRNHFRCQVQFYGLSENDEWTQDRAKPVTQIVSAEAWGRA | |
| | | DCGFTSVSYQQGVLSATILYEILLGKATLYAVLVSALVLMAMVKRKDF | |
| | TCRb full | MGTRLFFYVALCLLWAGHRDAEITQSPRHKITETGRQVTLACHQTWNHNNMF | 289 |
| | (DS) | WYRQDLGHGLRLIHYSYGVHDTNKGEVSDGYSVSRSNTEDLPLTLESAASSQ | |
| | | TSVYFCASSEGGSWGYTFGSGTRLTVVEDLNKVFPPEVAVFEPSEAEISHTQ | |
| | | KATLVCLATGFFPDHVELSWWVNGKEVHSGVCTDPQPLKEQPALNDSRYCLS | |
| | | SRLRVSATFWQNPRNHFRCQVQFYGLSENDEWTQDRAKPVTQIVSAEAWGRA | |
| | | DCGFTSVSYQQGVLSATILYEILLGKATLYAVLVSALVLMAMVKRKDF | |
| TCR-P | TCRa | STISGNEYV | 160 |
| | CDR1 | | |
| | TCRa | HGLKNNE | 161 |
| | CDR2 | | |
| | TCRa | CIVRVLYNNNDMRF | 162 |
| | CDR3 | | |
| | TCRa | DAKTTQPPSMDCAEGRAANLPCNHSTISGNEYVYWYRQIHSQGPQYIIHGLK | 163 |
| | variable | NNETNEMASLIITEDRKSSTLILPHATLRDTAVYYCIVRVLYNNNDMRFGAG | |
| | domain | TRLTVKP | |
| | TCRa full | MRLVARVTVFLTFGTIIDAKTTQPPSMDCAEGRAANLPCNHSTISGNEYVYW | 164 |
| | | YRQIHSQGPQYIIHGLKNNETNEMASLIITEDRKSSTLILPHATLRDTAVYY | |
| | | CIVRVLYNNNDMRFGAGTRLTVKPNIQNPDPAVYQLRDSKSSDKSVCLFTDF | |
| | | DSQTNVSQSKDSDVYITDKTVLDMRSMDFKSNSAVAWSNKSDFACANAFNNS | |
| | | IIPEDTFFPSPESSCDVKLVEKSFETDTNLNFQNLSVIGFRILLLKVAGFNL | |
| | | LMTLRLWSS | |
| | TCRa full | MRLVARVTVFLTFGTIIDAKTTQPPSMDCAEGRAANLPCNHSTISGNEYVYW | 290 |
| | (DS) | YRQIHSQGPQYIIHGLKNNETNEMASLIITEDRKSSTLILPHATLRDTAVYY | |
| | | CIVRVLYNNNDMRFGAGTRLTVKPNIQNPDPAVYQLRDSKSSDKSVCLFTDF | |
| | | DSQTNVSQSKDSDVYITDKCVLDMRSMDFKSNSAVAWSNKSDFACANAFNNS | |
| | | IIPEDTFFPSPESSCDVKLVEKSFETDTNLNFQNLSVIGFRILLLKVAGFNL | |
| | | LMTLRLWSS | |
| | TCRb | DMNHNYM | 165 |
| | CDR1 | | |
| | TCRb | YSVGAGIT | 166 |
| | CDR2 | | |
| | TCRb | CASSSTGPNTGELFF | 167 |
| | CDR3 | | |
| | TCRb | NAGVTQTPKFRILKIGQSMTLQCTQDMNHNYMYWYRQDPGMGLKLIYYSVGA | 168 |
| | variable | GITDKGEVPNGYNVSRSTTEDFPLRLELAAPSQTSVYFCASSSTGPNTGELF | |
| | domain | FGEGSRLTVL | |
| | TCRb full | MSISLLCCAAFPLLWAGPVNAGVTQTPKFRILKIGQSMTLQCTQDMNHNYMY | 169 |
| | | WYRQDPGMGLKLIYYSVGAGITDKGEVPNGYNVSRSTTEDFPLRLELAAPSQ | |
| | | TSVYFCASSSTGPNTGELFFGEGSRLTVLEDLKNVFPPEVAVFEPSEAEISH | |
| | | TQKATLVCLATGFYPDHVELSWWVNGKEVHSGVSTDPQPLKEQPALNDSRYC | |
| | | LSSRLRVSATFWQNPRNHFRCQVQFYGLSENDEWTQDRAKPVTQIVSAEAWG | |
| | | RADCGFTSESYQQGVLSATILYEILLGKATLYAVLVSALVLMAMVKRKDSRG | |
| | TCRb full | MSISLLCCAAFPLLWAGPVNAGVTQTPKFRILKIGQSMTLQCTQDMNHNYMY | 291 |
| | (DS) | WYRQDPGMGLKLIYYSVGAGITDKGEVPNGYNVSRSTTEDFPLRLELAAPSQ | |
| | | TSVYFCASSSTGPNTGELFFGEGSRLTVLEDLKNVFPPEVAVFEPSEAEISH | |
| | | TQKATLVCLATGFYPDHVELSWWVNGKEVHSGVCTDPQPLKEQPALNDSRYC | |
| | | LSSRLRVSATFWQNPRNHFRCQVQFYGLSENDEWTQDRAKPVTQIVSAEAWG | |
| | | RADCGFTSESYQQGVLSATILYEILLGKATLYAVLVSALVLMAMVKRKDSRG | |
| TCR-Q | TCRa | SVSNAYNF | 170 |
| | CDR1 | | |
| | TCRa | KGSKPS | 171 |
| | CDR2 | | |
| | TCRa | CAVGDTGGFKTIF | 172 |
| | CDR3 | | |
| | TCRa | KDQVFQPSTVASSEGAVVEIFCNHSVSNAYNFFWYLHFPGCAPRLLVKGSKP | 173 |
| | variable | SQQGRYNMTYERFSSSLLILQVREADAAVYYCAVGDTGGFKTIFGAGTRLFV | |
| | domain | KA | |
| | TCRa full | MALQSTLGAVWLGLLLNSLWKVAESKDQVFQPSTVASSEGAVVEIFCNHSVS | 174 |
| | | NAYNFFWYLHFPGCAPRLLVKGSKPSQQGRYNMTYERFSSSLLILQVREADA | |
| | | AVYYCAVGDTGGFKTIFGAGTRLFVKANIQNPDPAVYQLRDSKSSDKSVCLF | |
| | | TDFDSQTNVSQSKDSDVYITDKTVLDMRSMDFKSNSAVAWSNKSDFACANAF | |
| | | NNSIIPEDTFFPSPESSCDVKLVEKSFETDTNLNFQNLSVIGFRILLLKVAG | |
| | | FNLLMTLRLWSS | |
| | TCRa full | MALQSTLGAVWLGLLLNSLWKVAESKDQVFQPSTVASSEGAVVEIFCNHSVS | 292 |
| | (DS) | NAYNFFWYLHFPGCAPRLLVKGSKPSQQGRYNMTYERFSSSLLILQVREADA | |
| | | AVYYCAVGDTGGFKTIFGAGTRLFVKANIQNPDPAVYQLRDSKSSDKSVCLF | |
| | | TDFDSQTNVSQSKDSDVYITDKCVLDMRSMDFKSNSAVAWSNKSDFACANAF | |
| | | NNSIIPEDTFFPSPESSCDVKLVEKSFETDTNLNFQNLSVIGFRILLLKVAG | |
| | | FNLLMTLRLWSS | |
| | TCRb | RSGDLSV | 175 |
| | CDR1 | | |
| | TCRb | HYYNGEER | 176 |
| | CDR2 | | |
| | TCRb | CASSEIAGFSTDTQYF | 177 |
| | CDR3 | | |
| | TCRb | DSGVTQTPKHLITATGQRVTLRCSPRSGDLSVYWYQQSLDQGLQFLIHYYNG | 178 |
| | variable | EERAKGNILERFSAQQFPDLHSELNLSSLELGDSALYFCASSEIAGFSTDTQ | |
| | domain | YFGPGTRLTVL | |
| | TCRb full | MGFRLLCCVAFCLLGAGPVDSGVTQTPKHLITATGQRVTLRCSPRSGDLSVY | 179 |
| | | WYQQSLDQGLQFLIHYYNGEERAKGNILERFSAQQFPDLHSELNLSSLELGD | |
| | | SALYFCASSEIAGFSTDTQYFGPGTRLTVLEDLKNVFPPEVAVFEPSEAEIS | |
| | | HTQKATLVCLATGFYPDHVELSWWVNGKEVHSGVSTDPQPLKEQPALNDSRY | |
| | | CLSSRLRVSATFWQNPRNHFRCQVQFYGLSENDEWTQDRAKPVTQIVSAEAW | |
| | | GRADCGFTSESYQQGVLSATILYEILLGKATLYAVLVSALVLMAMVKRKDSR | |
| | | G | |
| | TCRb full | MGFRLLCCVAFCLLGAGPVDSGVTQTPKHLITATGQRVTLRCSPRSGDLSVY | 293 |
| | (DS) | WYQQSLDQGLQFLIHYYNGEERAKGNILERFSAQQFPDLHSELNLSSLELGD | |
| | | SALYFCASSEIAGFSTDTQYFGPGTRLTVLEDLKNVFPPEVAVFEPSEAEIS | |
| | | HTQKATLVCLATGFYPDHVELSWWVNGKEVHSGVCTDPQPLKEQPALNDSRY | |
| | | CLSSRLRVSATFWQNPRNHFRCQVQFYGLSENDEWTQDRAKPVTQIVSAEAW | |
| | | GRADCGFTSESYQQGVLSATILYEILLGKATLYAVLVSALVLMAMVKRKDSR | |
| | | G | |
| TCR-R | TCRa | SNSASQSF | 180 |
| | CDR1 | | |
| | TCRa | SVYSSGN | 181 |
| | CDR2 | | |
| | TCRa | CVVTASNSGNTPLVF | 182 |
| | CDR3 | | |
| | TCRa | RKEVEQDPGPFNVPEGATVAFNCTYSNSASQSFFWYRQDCRKEPKLLMSVYS | 183 |
| | variable | SGNEDGRFTAQLNRASQYISLLIRDSKLSDSATYLCVVTASNSGNTPLVFGK | |
| | domain | GTRLSVIA | |
| | TCRa full | MISLRVLLVILWLQLSWVWSQRKEVEQDPGPFNVPEGATVAFNCTYSNSASQ | 294 |
| | | SFFWYRQDCRKEPKLLMSVYSSGNEDGRFTAQLNRASQYISLLIRDSKLSDS | |
| | | ATYLCVVTASNSGNTPLVFGKGTRLSVIANIQNPDPAVYQLRDSKSSDKSVC | |
| | | LFTDFDSQTNVSQSKDSDVYITDKTVLDMRSMDFKSNSAVAWSNKSDFACAN | |
| | | AFNNSIIPEDTFFPSPESSCDVKLVEKSFETDTNLNFQNLSVIGFRILLLKV | |
| | | AGFNLLMTLRLWSS | |
| | TCRa full | MISLRVLLVILWLQLSWVWSQRKEVEQDPGPFNVPEGATVAFNCTYSNSASQ | 184 |
| | (DS) | SFFWYRQDCRKEPKLLMSVYSSGNEDGRFTAQLNRASQYISLLIRDSKLSDS | |
| | | ATYLCVVTASNSGNTPLVFGKGTRLSVIANIQNPDPAVYQLRDSKSSDKSVC | |
| | | LFTDFDSQTNVSQSKDSDVYITDKCVLDMRSMDFKSNSAVAWSNKSDFACAN | |
| | | AFNNSIIPEDTFFPSPESSCDVKLVEKSFETDTNLNFQNLSVIGFRILLLKV | |
| | | AGFNLLMTLRLWSS | |
| | TCRb | DSQVTMM | 185 |
| | CDR1 | | |
| | TCRb | TANQGSEAT | 186 |
| | CDR2 | | |
| | TCRb | CSVGGGDGYTF | 187 |
| | CDR3 | | |
| | TCRb | SAVISQKPSRDICQRGTSLTIQCQVDSQVTMMFWYRQQPGQSLTLIATANQG | 188 |
| | variable | SEATYESGFVIDKFPISRPNLTFSTLTVSNMSPEDSSIYLCSVGGGDGYTFG | |
| | domain | SGTRLTVV | |
| | TCRb full | MLSLLLLLLGLGSVFSAVISQKPSRDICQRGTSLTIQCQVDSQVTMMFWYRQ | 295 |
| | | QPGQSLTLIATANQGSEATYESGFVIDKFPISRPNLTFSTLTVSNMSPEDSS | |
| | | IYLCSVGGGDGYTFGSGTRLTVVEDLNKVFPPEVAVFEPSEAEISHTQKATL | |
| | | VCLATGFFPDHVELSWWVNGKEVHSGVSTDPQPLKEQPALNDSRYCLSSRLR | |
| | | VSATFWQNPRNHFRCQVQFYGLSENDEWTQDRAKPVTQIVSAEAWGRADCGF | |
| | | TSVSYQQGVLSATILYEILLGKATLYAVLVSALVLMAMVKRKDF | |
| | TCRb full | MLSLLLLLLGLGSVFSAVISQKPSRDICQRGTSLTIQCQVDSQVTMMFWYRQ | 189 |
| | (DS) | QPGQSLTLIATANQGSEATYESGFVIDKFPISRPNLTFSTLTVSNMSPEDSS | |
| | | IYLCSVGGGDGYTFGSGTRLTVVEDLNKVFPPEVAVFEPSEAEISHTQKATL | |
| | | VCLATGFFPDHVELSWWVNGKEVHSGVCTDPQPLKEQPALNDSRYCLSSRLR | |
| | | VSATFWQNPRNHFRCQVQFYGLSENDEWTQDRAKPVTQIVSAEAWGRADCGF | |
| | | TSVSYQQGVLSATILYEILLGKATLYAVLVSALVLMAMVKRKDF | |
| TCR-S | TCRa | SNSAFQYF | 190 |
| | CDR1 | | |
| | TCRa | YTYSSGNK | 191 |
| | CDR2 | | |
| | TCRa | CAMSEYSGYALNF | 192 |
| | CDR3 | | |
| | TCRa | QKEVEQDPGPLSVPEGAIVSLNCTYSNSAFQYFMWYRQYSRKGPELLMYTYS | 193 |
| | variable | SGNKEDGRFTAQVDKSSKYISLFIRDSQPSDSATYLCAMSEYSGYALNFGKG | |
| | domain | TSLLVTP | |
| | TCRa full | MMKSLRVLLVILWLQLSWVWSQQKEVEQDPGPLSVPEGAIVSLNCTYSNSAF | 296 |
| | | QYFMWYRQYSRKGPELLMYTYSSGNKEDGRFTAQVDKSSKYISLFIRDSQPS | |
| | | DSATYLCAMSEYSGYALNFGKGTSLLVTPHIQNPDPAVYQLRDSKSSDKSVC | |
| | | LFTDFDSQTNVSQSKDSDVYITDKTVLDMRSMDFKSNSAVAWSNKSDFACAN | |
| | | AFNNSIIPEDTFFPSPESSCDVKLVEKSFETDTNLNFQNLSVIGFRILLLKV | |
| | | AGFNLLMTLRLWSS | |
| | TCRa full | MMKSLRVLLVILWLQLSWVWSQQKEVEQDPGPLSVPEGAIVSLNCTYSNSAF | 194 |
| | (DS) | QYFMWYRQYSRKGPELLMYTYSSGNKEDGRFTAQVDKSSKYISLFIRDSQPS | |
| | | DSATYLCAMSEYSGYALNFGKGTSLLVTPHIQNPDPAVYQLRDSKSSDKSVC | |
| | | LFTDFDSQTNVSQSKDSDVYITDKCVLDMRSMDFKSNSAVAWSNKSDFACAN | |
| | | AFNNSIIPEDTFFPSPESSCDVKLVEKSFETDTNLNFQNLSVIGFRILLLKV | |
| | | AGFNLLMTLRLWSS | |
| | TCRb | NLNHDAM | 195 |
| | CDR1 | | |
| | TCRb | YSQIVNDF | 196 |
| | CDR2 | | |
| | TCRb | CASSNRDEQYF | 197 |
| | CDR3 | | |
| | TCRb | DGGITQSPKYLFRKEGQNVTLSCEQNLNHDAMYWYRQDPGQGLRLIYYSQIV | 198 |
| | variable | NDFQKGDIAEGYSVSREKKESFPLTVTSAQKNPTAFYLCASSNRDEQYFGPG | |
| | domain | TRLTVT | |
| | TCRb full | MSNQVLCCVVLCLLGANTVDGGITQSPKYLFRKEGQNVTLSCEQNLNHDAMY | 297 |
| | | WYRQDPGQGLRLIYYSQIVNDFQKGDIAEGYSVSREKKESFPLTVTSAQKNP | |
| | | TAFYLCASSNRDEQYFGPGTRLTVTEDLKNVFPPEVAVFEPSEAEISHTQKA | |
| | | TLVCLATGFYPDHVELSWWVNGKEVHSGVSTDPQPLKEQPALNDSRYCLSSR | |
| | | LRVSATFWQNPRNHFRCQVQFYGLSENDEWTQDRAKPVTQIVSAEAWGRADC | |
| | | GFTSESYQQGVLSATILYEILLGKATLYAVLVSALVLMAMVKRKDSRG | |
| | TCRb full | MSNQVLCCVVLCLLGANTVDGGITQSPKYLFRKEGQNVTLSCEQNLNHDAMY | 199 |
| | (DS) | WYRQDPGQGLRLIYYSQIVNDFQKGDIAEGYSVSREKKESFPLTVTSAQKNP | |
| | | TAFYLCASSNRDEQYFGPGTRLTVTEDLKNVFPPEVAVFEPSEAEISHTQKA | |
| | | TLVCLATGFYPDHVELSWWVNGKEVHSGVCTDPQPLKEQPALNDSRYCLSSR | |
| | | LRVSATFWQNPRNHFRCQVQFYGLSENDEWTQDRAKPVTQIVSAEAWGRADC | |
| | | GFTSESYQQGVLSATILYEILLGKATLYAVLVSALVLMAMVKRKDSRG | |
| TCR-T | TCRa | TVSPFSNL | 200 |
| | CDR1 | | |
| | TCRa | IMTFSENTK | 201 |
| | CDR2 | | |
| | TCRa | CVVSAKTGANNLFF | 202 |
| | CDR3 | | |
| | TCRa | KNQVEQSPQSLIILEGKNCTLQCNYTVSPFSNLRWYKQDTGRGPVSLTIMTF | 203 |
| | variable | SENTKSNGRYTATLDADTKQSSLHITASQLSDSASYICVVSAKTGANNLFFG | |
| | domain | TGTRLTVIP | |
| | TCRa full | MKKHLTTFLVILWLYFYRGNGKNQVEQSPQSLIILEGKNCTLQCNYTVSPFS | 298 |
| | | NLRWYKQDTGRGPVSLTIMTFSENTKSNGRYTATLDADTKQSSLHITASQLS | |
| | | DSASYICVVSAKTGANNLFFGTGTRLTVIPYIQNPDPAVYQLRDSKSSDKSV | |
| | | CLFTDFDSQTNVSQSKDSDVYITDKTVLDMRSMDFKSNSAVAWSNKSDFACA | |
| | | NAFNNSIIPEDTFFPSPESSCDVKLVEKSFETDTNLNFQNLSVIGFRILLLK | |
| | | VAGFNLLMTLRLWSS | |
| | TCRa full | MKKHLTTFLVILWLYFYRGNGKNQVEQSPQSLIILEGKNCTLQCNYTVSPFS | 204 |
| | (DS) | NLRWYKQDTGRGPVSLTIMTFSENTKSNGRYTATLDADTKQSSLHITASQLS | |
| | | DSASYICVVSAKTGANNLFFGTGTRLTVIPYIQNPDPAVYQLRDSKSSDKSV | |
| | | CLFTDFDSQTNVSQSKDSDVYITDKCVLDMRSMDFKSNSAVAWSNKSDFACA | |
| | | NAFNNSIIPEDTFFPSPESSCDVKLVEKSFETDTNLNFQNLSVIGFRILLLK | |
| | | VAGFNLLMTLRLWSS | |
| | TCRb | TWSHSYM | 205 |
| | CDR1 | | |
| | TCRb | YSAAADIT | 206 |
| | CDR2 | | |
| | TCRb | CASSETDYGYTF | 207 |
| | CDR3 | | |
| | TCRb | DAGITQSPRYKITETGRQVTLMCHQTWSHSYMFWYRQDLGHGLRLIYYSAAA | 208 |
| | variable | DITDKGEVPDGYVVSRSKTENFPLTLESATRSQTSVYFCASSETDYGYTFGS | |
| | domain | GTRLTVV | |
| | TCRb full | MGTRLFFYVALCLLWAGHRDAGITQSPRYKITETGRQVTLMCHQTWSHSYMF | 299 |
| | | WYRQDLGHGLRLIYYSAAADITDKGEVPDGYVVSRSKTENFPLTLESATRSQ | |
| | | TSVYFCASSETDYGYTFGSGTRLTVVEDLNKVFPPEVAVFEPSEAEISHTQK | |
| | | ATLVCLATGFFPDHVELSWWVNGKEVHSGVSTDPQPLKEQPALNDSRYCLSS | |
| | | RLRVSATFWQNPRNHFRCQVQFYGLSENDEWTQDRAKPVTQIVSAEAWGRAD | |
| | | CGFTSVSYQQGVLSATILYEILLGKATLYAVLVSALVLMAMVKRKDF | |
| | TCRb full | MGTRLFFYVALCLLWAGHRDAGITQSPRYKITETGRQVTLMCHQTWSHSYMF | 209 |
| | (DS) | WYRQDLGHGLRLIYYSAAADITDKGEVPDGYVVSRSKTENFPLTLESATRSQ | |
| | | TSVYFCASSETDYGYTFGSGTRLTVVEDLNKVFPPEVAVFEPSEAEISHTQK | |
| | | ATLVCLATGFFPDHVELSWWVNGKEVHSGVCTDPQPLKEQPALNDSRYCLSS | |
| | | RLRVSATFWQNPRNHFRCQVQFYGLSENDEWTQDRAKPVTQIVSAEAWGRAD | |
| | | CGFTSVSYQQGVLSATILYEILLGKATLYAVLVSALVLMAMVKRKDF | |
| TCR-U | TCRa | TVSGLRGL | 210 |
| | CDR1 | | |
| | TCRa | TLYSAGEEK | 211 |
| | CDR2 | | |
| | TCRa | CAVQVSRNTGKLIF | 212 |
| | CDR3 | | |
| | TCRa | EDQVTQSPEALRLQEGESSSLNCSYTVSGLRGLFWYRQDPGKGPEFLFTLYS | 213 |
| | variable | AGEEKEKERLKATLTKKESFLHITAPKPEDSATYLCAVQVSRNTGKLIFGQG | |
| | domain | TTLQVKP | |
| | TCRa full | MEKMLECAFIVLWLQLGWLSGEDQVTQSPEALRLQEGESSSLNCSYTVSGLR | 300 |
| | | GLFWYRQDPGKGPEFLFTLYSAGEEKEKERLKATLTKKESFLHITAPKPEDS | |
| | | ATYLCAVQVSRNTGKLIFGQGTTLQVKPDIQNPDPAVYQLRDSKSSDKSVCL | |
| | | FTDFDSQTNVSQSKDSDVYITDKTVLDMRSMDFKSNSAVAWSNKSDFACANA | |
| | | FNNSIIPEDTFFPSPESSCDVKLVEKSFETDTNLNFQNLSVIGFRILLLKVA | |
| | | GFNLLMTLRLWSS | |
| | TCRa full | MEKMLECAFIVLWLQLGWLSGEDQVTQSPEALRLQEGESSSLNCSYTVSGLR | 214 |
| | (DS) | GLFWYRQDPGKGPEFLFTLYSAGEEKEKERLKATLTKKESFLHITAPKPEDS | |
| | | ATYLCAVQVSRNTGKLIFGQGTTLQVKPDIQNPDPAVYQLRDSKSSDKSVCL | |
| | | FTDFDSQTNVSQSKDSDVYITDKCVLDMRSMDFKSNSAVAWSNKSDFACANA | |
| | | FNNSIIPEDTFFPSPESSCDVKLVEKSFETDTNLNFQNLSVIGFRILLLKVA | |
| | | GFNLLMTLRLWSS | |
| | TCRb | DMNHNSM | 215 |
| | CDR1 | | |
| | TCRb | YSASEGTT | 216 |
| | CDR2 | | |
| | TCRb | CASSELGQPIYGYTF | 217 |
| | CDR3 | | |
| | TCRb | NAGVTQTPKFQVLKTGQSMTLQCAQDMNHNSMYWYRQDPGMGLRLIYYSASE | 218 |
| | variable | GTTDKGEVPNGYNVSRLNKREFSLRLESAAPSQTSVYFCASSELGQPIYGYT | |
| | domain | FGSGTRLTVV | |
| | TCRb full | MSIGLLCCVAFSLLWASPVNAGVTQTPKFQVLKTGQSMTLQCAQDMNHNSMY | 301 |
| | | WYRQDPGMGLRLIYYSASEGTTDKGEVPNGYNVSRLNKREFSLRLESAAPSQ | |
| | | TSVYFCASSELGQPIYGYTFGSGTRLTVVEDLNKVFPPEVAVFEPSEAEISH | |
| | | TQKATLVCLATGFFPDHVELSWWVNGKEVHSGVSTDPQPLKEQPALNDSRYC | |
| | | LSSRLRVSATFWQNPRNHFRCQVQFYGLSENDEWTQDRAKPVTQIVSAEAWG | |
| | | RADCGFTSVSYQQGVLSATILYEILLGKATLYAVLVSALVLMAMVKRKDF | |
| | TCRb full | MSIGLLCCVAFSLLWASPVNAGVTQTPKFQVLKTGQSMTLQCAQDMNHNSMY | 219 |
| | (DS) | WYRQDPGMGLRLIYYSASEGTTDKGEVPNGYNVSRLNKREFSLRLESAAPSQ | |
| | | TSVYFCASSELGQPIYGYTFGSGTRLTVVEDLNKVFPPEVAVFEPSEAEISH | |
| | | TQKATLVCLATGFFPDHVELSWWVNGKEVHSGVCTDPQPLKEQPALNDSRYC | |
| | | LSSRLRVSATFWQNPRNHFRCQVQFYGLSENDEWTQDRAKPVTQIVSAEAWG | |
| | | RADCGFTSVSYQQGVLSATILYEILLGKATLYAVLVSALVLMAMVKRKDF | |
| TCR-V | TCRa | TATGYPSL | 220 |
| | CDR1 | | |
| | TCRa | KATKADDKG | 221 |
| | CDR2 | | |
| | TCRa | CALSSKTSYDKVIF | 222 |
| | CDR3 | | |
| | TCRa | GDSVTQMEGPVTLSEEAFLTINCTYTATGYPSLFWYVQYPGEGLQLLLKATK | 223 |
| | variable | ADDKGSNKGFEATYRKETTSFHLEKGSVQVSDSAVYFCALSSKTSYDKVIFG | |
| | domain | PGTSLSVIP | |
| | TCRa full | MNYSPGLVSLILLLLGRTRGDSVTQMEGPVTLSEEAFLTINCTYTATGYPSL | 302 |
| | | FWYVQYPGEGLQLLLKATKADDKGSNKGFEATYRKETTSFHLEKGSVQVSDS | |
| | | AVYFCALSSKTSYDKVIFGPGTSLSVIPNIQNPDPAVYQLRDSKSSDKSVCL | |
| | | FTDFDSQTNVSQSKDSDVYITDKTVLDMRSMDFKSNSAVAWSNKSDFACANA | |
| | | FNNSIIPEDTFFPSPESSCDVKLVEKSFETDTNLNFQNLSVIGFRILLLKVA | |
| | | GFNLLMTLRLWSS | |
| | TCRa full | MNYSPGLVSLILLLLGRTRGDSVTQMEGPVTLSEEAFLTINCTYTATGYPSL | 224 |
| | (DS) | FWYVQYPGEGLQLLLKATKADDKGSNKGFEATYRKETTSFHLEKGSVQVSDS | |
| | | AVYFCALSSKTSYDKVIFGPGTSLSVIPNIQNPDPAVYQLRDSKSSDKSVCL | |
| | | FTDFDSQTNVSQSKDSDVYITDKCVLDMRSMDFKSNSAVAWSNKSDFACANA | |
| | | FNNSIIPEDTFFPSPESSCDVKLVEKSFETDTNLNFQNLSVIGFRILLLKVA | |
| | | GFNLLMTLRLWSS | |
| | TCRb | DMNHNSM | 225 |
| | CDR1 | | |
| | TCRb | YSASEGTT | 226 |
| | CDR2 | | |
| | TCRb | CASRDRNSGNTIYF | 227 |
| | CDR3 | | |
| | TCRb | NAGVTQTPKFQVLKTGQSMTLQCAQDMNHNSMYWYRQDPGMGLRLIYYSASE | 228 |
| | variable | GTTDKGEVPNGYNVSRLNKREFSLRLESAAPSQTSVYFCASRDRNSGNTIYF | |
| | domain | GEGSWLTVV | |
| | TCRb full | MSIGLLCCVAFSLLWASPVNAGVTQTPKFQVLKTGQSMTLQCAQDMNHNSMY | 303 |
| | | WYRQDPGMGLRLIYYSASEGTTDKGEVPNGYNVSRLNKREFSLRLESAAPSQ | |
| | | TSVYFCASRDRNSGNTIYFGEGSWLTVVEDLNKVFPPEVAVFEPSEAEISHT | |
| | | QKATLVCLATGFFPDHVELSWWVNGKEVHSGVSTDPQPLKEQPALNDSRYCL | |
| | | SSRLRVSATFWQNPRNHFRCQVQFYGLSENDEWTQDRAKPVTQIVSAEAWGR | |
| | | ADCGFTSVSYQQGVLSATILYEILLGKATLYAVLVSALVLMAMVKRKDF | |
| | TCRb full | MSIGLLCCVAFSLLWASPVNAGVTQTPKFQVLKTGQSMTLQCAQDMNHNSMY | 229 |
| | (DS) | WYRQDPGMGLRLIYYSASEGTTDKGEVPNGYNVSRLNKREFSLRLESAAPSQ | |
| | | TSVYFCASRDRNSGNTIYFGEGSWLTVVEDLNKVFPPEVAVFEPSEAEISHT | |
| | | QKATLVCLATGFFPDHVELSWWVNGKEVHSGVCTDPQPLKEQPALNDSRYCL | |
| | | SSRLRVSATFWQNPRNHFRCQVQFYGLSENDEWTQDRAKPVTQIVSAEAWGR | |
| | | ADCGFTSVSYQQGVLSATILYEILLGKATLYAVLVSALVLMAMVKRKDF | |
| TCR-X | TCRa | SNSASDYF | 230 |
| | CDR1 | | |
| | TCRa | DIRSNMDKR | 231 |
| | CDR2 | | |
| | TCRa | CAESSAGGYNKLIF | 232 |
| | CDR3 | | |
| | TCRa | GESVGLHLPTLSVQEGDNSIINCAYSNSASDYFIWYKQESGKGPQFIIDIRS | 233 |
| | variable | NMDKRQGQRVTVLLNKTVKHLSLQIAATQPGDSAVYFCAESSAGGYNKLIFG | |
| | domain | AGTRLAVHP | |
| | TCRb full | MAGIRALFMYLWLQLDWVSRGESVGLHLPTLSVQEGDNSIINCAYSNSASDY | 304 |
| | | FIWYKQESGKGPQFIIDIRSNMDKRQGQRVTVLLNKTVKHLSLQIAATQPGD | |
| | | SAVYFCAESSAGGYNKLIFGAGTRLAVHPYIQNPDPAVYQLRDSKSSDKSVC | |
| | | LFTDFDSQTNVSQSKDSDVYITDKTVLDMRSMDFKSNSAVAWSNKSDFACAN | |
| | | AFNNSIIPEDTFFPSPESSCDVKLVEKSFETDTNLNFQNLSVIGFRILLLKV | |
| | | AGFNLLMTLRLWSS | |
| | TCRa full | MAGIRALFMYLWLQLDWVSRGESVGLHLPTLSVQEGDNSIINCAYSNSASDY | 234 |
| | (DS) | FIWYKQESGKGPQFIIDIRSNMDKRQGQRVTVLLNKTVKHLSLQIAATQPGD | |
| | | SAVYFCAESSAGGYNKLIFGAGTRLAVHPYIQNPDPAVYQLRDSKSSDKSVC | |
| | | LFTDFDSQTNVSQSKDSDVYITDKCVLDMRSMDFKSNSAVAWSNKSDFACAN | |
| | | AFNNSIIPEDTFFPSPESSCDVKLVEKSFETDTNLNFQNLSVIGFRILLLKV | |
| | | AGFNLLMTLRLWSS | |
| | TCRb | ISGHNSL | 235 |
| | CDR1 | | |
| | TCRb | YFNNNVPI | 236 |
| | CDR2 | | |
| | TCRb | CASRGQVGGYGYTF | 237 |
| | CDR3 | | |
| | TCRb | DAGVIQSPRHEVTEMGQEVTLRCKPISGHNSLFWYRQTMMRGLELLIYFNNN | 238 |
| | variable | VPIDDSGMPEDRFSAKMPNASFSTLKIQPSEPRDSAVYFCASRGQVGGYGYT | |
| | domain | FGSGTRLTVV | |
| | TCRb full | MDSWTFCCVSLCILVAKHTDAGVIQSPRHEVTEMGQEVTLRCKPISGHNSLF | 305 |
| | | WYRQTMMRGLELLIYFNNNVPIDDSGMPEDRFSAKMPNASFSTLKIQPSEPR | |
| | | DSAVYFCASRGQVGGYGYTFGSGTRLTVVEDLNKVFPPEVAVFEPSEAEISH | |
| | | TQKATLVCLATGFFPDHVELSWWVNGKEVHSGVSTDPQPLKEQPALNDSRYC | |
| | | LSSRLRVSATFWQNPRNHFRCQVQFYGLSENDEWTQDRAKPVTQIVSAEAWG | |
| | | RADCGFTSVSYQQGVLSATILYEILLGKATLYAVLVSALVLMAMVKRKDF | |
| | TCRb full | MDSWTFCCVSLCILVAKHTDAGVIQSPRHEVTEMGQEVTLRCKPISGHNSLF | 239 |
| | (DS) | WYRQTMMRGLELLIYFNNNVPIDDSGMPEDRFSAKMPNASFSTLKIQPSEPR | |
| | | DSAVYFCASRGQVGGYGYTFGSGTRLTVVEDLNKVFPPEVAVFEPSEAEISH | |
| | | TQKATLVCLATGFFPDHVELSWWVNGKEVHSGVCTDPQPLKEQPALNDSRYC | |
| | | LSSRLRVSATFWQNPRNHFRCQVQFYGLSENDEWTQDRAKPVTQIVSAEAWG | |
| | | RADCGFTSVSYQQGVLSATILYEILLGKATLYAVLVSALVLMAMVKRKDF | |
| TCR-Y | TCRa | ENTAFDYF | 240 |
| | CDR1 | | |
| | TCRa | AIRPDVSEK | 241 |
| | CDR2 | | |
| | TCRa | CAASSGTYKYIF | 242 |
| | CDR3 | | |
| | TCRa | QQQVKQSPQSLIVQKGGISIINCAYENTAFDYFPWYQQFPGKGPALLIAIRP | 243 |
| | variable | DVSEKKEGRFTISFNKSAKQFSLHIMDSQPGDSATYFCAASSGTYKYIFGTG | |
| | domain | TRLKVLA | |
| | TCRa full | MDKILGASFLVLWLQLCWVSGQQKEKSDQQQVKQSPQSLIVQKGGISIINCA | 306 |
| | | YENTAFDYFPWYQQFPGKGPALLIAIRPDVSEKKEGRFTISFNKSAKQFSLH | |
| | | IMDSQPGDSATYFCAASSGTYKYIFGTGTRLKVLANIQNPDPAVYQLRDSKS | |
| | | SDKSVCLFTDFDSQTNVSQSKDSDVYITDKTVLDMRSMDFKSNSAVAWSNKS | |
| | | DFACANAFNNSIIPEDTFFPSPESSCDVKLVEKSFETDTNLNFQNLSVIGFR | |
| | | ILLLKVAGFNLLMTLRLWSS | |
| | TCRa full | MDKILGASFLVLWLQLCWVSGQQKEKSDQQQVKQSPQSLIVQKGGISIINCA | 244 |
| | (DS) | YENTAFDYFPWYQQFPGKGPALLIAIRPDVSEKKEGRFTISFNKSAKQFSLH | |
| | | IMDSQPGDSATYFCAASSGTYKYIFGTGTRLKVLANIQNPDPAVYQLRDSKS | |
| | | SDKSVCLFTDFDSQTNVSQSKDSDVYITDKCVLDMRSMDFKSNSAVAWSNKS | |
| | | DFACANAFNNSIIPEDTFFPSPESSCDVKLVEKSFETDTNLNFQNLSVIGFR | |
| | | ILLLKVAGFNLLMTLRLWSS | |
| | TCRb | DMNHEYM | 245 |
| | CDR1 | | |
| | TCRb | YSVGEGTT | 246 |
| | CDR2 | | |
| | TCRb | CASSFAGANYGYTF | 247 |
| | CDR3 | | |
| | TCRb | NAGVTQTPKFRVLKTGQSMTLLCAQDMNHEYMYWYRQDPGMGLRLIHYSVGE | 248 |
| | variable | GTTAKGEVPDGYNVSRLKKQNFLLGLESAAPSQTSVYFCASSFAGANYGYTF | |
| | domain | GSGTRLTVV | |
| | TCRb full | MSLGLLCCAAFSLLWAGPVNAGVTQTPKFRVLKTGQSMTLLCAQDMNHEYMY | 307 |
| | | WYRQDPGMGLRLIHYSVGEGTTAKGEVPDGYNVSRLKKQNFLLGLESAAPSQ | |
| | | TSVYFCASSFAGANYGYTFGSGTRLTVVEDLNKVFPPEVAVFEPSEAEISHT | |
| | | QKATLVCLATGFFPDHVELSWWVNGKEVHSGVSTDPQPLKEQPALNDSRYCL | |
| | | SSRLRVSATFWQNPRNHFRCQVQFYGLSENDEWTQDRAKPVTQIVSAEAWGR | |
| | | ADCGFTSVSYQQGVLSATILYEILLGKATLYAVLVSALVLMAMVKRKDF | |
| | TCRb full | MSLGLLCCAAFSLLWAGPVNAGVTQTPKFRVLKTGQSMTLLCAQDMNHEYMY | 249 |
| | (DS) | WYRQDPGMGLRLIHYSVGEGTTAKGEVPDGYNVSRLKKQNFLLGLESAAPSQ | |
| | | TSVYFCASSFAGANYGYTFGSGTRLTVVEDLNKVFPPEVAVFEPSEAEISHT | |
| | | QKATLVCLATGFFPDHVELSWWVNGKEVHSGVCTDPQPLKEQPALNDSRYCL | |
| | | SSRLRVSATFWQNPRNHFRCQVQFYGLSENDEWTQDRAKPVTQIVSAEAWGR | |
| | | ADCGFTSVSYQQGVLSATILYEILLGKATLYAVLVSALVLMAMVKRKDF | |
| TCR-Z | TCRa | SNSASQSF | 250 |
| | CDR1 | | |
| | TCRa | SVYSSGN | 251 |
| | CDR2 | | |
| | TCRa | CVVSGSNSGNTPLVF | 252 |
| | CDR3 | | |
| | TCRa | RKEVEQDPGPFNVPEGATVAFNCTYSNSASQSFFWYRQDCRKEPKLLMSVYS | 253 |
| | variable | SGNEDGRFTAQLNRASQYISLLIRDSKLSDSATYLCVVSGSNSGNTPLVFGK | |
| | domain | GTRLSVIA | |
| | TCRa full | MISLRVLLVILWLQLSWVWSQRKEVEQDPGPFNVPEGATVAFNCTYSNSASQ | 308 |
| | | SFFWYRQDCRKEPKLLMSVYSSGNEDGRFTAQLNRASQYISLLIRDSKLSDS | |
| | | ATYLCVVSGSNSGNTPLVFGKGTRLSVIANIQNPDPAVYQLRDSKSSDKSVC | |
| | | LFTDFDSQTNVSQSKDSDVYITDKTVLDMRSMDFKSNSAVAWSNKSDFACAN | |
| | | AFNNSIIPEDTFFPSPESSCDVKLVEKSFETDTNLNFQNLSVIGFRILLLKV | |
| | | AGFNLLMTLRLWSS | |
| | TCRa full | MISLRVLLVILWLQLSWVWSQRKEVEQDPGPFNVPEGATVAFNCTYSNSASQ | 254 |
| | (DS) | SFFWYRQDCRKEPKLLMSVYSSGNEDGRFTAQLNRASQYISLLIRDSKLSDS | |
| | | ATYLCVVSGSNSGNTPLVFGKGTRLSVIANIQNPDPAVYQLRDSKSSDKSVC | |
| | | LFTDFDSQTNVSQSKDSDVYITDKCVLDMRSMDFKSNSAVAWSNKSDFACAN | |
| | | AFNNSIIPEDTFFPSPESSCDVKLVEKSFETDTNLNFQNLSVIGFRILLLKV | |
| | | AGFNLLMTLRLWSS | |
| | TCRb | DSQVTMM | 255 |
| | CDR1 | | |
| | TCRb | TANQGSEAT | 256 |
| | CDR2 | | |
| | TCRb | CSVGPTSGNTIYF | 257 |
| | CDR3 | | |
| | TCRb | SAVISQKPSRDICQRGTSLTIQCQVDSQVTMMFWYRQQPGQSLTLIATANQG | 258 |
| | variable | SEATYESGFVIDKFPISRPNLTFSTLTVSNMSPEDSSIYLCSVGPTSGNTIY | |
| | domain | FGEGSWLTVV | |
| | TCRb full | MLSLLLLLLGLGSVFSAVISQKPSRDICQRGTSLTIQCQVDSQVTMMFWYRQ | 309 |
| | | QPGQSLTLIATANQGSEATYESGFVIDKFPISRPNLTFSTLTVSNMSPEDSS | |
| | | IYLCSVGPTSGNTIYFGEGSWLTVVEDLNKVFPPEVAVFEPSEAEISHTQKA | |
| | | TLVCLATGFFPDHVELSWWVNGKEVHSGVSTDPQPLKEQPALNDSRYCLSSR | |
| | | LRVSATFWQNPRNHFRCQVQFYGLSENDEWTQDRAKPVTQIVSAEAWGRADC | |
| | | GFTSVSYQQGVLSATILYEILLGKATLYAVLVSALVLMAMVKRKDF | |
| | TCRb full | MLSLLLLLLGLGSVFSAVISQKPSRDICQRGTSLTIQCQVDSQVTMMFWYRQ | 259 |
| | (DS) | QPGQSLTLIATANQGSEATYESGFVIDKFPISRPNLTFSTLTVSNMSPEDSS | |
| | | IYLCSVGPTSGNTIYFGEGSWLTVVEDLNKVFPPEVAVFEPSEAEISHTQKA | |
| | | TLVCLATGFFPDHVELSWWVNGKEVHSGVCTDPQPLKEQPALNDSRYCLSSR | |
| | | LRVSATFWQNPRNHFRCQVQFYGLSENDEWTQDRAKPVTQIVSAEAWGRADC | |
| | | GFTSVSYQQGVLSATILYEILLGKATLYAVLVSALVLMAMVKRKDF | |
| | TRAC | NIQNPDPAVYQLRDSKSSDKSVCLFTDFDSQTNVSQSKDSDVYITDKTVLDM | 106 |
| | (alpha | RSMDFKSNSAVAWSNKSDFACANAFNNSIIPEDTFFPSPESSCDVKLVEKSF | |
| | constant | ETDTNLNFQNLSVIGFRILLLKVAGFNLLMTLRLWSS | |
| | domain) | | |
| | TRBC1 | EDLNKVFPPEVAVFEPSEAEISHTQKATLVCLATGFFPDHVELSWWVNGKEV | 107 |
| | (beta | HSGVSTDPQPLKEQPALNDSRYCLSSRLRVSATFWQNPRNHFRCQVQFYGLS | |
| | constant | ENDEWTQDRAKPVTQIVSAEAWGRADCGFTSVSYQQGVLSATILYEILLGKA | |
| | domain) | TLYAVLVSALVLMAMVKRKDF | |
| | TRBC2 | EDLKNVFPPEVAVFEPSEAEISHTQKATLVCLATGFYPDHVELSWWVNGKEV | 108 |
| | (beta | HSGVSTDPQPLKEQPALNDSRYCLSSRLRVSATFWQNPRNHFRCQVQFYGLS | |
| | constant | ENDEWTQDRAKPVTQIVSAEAWGRADCGFTSESYQQGVLSATILYEILLGKA | |
| | domain) | TLYAVLVSALVLMAMVKRKDSRG | |
| | Preproinsulin | GAGSLQPLALEGSLQKRG | 109 |
| | 73-90 | | |

In some embodiments, a TCR is an exogenous TCR. An exogenous TCR may be any TCR that is introduced to a T cell, e.g., a regulatory T cell, wherein the TCR is not endogenous to (i.e., naturally occurring in) that regulatory T cell. For example, in some embodiments, an exogenous TCR is encoded by a nucleic acid that is not endogenous to a regulatory T cell (i.e., not naturally occurring in the genome of the regulatory T cell). In some embodiments, the nucleic acid is an engineered nucleic acid, for example, a recombinant or synthetic nucleic acid. Thus, an exogenous TCR may also be referred to herein as an engineered TCR. A regulatory T cell comprising (e.g., expressing) an engineered TCR is considered an engineered regulatory T cell.

A TCR may be a human TCR (comprises human TCR nucleic acid and/or amino acid sequences). In some embodiments, a TCR is a TCR from a monkey, mouse, rat, or any other animal. In some embodiments, a TCR is chimeric (comprises nucleic acid and/or amino acid sequences from two or more different species).

A target peptide may be a peptide, or a portion of a peptide, that is associated with Type 1 Diabetes. A target peptide associated with Type 1 Diabetes may be a peptide belonging to a proinsulin. In some embodiments, a target peptide is a peptide that is overexpressed in a population of cells associated with Type 1 Diabetes relative to a control (e.g., relative to a population of cells that are not associated with Type 1 Diabetes). In some embodiments, a target peptide is a peptide that is expressed (or expressed at higher levels) at the site (e.g., tissue) of disease relative to other sites in the body.

In some embodiments, a target peptide is a proinsulin peptide. As described herein, a proinsulin peptide is a peptide of proinsulin, preproinsulin (PPI), or proinsulin C. In some embodiments, a proinsulin peptide comprises the sequence of GAGSLQPLALEGSLQKRG (SEQ ID NO: 109). In some embodiments, a proinsulin peptide consists of the sequence of SEQ ID NO: 109. Thus, in some embodiments, a TCR of the disclosure binds specifically to a peptide comprising or consisting of the sequence of SEQ ID NO: 109.

Preproinsulin (PPI) is a 110 amino acid protein that is used to synthesize insulin. PPI is an inactive precursor molecule that is translated directly into the rough endoplasmic reticulum (RER), where its signal peptide is removed by signal peptidase to form proinsulin. Proinsulin is the penultimate precursor to insulin and is produced in pancreatic beta cells. The proinsulin peptide is about 86 amino acid residues in humans and formed by three distinct chains (the A chain, B chain and the C peptide). Proinsulin is post-translationally cleaved into three peptides: the B chain and A chain peptides, which are covalently linked via two disulfide bonds to form insulin, and C-peptide. Proinsulin is cleaved by proprotein convertase 1/3 and proprotein convertase 2, that remove C-peptide and carboxypeptidase E that removes two pairs of amino acids from the protein's ends, resulting in active insulin. Interestingly, proinsulin demonstrates affinity for the insulin receptor. Proinsulin C-peptide (‘C-peptide’) is about 31 amino acids long. The C-peptide is excised from proinsulin as it is converted to insulin. The full-length C-peptide are amino acids 33-63 of proinsulin.

In some embodiments, a target peptide is subject to an increased autoimmune reaction in a subject having Type 1 Diabetes relative to a control. In some embodiments, a target peptide is a peptide that is present at a site associated with Type 1 Diabetes within a subject relative to unaffected sites within the same subject. In some embodiments, a target peptide is specifically presented by an MHC allele that is associated with the presence of Type 1 Diabetes in the subject.

In some embodiments, a target peptide is a peptide that is overexpressed in cells of a subject having Type 1 Diabetes relative to a control (e.g., relative to a healthy subject). A target peptide is considered to be overexpressed in cells (e.g., associated with Type 1 Diabetes) if expression of the target peptide in the cells is at least 10%, at least 15%, at least 20%, at least 25%, at least 30%, at least 40%, at least 50%, at least 60%, at least 70%, at least 80%, or at least 90% higher in the cells relative to control cells (e.g., a population of cells that are not associated with Type 1 Diabetes). In some embodiments, a target peptide is overexpressed in cells of a subject having Type 1 Diabetes if expression of the target peptide in cells of the subject having Type 1 Diabetes is at least 10%, at least 15%, at least 20%, at least 25%, at least 30%, at least 40%, at least 50%, at least 60%, at least 70%, at least 80%, or at least 90% higher in the subject having Type 1 Diabetes relative to a healthy subject (e.g., a subject that does not have Type 1 Diabetes).

In some embodiments, a target peptide is a peptide that is highly expressed in cells at the site of disease in a subject having Type 1 Diabetes relative to a control (e.g., target peptide expression in an unaffected/non-disease site in the subject). A target peptide is considered to be highly expressed in cells (e.g., associated with Type 1 Diabetes) at the site of disease if expression of the target peptide in the cells the site of disease is at least 10%, at least 15%, at least 20%, at least 25%, at least 30%, at least 40%, at least 50%, at least 60%, at least 70%, at least 80%, or at least 90% higher in the cells relative to control cells (e.g., cells not at the site of disease).

In some embodiments, a target peptide is a peptide that is complexed with an MHC having an HLA haplotype associated with Type 1 Diabetes.

A TCR, in some embodiments, is (or is encoded as) a single polypeptide (e.g., comprising a beta chain and an alpha chain). In some embodiments, a TCR comprises an N-terminal beta chain and a C-terminal alpha chain. In other embodiments, a TCR comprises an N-terminal alpha chain and a C-terminal beta chain.

A TCR may comprise a linker domain positioned between an alpha chain and a beta chain. In some embodiments, a linker domain comprises a self-cleaving peptide sequence (e.g., a self-cleaving peptide sequence positioned between an alpha chain and a beta chain). A self-cleaving peptide sequence is a peptide sequence that induces a polypeptide to separate into two peptides using a non-classical mechanism. In some embodiments, a self-cleaving peptide sequence can induce ribosomal skipping during translation of a polypeptide. In some embodiments, a self-cleaving peptide sequence is 10-30, 10-25, 15-30, 15-25, or 18-22 amino acids in length. In some embodiments, a self-cleaving peptide sequence may be a 2A peptide sequence. A 2A peptide sequence may comprise, for example, a DXEXNPGP (SEQ ID NO: 101) amino acid motif, wherein X can be any amino acid. In some embodiments, a 2A peptide sequence is a P2A (derived from porcine teschovirus-1 2A), E2A (derived from equine rhinitis A virus), F2A (derived from foot-and-mouth disease virus), or T2A (derived from Thosea asigna virus 2A) peptide sequence. A T2A peptide sequence may comprise, for example, the amino acid sequence of EGRGSLLTCGDVEENPGP (SEQ ID NO: 102). A P2A peptide sequence may comprise, for example, the amino acid sequence of ATNFSLLKQAGDVEENPGP (SEQ ID NO: 103). An E2A peptide sequence may comprise, for example, the amino acid sequence of QCTNYALLKLAGDVESNPGP (SEQ ID NO: 104). A F2A peptide sequence may comprise, for example, the amino acid sequence of VKQTLNFDLLKLAGDVESNPGP (SEQ ID NO: 105).

In some embodiments, a TCR comprises two or more polypeptides. For example, in some embodiments, a TCR comprises a first polypeptide comprising an alpha chain and a second polypeptide comprising a beta chain.

In some embodiments, a TCR comprises one or more cysteine residues present in the alpha chain of the TCR that are capable of forming one or more disulfide bonds with one or more cysteine residues in the beta chain of the TCR. In some embodiments, an exogenous TCR comprises one or more cysteine residues present in the alpha chain constant region of the TCR that form or are capable of forming one or more disulfide bonds with one or more cysteine residues in the beta chain constant region of the TCR.

In some embodiments, a TCR alpha chain constant region comprises an amino acid substitution at position 48 to introduce a cysteine (e.g., T48C) relative to a TCR alpha chain constant region comprising the amino acid sequence of SEQ ID NO: 106. In some embodiments, a TCR beta chain constant region comprises an amino acid substitution at position 57 to introduce a cysteine (e.g., S57C) amino acid substitution relative to a TCR beta chain constant region comprising the amino acid sequence of SEQ ID NO: 108. In some embodiments, a TCR alpha chain constant region comprises an amino acid substitution at position 48 to introduce a cysteine (e.g., T48C) relative to a TCR alpha chain constant region comprising the amino acid sequence of SEQ ID NO: 106, and a TCR beta chain constant region comprises an amino acid substitution at position 57 to introduce a cysteine (e.g., S57C) amino acid substitution relative to a TCR beta chain constant region comprising the amino acid sequence of SEQ ID NO: 108, wherein the cysteine residue at position 48 of the alpha chain is capable of forming a disulfide bond with the cysteine residue at position 57 of the beta chain.

In some embodiments, a TCR alpha chain constant region comprises an amino acid substitution at position 45 to introduce a cysteine (e.g., T45C) relative to a TCR alpha chain constant region comprising the amino acid sequence of SEQ ID NO: 106. In some embodiments, a TCR beta chain constant region comprises an amino acid substitution at position 77 to introduce a cysteine (e.g., S77C) amino acid substitution relative to a TCR beta chain constant region comprising the amino acid sequence of SEQ ID NO: 108. In some embodiments, a TCR alpha chain constant region comprises an amino acid substitution at position 45 to introduce a cysteine (e.g., T45C) relative to a TCR alpha chain constant region comprising the amino acid sequence of SEQ ID NO: 106, and a TCR beta chain constant region comprises an amino acid substitution at position 77 to introduce a cysteine (e.g., S77C) amino acid substitution relative to a TCR beta chain constant region comprising the amino acid sequence of SEQ ID NO: 108, wherein the cysteine residue at position 45 of the alpha chain is capable of forming a disulfide bond with the cysteine residue at position 77 of the beta chain.

In some embodiments, a TCR alpha chain constant region comprises an amino acid substitution at position 10 to introduce a cysteine (e.g., Y10C) relative to a TCR alpha chain constant region comprising the amino acid sequence of SEQ ID NO: 106. In some embodiments, a TCR beta chain constant region comprises an amino acid substitution at position 17 to introduce a cysteine (e.g., S17C) amino acid substitution relative to a TCR beta chain constant region comprising the amino acid sequence of SEQ ID NO: 108. In some embodiments, a TCR alpha chain constant region comprises an amino acid substitution at position 10 to introduce a cysteine (e.g., Y10C) relative to a TCR alpha chain constant region comprising the amino acid sequence of SEQ ID NO: 106, and a TCR beta chain constant region comprises an amino acid substitution at position 17 to introduce a cysteine (e.g., $17C) amino acid substitution relative to a TCR beta chain constant region comprising the amino acid sequence of SEQ ID NO: 108, wherein the cysteine residue at position 10 of the alpha chain is capable of forming a disulfide bond with the cysteine residue at position 17 of the beta chain.

In some embodiments, a TCR alpha chain constant region comprises an amino acid substitution at position 45 to introduce a cysteine (e.g., T45C) relative to a TCR alpha chain constant region comprising the amino acid sequence of SEQ ID NO: 106. In some embodiments, a TCR beta chain constant region comprises an amino acid substitution at position 59 to introduce a cysteine (e.g., D59C) amino acid substitution relative to a TCR beta chain constant region comprising the amino acid sequence of SEQ ID NO: 108. In some embodiments, a TCR alpha chain constant region comprises an amino acid substitution at position 45 to introduce a cysteine (e.g., T45C) relative to a TCR alpha chain constant region comprising the amino acid sequence of SEQ ID NO: 106, and a TCR beta chain constant region comprises an amino acid substitution at position 59 to introduce a cysteine (e.g., D59C) amino acid substitution relative to a TCR beta chain constant region comprising the amino acid sequence of SEQ ID NO: 108, wherein the cysteine residue at position 45 of the alpha chain is capable of forming a disulfide bond with the cysteine residue at position 59 of the beta chain.

In some embodiments, a TCR alpha chain constant region comprises an amino acid substitution at position 15 to introduce a cysteine (e.g., S15C) relative to a TCR alpha chain constant region comprising the amino acid sequence of SEQ ID NO: 106. In some embodiments, a TCR beta chain constant region comprises an amino acid substitution at position 15 to introduce a cysteine (e.g., E15C) amino acid substitution relative to a TCR beta chain constant region comprising the amino acid sequence of SEQ ID NO: 108. In some embodiments, a TCR alpha chain constant region comprises an amino acid substitution at position 15 to introduce a cysteine (e.g., S15C) relative to a TCR alpha chain constant region comprising the amino acid sequence of SEQ ID NO: 106, and a TCR beta chain constant region comprises an amino acid substitution at position 15 to introduce a cysteine (e.g., E15C) amino acid substitution relative to a TCR beta chain constant region comprising the amino acid sequence of SEQ ID NO: 108, wherein the cysteine residue at position 15 of the alpha chain is capable of forming a disulfide bond with the cysteine residue at position 15 of the beta chain.

### Nucleic Acids Encoding a TCR

In some embodiments, the disclosure provides nucleic acids encoding a TCR (e.g., a TCR as described in Table 1). Nucleic acids may be or may include deoxyribonucleic acid (DNA), ribonucleic acid (RNA) (e.g., messenger RNA), threose nucleic acid (TNA), glycol nucleic acid (GNA), peptide nucleic acid (PNA), locked nucleic acid (LNA), ethylene nucleic acid (ENA), cyclohexenyl nucleic acid (CeNA) and/or chimeras.

The nucleic acids used herein are generally engineered nucleic acids. An engineered nucleic acid is a polynucleotide (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

Engineered nucleic acids of the present disclosure may be produced using standard molecular biology methods (see, e.g., Green and Sambrook, Molecular Cloning, A Laboratory Manual, 2012. Cold Spring Harbor Press). In some embodiments, nucleic acids are produced using GIBSON ASSEMBLY® Cloning (see, e.g., Gibson. D. G, et al. Nature Methods, 343-345, 2009; and Gibson, D. G, et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. The MegaGate molecular cloning method may also be used. MegaGate is a toxin-less Gateway technology that eliminates the cedb toxin used in Gateway recombinase cloning and instead utilizes meganuclease-mediated digestion to eliminate background vectors during cloning (see, e.g., Kramme C, et al. STAR Protoc. 2021 Oct. 22; 2(4):100907, incorporated herein by reference). Other methods of producing engineered polynucleotides may be used in accordance with the present disclosure.

In some embodiments, an engineered nucleic acid comprises a promoter operably linked to an open reading frame. A promoter is (includes) a nucleotide sequence to which RNA polymerase binds to initial transcription (e.g., ATG). Promoters are typically located directly upstream from (at the 5′ end of) a transcription initiation site. In some embodiments, a promoter is a heterologous promoter. A heterologous promoter is not naturally associated with the open reading frame to which is it operably linked. In some embodiments, a promoter is an inducible promoter. An inducible promoter may be regulated in vivo by a chemical agent, temperature, or light, for example. Non-limiting examples of promoters that may be used as provided herein include the eukaryotic translation elongation factor 1 alpha (EF-1 alpha) promoter and the MND promoter (myeloproliferative sarcoma virus enhancer, negative control region deleted, d1587rev primer-binding site substituted) (see, e.g., Gill, D R, et al. Gene Ther. 2001; 8: 1539-46 and Astrakhan. A, et al. Blood 2012; 119: 4395-4407).

An open reading frame is (includes) a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

A nucleic acid encoding a TCR may be a vector or plasmid. In some embodiments, the vector is a viral vector. For example, the vector may be a lentiviral vector, an adenovirus vector, an adeno-associated viral (AAV) vector, a herpes viral vector, a retroviral vector, or a baculoviral vector. A viral vector provides efficient delivery of the exogenous TCR into regulatory T cells of the disclosure. Exemplary viral vectors may be derived from lentivirus, retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2. Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses. D-type viruses, oncoretroviruses, HTLV-BLV group, alpharetrovirus, gammaretrovirus, spumavirus, murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus. Rous sarcoma virus and lentiviruses.

In some embodiments, a nucleic acid encoding an exogenous TCR is an RNA (e.g., a messenger RNA (mRNA)). In some embodiments, an mRNA comprises a 5′ cap, a 5′ untranslated region (UTR), an ORF, a 3′ UTR, and/or a poly (A) tail.

In some embodiments, a nucleic acid is codon optimized. Codon optimization methods are known in the art. Codon optimization, in some embodiments, may be used to match codon frequencies in target and host organisms to ensure proper folding; bias GC content to increase RNA (e.g., mRNA) stability or reduce secondary structures; minimize tandem repeat codons or base runs that may impair gene construction or expression; customize transcriptional and translational control regions; insert or remove protein trafficking sequences; remove/add post-translation modification sites in encoded protein (e.g., glycosylation sites); add, remove or shuffle protein domains; insert or delete restriction sites; modify ribosome binding sites and RNA (e.g., mRNA) degradation sites; adjust translational rates to allow the various domains of the protein to fold properly; or reduce or eliminate problem secondary structures within the polynucleotide

In some embodiments, a nucleic acid encoding a TCR comprises a promoter operably linked to a coding sequence encoding the exogenous human TCR. A promoter may be a viral promoter or a natural TCR promoter. In some embodiments, a promoter is a constitutively active promoter or an inducible promoter. In some embodiments, a promoter is the eukaryotic translation elongation factor 1 alpha (EF-1 alpha) promoter and the MND promoter (myeloproliferative sarcoma virus enhancer, negative control region deleted, d1587rev primer-binding site substituted) (see, e.g., Gill, D R, et al. Gene Ther. 2001; 8: 1539-46 and Astrakhan. A, et al. Blood 2012; 119: 4395-4407).

A vector may also include a termination codon and/or expression enhancer elements. Any suitable vectors, promoters, enhancers and termination codons known in the art may be used. In some embodiments, an enhancer element is an optimized post-transcriptional regulatory element (oPRE), a woodchuck hepatitis virus post-transcriptional regulatory element (WPRE). A WPRE may be a wild-type WPRE or a WPRE mutant sequence (e.g., WPRE-mut6 with a mutated woodchuck hepatitis virus X protein open reading frame translation start site). In some embodiments, a WPRE (e.g., WPRE-mut6) is as described in Zanta-Boussif. M. A, et al., Gene Therapy volume 16, pages 605-619 (2009).

In some embodiments, a vector encoding a T cell receptor (TCR) is a recombinant lentiviral vector encoding the TCR. A recombinant lentiviral vector may be a third generation Vesicular Stomatitis Virus glycoprotein (VSV-G) pseudotyped, self-inactivating vector (e.g., that was generated using a split-genome four plasmid system (rev; gag+pol; VSV-G; gene encoding the TCR). In some embodiments, a recombinant lentiviral vector encodes any of the TCRs described herein. In some embodiments, a recombinant lentiviral vector encodes a TCR that comprises one or more amino acid sequences set forth in Table 1 (e.g., a CDR sequence belonging to any one of TCR-A, TCR-B, TCR-C, TCR-D, TCR-E, TCR-F, TCR-G, TCR-H, TCR-I, TCR-J, TCR-K, TCR-L, TCR-M, TCR-N, TCR-O, TCR-P, TCR-Q, TCR-R, TCR-S, TCR-T, TCR-U, TCR-V, TCR-X, TCR-Y and TCR-Z). In some embodiments, a recombinant lentiviral vector encodes the B and a chains of any one of the TCRs provided in Table 1 (e.g., TCR-A, TCR-B, TCR-C, TCR-D, TCR-E, TCR-F, TCR-G, TCR-H, TCR-I, TCR-J, TCR-K, TCR-L, TCR-M, TCR-N, TCR-O, TCR-P, TCR-Q, TCR-R, TCR-S, TCR-T, TCR-U, TCR-V. TCR-X, TCR-Y, or TCR-Z), wherein the B and a chains are linked by a ribosome skip P2A element.

In some embodiments, a nucleic acid encoding a TCR comprises a nucleotide sequence having greater than 90%, greater than 95%, or greater than 98% identity to SEQ ID NO: 326. In some embodiments, a nucleic acid encoding a TCR comprises the nucleotide sequence of SEQ ID NO: 326.

### Isolated Cell Regulatory T Cells

The present disclosure provides methods of producing regulatory T cells (e.g., stable regulatory T cells) that express a TCR described herein and compositions comprising such regulatory T cells. These regulatory T cells may be used, for example, to treat Type 1 Diabetes. A regulatory T cell, also referred to as a “T_{reg}”, is (includes) a T cell that modulates the immune system. Regulatory T cells are immunosuppressive and generally suppress or downregulate induction and proliferation of effector T cells. Regulatory T cells are thought to be derived from the same lineage as naïve CD4⁺ cells and express the biomarkers CD4, CD25, and FOXP3. In some embodiments, regulatory T cells (e.g., stable regulatory T cells) comprise an exogenous human TCR that binds specifically to proinsulin. This binding occurs when the proinsulin is complexed with an MHC molecule (e.g., MHC Class I or MHC Class II).

A stable, or thymic, regulatory T cell is a regulatory T cell that comprises a hypomethylated regulatory T cell-specific demethylation region (TSDR) at an endogenous FOXP3 locus and expresses CD4, CD25, and FOXP3. A regulatory T cell is considered “stable” (also referred to interchangeably as “phenotypically stable”) if it is terminally differentiated, i.e., it has lost its ability to change its cell fate. In thymic regulatory T cell development, the genome organizer SATB1 (special AT-rich sequence-binding protein) binds to specific genomic sites from the CD4⁺CD8 thymocyte stage to open up the chromatin and activate super-enhancers associated with many regulatory T cell signature genes such as FOXP3, IL2RA, (CD25), CTLA4, IKZF2 (HELIOS), and IFZF4 (EOS), SATB1 and MLL4 (myeloid/lymphoid or mixed-lineage leukemia 4), an enzyme involved in enhancer priming, commonly occupy the newly identified conserved enhancer region, designated conserved noncoding sequence 0 (CNS0), at the FOXP3 locus, with subsequent activation of the enhancers at CNS3 and CNS2, and then the promoter. This results in stable hypomethylation and expression of FOXP3 and other regulator T cell-associated genes, thereby resulting in a stable regulatory T cell phenotype. Epigenetic mechanisms, in particular. DNA methylation/demethylation, which is heritable through cell divisions, play an essential role for stable maintenance of regulatory T cell-specific gene expression. Hypomethylation of the TSDR, which is an evolutionary conserved CpG-rich regulatory element of the FOXP3 gene, is associated with expression of FOXP3. A TSDR of an endogenous FOXP3 locus is hypomethylated when the methyl group from one or more methylated cytosines in the TSDR have been removed to replace the methylated cytosine(s) with cytosine. In some embodiments, measurement of the methylation status of the TSDR of a FOXP3 locus is as described in Kressler et. Al. “Targeted De-Methylation of the FOXP3-TSDR Is Sufficient to Induce Physiological FOXP3 Expression but Not a Functional Regulatory T Phenotype” Frontiers in Immunology, 7 Jan. 2021.; or Schreiber, et. Al. “The Regulatory T-Specific Demethylated Region Stabilizes Foxp3 Expression Independently of NF-κB Signaling” PLOS One, Feb. 5, 2014. For example, genomic DNA may be isolated and bisulfite conversion performed. This converts, in some embodiments, all the unmethylated cysteines to uracils. The TSDR region can be amplified using primers that amplify the bisulfite-converted DNA, independent of methylation status. Two competing probes detect the abundance of methylated or unmethylated CpG, with the total and/or relative abundance being determined by ddPCR or pyrosequencing. Alternatively, next-generation sequencing (NGS) can be performed on the bisulfite-converted DNA to determine the sequence of the bisulfite-converted DNA at the TSDR locus.

In some embodiments, a stable regulatory T cell retains markers of stability in the presence of pro-inflammatory conditions (e.g., in presence of one or more pro-inflammatory cytokines). In some embodiments, a stable regulatory T cell comprises a hypomethylated regulatory T cell-specific demethylation region (TSDR) at an endogenous FOXP3 locus and expresses CD4, CD25, and FOXP3 in the presence of pro-inflammatory conditions (e.g., in presence of one or more pro-inflammatory cytokines). In some embodiments, a stable regulatory T cell comprises a hypomethylated regulatory T cell-specific demethylation region (TSDR) at an endogenous FOXP3 locus and expresses CD4, CD25, and FOXP3 in the presence of pro-inflammatory conditions for at least 3, 4, 5, 6, 7, 8, 9, 10, 11, 12, 13, 14, 15, 20, 21, 22, 23, 24, or 25 days.

A stable regulatory T cell that expresses an exogenous TCR (e.g., a TCR as described in Table 1) that binds specifically to a target peptide (e.g., Proinsulin) complexed with an MHC molecule further exhibits one or more of the following functions: (i) cytokine secretion activity (e.g., secretion of IL-10, IL-4, IL-6, IL-11, and/or IL-13) when the TCR is contacted with its target peptide; (ii) expression of activation markers associated with regulatory T cells (e.g., expression of CD69, 4-1BB, CD25, CD71, and/or CTLA-4) when the TCR is contacted with its target peptide; and/or (iii) suppression activity (e.g., the ability of the stable regulatory T cell to suppress the activation of conventional T cells having specificity towards a shared target peptide) when the TCR is contacted with the target peptide complexed with an MHC molecule.

Thus, regulatory T cells of the isolated cell populations provided herein may comprise a hypomethylated TSDR at an endogenous FOXP3 locus, expression of CD4, CD25, and FOXP3, and/or exhibit cytokine secretion, activation, and/or suppression activity.

In some embodiments, a regulatory T cell (e.g., a stable regulatory T cell) is CD25. In some embodiments, a regulatory T cell (e.g., a stable regulatory T cell) is CD25^{high}. As is used herein. “CD25^{+/high}” means that the cell is either CD25⁺ or CD25^{high} A CD25 cell is a cell that expresses a detectable level of CD25 (e.g., detectable by FACS). A CD25^{+/high} cell is a cell that expresses CD25 above a threshold or control level. For example, in the context of an isolated cell population that comprises CD25⁺ cells, a subpopulation of those cells may express higher levels of CD25 relative to another subpopulation of CD25⁺ cells in that population (or relative to all the other cells CD25+ cells in the population). The former subpopulation would be considered to be CD25^{high} while the latter subpopulation would be considered only CD25 (not CD25^{high}).

In some embodiments, a regulatory T cell (e.g., a stable regulatory T cell) is CD4⁺.

In some embodiments, a regulatory T cell (e.g., a stable regulatory T cell) is CD127^{-/lo}. As is used herein. “CD127^{-/lo}” means that the cell is either CD127⁻ or CD127^{lo}. A CD127⁻ cell is a cell that does not express CD127, does not express a detectable level of CD127 (e.g., by FACS), or expresses CD127 below a threshold or control level. For example, in the context of an isolated cell population that comprises at least some proportion of CD127⁺ cells (e.g., detectable by FACS), a subpopulation of those cells may express lower levels of CD127 relative to another subpopulation of CD127⁺ cells in that population (or relative to all the other cells CD127+ cells in the population). The former subpopulation would be considered to be CD127^{lo} (not CD127⁻).

In some embodiments, a regulatory T cell (e.g., a stable regulatory T cell) is CD45RA⁺.

In some embodiments, a regulatory T cell (e.g., a stable regulatory T cell) is FOXP3⁺.

In some embodiments, a regulatory T cell (e.g., a stable regulatory T cell) is a CD25^{+/high}CD4⁺CD127^{-/lo} cell. That is, the regulatory T cell expresses, or expresses a high level of CD25, expresses CD4, and does not express, or expresses a low level of, CD127. In some embodiments, a regulatory T cell does not express CD127. In some embodiments, a regulatory T cell (e.g., a stable regulatory T cell) is a CD25^{+/high}CD4⁺CD127^{-/lo}/CD45RA⁺ cell. Thus, the regulatory T cells expresses, or expresses a high level of CD25, expresses CD4 and CD45RA, and does not express, or expresses a low level of, CD127. In some embodiments, a regulatory T cell expresses FOXP3. In some embodiments, a regulatory T cell is a CD25^{+/high}CD4⁺CD127^{-/lo}FOXP3⁺ cell. Thus, the regulatory T cells expresses, or expresses a high level of CD25, expresses CD4 and FOXP3, and does not express, or expresses a low level of, CD127. In some embodiments, a regulatory T cell is a CD25^{+/high}CD4⁺CD45RA⁺CD127^{-/lo}FOXP3⁺ cell. Thus, the regulatory T cells expresses, or expresses a high level of CD25, expresses CD4, CD45RA and FOXP3, and do not express, or expresses a low level of, CD127.

A cell “expresses” a biomarker (e.g., CD4, CD25, CD45RA, and/or FOXP3) if the biomarker can be detected using a conventional protein expression assay, such as an antibody detection assay. An antibody detection assay in this context involves the detection (e.g., using fluorescence activated cell sorting (FACS) or Western blot) of an antibody that binds to the biomarker of interest (e.g., CD4, CD25, CD45RA, or FOXP3) when the biomarker is present within a cell or on the surface of a cell. A cell “does not express” a biomarker (e.g., CD127) if the expression of the biomarker cannot be detected using a conventional protein assay. A cell “expresses a low level” of a biomarker (e.g., CD127), if expression of that biomarker is lower than (e.g., at least 50%, at least 60%, at least 70%, at least 80%, or at least 90% lower than) a control level. In some embodiments, the control level of a biomarker in a regulatory T cell is the expression level of the biomarker (e.g., CD127) in a conventional T cell or a CD8⁺ T cell. For example, a regulatory T cell expresses a low level of CD127 if its expression of CD127 is lower than (e.g., at least 50%, at least 60%, at least 70%, at least 80%, or at least 90% lower than) expression of CD127 in a conventional T cell.

In some embodiments, a stable regulatory T cell of an isolated cell population provided herein maintains a hypomethylated TSDR at an endogenous FOXP3 locus over time (i.e., the TSDR at the endogenous FOXP3 locus of viable cells (e.g., in culture, in a cryoprotective agent, and/or in vivo) is hypomethylated for a certain measurable period of time). For example, a stable regulatory T cell of an isolated cell population may maintain a hypomethylated TSDR at an endogenous FOXP3 locus for at least 1, 2, 3, 4, 5, 6, 7, 8, 9, 10, 11, 12, 13, 14, 15, 16, 17, 18, 19, or 20 days (e.g., in culture, in a cryoprotective agent, and/or in vivo after being obtained from a subject or following transduction with a nucleic acid expressing a TCR). In some embodiments, a stable regulatory T cell maintains a hypomethylated TSDR at an endogenous FOXP3 locus for more than 5 days, more than 10 days, more than 15 days, or more than 20 days (e.g., in culture, in a cryoprotective agent, and/or in vivo after being obtained from a subject or following transduction with a nucleic acid expressing a TCR). In some embodiments, a stable regulatory T cell maintains a hypomethylated TSDR at an endogenous FOXP3 locus for 1-20 days, 1-10 days, 1-5 days, 5-30 days, 5-20 days, 10-40 days, or 25-50 days (e.g., in culture, in a cryoprotective agent, and/or in vivo after being obtained from a subject or following transduction with a nucleic acid expressing a TCR).

A regulatory T cell of an isolated cell population of the disclosure, in some embodiments, is an autologous cell. The term autologous in this context refers to cells that have been obtained from the same subject to which they are subsequently administered (e.g., following engineering to introduce an exogenous TCR into the cells). For example, a population of cells may be obtained from a subject, subjected to the methods described herein, and then administered to the same subject (from which the population of cells was originally obtained) to treat Type 1 Diabetes. In such embodiments, the population of cells administered to the subject comprise autologous regulatory T cells.

A regulatory T cell of the disclosure, in some embodiments, is an allogenic cell. The term allogenic in this context refers to cells that have been obtained from one subject and then administered to another subject. For example, a population of cells may be obtained from a subject, subjected to the methods described herein, and then administered to another subject in order to treat an autoimmune disease.

In some embodiments, regulatory T cells (e.g., a biological sample comprising regulatory T cells) are obtained from a subject diagnosed with, or suspected of having. Type 1 Diabetes.

Some aspects relate to an engineered regulatory T cell that comprises an exogenous TCR that binds to a proinsulin peptide complexed with a major histocompatibility complex (MHC) molecule, wherein the TCR comprises an alpha chain and a beta chain, and wherein the TCR comprises: a CDR1-alpha sequence comprising the amino acid sequence of SEQ ID NO: 1, a CDR2-alpha sequence comprising the amino acid sequence of SEQ ID NO: 2, a CDR3-alpha sequence comprising the amino acid sequence of SEQ ID NO: 3, a CDR1-beta sequence comprising the amino acid sequence of SEQ ID NO: 6, a CDR2-beta sequence comprising the amino acid sequence of SEQ ID NO: 7, and/or a CDR3-beta sequence comprising the amino acid sequence of SEQ ID NO: 8.

In some embodiments, a TCR comprises a CDR1-alpha sequence comprising the amino acid sequence of SEQ ID NO: 1, a CDR2-alpha sequence comprising the amino acid sequence of SEQ ID NO: 2, and a CDR3-alpha sequence comprising the amino acid sequence of SEQ ID NO: 3. In some embodiments, the alpha chain comprises an alpha variable region comprising an amino acid sequence having at least 90%, 95%, or 100% identity to the amino acid sequence of SEQ ID NO: 4. In some embodiments, the alpha chain comprises an amino acid sequence having at least 90%, 95%, or 100% identity to the amino acid sequence of SEQ ID NO: 5. In some embodiments, the alpha chain comprises an amino acid sequence having at least 90%, 95%, or 100% identity to the amino acid sequence of SEQ ID NO: 260.

In some embodiments, a TCR comprises a CDR1-beta sequence comprising the amino acid sequence of SEQ ID NO: 6, a CDR2-beta sequence comprising the amino acid sequence of SEQ ID NO: 7, and a CDR3-beta sequence comprising the amino acid sequence of SEQ ID NO: 8. In some embodiments, the beta chain comprises a beta variable region comprising an amino acid sequence having at least 90%, 95%, or 100% identity to the amino acid sequence of SEQ ID NO: 9. In some embodiments, the beta chain comprises an amino acid sequence having at least 90%, 95%, or 100% identity to the amino acid sequence of SEQ ID NO: 10. In some embodiments, the beta chain comprises an amino acid sequence having at least 90%, 95%, or 100% identity to the amino acid sequence of SEQ ID NO: 261.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 11, a CDR2-alpha sequence comprising the amino acid sequence of SEQ ID NO: 12, a CDR3-alpha sequence comprising the amino acid sequence of SEQ ID NO: 13, a CDR1-beta sequence comprising the amino acid sequence of SEQ ID NO: 16, a CDR2-beta sequence comprising the amino acid sequence of SEQ ID NO: 17, and/or a CDR3-beta sequence comprising the amino acid sequence of SEQ ID NO: 18.

In some embodiments, a TCR comprises a CDR1-alpha sequence comprising the amino acid sequence of SEQ ID NO: 11, a CDR2-alpha sequence comprising the amino acid sequence of SEQ ID NO: 12, and a CDR3-alpha sequence comprising the amino acid sequence of SEQ ID NO: 13. In some embodiments, the alpha chain comprises an alpha variable region comprising an amino acid sequence having at least 90%, 95%, or 100% identity to the amino acid sequence of SEQ ID NO: 14. In some embodiments, the alpha chain comprises an amino acid sequence having at least 90%, 95%, or 100% identity to the amino acid sequence of SEQ ID NO: 15. In some embodiments, the alpha chain comprises an amino acid sequence having at least 90%, 95%, or 100% identity to the amino acid sequence of SEQ ID NO: 262.

In some embodiments, a TCR comprises a CDR1-beta sequence comprising the amino acid sequence of SEQ ID NO: 16, a CDR2-beta sequence comprising the amino acid sequence of SEQ ID NO: 17, and a CDR3-beta sequence comprising the amino acid sequence of SEQ ID NO: 18. In some embodiments, the beta chain comprises an alpha variable region comprising an amino acid sequence having at least 90%, 95%, or 100% identity to the amino acid sequence of SEQ ID NO: 19. In some embodiments, the beta chain comprises an amino acid sequence having at least 90%, 95%, or 100% identity to the amino acid sequence of SEQ ID NO: 20. In some embodiments, the beta chain comprises an amino acid sequence having at least 90%, 95%, or 100% identity to the amino acid sequence of SEQ ID NO: 263.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 21, a CDR2-alpha sequence comprising the amino acid sequence of SEQ ID NO: 22, a CDR3-alpha sequence comprising the amino acid sequence of SEQ ID NO: 23, a CDR1-beta sequence comprising the amino acid sequence of SEQ ID NO: 26, a CDR2-beta sequence comprising the amino acid sequence of SEQ ID NO: 27, and/or a CDR3-beta sequence comprising the amino acid sequence of SEQ ID NO: 28.

In some embodiments, a TCR comprises a CDR1-alpha sequence comprising the amino acid sequence of SEQ ID NO: 21, a CDR2-alpha sequence comprising the amino acid sequence of SEQ ID NO: 22, and a CDR3-alpha sequence comprising the amino acid sequence of SEQ ID NO: 23. In some embodiments, the alpha chain comprises an alpha variable region comprising an amino acid sequence having at least 90%, 95%, or 100% identity to the amino acid sequence of SEQ ID NO: 24. In some embodiments, the alpha chain comprises an amino acid sequence having at least 90%, 95%, or 100% identity to the amino acid sequence of SEQ ID NO: 25. In some embodiments, the alpha chain comprises an amino acid sequence having at least 90%, 95%, or 100% identity to the amino acid sequence of SEQ ID NO: 264.

In some embodiments, a TCR comprises a CDR1-beta sequence comprising the amino acid sequence of SEQ ID NO: 26, a CDR2-beta sequence comprising the amino acid sequence of SEQ ID NO: 27, and a CDR3-beta sequence comprising the amino acid sequence of SEQ ID NO: 28. In some embodiments, the beta chain comprises a beta variable region comprising an amino acid sequence having at least 90%, 95%, or 100% identity to the amino acid sequence of SEQ ID NO: 29. In some embodiments, the beta chain comprises an amino acid sequence having at least 90%, 95%, or 100% identity to the amino acid sequence of SEQ ID NO: 30. In some embodiments, the beta chain comprises an amino acid sequence having at least 90%, 95%, or 100% identity to the amino acid sequence of SEQ ID NO: 265.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises a CDR1-alpha sequence comprising the amino acid sequence of SEQ ID NO: 31, a CDR2-alpha sequence comprising the amino acid sequence of SEQ ID NO: 32, a CDR3-alpha sequence comprising the amino acid sequence of SEQ ID NO: 33, a CDR1-beta sequence comprising the amino acid sequence of SEQ ID NO: 36, a CDR2-beta sequence comprising the amino acid sequence of SEQ ID NO: 37, and/or a CDR3-beta sequence comprising the amino acid sequence of SEQ ID NO: 38.

In some embodiments, a TCR comprises a CDR1-alpha sequence comprising the amino acid sequence of SEQ ID NO: 31, a CDR2-alpha sequence comprising the amino acid sequence of SEQ ID NO: 32, and a CDR3-alpha sequence comprising the amino acid sequence of SEQ ID NO: 33. In some embodiments, the alpha chain comprises an alpha variable region comprising an amino acid sequence having at least 90%, 95%, or 100% identity to the amino acid sequence of SEQ ID NO: 34. In some embodiments, the alpha chain comprises an amino acid sequence having at least 90%, 95%, or 100% identity to the amino acid sequence of SEQ ID NO: 35. In some embodiments, the alpha chain comprises an amino acid sequence having at least 90%, 95%, or 100% identity to the amino acid sequence of SEQ ID NO: 266.

In some embodiments, a TCR comprises a CDR1-beta sequence comprising the amino acid sequence of SEQ ID NO: 36, a CDR2-beta sequence comprising the amino acid sequence of SEQ ID NO: 37, and a CDR3-beta sequence comprising the amino acid sequence of SEQ ID NO: 38. In some embodiments, the beta chain comprises a beta variable region comprising an amino acid sequence having at least 90%, 95%, or 100% identity to the amino acid sequence of SEQ ID NO: 39. In some embodiments, the beta chain comprises an amino acid sequence having at least 90%, 95%, or 100% identity to the amino acid sequence of SEQ ID NO: 40. In some embodiments, the beta chain comprises an amino acid sequence having at least 90%, 95%, or 100% identity to the amino acid sequence of SEQ ID NO: 267.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 41, a CDR2-alpha sequence comprising the amino acid sequence of SEQ ID NO: 42, a CDR3-alpha sequence comprising the amino acid sequence of SEQ ID NO: 43, a CDR1-beta sequence comprising the amino acid sequence of SEQ ID NO: 46, a CDR2-beta sequence comprising the amino acid sequence of SEQ ID NO: 47, and/or a CDR3-beta sequence comprising the amino acid sequence of SEQ ID NO: 48.

In some embodiments, a TCR comprises a CDR1-alpha sequence comprising the amino acid sequence of SEQ ID NO: 41, a CDR2-alpha sequence comprising the amino acid sequence of SEQ ID NO: 42, and a CDR3-alpha sequence comprising the amino acid sequence of SEQ ID NO: 43. In some embodiments, the alpha chain comprises an alpha variable region comprising an amino acid sequence having at least 90%, 95%, or 100% identity to the amino acid sequence of SEQ ID NO: 44. In some embodiments, the alpha chain comprises an amino acid sequence having at least 90%, 95%, or 100% identity to the amino acid sequence of SEQ ID NO: 45. In some embodiments, the alpha chain comprises an amino acid sequence having at least 90%, 95%, or 100% identity to the amino acid sequence of SEQ ID NO: 268.

In some embodiments, a TCR comprises a CDR1-beta sequence comprising the amino acid sequence of SEQ ID NO: 46, a CDR2-beta sequence comprising the amino acid sequence of SEQ ID NO: 47, and a CDR3-beta sequence comprising the amino acid sequence of SEQ ID NO: 48. In some embodiments, the beta chain comprises a beta variable region comprising an amino acid sequence having at least 90%, 95%, or 100% identity to the amino acid sequence of SEQ ID NO: 49. In some embodiments, the beta chain comprises an amino acid sequence having at least 90%, 95%, or 100% identity to the amino acid sequence of SEQ ID NO: 50. In some embodiments, the beta chain comprises an amino acid sequence having at least 90%, 95%, or 100% identity to the amino acid sequence of SEQ ID NO: 269.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 51, a CDR2-alpha sequence comprising the amino acid sequence of SEQ ID NO: 52, a CDR3-alpha sequence comprising the amino acid sequence of SEQ ID NO: 53, a CDR1-beta sequence comprising the amino acid sequence of SEQ ID NO: 56, a CDR2-beta sequence comprising the amino acid sequence of SEQ ID NO: 57, and/or a CDR3-beta sequence comprising the amino acid sequence of SEQ ID NO: 58.

In some embodiments, a TCR comprises a CDR1-alpha sequence comprising the amino acid sequence of SEQ ID NO: 51, a CDR2-alpha sequence comprising the amino acid sequence of SEQ ID NO: 52, and a CDR3-alpha sequence comprising the amino acid sequence of SEQ ID NO: 53. In some embodiments, the alpha chain comprises an alpha variable region comprising an amino acid sequence having at least 90%, 95%, or 100% identity to the amino acid sequence of SEQ ID NO: 54. In some embodiments, the alpha chain comprises an amino acid sequence having at least 90%, 95%, or 100% identity to the amino acid sequence of SEQ ID NO: 55. In some embodiments, the alpha chain comprises an amino acid sequence having at least 90%, 95%, or 100% identity to the amino acid sequence of SEQ ID NO: 270.

In some embodiments, a TCR comprises a CDR1-beta sequence comprising the amino acid sequence of SEQ ID NO: 56, a CDR2-beta sequence comprising the amino acid sequence of SEQ ID NO: 57, and a CDR3-beta sequence comprising the amino acid sequence of SEQ ID NO: 58. In some embodiments, the beta chain comprises a beta variable region comprising an amino acid sequence having at least 90%, 95%, or 100% identity to the amino acid sequence of SEQ ID NO: 59. In some embodiments, the beta chain comprises an amino acid sequence having at least 90%, 95%, or 100% identity to the amino acid sequence of SEQ ID NO: 60. In some embodiments, the beta chain comprises an amino acid sequence having at least 90%, 95%, or 100% identity to the amino acid sequence of SEQ ID NO: 271.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 61, a CDR2-alpha sequence comprising the amino acid sequence of SEQ ID NO: 62, a CDR3-alpha sequence comprising the amino acid sequence of SEQ ID NO: 63, a CDR1-beta sequence comprising the amino acid sequence of SEQ ID NO: 66, a CDR2-beta sequence comprising the amino acid sequence of SEQ ID NO: 67, and/or a CDR3-beta sequence comprising the amino acid sequence of SEQ ID NO: 68.

In some embodiments, a TCR comprises a CDR1-alpha sequence comprising the amino acid sequence of SEQ ID NO: 61, a CDR2-alpha sequence comprising the amino acid sequence of SEQ ID NO: 62, and a CDR3-alpha sequence comprising the amino acid sequence of SEQ ID NO: 63. In some embodiments, the alpha chain comprises an alpha variable region comprising an amino acid sequence having at least 90%, 95%, or 100% identity to the amino acid sequence of SEQ ID NO: 64. In some embodiments, the alpha chain comprises an amino acid sequence having at least 90%, 95%, or 100% identity to the amino acid sequence of SEQ ID NO: 65. In some embodiments, the alpha chain comprises an amino acid sequence having at least 90%, 95%, or 100% identity to the amino acid sequence of SEQ ID NO: 272.

In some embodiments, a TCR comprises a CDR1-beta sequence comprising the amino acid sequence of SEQ ID NO: 66, a CDR2-beta sequence comprising the amino acid sequence of SEQ ID NO: 67, and a CDR3-beta sequence comprising the amino acid sequence of SEQ ID NO: 68. In some embodiments, the beta chain comprises a beta variable region comprising an amino acid sequence having at least 90%, 95%, or 100% identity to the amino acid sequence of SEQ ID NO: 69. In some embodiments, the beta chain comprises an amino acid sequence having at least 90%, 95%, or 100% identity to the amino acid sequence of SEQ ID NO: 70. In some embodiments, the beta chain comprises an amino acid sequence having at least 90%, 95%, or 100% identity to the amino acid sequence of SEQ ID NO: 273.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 71, a CDR2-alpha sequence comprising the amino acid sequence of SEQ ID NO: 72, a CDR3-alpha sequence comprising the amino acid sequence of SEQ ID NO: 73, a CDR1-beta sequence comprising the amino acid sequence of SEQ ID NO: 76, a CDR2-beta sequence comprising the amino acid sequence of SEQ ID NO: 77, and/or a CDR3-beta sequence comprising the amino acid sequence of SEQ ID NO: 78.

In some embodiments, a TCR comprises a CDR1-alpha sequence comprising the amino acid sequence of SEQ ID NO: 71, a CDR2-alpha sequence comprising the amino acid sequence of SEQ ID NO: 72, and a CDR3-alpha sequence comprising the amino acid sequence of SEQ ID NO: 73. In some embodiments, the alpha chain comprises an alpha variable region comprising an amino acid sequence having at least 90%, 95%, or 100% identity to the amino acid sequence of SEQ ID NO: 74. In some embodiments, the alpha chain comprises an amino acid sequence having at least 90%, 95%, or 100% identity to the amino acid sequence of SEQ ID NO: 75. In some embodiments, the alpha chain comprises an amino acid sequence having at least 90%, 95%, or 100% identity to the amino acid sequence of SEQ ID NO: 274.

In some embodiments, a TCR comprises a CDR1-beta sequence comprising the amino acid sequence of SEQ ID NO: 76, a CDR2-beta sequence comprising the amino acid sequence of SEQ ID NO: 77, and a CDR3-beta sequence comprising the amino acid sequence of SEQ ID NO: 78. In some embodiments, the beta chain comprises a beta variable region comprising an amino acid sequence having at least 90%, 95%, or 100% identity to the amino acid sequence of SEQ ID NO: 79. In some embodiments, the beta chain comprises an amino acid sequence having at least 90%, 95%, or 100% identity to the amino acid sequence of SEQ ID NO: 80. In some embodiments, the beta chain comprises an amino acid sequence having at least 90%, 95%, or 100% identity to the amino acid sequence of SEQ ID NO: 275.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 81, a CDR2-alpha sequence comprising the amino acid sequence of SEQ ID NO: 82, a CDR3-alpha sequence comprising the amino acid sequence of SEQ ID NO: 83, a CDR1-beta sequence comprising the amino acid sequence of SEQ ID NO: 86, a CDR2-beta sequence comprising the amino acid sequence of SEQ ID NO: 87, and/or a CDR3-beta sequence comprising the amino acid sequence of SEQ ID NO: 88.

In some embodiments, a TCR comprises a CDR1-alpha sequence comprising the amino acid sequence of SEQ ID NO: 81, a CDR2-alpha sequence comprising the amino acid sequence of SEQ ID NO: 82, and a CDR3-alpha sequence comprising the amino acid sequence of SEQ ID NO: 83. In some embodiments, the alpha chain comprises an alpha variable region comprising an amino acid sequence having at least 90%, 95%, or 100% identity to the amino acid sequence of SEQ ID NO: 84. In some embodiments, the alpha chain comprises an amino acid sequence having at least 90%, 95%, or 100% identity to the amino acid sequence of SEQ ID NO: 85. In some embodiments, the alpha chain comprises an amino acid sequence having at least 90%, 95%, or 100% identity to the amino acid sequence of SEQ ID NO: 276.

In some embodiments, a TCR comprises a CDR1-beta sequence comprising the amino acid sequence of SEQ ID NO: 86, a CDR2-beta sequence comprising the amino acid sequence of SEQ ID NO: 87, and a CDR3-beta sequence comprising the amino acid sequence of SEQ ID NO: 88. In some embodiments, the beta chain comprises a beta variable region comprising an amino acid sequence having at least 90%, 95%, or 100% identity to the amino acid sequence of SEQ ID NO: 89. In some embodiments, the beta chain comprises an amino acid sequence having at least 90%, 95%, or 100% identity to the amino acid sequence of SEQ ID NO: 90. In some embodiments, the beta chain comprises an amino acid sequence having at least 90%, 95%, or 100% identity to the amino acid sequence of SEQ ID NO: 277.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 91, a CDR2-alpha sequence comprising the amino acid sequence of SEQ ID NO: 92, a CDR3-alpha sequence comprising the amino acid sequence of SEQ ID NO: 93, a CDR1-beta sequence comprising the amino acid sequence of SEQ ID NO: 96, a CDR2-beta sequence comprising the amino acid sequence of SEQ ID NO: 97, and/or a CDR3-beta sequence comprising the amino acid sequence of SEQ ID NO: 98.

In some embodiments, a TCR comprises a CDR1-alpha sequence comprising the amino acid sequence of SEQ ID NO: 91, a CDR2-alpha sequence comprising the amino acid sequence of SEQ ID NO: 92, and a CDR3-alpha sequence comprising the amino acid sequence of SEQ ID NO: 93. In some embodiments, the alpha chain comprises an alpha variable region comprising an amino acid sequence having at least 90%, 95%, or 100% identity to the amino acid sequence of SEQ ID NO: 94. In some embodiments, the alpha chain comprises an amino acid sequence having at least 90%, 95%, or 100% identity to the amino acid sequence of SEQ ID NO: 95. In some embodiments, the alpha chain comprises an amino acid sequence having at least 90%, 95%, or 100% identity to the amino acid sequence of SEQ ID NO: 278.

In some embodiments, a TCR comprises a CDR1-beta sequence comprising the amino acid sequence of SEQ ID NO: 96, a CDR2-beta sequence comprising the amino acid sequence of SEQ ID NO: 97, and a CDR3-beta sequence comprising the amino acid sequence of SEQ ID NO: 98. In some embodiments, the beta chain comprises a beta variable region comprising an amino acid sequence having at least 90%, 95%, or 100% identity to the amino acid sequence of SEQ ID NO: 99. In some embodiments, the beta chain comprises an amino acid sequence having at least 90%, 95%, or 100% identity to the amino acid sequence of SEQ ID NO: 100. In some embodiments, the beta chain comprises an amino acid sequence having at least 90%, 95%, or 100% identity to the amino acid sequence of SEQ ID NO: 279.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 110, a CDR2-alpha sequence comprising the amino acid sequence of SEQ ID NO: 111, a CDR3-alpha sequence comprising the amino acid sequence of SEQ ID NO: 112, a CDR1-beta sequence comprising the amino acid sequence of SEQ ID NO: 115, a CDR2-beta sequence comprising the amino acid sequence of SEQ ID NO: 116, and/or a CDR3-beta sequence comprising the amino acid sequence of SEQ ID NO: 117.

In some embodiments, a TCR comprises a CDR1-alpha sequence comprising the amino acid sequence of SEQ ID NO: 110, a CDR2-alpha sequence comprising the amino acid sequence of SEQ ID NO: 111, and a CDR3-alpha sequence comprising the amino acid sequence of SEQ ID NO: 112. In some embodiments, the alpha chain comprises an alpha variable region comprising an amino acid sequence having at least 90%, 95%, or 100% identity to the amino acid sequence of SEQ ID NO: 113. In some embodiments, the alpha chain comprises an amino acid sequence having at least 90%, 95%, or 100% identity to the amino acid sequence of SEQ ID NO: 114. In some embodiments, the alpha chain comprises an amino acid sequence having at least 90%, 95%, or 100% identity to the amino acid sequence of SEQ ID NO: 280.

In some embodiments, a TCR comprises a CDR1-beta sequence comprising the amino acid sequence of SEQ ID NO: 115, a CDR2-beta sequence comprising the amino acid sequence of SEQ ID NO: 116, and a CDR3-beta sequence comprising the amino acid sequence of SEQ ID NO: 117. In some embodiments, the beta chain comprises a beta variable region comprising an amino acid sequence having at least 90%, 95%, or 100% identity to the amino acid sequence of SEQ ID NO: 118. In some embodiments, the beta chain comprises an amino acid sequence having at least 90%, 95%, or 100% identity to the amino acid sequence of SEQ ID NO: 119. In some embodiments, the beta chain comprises an amino acid sequence having at least 90%, 95%, or 100% identity to the amino acid sequence of SEQ ID NO: 281.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 120, a CDR2-alpha sequence comprising the amino acid sequence of SEQ ID NO: 121, a CDR3-alpha sequence comprising the amino acid sequence of SEQ ID NO: 122, a CDR1-beta sequence comprising the amino acid sequence of SEQ ID NO: 125, a CDR2-beta sequence comprising the amino acid sequence of SEQ ID NO: 126, and/or a CDR3-beta sequence comprising the amino acid sequence of SEQ ID NO: 127.

In some embodiments, a TCR comprises a CDR1-alpha sequence comprising the amino acid sequence of SEQ ID NO: 120, a CDR2-alpha sequence comprising the amino acid sequence of SEQ ID NO: 121, and a CDR3-alpha sequence comprising the amino acid sequence of SEQ ID NO: 122. In some embodiments, the alpha chain comprises an alpha variable region comprising an amino acid sequence having at least 90%, 95%, or 100% identity to the amino acid sequence of SEQ ID NO: 123. In some embodiments, the alpha chain comprises an amino acid sequence having at least 90%, 95%, or 100% identity to the amino acid sequence of SEQ ID NO: 124. In some embodiments, the alpha chain comprises an amino acid sequence having at least 90%, 95%, or 100% identity to the amino acid sequence of SEQ ID NO: 282.

In some embodiments, a TCR comprises a CDR1-beta sequence comprising the amino acid sequence of SEQ ID NO: 125, a CDR2-beta sequence comprising the amino acid sequence of SEQ ID NO: 126, and a CDR3-beta sequence comprising the amino acid sequence of SEQ ID NO: 127. In some embodiments, the beta chain comprises a beta variable region comprising an amino acid sequence having at least 90%, 95%, or 100% identity to the amino acid sequence of SEQ ID NO: 128. In some embodiments, the beta chain comprises an amino acid sequence having at least 90%, 95%, or 100% identity to the amino acid sequence of SEQ ID NO: 129. In some embodiments, the beta chain comprises an amino acid sequence having at least 90%, 95%, or 100% identity to the amino acid sequence of SEQ ID NO: 283.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 130, a CDR2-alpha sequence comprising the amino acid sequence of SEQ ID NO: 131, a CDR3-alpha sequence comprising the amino acid sequence of SEQ ID NO: 132, a CDR1-beta sequence comprising the amino acid sequence of SEQ ID NO: 135, a CDR2-beta sequence comprising the amino acid sequence of SEQ ID NO: 136, and/or a CDR3-beta sequence comprising the amino acid sequence of SEQ ID NO: 137.

In some embodiments, a TCR comprises a CDR1-alpha sequence comprising the amino acid sequence of SEQ ID NO: 130, a CDR2-alpha sequence comprising the amino acid sequence of SEQ ID NO: 131, and a CDR3-alpha sequence comprising the amino acid sequence of SEQ ID NO: 132. In some embodiments, the alpha chain comprises an alpha variable region comprising an amino acid sequence having at least 90%, 95%, or 100% identity to the amino acid sequence of SEQ ID NO: 133. In some embodiments, the alpha chain comprises an amino acid sequence having at least 90%, 95%, or 100% identity to the amino acid sequence of SEQ ID NO: 134. In some embodiments, the alpha chain comprises an amino acid sequence having at least 90%, 95%, or 100% identity to the amino acid sequence of SEQ ID NO: 284.

In some embodiments, a TCR comprises a CDR1-beta sequence comprising the amino acid sequence of SEQ ID NO: 135, a CDR2-beta sequence comprising the amino acid sequence of SEQ ID NO: 136, and a CDR3-beta sequence comprising the amino acid sequence of SEQ ID NO: 137. In some embodiments, the beta chain comprises a beta variable region comprising an amino acid sequence having at least 90%, 95%, or 100% identity to the amino acid sequence of SEQ ID NO: 138. In some embodiments, the beta chain comprises an amino acid sequence having at least 90%, 95%, or 100% identity to the amino acid sequence of SEQ ID NO: 139. In some embodiments, the beta chain comprises an amino acid sequence having at least 90%, 95%, or 100% identity to the amino acid sequence of SEQ ID NO: 285.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 140, a CDR2-alpha sequence comprising the amino acid sequence of SEQ ID NO: 141, a CDR3-alpha sequence comprising the amino acid sequence of SEQ ID NO: 142, a CDR1-beta sequence comprising the amino acid sequence of SEQ ID NO: 145, a CDR2-beta sequence comprising the amino acid sequence of SEQ ID NO: 146, and/or a CDR3-beta sequence comprising the amino acid sequence of SEQ ID NO: 147.

In some embodiments, a TCR comprises a CDR1-alpha sequence comprising the amino acid sequence of SEQ ID NO: 140, a CDR2-alpha sequence comprising the amino acid sequence of SEQ ID NO: 141, and a CDR3-alpha sequence comprising the amino acid sequence of SEQ ID NO: 142. In some embodiments, the alpha chain comprises an alpha variable region comprising an amino acid sequence having at least 90%, 95%, or 100% identity to the amino acid sequence of SEQ ID NO: 143. In some embodiments, the alpha chain comprises an amino acid sequence having at least 90%, 95%, or 100% identity to the amino acid sequence of SEQ ID NO: 144. In some embodiments, the alpha chain comprises an amino acid sequence having at least 90%, 95%, or 100% identity to the amino acid sequence of SEQ ID NO: 286.

In some embodiments, a TCR comprises a CDR1-beta sequence comprising the amino acid sequence of SEQ ID NO: 145, a CDR2-beta sequence comprising the amino acid sequence of SEQ ID NO: 146, and a CDR3-beta sequence comprising the amino acid sequence of SEQ ID NO: 147. In some embodiments, the beta chain comprises a beta variable region comprising an amino acid sequence having at least 90%, 95%, or 100% identity to the amino acid sequence of SEQ ID NO: 148. In some embodiments, the beta chain comprises an amino acid sequence having at least 90%, 95%, or 100% identity to the amino acid sequence of SEQ ID NO: 149. In some embodiments, the beta chain comprises an amino acid sequence having at least 90%, 95%, or 100% identity to the amino acid sequence of SEQ ID NO: 287.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 150, a CDR2-alpha sequence comprising the amino acid sequence of SEQ ID NO: 151, a CDR3-alpha sequence comprising the amino acid sequence of SEQ ID NO: 152, a CDR1-beta sequence comprising the amino acid sequence of SEQ ID NO: 155, a CDR2-beta sequence comprising the amino acid sequence of SEQ ID NO: 156, and/or a CDR3-beta sequence comprising the amino acid sequence of SEQ ID NO: 157.

In some embodiments, a TCR comprises aa CDR1-alpha sequence comprising the amino acid sequence of SEQ ID NO: 150, a CDR2-alpha sequence comprising the amino acid sequence of SEQ ID NO: 151, and a CDR3-alpha sequence comprising the amino acid sequence of SEQ ID NO: 152. In some embodiments, the alpha chain comprises an alpha variable region comprising an amino acid sequence having at least 90%, 95%, or 100% identity to the amino acid sequence of SEQ ID NO: 153. In some embodiments, the alpha chain comprises an amino acid sequence having at least 90%, 95%, or 100% identity to the amino acid sequence of SEQ ID NO: 154. In some embodiments, the alpha chain comprises an amino acid sequence having at least 90%, 95%, or 100% identity to the amino acid sequence of SEQ ID NO: 288.

In some embodiments, a TCR comprises a CDR1-beta sequence comprising the amino acid sequence of SEQ ID NO: 155, a CDR2-beta sequence comprising the amino acid sequence of SEQ ID NO: 156, and a CDR3-beta sequence comprising the amino acid sequence of SEQ ID NO: 157. In some embodiments, the beta chain comprises a beta variable region comprising an amino acid sequence having at least 90%, 95%, or 100% identity to the amino acid sequence of SEQ ID NO: 158. In some embodiments, the beta chain comprises an amino acid sequence having at least 90%, 95%, or 100% identity to the amino acid sequence of SEQ ID NO: 159. In some embodiments, the beta chain comprises an amino acid sequence having at least 90%, 95%, or 100% identity to the amino acid sequence of SEQ ID NO: 289.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 160, a CDR2-alpha sequence comprising the amino acid sequence of SEQ ID NO: 161, a CDR3-alpha sequence comprising the amino acid sequence of SEQ ID NO: 162, a CDR1-beta sequence comprising the amino acid sequence of SEQ ID NO: 165, a CDR2-beta sequence comprising the amino acid sequence of SEQ ID NO: 166, and/or a CDR3-beta sequence comprising the amino acid sequence of SEQ ID NO: 167.

In some embodiments, a TCR comprises a CDR1-alpha sequence comprising the amino acid sequence of SEQ ID NO: 160, a CDR2-alpha sequence comprising the amino acid sequence of SEQ ID NO: 161, and a CDR3-alpha sequence comprising the amino acid sequence of SEQ ID NO: 162. In some embodiments, the alpha chain comprises an alpha variable region comprising an amino acid sequence having at least 90%, 95%, or 100% identity to the amino acid sequence of SEQ ID NO: 163. In some embodiments, the alpha chain comprises an amino acid sequence having at least 90%, 95%, or 100% identity to the amino acid sequence of SEQ ID NO: 164. In some embodiments, the alpha chain comprises an amino acid sequence having at least 90%, 95%, or 100% identity to the amino acid sequence of SEQ ID NO: 290.

In some embodiments, a TCR comprises a CDR1-beta sequence comprising the amino acid sequence of SEQ ID NO: 165, a CDR2-beta sequence comprising the amino acid sequence of SEQ ID NO: 166, and a CDR3-beta sequence comprising the amino acid sequence of SEQ ID NO: 167. In some embodiments, the beta chain comprises a beta variable region comprising an amino acid sequence having at least 90%, 95%, or 100% identity to the amino acid sequence of SEQ ID NO: 168. In some embodiments, the beta chain comprises an amino acid sequence having at least 90%, 95%, or 100% identity to the amino acid sequence of SEQ ID NO: 169. In some embodiments, the beta chain comprises an amino acid sequence having at least 90%, 95%, or 100% identity to the amino acid sequence of SEQ ID NO: 291.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 170, a CDR2-alpha sequence comprising the amino acid sequence of SEQ ID NO: 171, a CDR3-alpha sequence comprising the amino acid sequence of SEQ ID NO: 172, a CDR1-beta sequence comprising the amino acid sequence of SEQ ID NO: 175, a CDR2-beta sequence comprising the amino acid sequence of SEQ ID NO: 176, and/or a CDR3-beta sequence comprising the amino acid sequence of SEQ ID NO: 177.

In some embodiments, a TCR comprises a CDR1-alpha sequence comprising the amino acid sequence of SEQ ID NO: 170, a CDR2-alpha sequence comprising the amino acid sequence of SEQ ID NO: 171, and a CDR3-alpha sequence comprising the amino acid sequence of SEQ ID NO: 172. In some embodiments, the alpha chain comprises an alpha variable region comprising an amino acid sequence having at least 90%, 95%, or 100% identity to the amino acid sequence of SEQ ID NO: 173. In some embodiments, the alpha chain comprises an amino acid sequence having at least 90%, 95%, or 100% identity to the amino acid sequence of SEQ ID NO: 174. In some embodiments, the alpha chain comprises an amino acid sequence having at least 90%, 95%, or 100% identity to the amino acid sequence of SEQ ID NO: 292.

In some embodiments, a TCR comprises a CDR1-beta sequence comprising the amino acid sequence of SEQ ID NO: 175, a CDR2-beta sequence comprising the amino acid sequence of SEQ ID NO: 176, and a CDR3-beta sequence comprising the amino acid sequence of SEQ ID NO: 177. In some embodiments, the beta chain comprises a beta variable region comprising an amino acid sequence having at least 90%, 95%, or 100% identity to the amino acid sequence of SEQ ID NO: 178. In some embodiments, the beta chain comprises an amino acid sequence having at least 90%, 95%, or 100% identity to the amino acid sequence of SEQ ID NO: 179. In some embodiments, the beta chain comprises an amino acid sequence having at least 90%, 95%, or 100% identity to the amino acid sequence of SEQ ID NO: 293.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 180, a CDR2-alpha sequence comprising the amino acid sequence of SEQ ID NO: 181, a CDR3-alpha sequence comprising the amino acid sequence of SEQ ID NO: 182, a CDR1-beta sequence comprising the amino acid sequence of SEQ ID NO: 185, a CDR2-beta sequence comprising the amino acid sequence of SEQ ID NO: 186, and/or a CDR3-beta sequence comprising the amino acid sequence of SEQ ID NO: 187.

In some embodiments, a TCR comprises a CDR1-alpha sequence comprising the amino acid sequence of SEQ ID NO: 180, a CDR2-alpha sequence comprising the amino acid sequence of SEQ ID NO: 181, and a CDR3-alpha sequence comprising the amino acid sequence of SEQ ID NO: 182. In some embodiments, the alpha chain comprises an alpha variable region comprising an amino acid sequence having at least 90%, 95%, or 100% identity to the amino acid sequence of SEQ ID NO: 183. In some embodiments, the alpha chain comprises an amino acid sequence having at least 90%, 95%, or 100% identity to the amino acid sequence of SEQ ID NO: 294. In some embodiments, the alpha chain comprises an amino acid sequence having at least 90%, 95%, or 100% identity to the amino acid sequence of SEQ ID NO: 184.

In some embodiments, a TCR comprises a CDR1-beta sequence comprising the amino acid sequence of SEQ ID NO: 185, a CDR2-beta sequence comprising the amino acid sequence of SEQ ID NO: 186, and a CDR3-beta sequence comprising the amino acid sequence of SEQ ID NO: 187. In some embodiments, the beta chain comprises a beta variable region comprising an amino acid sequence having at least 90%, 95%, or 100% identity to the amino acid sequence of SEQ ID NO: 188. In some embodiments, the beta chain comprises an amino acid sequence having at least 90%, 95%, or 100% identity to the amino acid sequence of SEQ ID NO: 295. In some embodiments, the beta chain comprises an amino acid sequence having at least 90%, 95%, or 100% identity to the amino acid sequence of SEQ ID NO: 189.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 190, a CDR2-alpha sequence comprising the amino acid sequence of SEQ ID NO: 191, a CDR3-alpha sequence comprising the amino acid sequence of SEQ ID NO: 192, a CDR1-beta sequence comprising the amino acid sequence of SEQ ID NO: 195, a CDR2-beta sequence comprising the amino acid sequence of SEQ ID NO: 196, and/or a CDR3-beta sequence comprising the amino acid sequence of SEQ ID NO: 197.

In some embodiments, a TCR comprises a CDR1-alpha sequence comprising the amino acid sequence of SEQ ID NO: 190, a CDR2-alpha sequence comprising the amino acid sequence of SEQ ID NO: 191, and a CDR3-alpha sequence comprising the amino acid sequence of SEQ ID NO: 192. In some embodiments, the alpha chain comprises an alpha variable region comprising an amino acid sequence having at least 90%, 95%, or 100% identity to the amino acid sequence of SEQ ID NO: 193. In some embodiments, the alpha chain comprises an amino acid sequence having at least 90%, 95%, or 100% identity to the amino acid sequence of SEQ ID NO: 296. In some embodiments, the alpha chain comprises an amino acid sequence having at least 90%, 95%, or 100% identity to the amino acid sequence of SEQ ID NO: 194.

In some embodiments, a TCR comprises a CDR1-beta sequence comprising the amino acid sequence of SEQ ID NO: 195, a CDR2-beta sequence comprising the amino acid sequence of SEQ ID NO: 196, and a CDR3-beta sequence comprising the amino acid sequence of SEQ ID NO: 197. In some embodiments, the beta chain comprises a beta variable region comprising an amino acid sequence having at least 90%, 95%, or 100% identity to the amino acid sequence of SEQ ID NO: 198. In some embodiments, the beta chain comprises an amino acid sequence having at least 90%, 95%, or 100% identity to the amino acid sequence of SEQ ID NO: 297. In some embodiments, the beta chain comprises an amino acid sequence having at least 90%, 95%, or 100% identity to the amino acid sequence of SEQ ID NO: 199.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 200, a CDR2-alpha sequence comprising the amino acid sequence of SEQ ID NO: 201, a CDR3-alpha sequence comprising the amino acid sequence of SEQ ID NO: 202, a CDR1-beta sequence comprising the amino acid sequence of SEQ ID NO: 205, a CDR2-beta sequence comprising the amino acid sequence of SEQ ID NO: 206, and/or a CDR3-beta sequence comprising the amino acid sequence of SEQ ID NO: 207.

In some embodiments, a TCR comprises a CDR1-alpha sequence comprising the amino acid sequence of SEQ ID NO: 200, a CDR2-alpha sequence comprising the amino acid sequence of SEQ ID NO: 201, and a CDR3-alpha sequence comprising the amino acid sequence of SEQ ID NO: 202. In some embodiments, the alpha chain comprises an alpha variable region comprising an amino acid sequence having at least 90%, 95%, or 100% identity to the amino acid sequence of SEQ ID NO: 203. In some embodiments, the alpha chain comprises an amino acid sequence having at least 90%, 95%, or 100% identity to the amino acid sequence of SEQ ID NO: 298. In some embodiments, the alpha chain comprises an amino acid sequence having at least 90%, 95%, or 100% identity to the amino acid sequence of SEQ ID NO: 204.

In some embodiments, a TCR comprises a CDR1-beta sequence comprising the amino acid sequence of SEQ ID NO: 205, a CDR2-beta sequence comprising the amino acid sequence of SEQ ID NO: 206, and a CDR3-beta sequence comprising the amino acid sequence of SEQ ID NO: 207. In some embodiments, the beta chain comprises a beta variable region comprising an amino acid sequence having at least 90%, 95%, or 100% identity to the amino acid sequence of SEQ ID NO: 208. In some embodiments, the beta chain comprises an amino acid sequence having at least 90%, 95%, or 100% identity to the amino acid sequence of SEQ ID NO: 299. In some embodiments, the beta chain comprises an amino acid sequence having at least 90%, 95%, or 100% identity to the amino acid sequence of SEQ ID NO: 209.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 210, a CDR2-alpha sequence comprising the amino acid sequence of SEQ ID NO: 211, a CDR3-alpha sequence comprising the amino acid sequence of SEQ ID NO: 212, a CDR1-beta sequence comprising the amino acid sequence of SEQ ID NO: 215, a CDR2-beta sequence comprising the amino acid sequence of SEQ ID NO: 216, and/or a CDR3-beta sequence comprising the amino acid sequence of SEQ ID NO: 217.

In some embodiments, a TCR comprises a CDR1-alpha sequence comprising the amino acid sequence of SEQ ID NO: 210, a CDR2-alpha sequence comprising the amino acid sequence of SEQ ID NO: 211, and a CDR3-alpha sequence comprising the amino acid sequence of SEQ ID NO: 212. In some embodiments, the alpha chain comprises an alpha variable region comprising an amino acid sequence having at least 90%, 95%, or 100% identity to the amino acid sequence of SEQ ID NO: 213. In some embodiments, the alpha chain comprises an amino acid sequence having at least 90%, 95%, or 100% identity to the amino acid sequence of SEQ ID NO: 300. In some embodiments, the alpha chain comprises an amino acid sequence having at least 90%, 95%, or 100% identity to the amino acid sequence of SEQ ID NO: 214.

In some embodiments, a TCR comprises a CDR1-beta sequence comprising the amino acid sequence of SEQ ID NO: 215, a CDR2-beta sequence comprising the amino acid sequence of SEQ ID NO: 216, and a CDR3-beta sequence comprising the amino acid sequence of SEQ ID NO: 217. In some embodiments, the beta chain comprises a beta variable region comprising an amino acid sequence having at least 90%, 95%, or 100% identity to the amino acid sequence of SEQ ID NO: 218. In some embodiments, the beta chain comprises an amino acid sequence having at least 90%, 95%, or 100% identity to the amino acid sequence of SEQ ID NO: 301. In some embodiments, the beta chain comprises an amino acid sequence having at least 90%, 95%, or 100% identity to the amino acid sequence of SEQ ID NO: 219.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 220, a CDR2-alpha sequence comprising the amino acid sequence of SEQ ID NO: 221, a CDR3-alpha sequence comprising the amino acid sequence of SEQ ID NO: 222, a CDR1-beta sequence comprising the amino acid sequence of SEQ ID NO: 225, a CDR2-beta sequence comprising the amino acid sequence of SEQ ID NO: 226, and/or a CDR3-beta sequence comprising the amino acid sequence of SEQ ID NO: 227.

In some embodiments, a TCR comprises a CDR1-alpha sequence comprising the amino acid sequence of SEQ ID NO: 220, a CDR2-alpha sequence comprising the amino acid sequence of SEQ ID NO: 221, and a CDR3-alpha sequence comprising the amino acid sequence of SEQ ID NO: 222. In some embodiments, the alpha chain comprises an alpha variable region comprising an amino acid sequence having at least 90%, 95%, or 100% identity to the amino acid sequence of SEQ ID NO: 223. In some embodiments, the alpha chain comprises an amino acid sequence having at least 90%, 95%, or 100% identity to the amino acid sequence of SEQ ID NO: 302. In some embodiments, the alpha chain comprises an amino acid sequence having at least 90%, 95%, or 100% identity to the amino acid sequence of SEQ ID NO: 224.

In some embodiments, a TCR comprises a CDR1-beta sequence comprising the amino acid sequence of SEQ ID NO: 225, a CDR2-beta sequence comprising the amino acid sequence of SEQ ID NO: 226, and a CDR3-beta sequence comprising the amino acid sequence of SEQ ID NO: 227. In some embodiments, the beta chain comprises a beta variable region comprising an amino acid sequence having at least 90%, 95%, or 100% identity to the amino acid sequence of SEQ ID NO: 228. In some embodiments, the beta chain comprises an amino acid sequence having at least 90%, 95%, or 100% identity to the amino acid sequence of SEQ ID NO: 303. In some embodiments, the beta chain comprises an amino acid sequence having at least 90%, 95%, or 100% identity to the amino acid sequence of SEQ ID NO: 229.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 230, a CDR2-alpha sequence comprising the amino acid sequence of SEQ ID NO: 231, a CDR3-alpha sequence comprising the amino acid sequence of SEQ ID NO: 232, a CDR1-beta sequence comprising the amino acid sequence of SEQ ID NO: 235, a CDR2-beta sequence comprising the amino acid sequence of SEQ ID NO: 236, and/or a CDR3-beta sequence comprising the amino acid sequence of SEQ ID NO: 237.

In some embodiments, a TCR comprises a CDR1-alpha sequence comprising the amino acid sequence of SEQ ID NO: 230, a CDR2-alpha sequence comprising the amino acid sequence of SEQ ID NO: 231, and a CDR3-alpha sequence comprising the amino acid sequence of SEQ ID NO: 232. In some embodiments, the alpha chain comprises an alpha variable region comprising an amino acid sequence having at least 90%, 95%, or 100% identity to the amino acid sequence of SEQ ID NO: 233. In some embodiments, the alpha chain comprises an amino acid sequence having at least 90%, 95%, or 100% identity to the amino acid sequence of SEQ ID NO: 304. In some embodiments, the alpha chain comprises an amino acid sequence having at least 90%, 95%, or 100% identity to the amino acid sequence of SEQ ID NO: 234.

In some embodiments, a TCR comprises a CDR1-beta sequence comprising the amino acid sequence of SEQ ID NO: 235, a CDR2-beta sequence comprising the amino acid sequence of SEQ ID NO: 236, and a CDR3-beta sequence comprising the amino acid sequence of SEQ ID NO: 237. In some embodiments, the beta chain comprises a beta variable region comprising an amino acid sequence having at least 90%, 95%, or 100% identity to the amino acid sequence of SEQ ID NO: 238. In some embodiments, the beta chain comprises an amino acid sequence having at least 90%, 95%, or 100% identity to the amino acid sequence of SEQ ID NO: 305. In some embodiments, the beta chain comprises an amino acid sequence having at least 90%, 95%, or 100% identity to the amino acid sequence of SEQ ID NO: 239.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 240, a CDR2-alpha sequence comprising the amino acid sequence of SEQ ID NO: 241, a CDR3-alpha sequence comprising the amino acid sequence of SEQ ID NO: 242, a CDR1-beta sequence comprising the amino acid sequence of SEQ ID NO: 245, a CDR2-beta sequence comprising the amino acid sequence of SEQ ID NO: 246, and/or a CDR3-beta sequence comprising the amino acid sequence of SEQ ID NO: 247.

In some embodiments, a TCR comprises a CDR1-alpha sequence comprising the amino acid sequence of SEQ ID NO: 240, a CDR2-alpha sequence comprising the amino acid sequence of SEQ ID NO: 241, and a CDR3-alpha sequence comprising the amino acid sequence of SEQ ID NO: 242. In some embodiments, the alpha chain comprises an alpha variable region comprising an amino acid sequence having at least 90%, 95%, or 100% identity to the amino acid sequence of SEQ ID NO: 243. In some embodiments, the alpha chain comprises an amino acid sequence having at least 90%, 95%, or 100% identity to the amino acid sequence of SEQ ID NO: 306. In some embodiments, the alpha chain comprises an amino acid sequence having at least 90%, 95%, or 100% identity to the amino acid sequence of SEQ ID NO: 244.

In some embodiments, a TCR comprises a CDR1-beta sequence comprising the amino acid sequence of SEQ ID NO: 245, a CDR2-beta sequence comprising the amino acid sequence of SEQ ID NO: 246, and a CDR3-beta sequence comprising the amino acid sequence of SEQ ID NO: 247. In some embodiments, the beta chain comprises a beta variable region comprising an amino acid sequence having at least 90%, 95%, or 100% identity to the amino acid sequence of SEQ ID NO: 248. In some embodiments, the beta chain comprises an amino acid sequence having at least 90%, 95%, or 100% identity to the amino acid sequence of SEQ ID NO: 307. In some embodiments, the beta chain comprises an amino acid sequence having at least 90%, 95%, or 100% identity to the amino acid sequence of SEQ ID NO: 249.

Some aspects relate to an engineered regulatory T cell that comprises an exogenous TCR that binds to a proinsulin peptide complexed with an MHC molecule, wherein the TCR comprises an alpha chain and a beta chain, and wherein the TCR comprises: a CDR1-alpha sequence comprising the amino acid sequence of SEQ ID NO: 250, a CDR2-alpha sequence comprising the amino acid sequence of SEQ ID NO: 251, a CDR3-alpha sequence comprising the amino acid sequence of SEQ ID NO: 252, a CDR1-beta sequence comprising the amino acid sequence of SEQ ID NO: 255, a CDR2-beta sequence comprising the amino acid sequence of SEQ ID NO: 256, and/or a CDR3-beta sequence comprising the amino acid sequence of SEQ ID NO: 257.

In some embodiments, a TCR comprises a CDR1-alpha sequence comprising the amino acid sequence of SEQ ID NO: 250, a CDR2-alpha sequence comprising the amino acid sequence of SEQ ID NO: 251, and a CDR3-alpha sequence comprising the amino acid sequence of SEQ ID NO: 252. In some embodiments, the alpha chain comprises an alpha variable region comprising an amino acid sequence having at least 90%, 95%, or 100% identity to the amino acid sequence of SEQ ID NO: 253. In some embodiments, the alpha chain comprises an amino acid sequence having at least 90%, 95%, or 100% identity to the amino acid sequence of SEQ ID NO: 308. In some embodiments, the alpha chain comprises an amino acid sequence having at least 90%, 95%, or 100% identity to the amino acid sequence of SEQ ID NO: 254.

In some embodiments, a TCR comprises a CDR1-beta sequence comprising the amino acid sequence of SEQ ID NO: 255, a CDR2-beta sequence comprising the amino acid sequence of SEQ ID NO: 256, and a CDR3-beta sequence comprising the amino acid sequence of SEQ ID NO: 257. In some embodiments, the beta chain comprises a beta variable region comprising an amino acid sequence having at least 90%, 95%, or 100% identity to the amino acid sequence of SEQ ID NO: 258. In some embodiments, the beta chain comprises an amino acid sequence having at least 90%, 95%, or 100% identity to the amino acid sequence of SEQ ID NO: 309. In some embodiments, the beta chain comprises an amino acid sequence having at least 90%, 95%, or 100% identity to the amino acid sequence of SEQ ID NO: 259.

### Populations

In some embodiments, the disclosure provides cell populations (e.g., isolated cell populations) comprising regulatory T cells, including stable regulatory T cells, transfected with an exogenous human TCR (e.g., a TCR as described in Table 1). Thus, in some embodiments, the disclosure provides isolated cell populations comprising stable regulatory T cells that comprise a hypomethylated TSDR at an endogenous FOXP3 locus. In some embodiments, stable regulatory T cells further comprise an exogenous human TCR that binds specifically to a target peptide complexed with an MHC molecule.

An isolated cell population is a cell population that is removed from a human body. Thus, it is considered “isolated” from the human body. A population of cells may be isolated (e.g., obtained from) a subject, or from a biological sample obtained from the subject, for example, using any known cell collection method, such as apheresis. An isolated cell population of the disclosure may be subjected to the methods described herein to produce an isolated cell population a higher number of regulatory T cells (e.g., stable regulatory T cells) relative to a population of cells obtained directly from a subject, or from a biological sample obtained from the subject, by apheresis, for example.

In some embodiments, an isolated cell population comprises

CD25^{+/high}CD4⁺CD127-1 regulatory T cells. In some embodiments, an isolated cell population comprises CD25^{+/high}CD4⁺CD127^{-/lo}/FOXP3′ regulatory T cells. In some embodiments, an isolated cell population comprises CD25^{+/high}CD4⁺CD127^{-/lo}/FOXP3⁺/CD45RA⁺ regulatory T cells.

In some embodiments, at least 50%, at least 60%, at least 70%, at least 75%, at least 80%, at least 85%, at least 90%, at least 95%, at least 96%, at least 97%, at least 98%, or at least 99% of the cells of an isolated cell population are stable regulatory T cells comprising a hypomethylated TSDR at an endogenous FOXP3 locus.

In some embodiments, at least 50%, at least 60%, at least 70%, at least 75%, at least 80%, at least 85%, at least 90%, at least 95%, at least 96%, at least 97%, at least 98%, or at least 99% of the cells of an isolated cell population are CD25^{+/high}CD4⁺CD127^{-/lo} regulatory T cells. In some embodiments, at least 50%, at least 60%, at least 70%, at least 75%, at least 80%, at least 85%, at least 90%, at least 95%, at least 96%, at least 97%, at least 98%, or at least 99% of the cells of an isolated cell population are CD25^{+/high}CD4⁺CD127^{-/lo}/FOXP3 regulatory T cells.

In some embodiments, at least 10%, at least 25%, at least 50%, at least 75%, at least 80%, at least 85%, at least 90%, at least 95%, at least 96%, at least 97%, at least 98%, or at least 99% of the stable regulatory T cells of a population are CD45RA. In some embodiments, at least 10%, at least 25%, at least 50%, at least 75%, at least 80%, at least 85%, at least 90%, at least 95%, at least 96%, at least 97%, at least 98%, or at least 99% of the stable regulatory T cells of a population are CD25^{+/high}CD4⁺CD127^{-/lo}/CD45RA regulatory T cells. In some embodiments, at least 10%, at least 25%, at least 50%, at least 75%, at least 80%, at least 85%, at least 90%, at least 95%, at least 96%, at least 97%, at least 98%, or at least 99% of the stable regulatory T cells of a population are CD25^{+/high}CD4⁺CD127^{-/lo}/FOXP3/CD45RA.

Stable regulatory T cells of a cell population, in some embodiments, are non-engineered at the endogenous FOXP3 locus. A FOXP3 locus is considered engineered if it has been edited (e.g., directly edited) using a gene editing technology such as, for example, homology-directed repair (HDR)—based gene editing, or if a cell has been otherwise genetically modified to increase or stabilize expression from the FOXP3 locus, relative to endogenous (naturally occurring) FOXP3 expression. In some embodiments, endogenous FOXP3 in the regulatory T cells of the population has not been gene edited. Thus, in some embodiments, fewer than (less than) 10% of the cells of the isolated cell population express FOXP3 protein from an engineered FOXP3 locus. In some embodiments, fewer than (less than) 9%, 8%, 7%, 6%, 5%, 4%, 3%, 2%, or 1% of the cells of the isolated cell population express FOXP3 protein from an engineered FOXP3 locus. In some embodiments, fewer than (less than) 0.5% or less than 0.1 of the cells of the isolated cell population express FOXP3 protein from an engineered FOXP3 locus.

In some embodiments, an isolated cell population comprises at least 1×10², at least 1×10³, at least 1×10⁴, at least 1×10⁵, at least 1×10⁶, at least 1×10⁷, at least 1×10⁸, at least 1×10⁹, or at least 1×10¹⁰ stable regulatory T cells. In some embodiments, an isolated cell population comprises 1×10² to 1×10¹⁰, 1×10³ to 1×10¹⁰, 1×10⁴ to 1×10¹⁰, 1×10⁵ to 1×10¹⁰, 1×10⁶ to 1×10¹⁰, 1×10⁷ to 1×10¹⁰, 1×10⁸ to 1×10¹⁰, 1×10⁵ to 1×10⁹, 1×10⁶ to 1×10⁸, 1×10⁷ to 1×10¹⁰, or 1×10⁴ to 1×10⁶ stable regulatory T cells. In some embodiments, an isolated cell population comprises 1×10⁶ to 1×10¹⁰ stable regulatory T cells.

In some embodiments, a subset of an isolated cell population are stable regulatory T cells (e.g., having a hypomethylated TSDR) at an endogenous FOXP3 and expressing CD4. CD25, and FOXP3). In some embodiments, stable regulatory T cells within an isolated cell population maintain a hypomethylated TSDR at an endogenous FOXP3 locus over time, as described elsewhere herein.

In some embodiments, stable regulatory T cells within an isolated cell population retain markers of stability in the presence of pro-inflammatory conditions (e.g., in presence of one or more pro-inflammatory cytokines). In some embodiments, stable regulatory T cells within an isolated cell population maintain a hypomethylated TSDR at an endogenous FOXP3 locus in the presence of pro-inflammatory conditions (e.g., in presence of one or more pro-inflammatory cytokines). In some embodiments, stable regulatory T cells within an isolated cell population maintain a hypomethylated TSDR at an endogenous FOXP3 locus and express CD4, CD25, and FOXP3 in the presence of pro-inflammatory conditions (e.g., in presence of one or more pro-inflammatory cytokines). In some embodiments, stable regulatory T cells within an isolated cell population maintain a hypomethylated TSDR at an endogenous FOXP3 locus in the presence of pro-inflammatory conditions (e.g., in presence of one or more pro-inflammatory cytokines) for at least 3, 4, 5, 6, 7, 8, 9, 10, 11, 12, 13, 14, 15, 20, 21, 22, 23, 24, or 25 days.

At least 50%, at least 60%, at least 70%, at least 80%, at least 90%, at least 95%, or at least 98% of the cells of an isolated cell population may be stable regulatory T cells. Thus, in some embodiments, at least 50%, at least 60%, at least 70%, at least 80%, at least 90%, at least 95%, or at least 98% of the cells of an isolated cell population comprises a hypomethylated TSDR at a FOXP3 locus. In some embodiments, at least 50%, at least 60%, at least 70%, at least 80%, at least 90%, at least 95%, or at least 98% of the cells of an isolated cell population comprises a hypomethylated TSDR at a FOXP3 locus for at least 1, 2, 3, 4, 5, 6, 7, 8, 9, 10, 11, 12, 13, 14, 15, 16, 17, 18, 19, or 20 days (e.g., after being obtained from a subject or following transduction with a nucleic acid expressing a TCR, for example, with 5 or fewer copies of a nucleic acid expressing a TCR).

In some embodiments, at least 50% of the cells of an isolated cell population comprises a hypomethylated TSDR at a FOXP3 locus for at least 5 days. In some embodiments, at least 50% of the cells of an isolated cell population comprises a hypomethylated TSDR at a FOXP3 locus for at least 10 days. In some embodiments, at least 50% of the cells of an isolated cell population comprises a hypomethylated TSDR at a FOXP3 locus for at least 15 days. In some embodiments, at least 60% of the cells of an isolated cell population comprises a hypomethylated TSDR at a FOXP3 locus for at least 5 days. In some embodiments, at least 60% of the cells of an isolated cell population comprises a hypomethylated TSDR at a FOXP3 locus for at least 10 days. In some embodiments, at least 60% of the cells of an isolated cell population comprises a hypomethylated TSDR at a FOXP3 locus for at least 15 days. In some embodiments, at least 70% of the cells of an isolated cell population comprises a hypomethylated TSDR at a FOXP3 locus for at least 5 days. In some embodiments, at least 70% of the cells of an isolated cell population comprises a hypomethylated TSDR at a FOXP3 locus for at least 10 days. In some embodiments, at least 70% of the cells of an isolated cell population comprises a hypomethylated TSDR at a FOXP3 locus for at least 15 days. In some embodiments, at least 80% of the cells of an isolated cell population comprises a hypomethylated TSDR at a FOXP3 locus for at least 5 days. In some embodiments, at least 80% of the cells of an isolated cell population comprises a hypomethylated TSDR at a FOXP3 locus for at least 10 days. In some embodiments, at least 80% of the cells of an isolated cell population comprises a hypomethylated TSDR at a FOXP3 locus for at least 15 days. In some embodiments, at least 90% of the cells of an isolated cell population comprises a hypomethylated TSDR at a FOXP3 locus for at least 5 days. In some embodiments, at least 90% of the cells of an isolated cell population comprises a hypomethylated TSDR at a FOXP3 locus for at least 10 days. In some embodiments, at least 90% of the cells of an isolated cell population comprises a hypomethylated TSDR at a FOXP3 locus for at least 15 days.

In some embodiments, the percentage of cells of the isolated cell population comprising the stable regulatory T cells does not decrease by more than 10, 9, 8, 7, 6, 5, 4, 3, 2, or 1 percentage point(s) during at least 5, 6, 7, 8, 9, 10, or 11 days of expansion following transduction of the stable regulatory T cells with a nucleic acid expressing an exogenous human TCR. In some embodiments, the percentage of cells of the isolated cell population comprising the stable regulatory T cells increases by 1, 2, 3, 4, or 5 percentage point(s) during at least 5. 6, 7, 8, 9, 10, or 11 days of expansion following transduction of the stable regulatory T cells with a nucleic acid expressing an exogenous human TCR. In some embodiments, the percentage of cells of the isolated cell population comprising the stable regulatory T cells does not decrease by more than 10 percentage points during at least 5 days of expansion following transduction of the stable regulatory T cells with a nucleic acid expressing an exogenous human TCR. In some embodiments, the percentage of cells of the isolated cell population comprising the stable regulatory T cells does not decrease by more than 10 percentage points during at least 10 days of expansion following transduction of the stable regulatory T cells with a nucleic acid expressing an exogenous human TCR. In some embodiments, the percentage of cells of the isolated cell population comprising the stable regulatory T cells does not decrease by more than 10 percentage points during at least 15 days of expansion following transduction of the stable regulatory T cells with a nucleic acid expressing an exogenous human TCR. In some embodiments, the percentage of cells of the isolated cell population comprising the stable regulatory T cells does not decrease by more than 5 percentage points during at least 5 days of expansion following transduction of the stable regulatory T cells with a nucleic acid expressing an exogenous human TCR. In some embodiments, the percentage of cells of the isolated cell population comprising the stable regulatory T cells does not decrease by more than 5 percentage points during at least 10 days of expansion following transduction of the stable regulatory T cells with a nucleic acid expressing an exogenous human TCR. In some embodiments, the percentage of cells of the isolated cell population comprising the stable regulatory T cells does not decrease by more than 5 percentage points during at least 15 days of expansion following transduction of the stable regulatory T cells with a nucleic acid expressing an exogenous human TCR. In some embodiments, the percentage of cells of the isolated cell population comprising the stable regulatory T cells does not decrease by more than 1 percentage point during at least 5 days of expansion following transduction of the stable regulatory T cells with a nucleic acid expressing an exogenous human TCR. In some embodiments, the percentage of cells of the isolated cell population comprising the stable regulatory T cells does not decrease by more than 1 percentage point during at least 10 days of expansion following transduction of the stable regulatory T cells with a nucleic acid expressing an exogenous human TCR. In some embodiments, the percentage of cells of the isolated cell population comprising the stable regulatory T cells does not decrease by more than 1 percentage point during at least 15 days of expansion following transduction of the stable regulatory T cells with a nucleic acid expressing an exogenous human TCR.

The percentage of stable regulatory T cells relative to total cells in an isolated cell population comprising regulatory T cells may be assessed about 1, about 6, about 12, about 24, about 36, about 48, about 72, about 96, or about 120 hours after transduction of cells with a nucleic acid expressing an exogenous human TCR. The percentage of stable regulatory T cells relative to total cells in an isolated cell population comprising regulatory T cells may be assessed 4, 5, 6, 7, 8, 9, 10, 11, 12, 13, or 14 days after transduction of cells with a nucleic acid expressing an exogenous human TCR. The percentage of stable regulatory T cells relative to total cells in an isolated cell population comprising regulatory T cells may be assessed about 1-21, 1-7, 4-14, 4-7, 7-10, 7-14, 10-21, or 14-21 days after transduction of cells with a nucleic acid expressing an exogenous human TCR.

In some embodiments, at least 10%, at least 25%, at least 50%, at least 60%, at least 70%, at least 80%, at least 90%, or at least 100% of the cells of an isolated cell population comprise an unmodified endogenous FOXP3 gene locus. In some embodiments, less than 25%, less than 20%, less than 15%, less than 10%, less than 5%, less than 2%, or less than 1% of the cells of the isolated cell population comprise a modified endogenous FOXP3 gene locus. An unmodified endogenous FOXP3 gene locus is a naturally occurring FOXP3 gene locus, which has not been edited, for example, using a gene editing or transgenic technique.

In some embodiments, at least 20%, at least 30%, at least 40%, at least 50%, at least 60%, at least 70%, at least 80%, at least 90%, or at least 100% of the cells of the isolated cell population express physiological levels of functional FOXP3 protein. A physiological level of functional FOXP3 protein is the protein level expressed from an unmodified (naturally occurring) FOXP3 gene locus.

In some embodiments, at least 20%, at least 30%, at least 40%, at least 50%, at least 60%, at least 70%, at least 80%, at least 90%, or at least 100% of the cells of the isolated cell population do not comprise an exogenous, e.g., ectopic, FOXP3 gene or an exogenous, e.g., ectopic, FOXP3 protein. In some embodiments, at least 20%, at least 30%, at least 40%, at least 50%, at least 60%, at least 70%, at least 80%, at least 90%, or at least 100% of the cells of the isolated cell population do not comprise ectopic expression of FOXP3. Ectopic expression of FOXP3 refers to expression that does not naturally occur in a cell. Thus, ectopic expression results, for example, from expression of an exogenous transgene or expression from an endogenous FOXP3 gene that has been modified at the FOXP3 gene locus, e.g., modified at the promoter region of the FOXP3 gene.

In some embodiments, less than 40%, less than 30%, less than 20%, less than 10%, less than 5%, less than 2%, or less than 1% of the cells of the isolated cell population comprise an exogenous, e.g., ectopic, FOXP3 gene or an exogenous, e.g., ectopic, FOXP3 protein. An exogenous gene or protein is one that has been introduced to a cell, for example, by transfection, transduction, or electroporation.

In some embodiments, at least 5%, at least 10%, at least 15%, at least 20%, at least 30%, at least 40%, at least 50%, at least 60%, at least 70%, at least 80%, at least 90%, or at least 95% of the regulatory T cells of an isolated cell population of regulatory T cells express an exogenous TCR (e.g., following transduction of the isolated cell population with a nucleic acid encoding an exogenous TCR). In some embodiments, 10%-60% or 20%-50% of the regulatory T cells of an isolated cell population of regulatory T cells express an exogenous TCR. In some embodiments, at least 5%, at least 10%, at least 15%, at least 20%, at least 30%, at least 40%, at least 50%, at least 60%, at least 70%, at least 80%, at least 90%, or at least 95% of the stable regulatory T cells of an isolated cell population of regulatory T cells express an exogenous TCR (e.g., following transduction of the isolated cell population with an exogenous TCR). In some embodiments, 10%-60% or 20%-50% of the stable regulatory T cells of an isolated cell population of regulatory T cells express an exogenous TCR.

In some embodiments, the TSDR at the endogenous FOXP3 locus of stable regulatory T cells of an isolated cell population of regulatory T cells remains hypomethylated until administration of the isolated cell population to a subject. In some embodiments, the TSDR at the endogenous FOXP3 locus of stable regulatory T cells of an isolated cell population of regulatory T cells remains hypomethylated following a cryopreservation freeze-thaw cycle.

In some embodiments, regulatory T cells (e.g., stable regulatory T cells) of an isolated cell population exhibit one or more cellular functions that are associated with regulatory T cells when activated by binding the pMHC. Non-limiting examples of such cellular functions include cytokine secretion activity, expression of certain activation markers, and suppression activity. Cytokine secretion activity simply refers to the secretion of certain anti-inflammatory cytokines, such as IL-10, IL-4, IL-6, IL-11, and IL-13. Activation markers include, but are not limited to CD69, 4-1BB, CD25, CD71, or CTLA-4. A regulatory T cell expresses one or more of these markers when it comes into contact with a target peptide complexed with MHC molecule, for example.

Suppression activity refers to the suppression of activation, proliferation and cytokine production of non-regulatory T cells (e.g., CD8⁺ T cells and CD4⁺ conventional T cells) in part to suppress the immune system from becoming overactive. Regulatory T cells exhibit suppression activity when contacted with a target peptide complexed with MHC molecule that binds to an exogenous TCR expressed by the regulatory T cells. In some embodiments, the target peptide is presented by a cell expressing the MHC molecule. For example, in some embodiments, regulatory T cells suppress the activation, proliferation and cytokine production of conventional T cells having specificity towards a shared target peptide complexed with MHC molecule, e.g., a shared target peptide presented by an Antigen Presenting Cell (APC). In some embodiments, regulatory T cells suppress proliferation and growth of non-regulatory T cells by at least 25%, at least 40%, at least 50%, at least 60%, at least 70%, or at least 80%, relative to a control (e.g., non-regulatory T cells in the absence of regulatory T cells).

In some embodiments, regulatory T cells suppress the production of interferon (IFN)-gamma from conventional T cells by at least 25%, at least 40%, at least 50%, at least 60%, at least 70%, or at least 80%, relative to a control (e.g., conventional T cells in the absence of regulatory T cells). In some embodiments, regulatory T cells suppress the production of IFN-gamma from conventional T cells by 50%-99%, 75%-99%, or 80%-100% relative to a control (e.g., when present in a population comprising a ratio of 1:1 to 1:8 regulatory T cells compared to conventional T cells). In some embodiments, regulatory T cells suppress the production of CD71 from conventional T cells by at least 20%, at least 30%, at least 40%, at least 50%, at least 60%, or at least 70%, relative to a control (e.g., conventional T cells in the absence of regulatory T cells). In some embodiments, regulatory T cells suppress the production of CD71 of conventional T cells by 20%-90% or 30%-80% relative to a control (e.g., when present in a population comprising a ratio of 1:1 to 1:8 regulatory T cells compared to conventional T cells). In some embodiments, regulatory T cells suppress the production of CD25 from conventional T cells by at least 20%, at least 30%, at least 40%, at least 50%, at least 60%, or at least 70%, relative to a control (e.g., conventional T cells in the absence of regulatory T cells). In some embodiments, regulatory T cells suppress the production of CD25 of conventional T cells by 40%-70% relative to a control (e.g., when present in a population comprising a ratio of 1:1 to 1:8 regulatory T cells compared to conventional T cells).

In some embodiments, regulatory T cells exhibit cytokine secretion activity (e.g., secretion of IL-10) when contacted with a target peptide complexed with MHC molecule, e.g., a target peptide presented by an APC, that binds to an exogenous TCR expressed by the regulatory T cells. In some embodiments, regulatory T cells exhibit expression of activation markers when contacted with a target peptide complexed with MHC molecule that binds to an exogenous TCR expressed by the regulatory T cells. For example, in some embodiments, regulatory T cells exhibit expression of CD69, 4-1BB, CD25, CD71, and/or CTLA-4 when contacted with a target peptide complexed with MHC molecule that binds to an exogenous TCR expressed by the regulatory T cells. In some embodiments, regulatory T cells exhibit suppression activity when contacted with a target peptide complexed with MHC molecule that binds to an exogenous TCR expressed by the regulatory T cells. For example, in some embodiments, regulatory T cells suppress the activation of conventional T cells having specificity towards a shared target peptide complexed with MHC molecule.

The cellular functions of stable regulatory T cells may be assessed about 1, about 6, about 12, about 24, about 36, about 48, about 72, about 96, or about 120 hours after transduction of cells with a nucleic acid expressing an exogenous human TCR. The cellular functions of stable regulatory T cells may be assessed 4, 5, 6, 7, 8, 9, 10, 11, 12, 13, or 14 days after transduction of cells with a nucleic acid expressing an exogenous human TCR. The cellular functions of stable regulatory T cells may be assessed about 1-21, 1-7, 4-14, 4-7, 7-10, 7-14, 10-21, or 14-21 days after transduction of cells with a nucleic acid expressing an exogenous human TCR.

In some embodiments, transduced regulatory T cells (e.g., transduced regulatory T cells of an isolated population) retain their cellular functionality (e.g., ability to be activated) following a cryopreservation freeze-thaw cycle. In some embodiments, transduced regulatory T cells can be activated and/or expanded following a cryopreservation freeze-thaw cycle. In some embodiments, regulatory T cells can exhibit cytokine secretion activity, expression of certain activation markers, and/or suppression activity following a cryopreservation freeze-thaw cycle.

In some embodiments, at least 5%, at least 10%, at least 25%, at least 50%, at least 75%, at least 80%, at least 85%, at least 90%, at least 95%, at least 96%, at least 97%, at least 98%, or at least 99% of a population of regulatory T cells are CD25^{+/high}CD4⁺CD127-lo prior to activation and/or transduction with a nucleic acid expressing an exogenous human T cell receptor. In some embodiments, at least 5%, at least 10%, at least 25%, at least 50%, at least 75%, at least 80%, at least 85%, at least 90%, at least 95%, at least 96%, at least 97%, at least 98%, or at least 99% of a population of regulatory T cells are CD25^{+/high}CD4⁺CD127^{-/lo}CD45RA⁺ prior to activation and/or transduction with a nucleic acid expressing an exogenous human T cell receptor.

An isolated cell population comprising regulatory T cells may comprise a minority amount of non-regulatory T cells (e.g., conventional T cells). Non-regulatory T cells may be NK T cells. B cells. CD8⁺ T cells, neutrophils, eosinophils. CD14⁺ cells, or conventional (CD4⁺) T cells that are derived from peripheral blood and lymph nodes. In some embodiments, less than 25%, less than 20%, less than 15%, less than 10%, less than 5%, less than 2%, less than 1%, less than 0.5%, less than 0.1%, or less than 0.01% of the cells of an isolated cell population comprising regulatory T cells are non-regulatory T cells. In some embodiments, about 0.01% to about 0.1%, about 0.1% to about 0.5%, about 0.5% to about 1%, about 0.5% to about 10%, about 2% to about 5%, or about 5% to about 10% of the cells of an isolated cell population comprising regulatory T cells are non-regulatory T cells. In some embodiments, less than 25%, less than 20%, less than 15%, less than 10%, less than 5%, less than 2%, less than 1%, less than 0.5%, less than 0.1%, or less than 0.01% of the cells of an isolated cell population comprising regulatory T cells are non-regulatory T cells that comprise an exogenous human TCR (e.g., an engineered human TCR of the disclosure). In some embodiments. 10% or fewer of the cells of an isolated cell population comprising regulatory T cells are non-regulatory T cells that comprise an exogenous human TCR.

A conventional T cell commonly produces IL-2 and other interleukin factors. In some embodiments, less than 25%, less than 20%, less than 15%, less than 10%, less than 5%, less than 2%, less than 1%, less than 0.5%, less than 0.1%, or less than 0.01% of the cells of an isolated cell population comprising regulatory T cells are conventional T cells. In some embodiments, about 0.01% to about 0.1%, about 0.1% to about 0.5%, about 0.5% to about 1%, about 0.5% to about 10%, about 2% to about 5%, or about 5% to about 10% of the cells of an isolated cell population comprising regulatory T cells are conventional T cells. In some embodiments, less than 25%, less than 20%, less than 15%, less than 10%, less than 5%, less than 2%, less than 1%, less than 0.5%, less than 0.1%, or less than 0.01% of the cells of an isolated cell population comprising regulatory T cells are conventional T cells that comprise an exogenous human TCR (e.g., an engineered human TCR of the disclosure). In some embodiments, an isolated cell population comprising regulatory T cells comprises an undetectable amount of conventional T cells. In some embodiments, less than 25%, less than 20%, less than 15%, less than 10%, less than 5%, less than 2%, less than 1%, less than 0.5%, less than 0.1%, or less than 0.01% of the cells of an isolated cell population comprising regulatory T cells are conventional CD4⁺ T cells. In some embodiments. 20% or fewer of the cells of an isolated cell population comprising regulatory T cells are conventional CD4⁺ T cells. In some embodiments, an isolated cell population comprising regulatory T cells comprises an undetectable amount of conventional CD4⁺ T cells. A cell type is considered undetectable if the presence of the cell in a sample cannot by detected by flow cytometry, for example, based on one or more cell-specific biomarker(s).

In some embodiments, an isolated cell population comprising regulatory T cells comprises an undetectable amount of B cells. In some embodiments, an isolated cell population comprising regulatory T cells comprises an undetectable amount of NK T cells. In some embodiments, an isolated cell population comprising regulatory T cells comprises an undetectable amount of CD14⁺ cells. In some embodiments, an isolated cell population comprising regulatory T cells comprises an undetectable number of eosinophils. In some embodiments, an isolated cell population comprising regulatory T cells comprises an undetectable number of neutrophils.

In some embodiments, less than 10%, less than 5%, less than 2%, less than 1%, less than 0.5%, less than 0.1%, or less than 0.01% of the cells of an isolated cell population comprising regulatory T cells are CD8⁺ T cells. In some embodiments, 5% or fewer of the cells of an isolated cell population are CD8⁺ T cells. In some embodiments, an isolated population of cells comprising regulatory T cells comprises an undetectable amount of CD8⁺ T cells.

The percentage of conventional T cells or other non-regulatory T cells relative to total cells (or relative to stable regulatory T cells) may be assessed about 1, about 6, about 12, about 24, about 36, about 48, about 72, about 96, or about 120 hours after transduction of cells with a nucleic acid expressing an exogenous human TCR. The percentage of conventional T cells or other non-regulatory T cells relative to total cells (or relative to stable regulatory T cells) may be assessed 4, 5, 6, 7, 8, 9, 10, 11, 12, 13, or 14 days after transduction of cells with a nucleic acid expressing an exogenous human TCR. The percentage of conventional T cells or other non-regulatory T cells relative to total cells (or relative to stable regulatory T cells) may be assessed about 1-21, 1-7, 4-14, 4-7, 7-10, 7-14, 10-21, or 14-21 days after transduction of cells with a nucleic acid expressing an exogenous human TCR.

In some embodiments, the ratio of regulatory T cells to conventional T cells in an isolate population of cells comprising regulatory T cells is at least 5:1, at least 10:1, at least 15:1, at least 20:1, at least 25:1, at least 30:1, at least 35:1, at least 40:1, at least 45:1, at least 50:1, at least 60:1, at least 70:1, at least 80:1, at least 90:1, or at least 100:1.

### Pharmaceutical Compositions

In some embodiments, the disclosure provides pharmaceutical compositions comprising any one of the TCRs described herein (e.g., a TCR as described in Table 1). In some embodiments, the disclosure provides pharmaceutical compositions comprising the isolated cell populations of regulatory T cells (e.g., stable regulatory T cells) described herein. In some embodiments, a pharmaceutical composition comprises a cell population of regulatory T cells (e.g., stable regulatory T cells) described herein and a pharmaceutically acceptable excipient.

As used herein, a pharmaceutically acceptable excipient may also be referred to as a pharmaceutically acceptable carrier, pharmaceutically acceptable diluent, or pharmaceutically acceptable adjuvant. Formulation of pharmaceutically-acceptable excipients and carrier solutions is well-known to those of skill in the art, as is the development of suitable dosing and treatment regimens for using the particular compositions described herein in a variety of treatment regimens.

The pharmaceutical compositions typically should be sterile and stable under the conditions of manufacture and storage. Sterile injectable formulations may be prepared using a non-toxic parenterally acceptable diluent or solvent. A pharmaceutical composition for use in accordance with the present invention may include pharmaceutically acceptable dispersing agents, wetting agents, suspending agents, isotonic agents, coatings, antibacterial and antifungal agents, carriers, excipients, salts, or stabilizers which are non-toxic to the subjects at the dosages and concentrations employed. In some embodiments, the pharmaceutical composition may comprise an organic solvent, such as but not limited to, methyl acetate, dimethyl sulfoxide (DMSO), N,N-dimethylformamide (DMF), dimethoxyethane (DME), and dimethylacetamide, including mixtures or combinations thereof.

A pharmaceutical composition may comprise an effective amount of stable regulatory T cells that is sufficient to elicit a desired biological response. For example, in some embodiments, an effective amount of stable regulatory T cells as described herein may refer to a number of cells that is sufficient to improve a symptom associated with Type 1 Diabetes. As will be appreciated by the skilled artisan, an effective amount of a solution or preparation provided herein may vary depending on various factors as, for example, on the desired biological response, e.g., on the specific disease being treated, the specific symptom to be alleviated, on the cell or tissue being targeted, and on the subject's age, gender, and general health status.

In some embodiments, an effective amount of stable regulatory T cells (e.g., stable regulatory T cells that (a) comprise an exogenous human T cell receptor (TCR) that binds specifically to a target peptide complexed with an MHC molecule and (b) comprise a hypomethylated TSDR at an endogenous FOXP3 locus) comprises at least 1×10², at least 1×10³, at least 1×10⁴, at least 1×10⁵, at least 1×10⁶, at least 1×10⁷, at least 1×10⁸, at least 1×10⁹, or at least 1×10¹⁰ stable regulatory T cells. In some embodiments, an effective amount of stable regulatory T cells (e.g., stable regulatory T cells that (a) comprise an exogenous human T cell receptor (TCR) that binds specifically to a target peptide complexed with an MHC molecule and (b) comprise a hypomethylated TSDR at an endogenous FOXP3 locus) is 1×10² to 1×10¹⁰, 1×10³ to 1×10¹⁰, 1×10⁴ to 1×10¹⁰, 1×10⁵ to 1×10¹⁰, 1×10⁶ to 1×10¹⁰, 1×10⁷ to 1×10¹⁰, 1×10⁸ to 1×10¹⁰, 1×10⁹ to 1×10¹⁰ stable regulatory T cells.

In some embodiments, the isolated cell populations (e.g., intended to be used in a pharmaceutical composition) are cryopreserved (e.g., subjected to one or more cryopreservation freeze-thaw cycles). That is, the isolated cell populations produced herein may be combined with a cryoprotecting agent, which lowers the melting temperature by forming chemical bonds with water and increasing the total concentration of solutes in the system. Non-limiting examples of cryoprotecting agents include glycerol, dimethyl sulfoxide (DMSO), ethanediol, and propanediol. While traditional methods that often include the use of serum and DMSO may be used, the disclosure also contemplates the use of cryopreservation medium manufactured under cGMP conditions and formulated serum-free and of non-animal origin (e.g., using <10% DMSO in the freeze cocktail). Other cryopreservation techniques are also provided herein, including more advanced techniques of cooling, for example, vitrifying cells without the use of cryoprotecting agents. See, e.g., Shinshu University. “A new way to ‘freeze’ cells promises to transform the common cell-freezing practice.” ScienceDaily.com April 2019. This process of ultrarapid cooling, utilizes inkjet cell printing to cool at a rate of 10.000 degrees Celsius/second, causing near-vitrification of the cells.

In some embodiments, the pharmaceutical composition comprises a population of stable regulatory T cells (e.g., stable regulatory T cells that (a) comprise an exogenous human T cell receptor (TCR) that binds specifically to a target peptide complexed with an MHC molecule and (b) comprise a hypomethylated TSDR at an endogenous FOXP3 locus) formulated with a cryoprotecting agent. In some embodiments, the cryoprotecting agent is a component of a cryopreservation medium (e.g., a cryopreservation medium containing 5% DMSO). Thus, in some embodiments, the pharmaceutical composition comprises a population of stable regulatory T cells (e.g., stable regulatory T cells that (a) comprise an exogenous human T cell receptor (TCR) that binds specifically to a target peptide complexed with an MHC molecule and (b) comprise a hypomethylated TSDR at an endogenous FOXP3 locus) formulated in a cryopreservation medium (e.g., a cryopreservation medium containing 5% DMSO). In some embodiments, a cryopreservation medium comprises 1-10%, 2-10%, 3-8%. 2-6%, 4-8%, or 4-6% DMSO. In some embodiments, a cryopreservation medium comprises 1%, 2%, 3%, 4%, 5%, 6%, 7%, 8%, 9%, or 10% DMSO.

### Methods

In some embodiments, the disclosure provides methods of administration of TCRs as described herein (and related pharmaceutical compositions) to a subject (e.g., a subject having Type 1 Diabetes). In some embodiments, the disclosure provides methods of administration of isolated cell populations comprising regulatory T cells as described herein (and related pharmaceutical compositions) to a subject (e.g., a subject having Type 1 Diabetes). Type I Diabetes, once known as juvenile diabetes or insulin-dependent diabetes, is a chronic condition. In this condition, the pancreas makes little or no insulin. Insulin is a hormone the body uses to allow sugar (glucose) to enter cells to produce energy. Different factors, such as genetics and some viruses, may cause Type 1 Diabetes. Although Type 1 Diabetes usually appears during childhood or adolescence, it can develop in adults. Even after much research, Type 1 Diabetes has no cure. Treatment is directed toward managing the amount of sugar in the blood using insulin, diet and lifestyle to prevent complications.

The natural progression of Type 1 Diabetes in a subject consists of three stages. Stage 1 is characterized by normoglycemia accompanied by loss of self-tolerance and the development of two or more islet-directed autoantibodies. It is estimated that beta cells are 100% functional at this stage. The risk to develop Stage 3 Type 1 Diabetes in a Stage 1 subject is 44% after 5 years, 70% after 10 years and 100% over the lifetime of that subject. Stage 2 is characterized by accompaniment of the autoantibodies by dysglycemia, which reflects inadequate insulin secretion after glucose challenge. Stage 2 is typically asymptomatic despite an estimated 20-50% reduction in functional beta cells via immune destruction. The risk to develop Stage 3 Type 1 Diabetes in a Stage 1 subject is 75% after 5 years and 100% over the lifetime of that subject. Clinical diagnosis occurs at Stage 3. Stage 3 is characterized by the onset of symptoms and the need for insulin therapy. Approximately 20% of beta cells remain functional at Stage 3. The residual beta cell function results from variable insulitis (inflammation of the islets of Langerhans) that spares beta cell mass.

In some embodiments, the disclosure provides methods of administration of isolated cell populations comprising regulatory T cells as described herein (and related pharmaceutical compositions) to a subject having Stage 3 Type I Diabetes. In some embodiments, methods of administration of isolated cell populations comprising regulatory T cells as described herein (and related pharmaceutical compositions) to a subject targets insulitis in the pancreas of the subject.

In some embodiments, the disclosure provides methods of administering a TCR (e.g., a TCR as described in Table 1) or a pharmaceutical composition as described herein to a subject in an effective amount to alleviate one or more symptom of Type 1 Diabetes. In some embodiments, the disclosure provides methods of administering a cell population comprising regulatory T cells or a pharmaceutical composition as described herein to a subject in an effective amount to alleviate one or more symptom of Type 1 Diabetes. Non-limiting examples of symptoms of Type 1 Diabetes include reduced or no production of insulin, build-up of glucose/sugar in the bloodstream, feeling more thirsty than usual, urinating a lot, bed-wetting in children who have never wet the bed during the night, feeling very hungry, losing weight without trying, feeling irritable or having other mood changes, feeling tired and weak, having blurry vision.

In some embodiments, the disclosure provides methods of treating Type 1 Diabetes in a subject comprising administering a cell population comprising regulatory T cells or a pharmaceutical composition as described herein to the subject. In some embodiments, at least a portion of the population of cells are autologous cells (i.e., obtained from the same subject to which they are subsequently administered). In some embodiments, a population of cells is isolated from a subject, subjected to a method of producing a cell population (e.g., to increase the relative concentration of regulatory T cells within the population) as described herein, engineered to express an exogenous TCR, and then administered to the same subject in order to treat a disease.

In some embodiments, treating (or treatment of) a disease refers to a clinical intervention aimed to reverse, alleviate, delay the onset of, or inhibit the progress of Type 1 Diabetes, or one or more symptoms thereof. In some embodiments, treatment may be administered after one or more symptoms have developed and/or after a disease has been diagnosed. In other embodiments, treatment may be administered in the absence of symptoms. e.g., to prevent or delay onset of a symptom or inhibit onset or progression of a disease. For example, treatment may be administered to a susceptible individual prior to the onset of symptoms (e.g., because of knowledge of genetic factors). Treatment may also be continued after symptoms have resolved, for example, to prevent or delay their recurrence.

A subject refers to an individual organism, for example, an individual human. In some embodiments, a subject is a human subject, such as a male subject or a female subject. In some embodiments, a human subject is an adult (e.g., 18 years of age or older). In some embodiments, a human subject is a juvenile (e.g., under 18 years of age). In some embodiments, a subject is a non-human mammal. In some embodiments, a subject is a non-human primate. In some embodiments, the subject is a rodent. In some embodiments, a subject is a sheep, a goat, a cow/cattle, a cat, or a dog. In some embodiments, a subject is a research animal. In some embodiments, a subject is genetically engineered, e.g., a genetically engineered non-human subject. A subject may be male or female.

A subject may have Type 1 Diabetes. In some embodiments, a subject has new-onset/adult-onset Type 1 Diabetes (e.g., new onset Stage 3 Type 1 Diabetes). A subject may have an HLA-DRB1*04:01 genetic haplotype. In some embodiments, a subject has an HLA-DRB1*04:01 genetic haplotype and has Type 1 Diabetes (e.g., new onset Stage 3 Type 1 Diabetes).

In some embodiments, a subject has residual beta cell function. In some embodiments, 5-50% of the beta cells within a subject are functional. In some embodiments, 5-40%, 5-30%, 5-25%, 10-50%, 10-40%, 10-30%, 15-30%, 15-25%, 15-20%, or 20-25% of the beta cells within a subject are functional. In some embodiments, about 5%, about 10%, about 15%, about 20%, about 25%, about 30%, about 35%, or about 40% of the beta cells within a subject are functional. A beta cell is considered functional if the beta cell secretes insulin and/or C-peptide.

In some embodiments, a subject has residual beta cell function if the C-peptide concentration in the plasma or blood of the subject is at least 300 pmol/L, 400 pmol/L, 450 pmol/L, 500 pmol/L, 550) pmol/L, 600 pmol/L, 700 pmol/L, 800 pmol/L, or 1000 pmol/L. In some embodiments, a subject has residual beta cell function if the C-peptide concentration in the plasma or blood of the subject is at least 0.1 ng/ml, 0.2 ng/ml, 0.3 ng/ml, 0.4 ng/ml, 0.5 ng/ml, 0.6 ng/ml, 0.7 ng/ml, 0.8 ng/ml, 0.9 ng/ml or 1 ng/mL. In some embodiments, a clinically relevant definition of residual beta cell function (e.g., contributing to glycemic control) is as described in Bonfanti R et al., Acta Diabetol 1998; 35: 91-95.; or Palmer, J P et, al., Diabetes. 2004; 53:250-264; the contents of each of which are incorporated herein by reference.

In some embodiments, a subject does not have residual beta cell function.

In some embodiments, endogenous insulin production by a subject is evidenced by circulating C-peptide levels, which are also a measure of preserved beta cell mass.

Conventional and pharmaceutically acceptable routes of administration of the include, but are not limited to, intravenous, subcutaneous, intravenous, intrathecal administration direct delivery to the selected organ (e.g., intraportal delivery to the liver), oral, inhalation (including intranasal and intratracheal delivery), intraocular, intramuscular, intradermal, intratumoral, and other parental routes of administration. Routes of administration may be combined, if desired.

A cell population comprising regulatory T cells or a pharmaceutical composition may be administered as a bolus administration. In some embodiments, administration of a cell population comprising regulatory T cells or a pharmaceutical composition comprises one or more infusion of the isolated cell population or pharmaceutical composition to the subject (e.g., cells are infused through a central line, similar to a blood transfusion).

In some embodiments, a cell population or a composition comprising a cell population (e.g., comprising regulatory T cells expressing a TCR that binds specifically to preproinsulin 73-90 complexed with MHC molecule) is administered to a subject in an amount effective to prevent beta (B) cell destruction and/or preserve beta cell mass in the pancreas of the subject. Beta cells are the insulin-producing cells located within the islets of Langerhans in the pancreas. Beta cell destruction refers to the damage or death of beta cells in the pancreas. Beta cells are important because they produce insulin, a hormone that regulates blood sugar levels by allowing cells in the body to take up glucose from the bloodstream. Beta cell destruction can result in reduced or no insulin production, leading to Type 1 Diabetes. Beta cell destruction decreases beta cell mass (i.e., the total number or volume of beta cells in the pancreas) in a subject. Maintaining an appropriate beta cell mass is important for normal glucose homeostasis. Both the functional ability of individual beta cells (how effectively each cell can produce and secrete insulin) and the overall beta cell mass influence the total insulin output of the pancreas. Prevention of beta cell destruction refers to a reduction in beta cell destruction or complete inhibition of beta cell destruction resulting from an autoimmune response. The reduction may be at least 20%, at least 30%, at least 40%, at least 50%, at least 60%, at least 70%, at least 80%, or at least 90%, relative to a control (e.g., baseline). Prevention of beta cell destruction may by assessed based on the number of live beta cells or the rate of beta cell destruction, for example.

In some embodiments, a cell population or a composition comprising a cell population (e.g., comprising regulatory T cells expressing a TCR that binds specifically to preproinsulin 73-90 complexed with MHC molecule) is administered to a subject in an amount effective to repair pancreatic tissue damage in a subject. The damage to pancreatic tissue in patients with Type 1 Diabetes results from an autoimmune response against insulin-producing beta cells, leading to a progressive decline in the pancreas's ability to produce insulin. The insulin-producing beta cells of the pancreas have a limited capacity for regeneration. Without being bound by theory, by preventing this autoimmune response, beta cell turnover can occur.

In some embodiments, a cell population or a composition comprising a cell population (e.g., comprising regulatory T cells expressing a TCR that binds specifically to preproinsulin 73-90 complexed with MHC molecule) is administered to a subject in an amount effective to prevent autoimmunity associated with Type 1 Diabetes in a subject. In the case of Type I Diabetes, the immune system specifically attacks the insulin-producing beta cells in the islets of Langerhans within the pancreas. The engineered cells provided herein can prevent this autoimmune response by specifically binding to autoantigen produced in the pancreas, for example, and in turn secreting anti-inflammatory cytokines to suppress the inflammatory response. Thus, in some embodiments, a cell population or a composition comprising a cell population (e.g., comprising regulatory T cells expressing a TCR that binds specifically to preproinsulin 73-90 complexed with MHC molecule) is administered to a subject in an amount effective to suppress pancreatic islet-associated inflammation in a subject. Prevention, as discussed above, refers to a reduction or complete inhibition. The reduction may be at least 20%, at least 30%, at least 40%, at least 50%, at least 60%, at least 70%, at least 80%, or at least 90%, relative to a control (e.g., baseline). Likewise, suppression refers to a reduction or complete inhibition. The prevention of autoimmunity associated with Type 1 Diabetes and/or the suppression of pancreatic islet-associated inflammation may be assessed by assaying for pancreatic beta cell death, insulin production, inflammatory cytokine production, or a combination thereof.

Currently, Type 1 Diabetes subjects having an established clinical diagnosis (e.g., a diagnosis of over five years) may be administered cell replacement therapies (e.g., pancreatic islet transplantations) in an attempt to restore beta cell function. However, subjects who are administered cell replacement therapies may exhibit a reemergence of autoimmunity. Accordingly, in some embodiments, a cell population or a composition comprising a cell population (e.g., comprising regulatory T cells expressing a TCR that binds specifically to preproinsulin 73-90) complexed with MHC molecule) is co-administered with a cell replacement therapy to a subject in an amount effective to prevent autoimmunity associated with Type 1 Diabetes in a subject. In some embodiments, a cell replacement therapy is a transplantation of insulin-secreting cells derived from allogeneic stem cells. The cell population or a composition comprising a cell population (e.g., comprising regulatory T cells expressing a TCR that binds specifically to preproinsulin 73-90 complexed with MHC molecule) may be concurrently administered to a subject receiving a cell replacement therapy (i.e., the cell population or composition comprising a cell population are administered to the subject at the same time as the cell replacement therapy). Alternatively, a cell population or a composition comprising a cell population (e.g., comprising regulatory T cells expressing a TCR that binds specifically to preproinsulin 73-90 complexed with MHC molecule) may be administered to a subject after the subject has already received a cell replacement therapy. In some embodiments, a cell population or a composition comprising a cell population is administered to a subject 1-28 days (e.g., 1-7, 5-10, 5-20, 10-28, or 14-28 days) after the cell replacement therapy. In some embodiments, co-administration of a cell population or a composition comprising a cell population with a cell replacement therapy (e.g., pancreatic islet transplantation) suppresses reactivation of autoimmunity in a subject (which may lead to sustained survival of the cell graft).

### Additional Embodiments

Additional embodiments of the present disclosure are encompassed by the following numbered paragraphs:
1. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 1,
   a CDR2-alpha sequence comprising the amino acid sequence of SEQ ID NO: 2,
   a CDR3-alpha sequence comprising the amino acid sequence of SEQ ID NO: 3,
   a CDR1-beta sequence comprising the amino acid sequence of SEQ ID NO: 6,
   a CDR2-beta sequence comprising the amino acid sequence of SEQ ID NO: 7, and/or
   a CDR3-beta sequence comprising the amino acid sequence of SEQ ID NO: 8.
2. The TCR of paragraph 1 comprising:
   a CDR1-alpha sequence comprising the amino acid sequence of SEQ ID NO: 1,
   a CDR2-alpha sequence comprising the amino acid sequence of SEQ ID NO: 2, and
   a CDR3-alpha sequence comprising the amino acid sequence of SEQ ID NO: 3.
3. The TCR of paragraph 1 or 2, wherein the alpha chain comprises an alpha variable region comprising an amino acid sequence having at least 90%, 95%, or 100% identity to the amino acid sequence of SEQ ID NO: 4.
4. The TCR of paragraph 3, wherein the alpha chain comprises an amino acid sequence having at least 90%, 95%, or 100% identity to the amino acid sequence of SEQ ID NO: 5.
5. The TCR of paragraph 3, wherein the alpha chain comprises an amino acid sequence having at least 90%, 95%, or 100% identity to the amino acid sequence of SEQ ID NO: 260.
6. The TCR of any one of the preceding paragraphs comprising:
   a CDR1-beta sequence comprising the amino acid sequence of SEQ ID NO: 6,
   a CDR2-beta sequence comprising the amino acid sequence of SEQ ID NO: 7, and
   a CDR3-beta sequence comprising the amino acid sequence of SEQ ID NO: 8.
7. The TCR of paragraph 6, wherein the beta chain comprises a beta variable region comprising an amino acid sequence having at least 90%, 95%, or 100% identity to the amino acid sequence of SEQ ID NO: 9.
8. The TCR of paragraph 7, wherein the beta chain comprises an amino acid sequence having at least 90%, 95%, or 100% identity to the amino acid sequence of SEQ ID NO: 10.
9. The TCR of paragraph 6, wherein the beta chain comprises an amino acid sequence having at least 90%, 95%, or 100% identity to the amino acid sequence of SEQ ID NO: 261.
10. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 11,
   a CDR2-alpha sequence comprising the amino acid sequence of SEQ ID NO: 12,
   a CDR3-alpha sequence comprising the amino acid sequence of SEQ ID NO: 13,
   a CDR1-beta sequence comprising the amino acid sequence of SEQ ID NO: 16,
   a CDR2-beta sequence comprising the amino acid sequence of SEQ ID NO: 17, and/or
   a CDR3-beta sequence comprising the amino acid sequence of SEQ ID NO: 18.
11. The TCR of paragraph 10 comprising:
   a CDR1-alpha sequence comprising the amino acid sequence of SEQ ID NO: 11,
   a CDR2-alpha sequence comprising the amino acid sequence of SEQ ID NO: 12, and
   a CDR3-alpha sequence comprising the amino acid sequence of SEQ ID NO: 13.
12. The TCR of paragraph 10 or 11, wherein the alpha chain comprises an alpha variable region comprising an amino acid sequence having at least 90%, 95%, or 100% identity to the amino acid sequence of SEQ ID NO: 14.
13. The TCR of paragraph 8, wherein the alpha chain comprises an amino acid sequence having at least 90%, 95%, or 100% identity to the amino acid sequence of SEQ ID NO: 15.
14. The TCR of paragraph 12, wherein the alpha chain comprises an amino acid sequence having at least 90%, 95%, or 100% identity to the amino acid sequence of SEQ ID NO: 262.
15. The TCR of any one of paragraphs 10-13 comprising:
   a CDR1-beta sequence comprising the amino acid sequence of SEQ ID NO: 16,
   a CDR2-beta sequence comprising the amino acid sequence of SEQ ID NO: 17, and
   a CDR3-beta sequence comprising the amino acid sequence of SEQ ID NO: 18.
16. The TCR of paragraph 15, wherein the beta chain comprises an alpha variable region comprising an amino acid sequence having at least 90%, 95%, or 100% identity to the amino acid sequence of SEQ ID NO: 19.
17. The TCR of paragraph 15, wherein the beta chain comprises an amino acid sequence having at least 90%, 95%, or 100% identity to the amino acid sequence of SEQ ID NO: 20.
18. The TCR of paragraph 14, wherein the beta chain comprises an amino acid sequence having at least 90%, 95%, or 100% identity to the amino acid sequence of SEQ ID NO: 263.
19. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 21,
   a CDR2-alpha sequence comprising the amino acid sequence of SEQ ID NO: 22,
   a CDR3-alpha sequence comprising the amino acid sequence of SEQ ID NO: 23,
   a CDR1-beta sequence comprising the amino acid sequence of SEQ ID NO: 26,
   a CDR2-beta sequence comprising the amino acid sequence of SEQ ID NO: 27, and/or
   a CDR3-beta sequence comprising the amino acid sequence of SEQ ID NO: 28.
20. The TCR of paragraph 19 comprising:
   a CDR1-alpha sequence comprising the amino acid sequence of SEQ ID NO: 21,
   a CDR2-alpha sequence comprising the amino acid sequence of SEQ ID NO: 22, and
   a CDR3-alpha sequence comprising the amino acid sequence of SEQ ID NO: 23.
21. The TCR of paragraph 19 or 20, wherein:
   the alpha chain comprises an alpha variable region comprising an amino acid sequence having at least 90%, 95%, or 100% identity to the amino acid sequence of SEQ ID NO: 24; or
   the alpha chain comprises an amino acid sequence having at least 90%, 95%, or 100% identity to the amino acid sequence of SEQ ID NO: 25; or
   the alpha chain comprises an amino acid sequence having at least 90%, 95%, or 100% identity to the amino acid sequence of SEQ ID NO: 264.
22. The TCR of any one of paragraphs 19-21 comprising:
   a CDR1-beta sequence comprising the amino acid sequence of SEQ ID NO: 26,
   a CDR2-beta sequence comprising the amino acid sequence of SEQ ID NO: 27, and
   a CDR3-beta sequence comprising the amino acid sequence of SEQ ID NO: 28.
23. The TCR of paragraph 22, wherein the beta chain comprises a beta variable region comprising an amino acid sequence having at least 90%, 95%, or 100% identity to the amino acid sequence of SEQ ID NO: 29.
24. The TCR of paragraph 23, wherein the beta chain comprises an amino acid sequence having at least 90%, 95%, or 100% identity to the amino acid sequence of SEQ ID NO: 30.
25. The TCR of paragraph 23, wherein the beta chain comprises an amino acid sequence having at least 90%, 95%, or 100% identity to the amino acid sequence of SEQ ID NO: 265.
26. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 31,
   a CDR2-alpha sequence comprising the amino acid sequence of SEQ ID NO: 32,
   a CDR3-alpha sequence comprising the amino acid sequence of SEQ ID NO: 33,
   a CDR1-beta sequence comprising the amino acid sequence of SEQ ID NO: 36,
   a CDR2-beta sequence comprising the amino acid sequence of SEQ ID NO: 37, and/or
   a CDR3-beta sequence comprising the amino acid sequence of SEQ ID NO: 38.
27. The TCR of paragraph 26 comprising:
   a CDR1-alpha sequence comprising the amino acid sequence of SEQ ID NO: 31,
   a CDR2-alpha sequence comprising the amino acid sequence of SEQ ID NO: 32, and
   a CDR3-alpha sequence comprising the amino acid sequence of SEQ ID NO: 33.
28. The TCR of paragraph 26 or 27, wherein the alpha chain comprises an alpha variable region comprising an amino acid sequence having at least 90%, 95%, or 100% identity to the amino acid sequence of SEQ ID NO: 34.
29. The TCR of paragraph 28, wherein the alpha chain comprises an amino acid sequence having at least 90%, 95%, or 100% identity to the amino acid sequence of SEQ ID NO: 35.
30. The TCR of paragraph 28, wherein the alpha chain comprises an amino acid sequence having at least 90%, 95%, or 100% identity to the amino acid sequence of SEQ ID NO: 266.
31. The TCR of any one of paragraphs 26-29 comprising:
   a CDR1-beta sequence comprising the amino acid sequence of SEQ ID NO: 36,
   a CDR2-beta sequence comprising the amino acid sequence of SEQ ID NO: 37, and
   a CDR3-beta sequence comprising the amino acid sequence of SEQ ID NO: 38.
32. The TCR of paragraph 22, wherein the beta chain comprises a beta variable region comprising an amino acid sequence having at least 90%, 95%, or 100% identity to the amino acid sequence of SEQ ID NO: 39.
33. The TCR of paragraph 32, wherein the beta chain comprises an amino acid sequence having at least 90%, 95%, or 100% identity to the amino acid sequence of SEQ ID NO: 40.
34. The TCR of paragraph 32, wherein the beta chain comprises an amino acid sequence having at least 90%, 95%, or 100% identity to the amino acid sequence of SEQ ID NO: 267.
35. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 41,
   a CDR2-alpha sequence comprising the amino acid sequence of SEQ ID NO: 42,
   a CDR3-alpha sequence comprising the amino acid sequence of SEQ ID NO: 43,
   a CDR1-beta sequence comprising the amino acid sequence of SEQ ID NO: 46,
   a CDR2-beta sequence comprising the amino acid sequence of SEQ ID NO: 47, and/or
   a CDR3-beta sequence comprising the amino acid sequence of SEQ ID NO: 48.
36. The TCR of paragraph 35 comprising:
   a CDR1-alpha sequence comprising the amino acid sequence of SEQ ID NO: 41,
   a CDR2-alpha sequence comprising the amino acid sequence of SEQ ID NO: 42, and
   a CDR3-alpha sequence comprising the amino acid sequence of SEQ ID NO: 43.
37. The TCR of paragraph 35 or 36, wherein the alpha chain comprises an alpha variable region comprising an amino acid sequence having at least 90%, 95%, or 100% identity to the amino acid sequence of SEQ ID NO: 44.
38. The TCR of paragraph 37, wherein the alpha chain comprises an amino acid sequence having at least 90%, 95%, or 100% identity to the amino acid sequence of SEQ ID NO: 45.
39. The TCR of paragraph 37, wherein the alpha chain comprises an amino acid sequence having at least 90%, 95%, or 100% identity to the amino acid sequence of SEQ ID NO: 268.
40. The TCR of any one of paragraphs 35-38 comprising:
   a CDR1-beta sequence comprising the amino acid sequence of SEQ ID NO: 46,
   a CDR2-beta sequence comprising the amino acid sequence of SEQ ID NO: 47, and
   a CDR3-beta sequence comprising the amino acid sequence of SEQ ID NO: 48.
41. The TCR of paragraph 40, wherein the beta chain comprises a beta variable region comprising an amino acid sequence having at least 90%, 95%, or 100% identity to the amino acid sequence of SEQ ID NO: 49.
42. The TCR of paragraph 41, wherein the beta chain comprises an amino acid sequence having at least 90%, 95%, or 100% identity to the amino acid sequence of SEQ ID NO: 50.
43. The TCR of paragraph 41, wherein the beta chain comprises an amino acid sequence having at least 90%, 95%, or 100% identity to the amino acid sequence of SEQ ID NO: 269.
44. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 51,
   a CDR2-alpha sequence comprising the amino acid sequence of SEQ ID NO: 52,
   a CDR3-alpha sequence comprising the amino acid sequence of SEQ ID NO: 53,
   a CDR1-beta sequence comprising the amino acid sequence of SEQ ID NO: 56,
   a CDR2-beta sequence comprising the amino acid sequence of SEQ ID NO: 57, and/or
   a CDR3-beta sequence comprising the amino acid sequence of SEQ ID NO: 58.
45. The TCR of paragraph 44 comprising:
   a CDR1-alpha sequence comprising the amino acid sequence of SEQ ID NO: 51,
   a CDR2-alpha sequence comprising the amino acid sequence of SEQ ID NO: 52, and
   a CDR3-alpha sequence comprising the amino acid sequence of SEQ ID NO: 53.
46. The TCR of paragraph 44 or 45, wherein the alpha chain comprises an alpha variable region comprising an amino acid sequence having at least 90%, 95%, or 100% identity to the amino acid sequence of SEQ ID NO: 54.
47. The TCR of paragraph 46, wherein the alpha chain comprises an amino acid sequence having at least 90%, 95%, or 100% identity to the amino acid sequence of SEQ ID NO: 55.
48. The TCR of paragraph 46, wherein the alpha chain comprises an amino acid sequence having at least 90%, 95%, or 100% identity to the amino acid sequence of SEQ ID NO: 270.
49. The TCR of any one of paragraphs 44-47 comprising:
   a CDR1-beta sequence comprising the amino acid sequence of SEQ ID NO: 56,
   a CDR2-beta sequence comprising the amino acid sequence of SEQ ID NO: 57, and
   a CDR3-beta sequence comprising the amino acid sequence of SEQ ID NO: 58.
50. The TCR of paragraph 49, wherein the beta chain comprises a beta variable region comprising an amino acid sequence having at least 90%, 95%, or 100% identity to the amino acid sequence of SEQ ID NO: 59.
51. The TCR of paragraph 50, wherein the beta chain comprises an amino acid sequence having at least 90%, 95%, or 100% identity to the amino acid sequence of SEQ ID NO: 60.
52. The TCR of paragraph 50, wherein the beta chain comprises an amino acid sequence having at least 90%, 95%, or 100% identity to the amino acid sequence of SEQ ID NO: 271.
53. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 61,
   a CDR2-alpha sequence comprising the amino acid sequence of SEQ ID NO: 62,
   a CDR3-alpha sequence comprising the amino acid sequence of SEQ ID NO: 63,
   a CDR1-beta sequence comprising the amino acid sequence of SEQ ID NO: 66,
   a CDR2-beta sequence comprising the amino acid sequence of SEQ ID NO: 67, and/or
   a CDR3-beta sequence comprising the amino acid sequence of SEQ ID NO: 68.
54. The TCR of paragraph 53 comprising:
   a CDR1-alpha sequence comprising the amino acid sequence of SEQ ID NO: 61,
   a CDR2-alpha sequence comprising the amino acid sequence of SEQ ID NO: 62, and
   a CDR3-alpha sequence comprising the amino acid sequence of SEQ ID NO: 63.
55. The TCR of paragraph 53 or 54, wherein the alpha chain comprises an alpha variable region comprising an amino acid sequence having at least 90%, 95%, or 100% identity to the amino acid sequence of SEQ ID NO: 64.
56. The TCR of paragraph 55, wherein the alpha chain comprises an amino acid sequence having at least 90%, 95%, or 100% identity to the amino acid sequence of SEQ ID NO: 65.
57. The TCR of paragraph 55, wherein the alpha chain comprises an amino acid sequence having at least 90%, 95%, or 100% identity to the amino acid sequence of SEQ ID NO: 272.
58. The TCR of any one of paragraphs 53-56 comprising:
   a CDR1-beta sequence comprising the amino acid sequence of SEQ ID NO: 66,
   a CDR2-beta sequence comprising the amino acid sequence of SEQ ID NO: 67, and
   a CDR3-beta sequence comprising the amino acid sequence of SEQ ID NO: 68.
59. The TCR of paragraph 58, wherein the beta chain comprises a beta variable region comprising an amino acid sequence having at least 90%, 95%, or 100% identity to the amino acid sequence of SEQ ID NO: 69.
60. The TCR of paragraph 59, wherein the beta chain comprises an amino acid sequence having at least 90%, 95%, or 100% identity to the amino acid sequence of SEQ ID NO: 70.
61. The TCR of paragraph 59, wherein the beta chain comprises an amino acid sequence having at least 90%, 95%, or 100% identity to the amino acid sequence of SEQ ID NO: 273.
62. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 71,
   a CDR2-alpha sequence comprising the amino acid sequence of SEQ ID NO: 72,
   a CDR3-alpha sequence comprising the amino acid sequence of SEQ ID NO: 73,
   a CDR1-beta sequence comprising the amino acid sequence of SEQ ID NO: 76,
   a CDR2-beta sequence comprising the amino acid sequence of SEQ ID NO: 77, and/or
   a CDR3-beta sequence comprising the amino acid sequence of SEQ ID NO: 78.
63. The TCR of paragraph 62 comprising:
   a CDR1-alpha sequence comprising the amino acid sequence of SEQ ID NO: 71,
   a CDR2-alpha sequence comprising the amino acid sequence of SEQ ID NO: 72, and
   a CDR3-alpha sequence comprising the amino acid sequence of SEQ ID NO: 73.
64. The TCR of paragraph 62 or 63, wherein the alpha chain comprises an alpha variable region comprising an amino acid sequence having at least 90%, 95%, or 100% identity to the amino acid sequence of SEQ ID NO: 74.
65. The TCR of paragraph 64, wherein the alpha chain comprises an amino acid sequence having at least 90%, 95%, or 100% identity to the amino acid sequence of SEQ ID NO: 75.
66. The TCR of paragraph 64, wherein the alpha chain comprises an amino acid sequence having at least 90%, 95%, or 100% identity to the amino acid sequence of SEQ ID NO: 274.
67. The TCR of any one of paragraphs 62-65 comprising:
   a CDR1-beta sequence comprising the amino acid sequence of SEQ ID NO: 76,
   a CDR2-beta sequence comprising the amino acid sequence of SEQ ID NO: 77, and
   a CDR3-beta sequence comprising the amino acid sequence of SEQ ID NO: 78.
68. The TCR of paragraph 67, wherein the beta chain comprises a beta variable region comprising an amino acid sequence having at least 90%, 95%, or 100% identity to the amino acid sequence of SEQ ID NO: 79.
69. The TCR of paragraph 68, wherein the beta chain comprises an amino acid sequence having at least 90%, 95%, or 100% identity to the amino acid sequence of SEQ ID NO: 80.
70. The TCR of paragraph 68, wherein the beta chain comprises an amino acid sequence having at least 90%, 95%, or 100% identity to the amino acid sequence of SEQ ID NO: 275.
71. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 81,
   a CDR2-alpha sequence comprising the amino acid sequence of SEQ ID NO: 82,
   a CDR3-alpha sequence comprising the amino acid sequence of SEQ ID NO: 83,
   a CDR1-beta sequence comprising the amino acid sequence of SEQ ID NO: 86,
   a CDR2-beta sequence comprising the amino acid sequence of SEQ ID NO: 87, and/or
   a CDR3-beta sequence comprising the amino acid sequence of SEQ ID NO: 88.
72. The TCR of paragraph 71 comprising:
   a CDR1-alpha sequence comprising the amino acid sequence of SEQ ID NO: 81,
   a CDR2-alpha sequence comprising the amino acid sequence of SEQ ID NO: 82, and
   a CDR3-alpha sequence comprising the amino acid sequence of SEQ ID NO: 83.
73. The TCR of paragraph 71 or 73, wherein the alpha chain comprises an alpha variable region comprising an amino acid sequence having at least 90%, 95%, or 100% identity to the amino acid sequence of SEQ ID NO: 84.
74. The TCR of paragraph 73, wherein the alpha chain comprises an amino acid sequence having at least 90%, 95%, or 100% identity to the amino acid sequence of SEQ ID NO: 85.
75. The TCR of paragraph 73, wherein the alpha chain comprises an amino acid sequence having at least 90%, 95%, or 100% identity to the amino acid sequence of SEQ ID NO: 276.
76. The TCR of any one of paragraphs 71-74 comprising:
   a CDR1-beta sequence comprising the amino acid sequence of SEQ ID NO: 86,
   a CDR2-beta sequence comprising the amino acid sequence of SEQ ID NO: 87, and
   a CDR3-beta sequence comprising the amino acid sequence of SEQ ID NO: 88.
77. The TCR of paragraph 76, wherein the beta chain comprises a beta variable region comprising an amino acid sequence having at least 90%, 95%, or 100% identity to the amino acid sequence of SEQ ID NO: 89.
78. The TCR of paragraph 77, wherein the beta chain comprises an amino acid sequence having at least 90%, 95%, or 100% identity to the amino acid sequence of SEQ ID NO: 90.
79. The TCR of paragraph 77, wherein the beta chain comprises an amino acid sequence having at least 90%, 95%, or 100% identity to the amino acid sequence of SEQ ID NO: 277.
80. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 91,
   a CDR2-alpha sequence comprising the amino acid sequence of SEQ ID NO: 92,
   a CDR3-alpha sequence comprising the amino acid sequence of SEQ ID NO: 93,
   a CDR1-beta sequence comprising the amino acid sequence of SEQ ID NO: 96,
   a CDR2-beta sequence comprising the amino acid sequence of SEQ ID NO: 97, and/or
   a CDR3-beta sequence comprising the amino acid sequence of SEQ ID NO: 98.
81. The TCR of paragraph 80 comprising:
   a CDR1-alpha sequence comprising the amino acid sequence of SEQ ID NO: 91,
   a CDR2-alpha sequence comprising the amino acid sequence of SEQ ID NO: 92, and
   a CDR3-alpha sequence comprising the amino acid sequence of SEQ ID NO: 93.
82. The TCR of paragraph 80 or 81, wherein the alpha chain comprises an alpha variable region comprising an amino acid sequence having at least 90%, 95%, or 100% identity to the amino acid sequence of SEQ ID NO: 94.
83. The TCR of paragraph 82, wherein the alpha chain comprises an amino acid sequence having at least 90%, 95%, or 100% identity to the amino acid sequence of SEQ ID NO: 95.
84. The TCR of paragraph 82, wherein the alpha chain comprises an amino acid sequence having at least 90%, 95%, or 100% identity to the amino acid sequence of SEQ ID NO: 278.
85. The TCR of any one of paragraphs 80-83 comprising:
   a CDR1-beta sequence comprising the amino acid sequence of SEQ ID NO: 96,
   a CDR2-beta sequence comprising the amino acid sequence of SEQ ID NO: 97, and
   a CDR3-beta sequence comprising the amino acid sequence of SEQ ID NO: 98.
86. The TCR of paragraph 85, wherein the beta chain comprises a beta variable region comprising an amino acid sequence having at least 90%, 95%, or 100% identity to the amino acid sequence of SEQ ID NO: 99.
87. The TCR of paragraph 86, wherein the beta chain comprises an amino acid sequence having at least 90%, 95%, or 100% identity to the amino acid sequence of SEQ ID NO: 100.
88. The TCR of paragraph 86, wherein the beta chain comprises an amino acid sequence having at least 90%, 95%, or 100% identity to the amino acid sequence of SEQ ID NO: 279.
89. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 110,
   a CDR2-alpha sequence comprising the amino acid sequence of SEQ ID NO: 111,
   a CDR3-alpha sequence comprising the amino acid sequence of SEQ ID NO: 112,
   a CDR1-beta sequence comprising the amino acid sequence of SEQ ID NO: 115,
   a CDR2-beta sequence comprising the amino acid sequence of SEQ ID NO: 116, and/or
   a CDR3-beta sequence comprising the amino acid sequence of SEQ ID NO: 117.
90. The TCR of paragraph 89 comprising:
   a CDR1-alpha sequence comprising the amino acid sequence of SEQ ID NO: 110,
   a CDR2-alpha sequence comprising the amino acid sequence of SEQ ID NO: 111, and
   a CDR3-alpha sequence comprising the amino acid sequence of SEQ ID NO: 112.
91. The TCR of paragraph 89 or 90, wherein the alpha chain comprises an alpha variable region comprising an amino acid sequence having at least 90%, 95%, or 100% identity to the amino acid sequence of SEQ ID NO: 113.
92. The TCR of paragraph 91, wherein the alpha chain comprises an amino acid sequence having at least 90%, 95%, or 100% identity to the amino acid sequence of SEQ ID NO: 114.
93. The TCR of paragraph 91, wherein the alpha chain comprises an amino acid sequence having at least 90%, 95%, or 100% identity to the amino acid sequence of SEQ ID NO: 280.
94. The TCR of any one of paragraphs 89-93 comprising:
   a CDR1-beta sequence comprising the amino acid sequence of SEQ ID NO: 115,
   a CDR2-beta sequence comprising the amino acid sequence of SEQ ID NO: 116, and
   a CDR3-beta sequence comprising the amino acid sequence of SEQ ID NO: 117.
95. The TCR of paragraph 94, wherein the beta chain comprises a beta variable region comprising an amino acid sequence having at least 90%, 95%, or 100% identity to the amino acid sequence of SEQ ID NO: 118.
96. The TCR of paragraph 95, wherein the beta chain comprises an amino acid sequence having at least 90%, 95%, or 100% identity to the amino acid sequence of SEQ ID NO: 119.
97. The TCR of paragraph 95, wherein the beta chain comprises an amino acid sequence having at least 90%, 95%, or 100% identity to the amino acid sequence of SEQ ID NO: 281.
98. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 120,
   a CDR2-alpha sequence comprising the amino acid sequence of SEQ ID NO: 121,
   a CDR3-alpha sequence comprising the amino acid sequence of SEQ ID NO: 122,
   a CDR1-beta sequence comprising the amino acid sequence of SEQ ID NO: 125,
   a CDR2-beta sequence comprising the amino acid sequence of SEQ ID NO: 126, and/or
   a CDR3-beta sequence comprising the amino acid sequence of SEQ ID NO: 127.
99. The TCR of paragraph 98 comprising:
   a CDR1-alpha sequence comprising the amino acid sequence of SEQ ID NO: 120,
   a CDR2-alpha sequence comprising the amino acid sequence of SEQ ID NO: 121, and
   a CDR3-alpha sequence comprising the amino acid sequence of SEQ ID NO: 122.
100. The TCR of paragraph 98 or 99, wherein the alpha chain comprises an alpha variable region comprising an amino acid sequence having at least 90%, 95%, or 100% identity to the amino acid sequence of SEQ ID NO: 123.
101. The TCR of paragraph 100, wherein the alpha chain comprises an amino acid sequence having at least 90%, 95%, or 100% identity to the amino acid sequence of SEQ ID NO: 124.
102. The TCR of paragraph 100, wherein the alpha chain comprises an amino acid sequence having at least 90%, 95%, or 100% identity to the amino acid sequence of SEQ ID NO: 282.
103. The TCR of any one of paragraphs 98-101 comprising:
   a CDR1-beta sequence comprising the amino acid sequence of SEQ ID NO: 125,
   a CDR2-beta sequence comprising the amino acid sequence of SEQ ID NO: 126, and
   a CDR3-beta sequence comprising the amino acid sequence of SEQ ID NO: 127.
104. The TCR of paragraph 103, wherein the beta chain comprises a beta variable region comprising an amino acid sequence having at least 90%, 95%, or 100% identity to the amino acid sequence of SEQ ID NO: 128.
105. The TCR of paragraph 104, wherein the beta chain comprises an amino acid sequence having at least 90%, 95%, or 100% identity to the amino acid sequence of SEQ ID NO: 129.
106. The TCR of paragraph 104, wherein the beta chain comprises an amino acid sequence having at least 90%, 95%, or 100% identity to the amino acid sequence of SEQ ID NO: 283.
107. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 130,
   a CDR2-alpha sequence comprising the amino acid sequence of SEQ ID NO: 131,
   a CDR3-alpha sequence comprising the amino acid sequence of SEQ ID NO: 132,
   a CDR1-beta sequence comprising the amino acid sequence of SEQ ID NO: 135,
   a CDR2-beta sequence comprising the amino acid sequence of SEQ ID NO: 136, and/or
   a CDR3-beta sequence comprising the amino acid sequence of SEQ ID NO: 137.
108. The TCR of paragraph 107 comprising:
   a CDR1-alpha sequence comprising the amino acid sequence of SEQ ID NO: 130,
   a CDR2-alpha sequence comprising the amino acid sequence of SEQ ID NO: 131, and
   a CDR3-alpha sequence comprising the amino acid sequence of SEQ ID NO: 132.
109. The TCR of paragraph 108, wherein the alpha chain comprises an alpha variable region comprising an amino acid sequence having at least 90%, 95%, or 100% identity to the amino acid sequence of SEQ ID NO: 133.
110. The TCR of paragraph 108, wherein the alpha chain comprises an amino acid sequence having at least 90%, 95%, or 100% identity to the amino acid sequence of SEQ ID NO: 134.
111. The TCR of paragraph 109, wherein the alpha chain comprises an amino acid sequence having at least 90%, 95%, or 100% identity to the amino acid sequence of SEQ ID NO: 284.
112. The TCR of any one of paragraphs 107-110 comprising:
   a CDR1-beta sequence comprising the amino acid sequence of SEQ ID NO: 135,
   a CDR2-beta sequence comprising the amino acid sequence of SEQ ID NO: 136, and
   a CDR3-beta sequence comprising the amino acid sequence of SEQ ID NO: 137.
113. The TCR of paragraph 112, wherein the beta chain comprises a beta variable region comprising an amino acid sequence having at least 90%, 95%, or 100% identity to the amino acid sequence of SEQ ID NO: 138.
114. The TCR of paragraph 113, wherein the beta chain comprises an amino acid sequence having at least 90%, 95%, or 100% identity to the amino acid sequence of SEQ ID NO: 139.
115. The TCR of paragraph 113, wherein the beta chain comprises an amino acid sequence having at least 90%, 95%, or 100% identity to the amino acid sequence of SEQ ID NO: 285.
116. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 140,
   a CDR2-alpha sequence comprising the amino acid sequence of SEQ ID NO: 141,
   a CDR3-alpha sequence comprising the amino acid sequence of SEQ ID NO: 142,
   a CDR1-beta sequence comprising the amino acid sequence of SEQ ID NO: 145,
   a CDR2-beta sequence comprising the amino acid sequence of SEQ ID NO: 146, and/or
   a CDR3-beta sequence comprising the amino acid sequence of SEQ ID NO: 147.
117. The TCR of paragraph 116 comprising:
   a CDR1-alpha sequence comprising the amino acid sequence of SEQ ID NO: 140,
   a CDR2-alpha sequence comprising the amino acid sequence of SEQ ID NO: 141, and
   a CDR3-alpha sequence comprising the amino acid sequence of SEQ ID NO: 142.
118. The TCR of paragraph 117, wherein the alpha chain comprises an alpha variable region comprising an amino acid sequence having at least 90%, 95%, or 100% identity to the amino acid sequence of SEQ ID NO: 143.
119. The TCR of paragraph 118, wherein the alpha chain comprises an amino acid sequence having at least 90%, 95%, or 100% identity to the amino acid sequence of SEQ ID NO: 144.
120. The TCR of paragraph 118, wherein the alpha chain comprises an amino acid sequence having at least 90%, 95%, or 100% identity to the amino acid sequence of SEQ ID NO: 286.
121. The TCR of any one of paragraphs 116-119 comprising:
   a CDR1-beta sequence comprising the amino acid sequence of SEQ ID NO: 145,
   a CDR2-beta sequence comprising the amino acid sequence of SEQ ID NO: 146, and
   a CDR3-beta sequence comprising the amino acid sequence of SEQ ID NO: 147.
122. The TCR of paragraph 97, wherein the beta chain comprises a beta variable region comprising an amino acid sequence having at least 90%, 95%, or 100% identity to the amino acid sequence of SEQ ID NO: 148.
123. The TCR of paragraph 122, wherein the beta chain comprises an amino acid sequence having at least 90%, 95%, or 100% identity to the amino acid sequence of SEQ ID NO: 149.
124. The TCR of paragraph 122, wherein the beta chain comprises an amino acid sequence having at least 90%, 95%, or 100% identity to the amino acid sequence of SEQ ID NO: 287.
125. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 150,
   a CDR2-alpha sequence comprising the amino acid sequence of SEQ ID NO: 151,
   a CDR3-alpha sequence comprising the amino acid sequence of SEQ ID NO: 152,
   a CDR1-beta sequence comprising the amino acid sequence of SEQ ID NO: 155,
   a CDR2-beta sequence comprising the amino acid sequence of SEQ ID NO: 156, and/or
   a CDR3-beta sequence comprising the amino acid sequence of SEQ ID NO: 157.
126. The TCR of paragraph 125 comprising:
   a CDR1-alpha sequence comprising the amino acid sequence of SEQ ID NO: 150,
   a CDR2-alpha sequence comprising the amino acid sequence of SEQ ID NO: 151, and
   a CDR3-alpha sequence comprising the amino acid sequence of SEQ ID NO: 152.
127. The TCR of paragraph 126, wherein the alpha chain comprises an alpha variable region comprising an amino acid sequence having at least 90%, 95%, or 100% identity to the amino acid sequence of SEQ ID NO: 153.
128. The TCR of paragraph 127, wherein the alpha chain comprises an amino acid sequence having at least 90%, 95%, or 100% identity to the amino acid sequence of SEQ ID NO: 154.
129. The TCR of paragraph 130, wherein the alpha chain comprises an amino acid sequence having at least 90%, 95%, or 100% identity to the amino acid sequence of SEQ ID NO: 288.
130. The TCR of any one of paragraphs 125-128 comprising:
   a CDR1-beta sequence comprising the amino acid sequence of SEQ ID NO: 155,
   a CDR2-beta sequence comprising the amino acid sequence of SEQ ID NO: 156, and
   a CDR3-beta sequence comprising the amino acid sequence of SEQ ID NO: 157.
131. The TCR of paragraph 130, wherein the beta chain comprises a beta variable region comprising an amino acid sequence having at least 90%, 95%, or 100% identity to the amino acid sequence of SEQ ID NO: 158.
132. The TCR of paragraph 131, wherein the beta chain comprises an amino acid sequence having at least 90%, 95%, or 100% identity to the amino acid sequence of SEQ ID NO: 159.
133. The TCR of paragraph 131, wherein the beta chain comprises an amino acid sequence having at least 90%, 95%, or 100% identity to the amino acid sequence of SEQ ID NO: 289.
134. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 160,
   a CDR2-alpha sequence comprising the amino acid sequence of SEQ ID NO: 161,
   a CDR3-alpha sequence comprising the amino acid sequence of SEQ ID NO: 162,
   a CDR1-beta sequence comprising the amino acid sequence of SEQ ID NO: 165,
   a CDR2-beta sequence comprising the amino acid sequence of SEQ ID NO: 166, and/or
   a CDR3-beta sequence comprising the amino acid sequence of SEQ ID NO: 167.
135. The TCR of paragraph 134 comprising:
   a CDR1-alpha sequence comprising the amino acid sequence of SEQ ID NO: 160,
   a CDR2-alpha sequence comprising the amino acid sequence of SEQ ID NO: 161, and
   a CDR3-alpha sequence comprising the amino acid sequence of SEQ ID NO: 162.
136. The TCR of paragraph 135, wherein the alpha chain comprises an alpha variable region comprising an amino acid sequence having at least 90%, 95%, or 100% identity to the amino acid sequence of SEQ ID NO: 163.
137. The TCR of paragraph 136, wherein the alpha chain comprises an amino acid sequence having at least 90%, 95%, or 100% identity to the amino acid sequence of SEQ ID NO: 164.
138. The TCR of paragraph 136, wherein the alpha chain comprises an amino acid sequence having at least 90%, 95%, or 100% identity to the amino acid sequence of SEQ ID NO: 290.
139. The TCR of any one of paragraphs 134-137 comprising:
   a CDR1-beta sequence comprising the amino acid sequence of SEQ ID NO: 165,
   a CDR2-beta sequence comprising the amino acid sequence of SEQ ID NO: 166, and
   a CDR3-beta sequence comprising the amino acid sequence of SEQ ID NO: 167.
140. The TCR of paragraph 139, wherein the beta chain comprises a beta variable region comprising an amino acid sequence having at least 90%, 95%, or 100% identity to the amino acid sequence of SEQ ID NO: 168.
141. The TCR of paragraph 140, wherein the beta chain comprises an amino acid sequence having at least 90%, 95%, or 100% identity to the amino acid sequence of SEQ ID NO: 169.
142. The TCR of paragraph 141, wherein the beta chain comprises an amino acid sequence having at least 90%, 95%, or 100% identity to the amino acid sequence of SEQ ID NO: 291.
143. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 170,
   a CDR2-alpha sequence comprising the amino acid sequence of SEQ ID NO: 171,
   a CDR3-alpha sequence comprising the amino acid sequence of SEQ ID NO: 172,
   a CDR1-beta sequence comprising the amino acid sequence of SEQ ID NO: 175,
   a CDR2-beta sequence comprising the amino acid sequence of SEQ ID NO: 176, and/or
   a CDR3-beta sequence comprising the amino acid sequence of SEQ ID NO: 177.
144. The TCR of paragraph 143 comprising:
   a CDR1-alpha sequence comprising the amino acid sequence of SEQ ID NO: 170,
   a CDR2-alpha sequence comprising the amino acid sequence of SEQ ID NO: 171, and
   a CDR3-alpha sequence comprising the amino acid sequence of SEQ ID NO: 172.
145. The TCR of paragraph 144, wherein the alpha chain comprises an alpha variable region comprising an amino acid sequence having at least 90%, 95%, or 100% identity to the amino acid sequence of SEQ ID NO: 173.
146. The TCR of paragraph 145, wherein the alpha chain comprises an amino acid sequence having at least 90%, 95%, or 100% identity to the amino acid sequence of SEQ ID NO: 174.
147. The TCR of paragraph 145, wherein the alpha chain comprises an amino acid sequence having at least 90%, 95%, or 100% identity to the amino acid sequence of SEQ ID NO: 292.
148. The TCR of any one of paragraphs 143-147 comprising:
   a CDR1-beta sequence comprising the amino acid sequence of SEQ ID NO: 175,
   a CDR2-beta sequence comprising the amino acid sequence of SEQ ID NO: 176, and
   a CDR3-beta sequence comprising the amino acid sequence of SEQ ID NO: 177.
149. The TCR of paragraph 148, wherein the beta chain comprises a beta variable region comprising an amino acid sequence having at least 90%, 95%, or 100% identity to the amino acid sequence of SEQ ID NO: 178.
150. The TCR of paragraph 149, wherein the beta chain comprises an amino acid sequence having at least 90%, 95%, or 100% identity to the amino acid sequence of SEQ ID NO: 179.
151. The TCR of paragraph, wherein the beta chain comprises an amino acid sequence having at least 90%, 95%, or 100% identity to the amino acid sequence of SEQ ID NO: 293.
152. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 180,
   a CDR2-alpha sequence comprising the amino acid sequence of SEQ ID NO: 181,
   a CDR3-alpha sequence comprising the amino acid sequence of SEQ ID NO: 182,
   a CDR1-beta sequence comprising the amino acid sequence of SEQ ID NO: 185,
   a CDR2-beta sequence comprising the amino acid sequence of SEQ ID NO: 186, and/or
   a CDR3-beta sequence comprising the amino acid sequence of SEQ ID NO: 187.
153. The TCR of paragraph 152 comprising:
   a CDR1-alpha sequence comprising the amino acid sequence of SEQ ID NO: 180,
   a CDR2-alpha sequence comprising the amino acid sequence of SEQ ID NO: 181, and
   a CDR3-alpha sequence comprising the amino acid sequence of SEQ ID NO: 182.
154. The TCR of paragraph 153, wherein the alpha chain comprises an alpha variable region comprising an amino acid sequence having at least 90%, 95%, or 100% identity to the amino acid sequence of SEQ ID NO: 183.
155. The TCR of paragraph 154, wherein the alpha chain comprises an amino acid sequence having at least 90%, 95%, or 100% identity to the amino acid sequence of SEQ ID NO: 294.
156. The TCR of paragraph 154, wherein the alpha chain comprises an amino acid sequence having at least 90%, 95%, or 100% identity to the amino acid sequence of SEQ ID NO: 184.
157. The TCR of any one of paragraphs 152-155 comprising:
   a CDR1-beta sequence comprising the amino acid sequence of SEQ ID NO: 185,
   a CDR2-beta sequence comprising the amino acid sequence of SEQ ID NO: 186, and
   a CDR3-beta sequence comprising the amino acid sequence of SEQ ID NO: 187.
158. The TCR of paragraph 157, wherein the beta chain comprises a beta variable region comprising an amino acid sequence having at least 90%, 95%, or 100% identity to the amino acid sequence of SEQ ID NO: 188.
159. The TCR of paragraph 158, wherein the beta chain comprises an amino acid sequence having at least 90%, 95%, or 100% identity to the amino acid sequence of SEQ ID NO: 295.
160. The TCR of paragraph 158, wherein the beta chain comprises an amino acid sequence having at least 90%, 95%, or 100% identity to the amino acid sequence of SEQ ID NO: 189.
161. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 190,
   a CDR2-alpha sequence comprising the amino acid sequence of SEQ ID NO: 191,
   a CDR3-alpha sequence comprising the amino acid sequence of SEQ ID NO: 192,
   a CDR1-beta sequence comprising the amino acid sequence of SEQ ID NO: 195,
   a CDR2-beta sequence comprising the amino acid sequence of SEQ ID NO: 196, and/or
   a CDR3-beta sequence comprising the amino acid sequence of SEQ ID NO: 197.
162. The TCR of paragraph 161 comprising:
   a CDR1-alpha sequence comprising the amino acid sequence of SEQ ID NO: 190,
   a CDR2-alpha sequence comprising the amino acid sequence of SEQ ID NO: 191, and
   a CDR3-alpha sequence comprising the amino acid sequence of SEQ ID NO: 192.
163. The TCR of paragraph 162, wherein the alpha chain comprises an alpha variable region comprising an amino acid sequence having at least 90%, 95%, or 100% identity to the amino acid sequence of SEQ ID NO: 193.
164. The TCR of paragraph 163, wherein the alpha chain comprises an amino acid sequence having at least 90%, 95%, or 100% identity to the amino acid sequence of SEQ ID NO: 296.
165. The TCR of paragraph 163, wherein the alpha chain comprises an amino acid sequence having at least 90%, 95%, or 100% identity to the amino acid sequence of SEQ ID NO: 194.
166. The TCR of any one of paragraphs 161-164 comprising:
   a CDR1-beta sequence comprising the amino acid sequence of SEQ ID NO: 195,
   a CDR2-beta sequence comprising the amino acid sequence of SEQ ID NO: 196, and
   a CDR3-beta sequence comprising the amino acid sequence of SEQ ID NO: 197.
167. The TCR of paragraph 166, wherein the beta chain comprises a beta variable region comprising an amino acid sequence having at least 90%, 95%, or 100% identity to the amino acid sequence of SEQ ID NO: 198.
168. The TCR of paragraph 167, wherein the beta chain comprises an amino acid sequence having at least 90%, 95%, or 100% identity to the amino acid sequence of SEQ ID NO: 297.
169. The TCR of paragraph 167, wherein the beta chain comprises an amino acid sequence having at least 90%, 95%, or 100% identity to the amino acid sequence of SEQ ID NO: 199.
170. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 200,
   a CDR2-alpha sequence comprising the amino acid sequence of SEQ ID NO: 201,
   a CDR3-alpha sequence comprising the amino acid sequence of SEQ ID NO: 202,
   a CDR1-beta sequence comprising the amino acid sequence of SEQ ID NO: 205,
   a CDR2-beta sequence comprising the amino acid sequence of SEQ ID NO: 206, and/or
   a CDR3-beta sequence comprising the amino acid sequence of SEQ ID NO: 207.
171. The TCR of paragraph 170 comprising:
   a CDR1-alpha sequence comprising the amino acid sequence of SEQ ID NO: 200,
   a CDR2-alpha sequence comprising the amino acid sequence of SEQ ID NO: 201, and
   a CDR3-alpha sequence comprising the amino acid sequence of SEQ ID NO: 202.
172. The TCR of paragraph 171, wherein the alpha chain comprises an alpha variable region comprising an amino acid sequence having at least 90%, 95%, or 100% identity to the amino acid sequence of SEQ ID NO: 203.
173. The TCR of paragraph 172, wherein the alpha chain comprises an amino acid sequence having at least 90%, 95%, or 100% identity to the amino acid sequence of SEQ ID NO: 298.
174. The TCR of paragraph 172, wherein the alpha chain comprises an amino acid sequence having at least 90%, 95%, or 100% identity to the amino acid sequence of SEQ ID NO: 204.
175. The TCR of any one of paragraphs 170-173 comprising:
   a CDR1-beta sequence comprising the amino acid sequence of SEQ ID NO: 205,
   a CDR2-beta sequence comprising the amino acid sequence of SEQ ID NO: 206, and
   a CDR3-beta sequence comprising the amino acid sequence of SEQ ID NO: 207.
176. The TCR of paragraph 175, wherein the beta chain comprises a beta variable region comprising an amino acid sequence having at least 90%, 95%, or 100% identity to the amino acid sequence of SEQ ID NO: 208.
177. The TCR of paragraph 176, wherein the beta chain comprises an amino acid sequence having at least 90%, 95%, or 100% identity to the amino acid sequence of SEQ ID NO: 299.
178. The TCR of paragraph 176, wherein the beta chain comprises an amino acid sequence having at least 90%, 95%, or 100% identity to the amino acid sequence of SEQ ID NO: 209.
179. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 210,
   a CDR2-alpha sequence comprising the amino acid sequence of SEQ ID NO: 211,
   a CDR3-alpha sequence comprising the amino acid sequence of SEQ ID NO: 212,
   a CDR1-beta sequence comprising the amino acid sequence of SEQ ID NO: 215,
   a CDR2-beta sequence comprising the amino acid sequence of SEQ ID NO: 216, and/or
   a CDR3-beta sequence comprising the amino acid sequence of SEQ ID NO: 217.
180. The TCR of paragraph 179 comprising:
   a CDR1-alpha sequence comprising the amino acid sequence of SEQ ID NO: 210,
   a CDR2-alpha sequence comprising the amino acid sequence of SEQ ID NO: 211, and
   a CDR3-alpha sequence comprising the amino acid sequence of SEQ ID NO: 212.
181. The TCR of paragraph 180, wherein the alpha chain comprises an alpha variable region comprising an amino acid sequence having at least 90%, 95%, or 100% identity to the amino acid sequence of SEQ ID NO: 213.
182. The TCR of paragraph 181, wherein the alpha chain comprises an amino acid sequence having at least 90%, 95%, or 100% identity to the amino acid sequence of SEQ ID NO: 300.
183. The TCR of paragraph 181, wherein the alpha chain comprises an amino acid sequence having at least 90%, 95%, or 100% identity to the amino acid sequence of SEQ ID NO: 214.
184. The TCR of any one of paragraphs 179-182 comprising:
   a CDR1-beta sequence comprising the amino acid sequence of SEQ ID NO: 215,
   a CDR2-beta sequence comprising the amino acid sequence of SEQ ID NO: 216, and
   a CDR3-beta sequence comprising the amino acid sequence of SEQ ID NO: 217.
185. The TCR of paragraph 184, wherein the beta chain comprises a beta variable region comprising an amino acid sequence having at least 90%, 95%, or 100% identity to the amino acid sequence of SEQ ID NO: 218.
186. The TCR of paragraph 185, wherein the beta chain comprises an amino acid sequence having at least 90%, 95%, or 100% identity to the amino acid sequence of SEQ ID NO: 301.
187. The TCR of paragraph 186, wherein the beta chain comprises an amino acid sequence having at least 90%, 95%, or 100% identity to the amino acid sequence of SEQ ID NO: 219.
188. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 220,
   a CDR2-alpha sequence comprising the amino acid sequence of SEQ ID NO: 221,
   a CDR3-alpha sequence comprising the amino acid sequence of SEQ ID NO: 222,
   a CDR1-beta sequence comprising the amino acid sequence of SEQ ID NO: 225,
   a CDR2-beta sequence comprising the amino acid sequence of SEQ ID NO: 226, and/or
   a CDR3-beta sequence comprising the amino acid sequence of SEQ ID NO: 227.
189. The TCR of paragraph 188 comprising:
   a CDR1-alpha sequence comprising the amino acid sequence of SEQ ID NO: 220,
   a CDR2-alpha sequence comprising the amino acid sequence of SEQ ID NO: 221, and
   a CDR3-alpha sequence comprising the amino acid sequence of SEQ ID NO: 222.
190. The TCR of paragraph 189, wherein the alpha chain comprises an alpha variable region comprising an amino acid sequence having at least 90%, 95%, or 100% identity to the amino acid sequence of SEQ ID NO: 223.
191. The TCR of paragraph 190, wherein the alpha chain comprises an amino acid sequence having at least 90%, 95%, or 100% identity to the amino acid sequence of SEQ ID NO: 302.
192. The TCR of paragraph 191, wherein the alpha chain comprises an amino acid sequence having at least 90%, 95%, or 100% identity to the amino acid sequence of SEQ ID NO: 224.
193. The TCR of any one of paragraphs 188-191 comprising:
   a CDR1-beta sequence comprising the amino acid sequence of SEQ ID NO: 225,
   a CDR2-beta sequence comprising the amino acid sequence of SEQ ID NO: 226, and
   a CDR3-beta sequence comprising the amino acid sequence of SEQ ID NO: 227.
194. The TCR of paragraph 193, wherein the beta chain comprises a beta variable region comprising an amino acid sequence having at least 90%, 95%, or 100% identity to the amino acid sequence of SEQ ID NO: 228.
195. The TCR of paragraph 194, wherein the beta chain comprises an amino acid sequence having at least 90%, 95%, or 100% identity to the amino acid sequence of SEQ ID NO: 303.
196. The TCR of paragraph 194, wherein the beta chain comprises an amino acid sequence having at least 90%, 95%, or 100% identity to the amino acid sequence of SEQ ID NO: 229.
197. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 230,
   a CDR2-alpha sequence comprising the amino acid sequence of SEQ ID NO: 231,
   a CDR3-alpha sequence comprising the amino acid sequence of SEQ ID NO: 232,
   a CDR1-beta sequence comprising the amino acid sequence of SEQ ID NO: 235,
   a CDR2-beta sequence comprising the amino acid sequence of SEQ ID NO: 236, and/or
   a CDR3-beta sequence comprising the amino acid sequence of SEQ ID NO: 237.
198. The TCR of paragraph 197 comprising:
   a CDR1-alpha sequence comprising the amino acid sequence of SEQ ID NO: 230,
   a CDR2-alpha sequence comprising the amino acid sequence of SEQ ID NO: 231, and
   a CDR3-alpha sequence comprising the amino acid sequence of SEQ ID NO: 232.
199. The TCR of paragraph 198, wherein the alpha chain comprises an alpha variable region comprising an amino acid sequence having at least 90%, 95%, or 100% identity to the amino acid sequence of SEQ ID NO: 233.
200. The TCR of paragraph 199, wherein the alpha chain comprises an amino acid sequence having at least 90%, 95%, or 100% identity to the amino acid sequence of SEQ ID NO: 304.
201. The TCR of paragraph 199, wherein the alpha chain comprises an amino acid sequence having at least 90%, 95%, or 100% identity to the amino acid sequence of SEQ ID NO: 234.
202. The TCR of any one of paragraphs 197-200 comprising:
   a CDR1-beta sequence comprising the amino acid sequence of SEQ ID NO: 235,
   a CDR2-beta sequence comprising the amino acid sequence of SEQ ID NO: 236, and
   a CDR3-beta sequence comprising the amino acid sequence of SEQ ID NO: 237.
203. The TCR of paragraph 202, wherein the beta chain comprises a beta variable region comprising an amino acid sequence having at least 90%, 95%, or 100% identity to the amino acid sequence of SEQ ID NO: 238.
204. The TCR of paragraph 203, wherein the beta chain comprises an amino acid sequence having at least 90%, 95%, or 100% identity to the amino acid sequence of SEQ ID NO: 305.
205. The TCR of paragraph 203, wherein the beta chain comprises an amino acid sequence having at least 90%, 95%, or 100% identity to the amino acid sequence of SEQ ID NO: 239.
206. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 240,
   a CDR2-alpha sequence comprising the amino acid sequence of SEQ ID NO: 241,
   a CDR3-alpha sequence comprising the amino acid sequence of SEQ ID NO: 242,
   a CDR1-beta sequence comprising the amino acid sequence of SEQ ID NO: 245,
   a CDR2-beta sequence comprising the amino acid sequence of SEQ ID NO: 246, and/or
   a CDR3-beta sequence comprising the amino acid sequence of SEQ ID NO: 247.
207. The TCR of paragraph 162 comprising:
   a CDR1-alpha sequence comprising the amino acid sequence of SEQ ID NO: 240,
   a CDR2-alpha sequence comprising the amino acid sequence of SEQ ID NO: 241, and
   a CDR3-alpha sequence comprising the amino acid sequence of SEQ ID NO: 242.
208. The TCR of paragraph 207, wherein the alpha chain comprises an alpha variable region comprising an amino acid sequence having at least 90%, 95%, or 100% identity to the amino acid sequence of SEQ ID NO: 243.
209. The TCR of paragraph 208, wherein the alpha chain comprises an amino acid sequence having at least 90%, 95%, or 100% identity to the amino acid sequence of SEQ ID NO: 306.
210. The TCR of paragraph 208, wherein the alpha chain comprises an amino acid sequence having at least 90%, 95%, or 100% identity to the amino acid sequence of SEQ ID NO: 244.
211. The TCR of any one of paragraphs 206-209 comprising:
   a CDR1-beta sequence comprising the amino acid sequence of SEQ ID NO: 245,
   a CDR2-beta sequence comprising the amino acid sequence of SEQ ID NO: 246, and
   a CDR3-beta sequence comprising the amino acid sequence of SEQ ID NO: 247.
212. The TCR of paragraph 211, wherein the beta chain comprises a beta variable region comprising an amino acid sequence having at least 90%, 95%, or 100% identity to the amino acid sequence of SEQ ID NO: 248.
213. The TCR of paragraph 212, wherein the beta chain comprises an amino acid sequence having at least 90%, 95%, or 100% identity to the amino acid sequence of SEQ ID NO: 307.
214. The TCR of paragraph 212, wherein the beta chain comprises an amino acid sequence having at least 90%, 95%, or 100% identity to the amino acid sequence of SEQ ID NO: 249.
215. A T cell receptor (TCR) that binds to a proinsulin peptide complexed with a major histocompatibility complex (MHC), wherein the TCR comprises an alpha chain and a beta chain, and wherein the TCR comprises:
   a CDR1-alpha sequence comprising the amino acid sequence of SEQ ID NO: 250,
   a CDR2-alpha sequence comprising the amino acid sequence of SEQ ID NO: 251,
   a CDR3-alpha sequence comprising the amino acid sequence of SEQ ID NO: 252,
   a CDR1-beta sequence comprising the amino acid sequence of SEQ ID NO: 255,
   a CDR2-beta sequence comprising the amino acid sequence of SEQ ID NO: 256, and/or
   a CDR3-beta sequence comprising the amino acid sequence of SEQ ID NO: 257.
216. The TCR of paragraph 215 comprising:
   a CDR1-alpha sequence comprising the amino acid sequence of SEQ ID NO: 250,
   a CDR2-alpha sequence comprising the amino acid sequence of SEQ ID NO: 251, and
   a CDR3-alpha sequence comprising the amino acid sequence of SEQ ID NO: 252.
217. The TCR of paragraph 216, wherein the alpha chain comprises an alpha variable region comprising an amino acid sequence having at least 90%, 95%, or 100% identity to the amino acid sequence of SEQ ID NO: 253.
218. The TCR of paragraph 217, wherein the alpha chain comprises an amino acid sequence having at least 90%, 95%, or 100% identity to the amino acid sequence of SEQ ID NO: 308.
219. The TCR of paragraph 218, wherein the alpha chain comprises an amino acid sequence having at least 90%, 95%, or 100% identity to the amino acid sequence of SEQ ID NO: 254.
220. The TCR of any one of paragraphs 215-218 comprising:
   a CDR1-beta sequence comprising the amino acid sequence of SEQ ID NO: 255,
   a CDR2-beta sequence comprising the amino acid sequence of SEQ ID NO: 256, and
   a CDR3-beta sequence comprising the amino acid sequence of SEQ ID NO: 257.
221. The TCR of paragraph 220, wherein the beta chain comprises a beta variable region comprising an amino acid sequence having at least 90%, 95%, or 100% identity to the amino acid sequence of SEQ ID NO: 258.
222. The TCR of paragraph 221, wherein the beta chain comprises an amino acid sequence having at least 90%, 95%, or 100% identity to the amino acid sequence of SEQ ID NO: 309.
223. The TCR of paragraph 221, wherein the beta chain comprises an amino acid sequence having at least 90%, 95%, or 100% identity to the amino acid sequence of SEQ ID NO: 259.
224. The TCR of any one of the preceding paragraphs, wherein the proinsulin peptide is a preproinsulin 73-90 peptide comprising the amino acid sequence of GAGSLQPLALEGSLQKRG (SEQ ID NO: 109).
225. The TCR of any one of the preceding paragraphs, wherein the MHC comprises HLA-DRB1*04:01.
226. The TCR of paragraph 225, wherein the MHC further comprises HLA-DRA*0101.
227. The TCR of any one of the preceding paragraphs, wherein the TCR is encoded as a single polypeptide, optionally wherein the polypeptide comprises an N-terminal beta domain and a C-terminal alpha domain.
228. The TCR of paragraph 227, wherein the single polypeptide comprises a self-cleaving peptide sequence positioned between the alpha chain and the beta chain.
229. The TCR of paragraph 228, wherein the self-cleaving peptide sequence is a 2A peptide sequence, optionally wherein the 2A peptide sequence is a P2A, E2A, F2A, or T2A peptide sequence.
230. The TCR of any one of the preceding paragraphs, wherein the alpha chain comprises an alpha constant region comprising an amino acid sequence having at least 90%, 95%, or 100% identity to the amino acid sequence of SEQ ID NO: 106.
231. The TCR of any one of the preceding paragraphs, wherein the beta chain comprises a beta constant region comprising an amino acid sequence having at least 90%, 95%, or 100% identity to the amino acid sequence of SEQ ID NO: 107 or 108.
232. The TCR of any one of the preceding paragraphs, wherein TCR comprises one or more amino acid substitutions to cysteine residues in the alpha chain constant region and the beta chain constant region, and wherein the cysteine residues are capable of forming one or more disulfide bridges.
233. The TCR of any one of the preceding paragraphs, wherein the alpha chain constant region comprises a T48C amino acid substitution relative to an alpha chain constant region comprising the amino acid sequence of SEQ ID NO: 106, and wherein the beta chain constant region comprises a S57C amino acid substitution relative to a beta chain constant region comprising the amino acid sequence of SEQ ID NO: 108.
234. An engineered nucleic acid encoding the TCR of any one of the preceding paragraphs.
235. The engineered nucleic acid of paragraph 234, wherein the nucleic acid is a vector.
236. The engineered nucleic acid of paragraph 235, wherein the vector is a viral vector.
237. The engineered nucleic acid of paragraph 236, wherein the viral vector is a lentiviral vector.
238. The engineered nucleic acid of any one of paragraphs 234-237, wherein the nucleic acid comprises a promoter operably linked to a coding sequence encoding the TCR, optionally wherein the promoter is an EF-1 alpha promoter or an MND promoter.
239. The engineered nucleic acid of any one of paragraphs 234-238, wherein the nucleic acid further comprises an enhancer element, optionally an optimized post-transcriptional regulatory element (oPRE) or a woodchuck hepatitis virus post-transcriptional regulatory element (WPRE), further optionally WPRE-mut6.
240. The engineered nucleic acid of any one of paragraphs 234-239, wherein the coding sequence is codon-optimized.
241. An engineered cell comprising a TCR that specifically binds preproinsulin 73-90 complexed with a major histocompatibility complex (MHC), wherein the TCR is exogenous to the engineered cell.
242. The engineered cell of paragraph 241, wherein the TCR comprises the TCR of any one of paragraphs 1-234 or the nucleic acid of any one of paragraphs 89-94.
243. The engineered cell of paragraph 241 or 242, wherein the cell is a T cell, optionally a regulatory T cell.
244. An engineered regulatory T cell comprising a TCR that specifically binds preproinsulin 73-90, complexed with a major histocompatibility complex (MHC), wherein the TCR is exogenous to the engineered cell.
245. The engineered regulatory T cell of paragraph 244, wherein the TCR comprises the TCR of any one of paragraphs 1-234 or the nucleic acid of any one of paragraphs 89-94.
246. The engineered cell of any one of paragraphs 241-245, wherein the cell is derived from a subject, optionally a subject having or suspected of having Type 1 Diabetes.
247. A cell population comprising a plurality of the engineered cell of any one of paragraphs 241-246.
248. A pharmaceutical composition comprising the cell population of paragraph 247 and a pharmaceutically acceptable excipient.
249. A composition comprising the cell population of paragraph 247 and a cryopreservative.
250. A method comprising administering to a subject the cell population of paragraph 247, the pharmaceutical composition of paragraph 248, or the composition of paragraph 249, wherein the subject has Type 1 Diabetes.
251. The method of paragraph 250, wherein the cell population, the pharmaceutical composition, or the composition is administered in an effective amount to alleviate one or more symptom of Type 1 Diabetes.
252. The method of paragraph 247 or 248, wherein the administering comprises intravenous administration.
253. The method of any one of paragraphs 247-249, wherein the administering comprises one or more infusions.
254. The method of any one of paragraphs 250-253, wherein the TCR specifically binds preproinsulin 73-90 complexed with MHC in the pancreas and/or draining lymph node(s) of the subject.
255. The method of any one of paragraphs 250-254, wherein the cell population, the pharmaceutical composition, or the composition is administered in an amount effective to prevent B cell destruction and/or preserve B cell mass in the pancreas of the subject.
256. The method of any one of paragraphs 250-255, wherein the cell population, the pharmaceutical composition, or the composition is administered in an amount effective to repair pancreatic tissue damage in the subject.
257. The method of any one of paragraphs 250-256, wherein the cell population, the pharmaceutical composition, or the composition is administered in an amount effective to preventing autoimmunity associated with Type 1 Diabetes in the subject.
258. The method of any one of paragraphs 250-257, wherein the cell population, the pharmaceutical composition, or the composition is administered in an amount effective to suppress pancreatic islet-associated inflammation in the subject.
259. A method of preventing β cell destruction and/or preserving β cell mass in a subject in need thereof, comprising administering to the subject a composition comprising the cell population of paragraph 247, wherein the TCR specifically binds preproinsulin 73-90 complexed with MHC in the pancreas and/or draining lymph node(s) of the subject, thereby preventing β cell destruction and/or preserving β cell mass in the subject.
260. A method of repairing pancreatic tissue damaged tissue in a subject in need thereof, comprising administering to the subject a composition comprising the cell population of paragraph 247, wherein the TCR specifically binds preproinsulin 73-90 complexed with MHC in the pancreas and/or draining lymph node(s) of the subject, thereby repairing pancreatic tissue damaged tissue in the subject.
261. A method of preventing autoimmunity associated with Type 1 Diabetes in a subject in need thereof, comprising administering to the subject a composition comprising the cell population of paragraph 247, wherein the TCR specifically binds preproinsulin 73-90 complexed with MHC in the pancreas and/or draining lymph node(s) of the subject, thereby preventing autoimmunity associated with Type 1 Diabetes in the subject.
262. A method of suppressing pancreatic islet-associated inflammation in a subject in need thereof, comprising administering to the subject a composition comprising the cell population of paragraph 247, wherein the TCR specifically binds preproinsulin 73-90 complexed with MHC in the pancreas and/or draining lymph node(s) of the subject, thereby suppressing pancreatic islet-associated inflammation in the subject.
263. The method of any one of paragraphs 259-262, wherein the subject has Type 1 Diabetes.
264. The method of any one of paragraphs 259-263, wherein the composition further comprises a pharmaceutically acceptable excipient or a cryopreservative.
265. The method of any one of paragraphs 259-264, wherein the administering comprises intravenous administration.
266. The method of any one of paragraphs 259-265, wherein the administering comprises one or more infusions.
267. The method of any one of paragraphs 250-266, wherein the subject has an HLA-DRB1*04:01 genetic haplotype.
268. The method of any one of paragraphs 250-267, wherein the subject has new-onset/adult-onset Type 1 Diabetes.
269. The method of any one of paragraphs 250-268, wherein the subject has residual beta cell function.
270. A method of treating Type 1 Diabetes in a subject in need thereof, the method comprising:
   administering to a subject diagnosed with Type 1 Diabetes a therapeutically effective amount of population of cells comprising regulatory T cells, wherein regulatory T cells of the population express an engineered T cell receptor comprising a TCR alpha chain comprising the sequence of SEQ ID NO: 4 and a TCR beta chain comprising the sequence of SEQ ID NO: 9, wherein the regulatory T cells specifically binds preproinsulin 73-90 complexed with an MHC molecule in the pancreas and/or pancreatic draining lymph node(s) of the subject.
271. A method of treating Type 1 Diabetes in a subject in need thereof, the method comprising:
   administering to a subject diagnosed with Type 1 Diabetes a therapeutically effective amount of population of cells comprising regulatory T cells, wherein regulatory T cells of the population express an engineered T cell receptor comprising a TCR alpha chain comprising the sequence of SEQ ID NO: 4 and a TCR beta chain comprising the sequence of SEQ ID NO: 9, wherein the regulatory T cells specifically binds preproinsulin 73-90 complexed with an MHC molecule in the pancreas and/or pancreatic draining lymph node(s) of the subject.
272. A method of treating Type 1 Diabetes in a subject in need thereof, the method comprising:
   administering to a subject diagnosed with Type 1 Diabetes a therapeutically effective amount of population of cells comprising regulatory T cells, wherein regulatory T cells of the population express an engineered T cell receptor comprising a TCR alpha chain comprising the sequence of SEQ ID NO: 5 and a TCR beta chain comprising the sequence of SEQ ID NO: 10, wherein the regulatory T cells specifically binds preproinsulin 73-90 complexed with an MHC molecule in the pancreas and/or pancreatic draining lymph node(s) of the subject.
273. A method of treating Type 1 Diabetes in a subject in need thereof, the method comprising:
   administering to a subject diagnosed with Type 1 Diabetes a therapeutically effective amount of population of cells comprising regulatory T cells, wherein regulatory T cells of the population express an engineered T cell receptor comprising a TCR alpha chain comprising the sequence of SEQ ID NO: 260 and a TCR beta chain comprising the sequence of SEQ ID NO:
261, wherein the regulatory T cells specifically binds preproinsulin 73-90 complexed with an MHC molecule in the pancreas and/or pancreatic draining lymph node(s) of the subject.
274. The method of paragraph 272, wherein the engineered T cell receptor comprises the amino acid sequence of SEQ ID NO: 310.
275. The method of paragraph 273, wherein the engineered T cell receptor comprises the amino acid sequence of SEQ ID NO: 311.
276. The method of any one of paragraphs 270-275, wherein the population of cells is autologous to the subject.
277. The method of any one of paragraphs 270-276, wherein fewer than 1% of the regulatory T cells of the population have been gene edited at the endogenous FOXP3 locus.
278. The method of any one of paragraphs 270-277, wherein the therapeutically effective amount is sufficient to preserve function of at least 5%, at least 10%, at least 15%, or at least 20% of pancreatic beta islet cells in the subject.
279. The method of any one of paragraphs 270-278, wherein the subject is an HLA-DRB1*04:01-positive subject.
280. The method of any one of paragraphs 270-279, wherein the subject is diagnosed with Stage 3 Type 1 Diabetes.
281. The method of any one of paragraphs 270-280, wherein regulatory T cells of the population are phenotypically stable regulatory T cells.
282. The method of any one of paragraphs 270-281, wherein at least 80% of the cells of the population are phenotypically stable regulatory T cells.
283. The method of any one of paragraphs 270-282, wherein no more than 20% of the cells of the population are conventional CD4+ T cells; and/or no more than 5% of the cells of the population are CD8+ T cells.
284. The method of any one of paragraphs 270-283, wherein no more than 10% of the cells of the population are non-regulatory T cells that express the engineered T cell receptor.
285. The method of any one of paragraphs 270-284, wherein at least 40% of the cells of the population are CD4+CD25hi/+CD127−/low regulatory T cells that express the engineered T cell receptor.
286. The method of any one of paragraphs 270-285, wherein at least 70% of the cells of the population are viable.
287. The method of any one of paragraphs 270-286 further comprising selecting a subject who is HLA-DRB1*04:01 positive and has been diagnosed with Type 1 Diabetes, optionally Stage 3 Type 1 Diabetes, prior to administering the population of cells to the subject.
288. The method of any one of paragraphs 270-287 further comprising, prior to the administering, isolating cells from the subject, sorting the cells to obtain phenotypically stable regulatory T cells, and expanding the phenotypically stable regulatory T cells, thereby producing the phenotypically stable regulatory T cells of the population of cells.

### EXAMPLES

### Example 1. TCR Characterization Studies

T-cell receptors that are specific to preproinsulin 73-90 (SEQ ID NO: 109) were identified by repeated stimulation of autologously matched CD4 T cells and monocytes isolated from peripheral blood mononuclear cells (PBMCs) of a healthy HLA-DRB1*04:01 donor leukopak. TCRs of clonal population of cells that expanded in response to antigen were selected for further analysis.

One hundred nineteen (119) TCRs were selected for further analysis and cloned into a lentiviral vector. Each of the TCRs was encoded by lentiviral vector constructs that comprised an N-terminal TCR beta chain and a C-terminal TCR alpha chain with a linker domain comprising a GSG amino acid sequence followed by a P2A self-cleaving peptide. The TCR beta-GSG-P2A-TCR alpha fusion constructs were expressed under the EF1a promoter. Additionally, vectors encoding one of several TCRs were generated as negative controls (“Control TCR”). In some instances, the TCR sequences were engineered to introduce an additional cysteine bridge between the alpha and beta constant regions (DS, or disulfide modified).

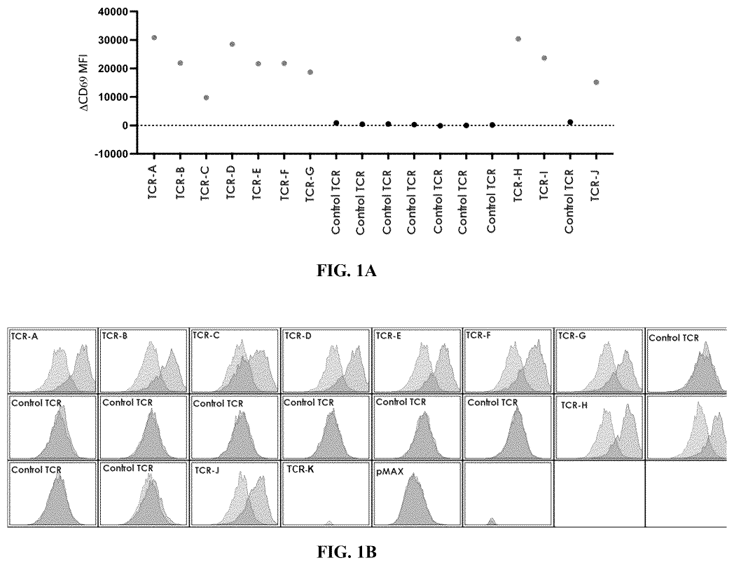

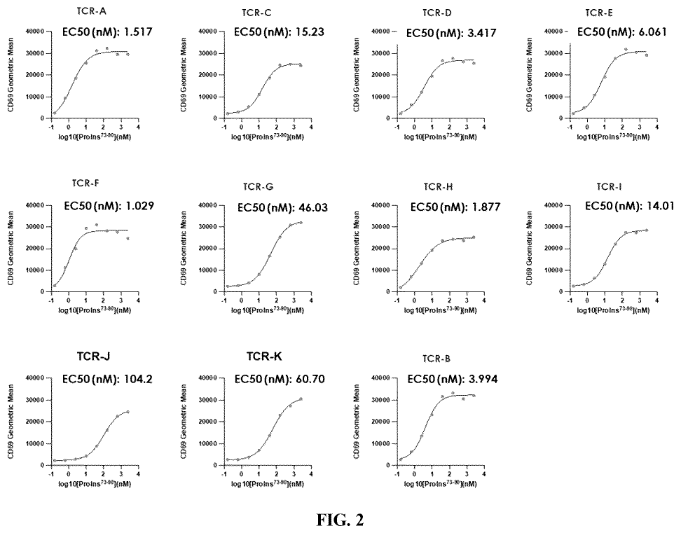

The vectors were transduced into TCR-null Jurkat cells by electroporation and the ability of the TCRs to activate Jurkat cells in response to antigen (preproinsulin 73-90) stimulation was tested. One day following transfection, Jurkat cells were co-cultured at a 1:1 ratio with BLS-DRB1*04:01 cells (HLA-DR monogenic B cell line) and 10 μM preproinsulin 73-90 peptide. Flow cytometry was used to confirm TCR expression and measure upregulation of CD69 surface expression (FIGS. 1A-1B). As is shown in FIGS. 1A-1B, Jurkat cells transduced with any one of TCR-A, TCR-B, TCR-C, TCR-D, TCR-E, TCR-F, TCR-G, TCR-H. TCR-I, TCR-J, and TCR-K showed increased CD69 expression in response to antigen stimulation (a measure of cellular activation). The control TCR experiments did not demonstrate upregulation of CD69 antigen stimulation. These data demonstrate that TCR-A. TCR-B, TCR-C, TCR-D, TCR-E, TCR-F, TCR-G, TCR-H, TCR-I, TCR-J, and TCR-K are capable of activating cells in the presence of preproinsulin 73-90 peptide.

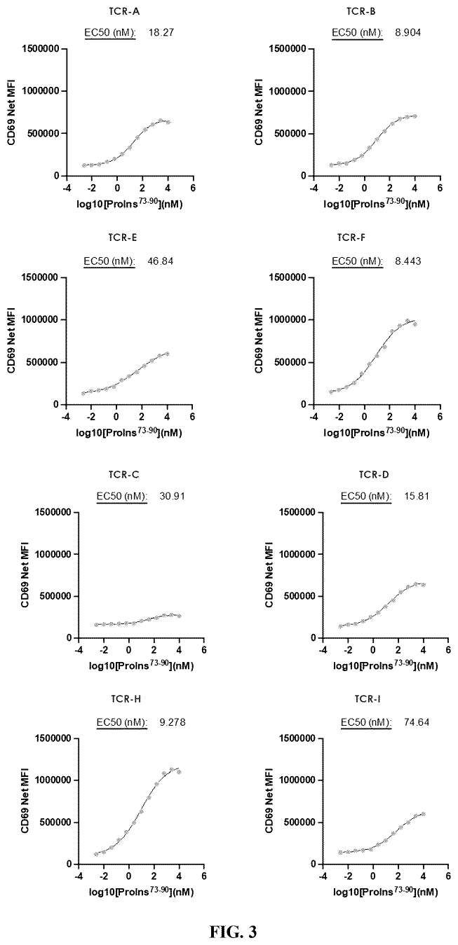

In total, 25 of the 119 TCRs selected for screening in TCR-null Jurkat cells showed reactivity towards preproinsulin TCRs (TCR-A, TCR-B, TCR-C, TCR-D, TCR-E, TCR-F. TCR-G, TCR-H, TCR-I, TCR-J, TCR-K, TCR-L, TCR-M, TCR-N, TCR-O, TCR-P, TCR-Q, TCR-R, TCR-S, TCR-T, TCR-U, TCR-V, TCR-X, TCR-Y and TCR-Z) and were further analyzed to determine half-maximal effective concentrations (EC-50 values). Four days following transduction. TCR expression was confirmed by flow cytometry and transduced Jurkat cells were co-cultured with BLS-DRB1*04:01 cells at a 1:1 ratio in the presence of varying concentrations of preproinsulin 73-90 peptide (serial dilutions starting at 2.5 μM). Representative data for TCRs TCR-A, TCR-B, TCR-C, TCR-D, TCR-E, TCR-F, TCR-G, TCR-H, TCR-I, TCR-J, and TCR-K is shown in FIG. 2, demonstrating that each of these TCRs are capable of activating cells even at low concentrations of preproinsulin 73-90 peptide (EC-50 values from 1-105 nM).

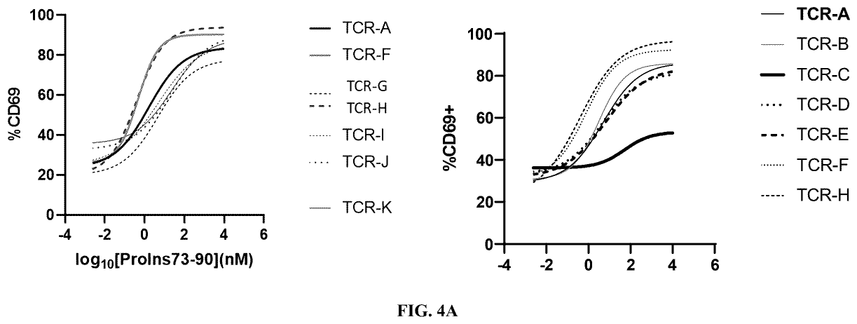

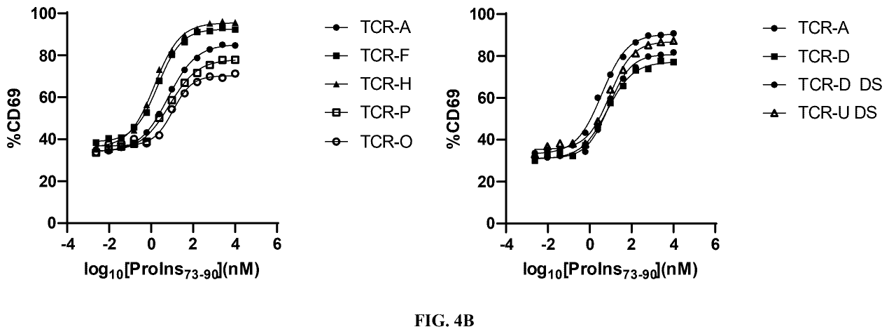

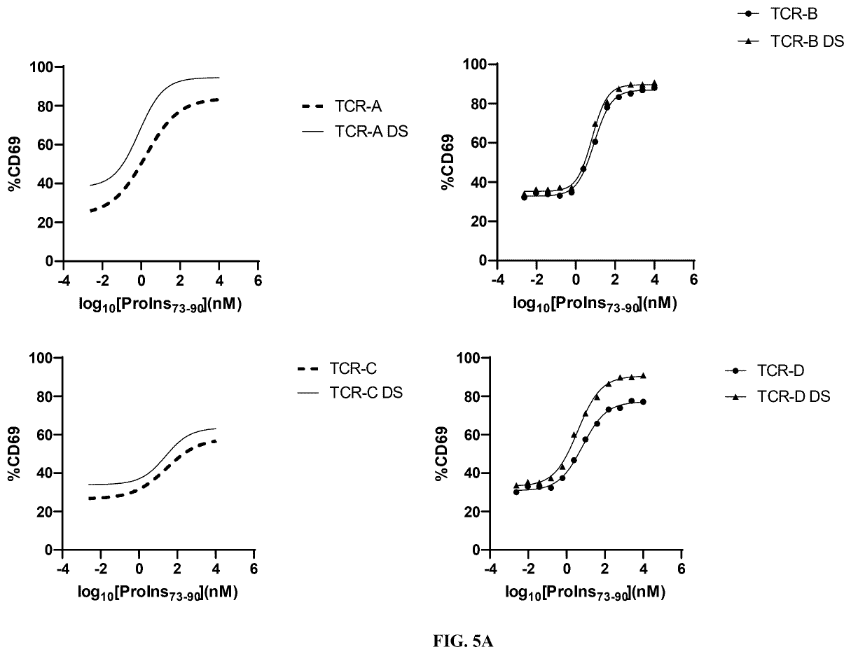

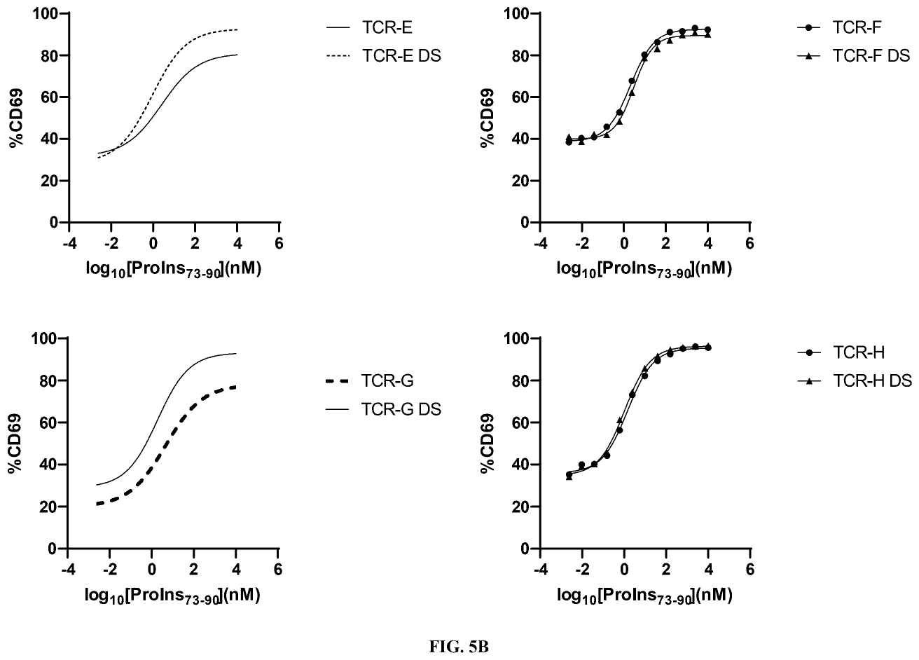

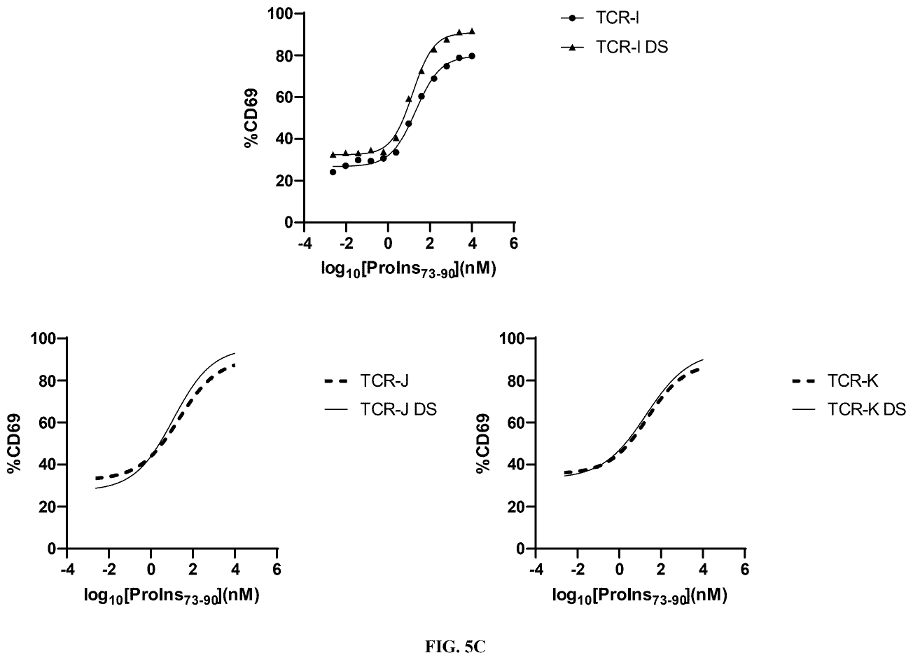

Eighteen (18) of the 25 reactive TCRs were carried forward for further screening, in conventional CD4 T cells (which are expressing endogenous TCRs). TCRs tested were either unmodified, disulfide modified, or both versions were tested. Conventional CD4⁺ T cells were isolated from peripheral blood mononuclear cells (PBMC) of healthy HLA-DRB1*04:01+ donors. 3×10⁵ CD4⁺ conventional T cells were seeded in a 24-well plate and activated in a 3:1 ratio with anti-CD3/CD28 beads (Dyna beads; ThermoFisher). After 24 hours, beads were removed from the cells and cells were transduced with lentivirus expressing the proinsulin-specific TCRs in the presence of IL-7 and IL-2. Four days following transduction. TCR expression was confirmed by flow cytometry and CD4⁺ T cells were co-cultured with BLS-DRB1*04:01 cells (for 18 hours) in the presence of varying concentrations of preproinsulin 73-90 peptide (serial dilutions starting from 10 μM). Flow cytometry was used to measure surface level expression of CD69 following this co-culture. Representative EC50 curves are shown in FIG. 3 and FIG. 4. These data demonstrate that the identified TCRs specific for preproinsulin peptide are capable of activating CD4 T cells even at low concentrations of preproinsulin 73-90 peptide. FIG. 5 shows a comparison of EC50 curves for disulfide-modified and unmodified versions of the same TCR for TCRs A-K.

The introduction of the disulfide to each of the tested TCRs provided increased binding affinity for the preproinsulin 73-90 peptide. For example, the disulfide-modified TCR-A DS demonstrated increased binding and sensitivity to preproinsulin 73-90 peptide in CD4 T cells with a lower half maximal effective concentration (EC50) (2.77 nM, n=3) than TCR-A (6.14 nM, n=9). Furthermore, the maximal functional response (upregulation of CD69) of the disulfide-modified TCR-A DS was 1.11-fold greater on average than the unmodified TCR-A (92.5% of maximal response for TCR-A DS; 83.6% of maximal response for TCR-A).

The EC50 values (nM preproinsulin peptide) for the 18 TCRs in conventional CD4⁺ T cells, with and/or without disulfide modification (DS) are shown in Table 2.

TABLE 2

| TCR | EC50 (nM) | N (experimental replicates) |
|---|---|---|
| TCR-A | 6.14 | 9 |
| TCR-A DS | 2.77 | 3 |
| TCR-B | 5.68 | 3 |
| TCR-B DS | 3.78 | 2 |
| TCR-C | 155.77 | 2 |
| TCR-C DS | 24.16 | 1 |
| TCR-D | 4.42 | 5 |
| TCR-D DS | 2.18 | 3 |
| TCR-E | 7.78 | 4 |
| TCR-E DS | 3.47 | 2 |
| TCR-F | 1.50 | 5 |
| TCR-F DS | 1.36 | 3 |
| TCR-G | 11.24 | 2 |
| TCR-G DS | 1.89 | 1 |
| TCR-H | 0.93 | 5 |
| TCR-H DS | 0.62 | 4 |
| TCR-I | 14.88 | 3 |
| TCR-I DS | 8.03 | 2 |
| TCR-J | 47.96 | 2 |
| TCR-J DS | 11.78 | 1 |
| TCR-K | 47.73 | 2 |
| TCR-K DS | 19.10 | 1 |
| TCR-L | 497.70 | 1 |
| TCR-M | 244.50 | 1 |
| TCR-N | 28.22 | 1 |
| TCR-O | 6.34 | 2 |
| TCR-P | 6.40 | 3 |
| TCR-Q | 409.40 | 1 |
| TCR-S DS | 10000 | 1 |

The unmodified and disulfide-modified versions of TCR-A, TCR-D, TCR-F and TCR-K were carried forward for further evaluation of activity in regulatory T cells. Human thymic regulatory T cells were isolated from PBMC by selecting CD45RA⁺/CD4⁺/CD25^{high}/CD127^{lo} cells. Isolated Treg were then activated with anti-CD3/anti-CD28 dynabeads followed by transduction with lentiviral vector encoding the TCR indicated. The transduced regulatory T cells were then expanded for 96 hours. TCR expression was confirmed via flow cytometry (data not shown).

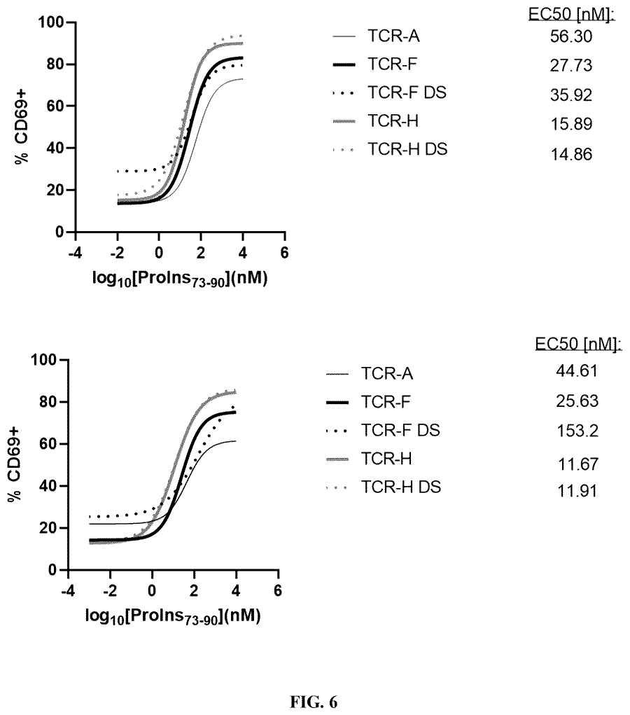

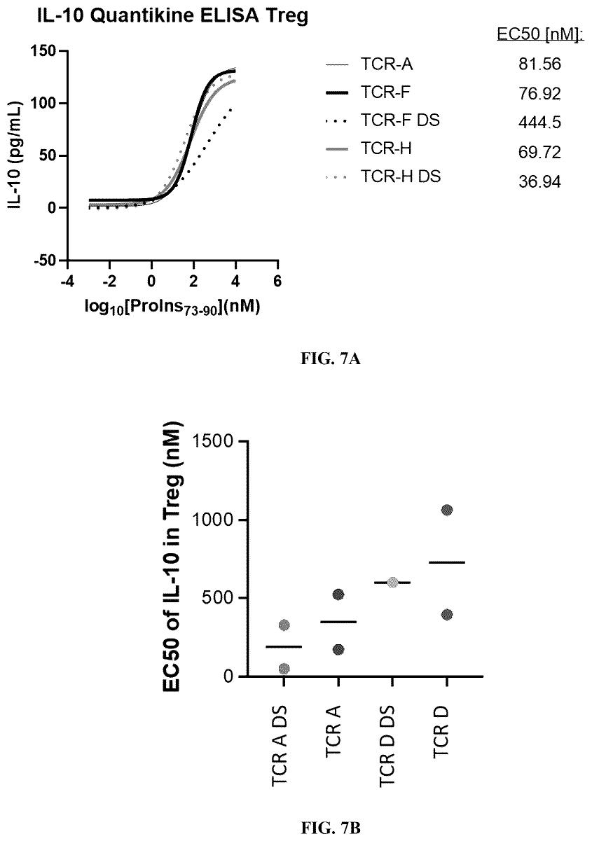

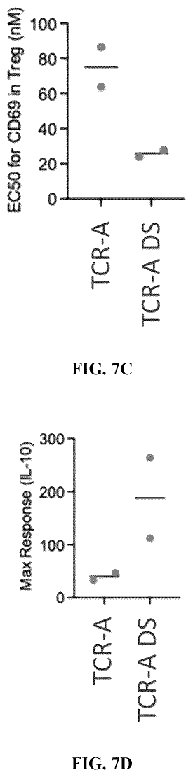

To test whether transduced regulatory T cells could be activated in response to stimulation with preproinsulin, a lentiviral transduced population of cells comprising regulatory T cells, or untransduced controls, were incubated at 37° C. with donor-matched peripheral blood mononuclear cells (PBMCs), preproinsulin peptide 73-90 at a concentration of 0-10 μM in an 11-point dose response curve at a 4× dilution series (at 0, 0.000001, 0.00001, 0.0001, 0.001, 0.01, 0.1, 1, 10, and 10 μM) in complete media having 1000 U/ml IL-2 and 50 ng/ml IL-7. The ratio of PBMC to regulatory T cells was 2:1. Transduced regulatory T cells were differentiated from untransduced regulatory T cells by a GFP integrated in the lentiviral vector. Following incubation, regulatory T activation markers were measured by FACS after 1 day and cytokine levels were measured by LegendPlex after 2 days. FIG. 6 shows that Treg transduced with each of the TCRs show increased activity, as measured by the proportion of CD69+ cells at increasing concentrations of preproinsulin. FIGS. 7A-7B show that Treg transduced with each of the TCRs show increased suppressive activity, as measured by IL-10 levels.

In addition, when Tregs transduced with TCR-A or TCR-A DS were incubated at 37° C. with donor-matched peripheral blood mononuclear cells (PBMCs), recombinant IL-2, and preproinsulin peptide 73-90) at a concentration 1 nM-10 μM in dose response curve at a 4× dilution series, Tregs transduced with TCR-A and Tregs transduced with TCR-A DS demonstrated high functional avidity with EC50 values of 75.22 nM and 25.99 nM, respectively, for CD69 upregulation (FIG. 7C). Tregs transduced with TCR-A DS exhibited an increased maximal functional response as measured by IL-10 production (4.71-fold greater) (FIG. 7D) and CD69 upregulation (1.18-fold greater) (FIG. 7E) compared with Tregs transduced with TCR-A.

### Example 2. Regulatory T Cell Suppression Studies

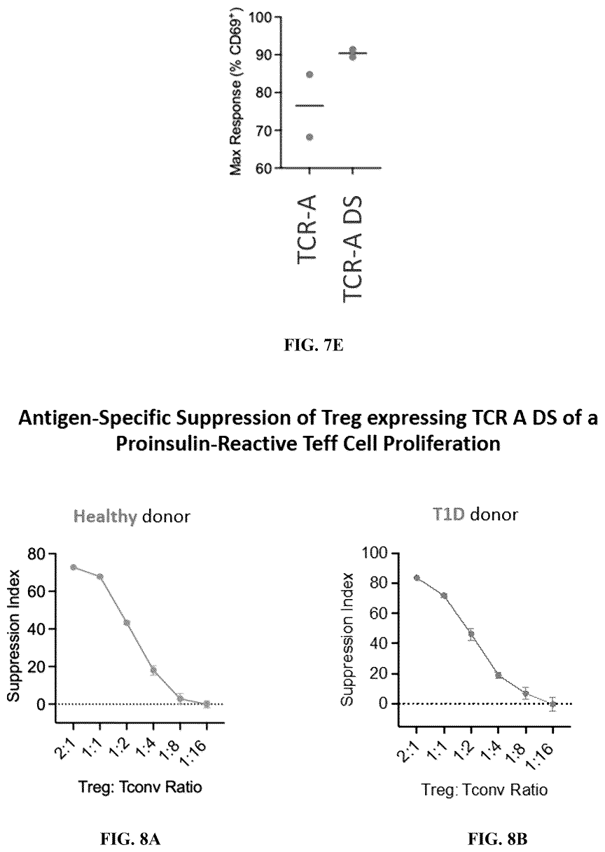

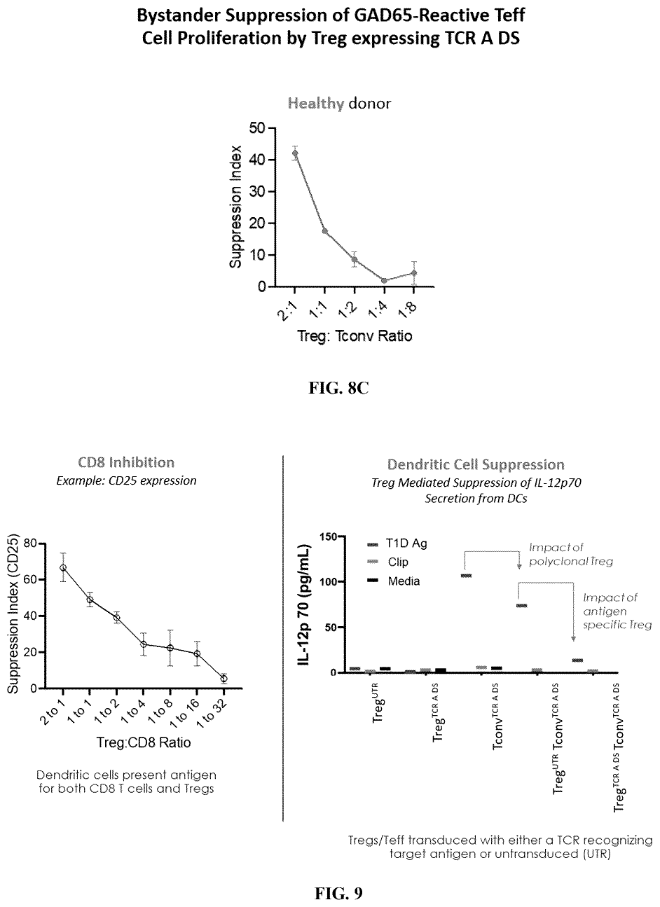

The ability of regulatory T cells expressing the TCRs described herein to suppress conventional T cells targeting TID-associated antigens was measured in regulatory T cell suppression assays. Regulatory T cells isolated from a healthy donor or from a donor having TID transduced with preproinsulin TCR A DS were incubated at 37° C. with Mitomycin C treated peripheral blood mononuclear cells (PBMCs) isolated from HLA-matched donors, conventional T cells transduced with an alternate preproinsulin targeting TCR (TCR-J) or with a GAD65-targeting TCR, preproinsulin 73-90, and the target peptide of the respective conventional T cells at the ratios shown. Results are shown in FIGS. 8A-8C.

The ability of regulatory cells expressing the TCRs described herein to suppress dendritic cells and CD8+ T cells was also evaluated in a DC delicensing assay. The ability of regulatory T cells expressing TCR A DS to suppress CD8+ T cells was tested by co-culturing dendritic cells, regulatory T cells expressing TCR A DS. CD8+ T cells expressing a TCR targeting proinsulin antigen, and preproinsulin 73-90) at the Treg:CD8+ T cell ratios shown in FIG. 9. Antigen was presented by dendritic cells, activating both CD8+ T cells and Tregs. The ability of Tregs to suppress CD8+ T cells was assessed by CD25 expression. As is shown, the TCR A DS Tregs suppress the CD8+ T cells in a manner that is dependent on the Treg:CD8+ T cell ratio.

Tregs can suppress conventional T cells (Tcons) indirectly by downregulating costimulatory molecules on APCs via CTLA-4 or reduction of contact formation between Tcons and DCs (Tadokoro et al . . . 2006). Tregs support an immunosuppressive cytokine milieu by reducing IL-6 & IL-12p70 production by DCs. This assay aims to highlight the suppressive mechanisms of Tregs on DCs. In this assay. HLA-DRB1*14:01 DCs are first incubated with preproinsulin 73-90, a CLIP control antigen, or no antigen control. After incubation. HLA-DRB1*14:01 Tregs expressing TCR A DS or untransduced Tregs. HLA-DRB1*14:01 Tcons expressing TCR A DS, or both are added to the DCs. The results in FIG. 9 show that IL-12p70 production by DCs was induced in the presence of TCR A DS Tcon and preproinsulin 73-90 but not CLIP control antigen or no antigen, demonstrating that this is via a TCR specific interaction. While the introduction of untransduced Tregs slightly suppresses IL-12p70 production by DC, the suppression achieved by TCR A DS is far greater. This suggests that TCR A DS Tregs are able to suppress antigen-specific conventional T cell activation in an antigen specific manner indirectly via suppression of IL-12p70 production by DC.

Cross reactivity of selected TCRs of the disclosure (TCR-A DS and TCR-D DS) with off-target or alternative peptide targets was evaluated by performing an X scan analysis (X-scan: Systematic substitution at each position of the antigenic peptide sequence using all 20 AA; >300 single mutants screened; Core recognition motif identified). This experiment was utilized to identify the specific residues of the target peptide (preproinsulin peptide 73-90) that are required to activate TCR-expressing cells. A library of peptides containing single substitutions of every possible amino acid at each residue of preproinsulin peptide 73-90 was developed to evaluate the residues required for activation of TCR-transduced TCR^{null} Jurkat T cells expressing secreted luciferase enzyme (expression driven by the nuclear factor of activated T cells (NFAT) promoter). To assess their activation in response to X-scan library peptides. TCR-Jurkats were co-cultured with antigen presenting cells that express only HLA-DRB1*04:01 (BLS-DR4 cells) loaded in an arrayed fashion with peptides from the X-scan library so that the ability of each peptide to activate TCR-Jurkats was assessed individually (not in pooled fashion). The relative activation of TCR-Jurkats in response to each peptide was measured by luciferase assay to read out NFAT activation. The required residues were used to identify potential off-target peptides using ScanProsite. The search was limited to peptides expressed in human cells as well as those from pathogens for which cross-reactivity would pose the highest potential risk to the proposed patient population.

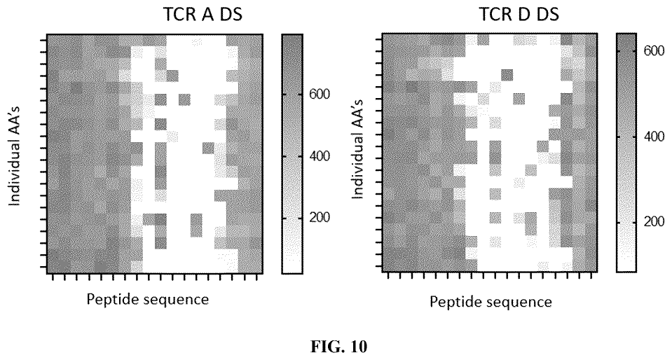

Results for TCR A DS and TCR D DS are shown in FIG. 10. Both TCRs exhibited high on target specificity and low cross-reactivity.

### Example 3. Antigen-Specific Regulatory T Cell Suppression Studies

Human regulatory T cells (Tregs) exert their anti-inflammatory functions in part through suppression of conventional T cells (Tconvs) activation and proliferation. TCR-transduced Tregs (transduced with TCR-A or TCR-A DS) can be activated in an antigen-specific manner by the presentation of preproinsulin peptide 73-90 by HLA-DRB1*04:01⁺ APCs.

For antigen-specific suppression studies, both Tregs and Tconvs were transduced with preproinsulin peptide 73-90 specific TCRs. Isolated CD4⁺CD25⁺ CD127^{low} Tregs were transduced with TCR-A or TCR-A DS. CD4⁺ CD25⁻Tconv cells isolated from healthy donors or patient donors with TID disease were transduced with an alternative TCR specific for preproinsulin peptide 73-90. The percentage of TCR cells was assessed by flow cytometry measurement of GFP⁺ cells as the lentiviral vector encoding TCR-A and TCR-A DS includes an integrated GFP. The proportion of TCR+ cells was then normalized to 32% for all samples. TCR-transduced Tregs and TCR-transduced Tconvs were co-cultured and stimulated with preproinsulin peptide 73-90) peptide presented by HLA-DRBI*04:01⁺ PBMCs. After four days, the culture supernatants were collected for IFN-γ measurement, and cells were collected for flow cytometry analysis.

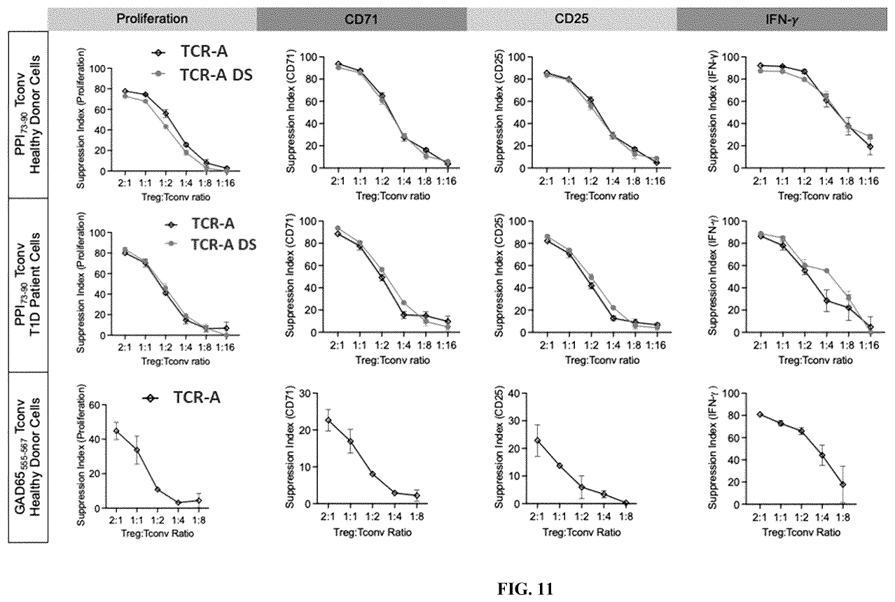

In response to antigen stimulation, the TCR-transduced Tregs suppressed Tconv activation as measured by the inhibition of activation markers CD25 and CD71, suppressed Tconv cell proliferation as measured by CellTrace Violet dye dilution, and inhibited IFN-γ secretion by Tconv cells (FIG. 11). Tregs transduced with TCR-A or TCR-A DS suppressed proliferation, expression of activation associated markers CD71 and CD25, and the release of inflammatory cytokine IFN y by preproinsulin peptide 73-90 reactive Tconv at indicated Treg:Tconv ratios in the antigen-specific suppression study. Tregs and Tconv cells were sorted from the peripheral blood of a healthy donor or a Type 1 Diabetes patient.

Bystander suppression studies were also performed to determine the ability of TCR-transduced Tregs to suppress other islet antigen-reactive Tconvs. Tregs transduced with TCR-A DS or TCR-A were co-cultured with Tconv cells transduced with a GAD65-targeting TCR. The co-cultured Treg and Tconv cells were stimulated by preproinsulin peptide 73-90 and GAD65555-567 peptide presented by mitomycin-C treated PBMCs. After four days, the IFN-γ release, proliferation, and activation of TCR-transduced Tconv cells were measured (FIG. 11). Tregs transduced with TCR-A DS or TCR-A suppressed multiple effector functions of Tconv cells across different Treg:Tconv ratios.

Treg suppression of Tconv cell proliferation at indicated Treg:Tconv ratios was calculated as follows:

$$100 * \frac{\begin{matrix}[ Average \% of divided Tconv cells without Tregs ] - \\ [ \% of divided Tconv cells with Tregs ]\end{matrix}}{[ Average \% of divided Tconv cells without Tregs ]}$$

Treg suppression of Tconv cell activation at indicated Treg:Tconv ratios was calculated as follows:

$$100 * \frac{\begin{matrix}[ Average \% of activated Tconv cells without Tregs ] - \\ [ \% of activated Tconv cells with Tregs ]\end{matrix}}{[ Average \% of activated Tconv cells without Tregs ]}$$

Treg suppression index of inflammatory cytokine release was calculated as:

$$100 * \frac{\begin{matrix}[ Average concentration of inflammatory cytokine without Tregs ] - \\ [ Concentration of inflammatory cytokine with Tregs ]\end{matrix}}{[ Average concentration of inflammatory cytokine without Tregs ]}$$

### Example 4. T Cell Receptor Alloreactivity Studies

This Example describes the evaluation of potential alloreactivity of a selected TCR of the disclosure (TCR-A DS). TCR^{null} Jurkat cells transduced with TCR-A DS (TCR-A DS Jurkats) were co-cultured with a panel of selected cell lines that express HLA-DRB1*04:01 (among other DRBI haplotypes) and with a variety of exogenously presented peptides. The use of TCR^{null} Jurkat cells, which are absent of endogenous TCRs prior to engineering, avoids any potential mispairing of the TCR and allows for specific examination of the reactivity of the TCR with alternative DRBI alleles in a TCR specific context.

Response to exogenously loaded target peptide (preproinsulin peptide 73-90) was assessed by measuring Jurkat cell activation via the CD69 marker. TCR-A DS Jurkat cells were co-cultured with each cell line in the presence of preproinsulin peptide 73-90 at a 1 μM concentration or in the absence of antigen peptide (control). In addition, a negative control in which no antigen presenting cells were present was included. Cells were incubated overnight (16-20 hours) and CD69 expression was assessed by flow cytometry as a readout of activation in response to antigenic stimulation.

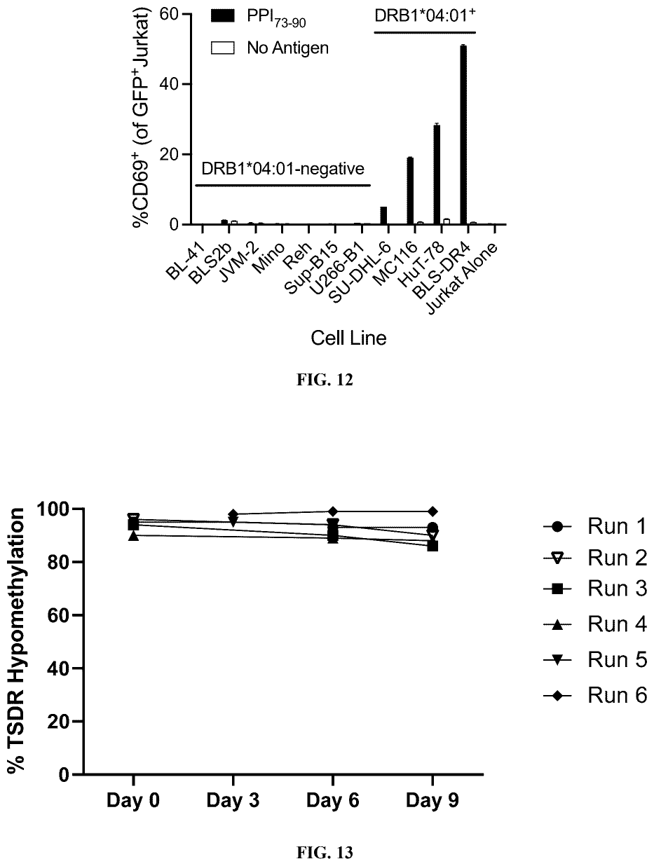

TCR-A DS Jurkat cells only showed upregulation of CD69 when co-cultured with cells that express HLA-DRB*04:01 and stimulated with antigen peptide (FIG. 12). No signs of activation were observed with alternate DRBI haplotypes. Based on these data, no alloreactivity was observed with TCR-A DS against any of the cell lines in the HLA-DRBI haplotype-expressing panel.

### Example 5. Regulatory T Cell Stability Studies

Cryopreserved regulatory T cells (Tregs) are thawed and plated into 96-well tissue-culture-treated plates at 50.000 cells per well with or without anti-CD3/CD28 beads and supplemented with human recombinant IL-2 at varying concentrations (0 units/mL, 1 units/mL, 10 units/mL, 100 units/mL, 1000 units/mL). Every three days, half of the culture is sampled for cell number via CellTiter Glo and analyzed for cell viability. For the remaining cells, the media are refreshed, and fresh activation beads and IL-2 are added as appropriate. Representative data for Tregs expressing a control TCR showed that with anti-CD3/CD28 bead activation, the number of Tregs increased from Day 3 to Day 9 in cultures with at least 10 units/ml recombinant IL-2. However, despite TCR activation, the number of Tregs was decreased in cultures with no or minimal (1 unit/mL) IL-2 added. Without TCR activation. Treg cell numbers measured by ATP levels in culture decreased over time from Day 3 to Day 9 at all concentrations of IL-2. The magnitude of this decrease in Treg number was IL-2 concentration dependent. In summary, this study demonstrated that, after lentiviral transduction to introduce an exogenous TCR and subsequent expansion, the growth and survival of Treg cells continued to be dependent on the presence of IL-2. In the setting of this experiment, no evidence of malignant transformation due to Treg cell engineering was observed.

It is anticipated that Tregs transduced with the TCRs of the disclosure (e.g., TCRs that binds to a proinsulin peptide complexed with a major histocompatibility complex (MHC)) will perform similarly in this assay and demonstrate robust stability profiles.

### Example 6. Regulatory T Cell Product Manufacturing

### Stage 1: Preparation of Starting Material and Initial Sorting Under cGMP Conditions

Regulatory T cell populations are prepared with receipt of an autologous apheresis leukopak (following leukapheresis performed under sterile conditions to collect the PBMCs into a sterile bag). Autologous Tregs are isolated using a series of sorting operations: antibody-bound magnetic bead-based depletion and enrichment followed by FACS. In Stage 1, initial removal of undesired cell types occurs by depletion of CD8⁺, CD14⁺, and CD19⁺ cells using an automated cell processing system with anti-CD8, anti-CD14, and anti-CD19 magnetic microbeads.

### Stage 2-3: Sorting for and Activation of Autologous Thymically-Derived Stable Tregs

CD25 cells are isolated using an automated cell processing system and anti-CD25-PE-Biotin reagent plus anti-Biotin beads. For FACS sorting, the CD25-enriched cells from the previous step are stained with fluorescently labeled anti-CD4, anti-CD45RA, and anti-CD127 antibodies. A first “debulk” sort occurs using a cell sorter system followed by a “purity” sort using the same cell sorter system. A gating strategy is used to enrich the cell population for thymically-derived stable Tregs.

Following sorting, the Treg cells are suspended in serum-free culture media supplemented with human recombinant IL-2 cytokine. Treg cells are activated by addition of activation reagent followed by incubation in a temperature-controlled CO₂ incubator for a specified time.

### Stage 4: Transduction

Activated cells are transduced by addition of a calculated quantity of recombinant lentiviral vector (LVV) drug substance and incubated for a specified time.

### Stage 5-6: Scale Up and Harvest

Cells are aseptically transferred to fresh, single use, sterile expansion vessels for expansion until harvest. The percent TSDR hypomethylation over expansion of a cell product prepared according to these methods from 6 donors and transduced with an unrelated TCR is shown in FIG. 13. This demonstrates that regulatory T cells isolated according to these methods maintain a stable Treg phenotype.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed is:

1. An engineered T cell receptor that specifically binds a preproinsulin 73-90 peptide complexed with a major histocompatibility complex (MHC) molecule.

2. The engineered T cell receptor of claim 1 comprising an alpha chain and a beta chain, wherein the alpha chain comprises a CDR3-alpha sequence comprising the amino acid sequence of any one of SEQ ID NOs: 3, 33, 53, and 73 and/or the beta chain comprises a CDR3-beta sequence comprising the amino acid sequence of any one of SEQ ID NOs: 8, 38, 58, and 78.

3. The engineered T cell receptor of claim 2, wherein the alpha chain comprises a CDR3-alpha sequence comprising the amino acid sequence of SEQ ID NO: 3 and the beta chain comprises a CDR3-beta sequence comprising the amino acid sequence of SEQ ID NO: 8.

4. The engineered T cell receptor of claim 3, wherein the alpha chain comprises a CDR1-alpha sequence comprising the amino acid sequence of SEQ ID NO: 1 and a CDR2-alpha sequence comprising the amino acid sequence of SEQ ID NO: 2, and the beta chain comprises a CDR1-beta sequence comprising the amino acid sequence of SEQ ID NO: 6 and a CDR2-beta sequence comprising the amino acid sequence of SEQ ID NO: 7.

5. The engineered T cell receptor of claim 3 or 4, wherein the alpha chain comprises an alpha variable domain comprising an amino acid sequence having at least 90%, 95%, or 100% identity to the amino acid sequence of SEQ ID NO: 4, and the beta chain comprises a beta variable domain comprising an amino acid sequence having at least 90%, 95%, or 100% identity to the amino acid sequence of SEQ ID NO: 9.

6. The engineered T cell receptor of any one of claims 3-5, wherein the alpha chain comprises an amino acid sequence having at least 90%, 95%, or 100% identity to the amino acid sequence of SEQ ID NO: 5 or 260, and the beta chain comprises an amino acid sequence having at least 90%, 95%, or 100% identity to the amino acid sequence of SEQ ID NO: 10 or 261.

7. The engineered T cell receptor of claim 6 comprising the sequence of SEQ ID NO: 310 or 311.

8. The engineered T cell receptor of claim 2, wherein the alpha chain comprises a CDR3-alpha sequence comprising the amino acid sequence of SEQ ID NO: 33 and the beta chain comprises a CDR3-beta sequence comprising the amino acid sequence of SEQ ID NO: 38.

9. The engineered T cell receptor of claim 8, wherein the alpha chain comprises a CDR1-alpha sequence comprising the amino acid sequence of SEQ ID NO: 31 and a CDR2-alpha sequence comprising the amino acid sequence of SEQ ID NO: 32, and the beta chain comprises a CDR1-beta sequence comprising the amino acid sequence of SEQ ID NO: 36 and a CDR2-beta sequence comprising the amino acid sequence of SEQ ID NO: 37.

10. The engineered T cell receptor of claim 8 or 9, wherein the alpha chain comprises an alpha variable domain comprising an amino acid sequence having at least 90%, 95%, or 100% identity to the amino acid sequence of SEQ ID NO: 34, and the beta chain comprises a beta variable domain comprising an amino acid sequence having at least 90%, 95%, or 100% identity to the amino acid sequence of SEQ ID NO: 39.

11. The engineered T cell receptor of any one of claims 8-10, wherein the alpha chain comprises an amino acid sequence having at least 90%, 95%, or 100% identity to the amino acid sequence of SEQ ID NO: 35 or 266, and the beta chain comprises an amino acid sequence having at least 90%, 95%, or 100% identity to the amino acid sequence of SEQ ID NO: 40 or 267.

12. The engineered T cell receptor of claim 11 comprising the sequence of SEQ ID NO: 312 or 313.

13. The engineered T cell receptor of claim 2, wherein the alpha chain comprises a CDR3-alpha sequence comprising the amino acid sequence of SEQ ID NO: 53 and the beta chain comprises a CDR3-beta sequence comprising the amino acid sequence of SEQ ID NO: 58.

14. The engineered T cell receptor of claim 13, wherein the alpha chain comprises a CDR1-alpha sequence comprising the amino acid sequence of SEQ ID NO: 51 and a CDR2-alpha sequence comprising the amino acid sequence of SEQ ID NO: 52, and the beta chain comprises a CDR1-beta sequence comprising the amino acid sequence of SEQ ID NO: 56 and a CDR2-beta sequence comprising the amino acid sequence of SEQ ID NO: 57.

15. The engineered T cell receptor of claim 13 or 14, wherein the alpha chain comprises an alpha variable domain comprising an amino acid sequence having at least 90%, 95%, or 100% identity to the amino acid sequence of SEQ ID NO: 54, and the beta chain comprises a beta variable domain comprising an amino acid sequence having at least 90%, 95%, or 100% identity to the amino acid sequence of SEQ ID NO: 59.

16. The engineered T cell receptor of any one of claims 13-15, wherein the alpha chain comprises an amino acid sequence having at least 90%, 95%, or 100% identity to the amino acid sequence of SEQ ID NO: 55 or 270, and the beta chain comprises an amino acid sequence having at least 90%, 95%, or 100% identity to the amino acid sequence of SEQ ID NO: 60 or 271.

17. The engineered T cell receptor of claim 16 comprising the sequence of SEQ ID NO: 314 or 315.

18. The engineered T cell receptor of claim 2, wherein the alpha chain comprises a CDR3-alpha sequence comprising the amino acid sequence of SEQ ID NO: 73 and the beta chain comprises a CDR3-beta sequence comprising the amino acid sequence of SEQ ID NO: 78.

19. The engineered T cell receptor of claim 18, wherein the alpha chain comprises a CDR1-alpha sequence comprising the amino acid sequence of SEQ ID NO: 71 and a CDR2-alpha sequence comprising the amino acid sequence of SEQ ID NO: 72, and the beta chain comprises a CDR1-beta sequence comprising the amino acid sequence of SEQ ID NO: 76 and a CDR2-beta sequence comprising the amino acid sequence of SEQ ID NO: 77.

20. The engineered T cell receptor of claim 18 or 19, wherein the alpha chain comprises an alpha variable domain comprising an amino acid sequence having at least 90%, 95%, or 100% identity to the amino acid sequence of SEQ ID NO: 74, and the beta chain comprises a beta variable domain comprising an amino acid sequence having at least 90%, 95%, or 100% identity to the amino acid sequence of SEQ ID NO: 79.

21. The engineered T cell receptor of any one of claims 18-20, wherein the alpha chain comprises an amino acid sequence having at least 90%, 95%, or 100% identity to the amino acid sequence of SEQ ID NO: 75 or 274, and the beta chain comprises an amino acid sequence having at least 90%, 95%, or 100% identity to the amino acid sequence of SEQ ID NO: 80 or 275.

22. The engineered T cell receptor of claim 21 comprising the sequence of SEQ ID NO: 316 or 317.

23. The engineered T cell receptor of any one of claims 1-22 encoded as a single polypeptide.

24. The engineered T cell receptor of any one of claims 2-23 further comprising a self-cleaving peptide sequence positioned between the alpha chain and the beta chain.

25. The engineered T cell receptor of claim 24, wherein the self-cleaving peptide sequence is a 2A peptide sequence, optionally wherein the 2A peptide sequence is a P2A, E2A, F2A, or T2A peptide sequence.

26. The engineered T cell receptor of any one of claims 1-25, wherein the preproinsulin 73-90 peptide comprises the amino acid sequence of GAGSLQPLALEGSLQKRG (SEQ ID NO: 109).

27. The engineered T cell receptor of any one of claims 1-26, wherein the MHC molecule comprises an HLA-DRB1*04:01 molecule.

28. An engineered nucleic acid encoding the engineered T cell receptor of any one of claims 1-27.

29. The engineered nucleic acid of claim 28, wherein the engineered nucleic acid is a viral vector, optionally a lentiviral vector.

30. The engineered nucleic acid of claim 28 or 29, wherein the engineered nucleic acid comprises an EF-1 alpha promoter or an MND promoter operably linked to a sequence encoding the engineered T cell receptor.

31. The engineered nucleic acid of any one of claims 28-30, wherein the engineered nucleic acid further comprises an enhancer element, optionally an optimized post-transcriptional regulatory element (oPRE) or a woodchuck hepatitis virus post-transcriptional regulatory element (WPRE), further optionally WPRE-mut6.

32. A regulatory T cell comprising an engineered T cell receptor that specifically binds a preproinsulin 73-90 peptide complexed with a major histocompatibility complex (MHC) molecule.

33. The regulatory T cell of claim 32, wherein the engineered T cell receptor is the engineered T cell receptor of any one of claims 1-27.

34. The regulatory T cell of claim 32, wherein the engineered T cell receptor expresses the engineered nucleic acid of any one of claims 28-31.

35. The regulatory T cell of any one of claims 32-34, wherein the regulatory T cell is phenotypically stable.

36. The regulatory T cell of any one of claims 32-35, wherein the regulatory T cell has not been gene edited at an endogenous FOXP3 locus.

37. A method of treating Type 1 Diabetes in a subject in need thereof, the method comprising administering to the subject a therapeutically effective amount of composition comprising the regulatory T cell of any one of claims 32-36.

38. The method of claim 37, wherein the regulatory T cell is autologous to the subject.

39. The method of claim 37 or 38, wherein the subject has Type 1 Diabetes, optionally Stage 3 Type 1 Diabetes.

40. The method of any one of claims 37-39, wherein the subject is an HLA-DRB1*04:01-positive subject.
